(19) United States
(12) Patent Application Publication
(10) Pub. No.: US 2013/0296328 A1
(43) Pub. Date: Nov. 7, 2013

(54) EPIGENETIC PORTRAITS OF HUMAN BREAST CANCERS

(71) Applicants: François Fuks, Bruxelles (BE); Sarah Dedeurwaerder, Vendeuil (FR); Christos Sotiriou, Bruxelles (BE); Christine Desmedt, Meise (BE)

(72) Inventors: François Fuks, Bruxelles (BE); Sarah Dedeurwaerder, Vendeuil (FR); Christos Sotiriou, Bruxelles (BE); Christine Desmedt, Meise (BE)

(73) Assignee: Université Libre de Bruxelles, Bruxelles (BE)

(21) Appl. No.: 13/980,809

(22) PCT Filed: Jan. 20, 2012
(86) PCT No.: PCT/EP2012/050836

§ 371 Date: Jul. 19, 2013

(30) Foreign Application Priority Data
Jan. 20, 2011 (EP) 11151583.9

(51) Int. Cl. C12Q 1/68

(52) U.S. Cl. CPC C12Q 1/6886
USPC 514/249; 435/6.11; 506/17; 506/2; 514/562; 514/642

(57) ABSTRACT

The present invention provides new target gene regions for use in prediction, prognosis, diagnosis and therapy of breast cancer, based on the differential methylation profile of said targets in samples from subjects with breast cancer and healthy subjects.

### FIELD OF THE INVENTION

The present invention is situated in the medical diagnostics, therapeutics field, more particular in the field of diagnosis of cancer, and methods for treating cancer, based on the new diagnostic tools and targets identified herein.

### BACKGROUND OF THE INVENTION

Breast cancer is a molecularly, biologically and clinically heterogeneous group of disorders. Understanding this diversity is essential to improving diagnosis and optimising treatment. Both genetic and acquired epigenetic abnormalities participate in cancer (Jones P. A. and Baylin S. B. 2007 Cell 128, 683-692; Feinberg, A. P. 2007 Nature 447, 433-440) but information is scant on the involvement of the epigenome in breast cancer and its contribution to the complexity of the disease.

Previous studies have documented aberrant methylation events in breast carcinogenesis (Sunami, E. et al. 2008 Breast Cancer Res. 10:R46; Feng, W. et al. 2007 Breast Cancer Res. 9:R57; Widschwendter, M. et al. 2004 Cancer Res. 64,3807-3813; Ordway, J. M. et al. PLoS One 19:e1314), but such events have never been precisely related to specific tumour traits. The goal of the present invention is thus to explore the DNA methylation landscapes of phenotypically heterogeneous tumours, to relate this diversity to landscape features, and extract biological and clinical meaningful information.

DNA methylation occurs as 5-methyl cytosine mostly in the context of CpG dinucleotides, so-called CpG sites. It is the best-studied epigenetic modification and governs transcriptional regulation and silencing (for review see Suzuki M M and Bird A 2008 Nat Rev Genet 9: 465-476). Unlike the relatively sturdy genome, the methylome changes in a dynamic way during development, tissue differentiation and aging. Pathologically altered DNA methylation is well described in various cancers (reviewed in Jones P A and Baylin S B 2007 Cell 128: 683-692). About 75% of human gene promoters are associated with CpG islands, which are clusters of 500 bp to 2 kb length with a comparatively high frequency of CpG dinucleotides. They usually harbour low levels of DNA methylation but can become hypermethylated; this CpG island hypermethylation was demonstrated to abrogate tumour suppressor gene transcription during tumourigenesis. Lately, DNA methylation changes in CpG sites adjoining yet outside of CpG islands, so-called CpG island shores (Irizarry R A et al., 2009 Nat Genet 41: 178-186), are gaining increased attention. Intriguingly, CpG sites in these shore sequences, in addition to those within CpG islands, are proposed to display differential DNA methylation between cancer and normal cells as well as between cells of different tissues.

The goal of the present invention is to clarify the hitherto poorly understood connection between the global DNA methylation status of the genome of breast cancer patients, i.e. both hyper- and/or hypomethylation with respect to a healthy subject. The invention aims at providing new prognostic and diagnostic tools for identifying breast cancer at a very early stage, for stratifying breast cancer patients. The invention further provides new targets for treatment of breast cancer.

### SUMMARY OF THE INVENTION

The present invention is based on information gathered by the Infinium® Methylation Platform with which 248 frozen breast tissues were profiled: a “main set” of 123 samples (4 normal and 119 infiltrating ductal carcinomas, IDCs), and a “validation set” of 125 samples (8 normal and 117 IDCs) (see Table 1).

Firstly, the invention shows that the two major phenotypes of breast cancers determined by ER status are widely epigenetically controlled.

Secondly, the present invention validates 6 methylation-profile-based tumour groups in an independent set of tumours, some of which coinciding with known gene expression tumor subtypes (Perou, C. M. et al. 2000 Nature 406, 747-752; Sørlie, T. et al. 2001 Proc. Natl Acad. Sci. USA 98, 10869-10874; van't Veer, L. J. et al. 2002 Nature 415, 530-535 ; Sotiriou, C. et al. 2003 Proc. Natl Acad. Sci. USA 100, 10393-10398) but also new entities that provides a meaningful basis for refining breast tumour taxonomy.

Thirdly, the invention shows that DNA methylation profiling can reflect the cell type composition of the tumour microenvironment.

Lastly, an unexpected strong epigenetic component was highlighted in the regulation of key immune pathways. The invention thus provides a set of immune genes having high prognostic value in specific tumour categories.

Taken together, by laying the ground for better understanding of breast cancer heterogeneity and improved tumour taxonomy, the precise epigenetic portraits provided by the present invention will contribute to better management of breast cancer patients.

The invention thus provides a method for the stratification and prognosis of breast cancer comprising the steps of:

a) analyzing the methylation status of one or more of the genes selected from the group comprising: LCK, CD3D, CD6, ICOS, CD3G, SIT1, CCL5, HCLS1, CD79B, UBASH3A, and LAX1, in a sample of the subject that has a breast cancer, and

b) comparing the methylation status of said one or more genes obtained from step a) with the methylation status of a control sample,

wherein a difference in methylation status as detected in step b) indicates the subject has a good or a bad clinical outcome. Preferably, the methylation status of one or more CpG regions or sites as defined by SEQ ID Nos 500-512 is analysed.

Alternatively, the invention provides a method for the stratification, diagnosis, prognosis or prediction of breast cancer comprising the steps of:

a) analyzing the methylation status of all 86 CpG regions defined in Table 2 (SEQ ID Nos 1 to 86) in a sample of the subject, and

b) comparing the methylation status of said one or more regions obtained from step a) with the methylation status of a control sample,

wherein a difference in methylation status as detected in step b) indicates the subject has or is at risk of developing breast cancer.

Furthermore, the invention provides a method for the stratification, prognosis or prediction of breast cancer as well as an indication for hormonotherapy response comprising the steps of:

a) analyzing the methylation status of one or more of the CpG regions defined in Table 5b (ESR1-positive module) and 5c (ESR1-negative module), respectively defined by (SEQ ID Nos 87 to 321 and 322 to 499), in a sample of the subject, and

b) comparing the methylation status of said one or more regions obtained from step a) with the methylation status of a control sample,

wherein a difference in methylation status as detected in step b) indicates the susceptibility of the subject to respond to hormonotherapy.

Preferably, all CpG islands or regions of either the ESR1-positive or -negative modules are analysed. Even more preferably, all regions or islands of both modules are analysed.

In any of the methods according to the present invention, the difference in methylation status can be due to either hypermethylation or hypomethylation.

In a preferred embodiment, the sample of the subject is selected from the group comprising: a tissue, cells, a cell pellet, a cell extract, a surgical sample, a biopsy or fine needle aspirate, or is a biological fluid such as: urine, whole blood, plasma, serum, ductal fluid, lymph node fluid, tumour exudate or tumour cavity fluid.

In a preferred embodiment, the methylation status of the genes selected from the group comprising: LCK, CD3D, CD6, ICOS, CD3G, SIT1, CCL5, HCLS1, CD79B, UBASH3A, and LAX1, is determined. Preferably, the methylation status of one or more of the CpG region of each of said genes is analysed. In one embodiment, said CpG regions are defined by SEQ ID Nos 500 to 512 (Table 13b).

In a further preferred embodiment, the breast cancer is of the HER-2-positive type, or luminal B-type. In a preferred embodiment of the method of the present invention, the methylation status is analysed by one or more techniques selected from the group consisting of nucleic acid amplification, polymerase chain reaction (PCR), methylation specific PCR (MCP), methylated-CpG island recovery assay (MIRA), combined bisulfite-restriction analysis (COBRA), bisulfite pyrosequenceing, single-strand conformation polymorphism (SSCP) analysis, restriction analysis, microarray analysis, or bead-chip technology.

The invention further provides for a method of treating breast cancer by targeting one or more genes having aberrant methylation in breast cancer, defined by one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, or 13b.

In a specific embodiment of said method of treatment, said targeting implies changing the methylation status by using demethylating or methylating agents, by changing the expression level, or by changing the protein activity of the protein encoded by said one or more genes. In preferred embodiments, said methylating agents are methyl donors such as folic acid, methionine, choline or any other chemicals capable of elevating DNA methylation.

The invention further provides for a method for identifying an agent that modulates the methylation status of one or more of the genes or gene products having aberrant methylation in breast cancer, defined by one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, or 13b, comprising the steps of:

a) contacting the candidate agent with said one or more genes, and

c) analysing the modulation of said one or more gene by the candidate agent. In a preferred embodiment of such a method, said agent modulates the methylation status, the expression level or the activity of said one or more gene.

The invention furthermore provides for a method for establishing a reference methylation status profile comprising the steps of: measuring the methylation status of one or more genes having aberrant methylation in breast cancer, defined by one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, or 13b in a sample of subject. Preferably, said subject is healthy, thereby producing a reference profile of a healthy subject, or said subject is suffering from breast cancer, or Basal-like, Luminal A, luminal B, HER2-plus or HER2-minus breast cancer, thereby producing a specific breast cancer type reference profile.

The invention also provides a methylation status profile for the stratification, prognosis, diagnosis or prediction of breast cancer comprising the methylation status of one or more CpG regions from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, or 13b, obtainable according to the method of the present invention.

The invention also provides a microarray or chip comprising one or more breast cancer specific CpG regions from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, or 13b.

In addition, the invention provides for the use of the methylation status of one or more of the CpG islands or regions from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, or 13b in the stratification, prognosis, diagnosis or prediction of breast cancer.

The invention further provides a method of stratifying breast cancer patients comprising the steps of:

a) analyzing the methylation status of one or more of the CpG islands or regions from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, or 13b, in a sample of the subject, and

b) comparing the methylation status of said one or more genes obtained from step a) with the methylation status of a control sample selected from the group of healthy, or Basal-like, Luminal A, luminal B, HER2-plus or HER2-minus breast cancer,

wherein a corresponding methylation status in steps a) and b) results in the identification of the type of breast cancer.

The invention further provides a method of selecting a breast cancer therapy comprising the steps of

a) analyzing the methylation status of one or more of the CpG islands or regions from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, or 13b, in a sample of the subject, and

b) comparing the methylation status of said one or more genes obtained from step a) with the methylation status of a control sample selected from the group of healthy, or Basal-like, Luminal A, luminal B, HER2-plus or HER2-minus breast cancer,

wherein a corresponding methylation status in steps a and b results in the identification of the type of breast cancer, and

c) identifying the appropriate treatment of the breast cancer in view of the type of cancer identified.

Finally, the invention provides a kit for the stratification, prognosis, diagnosis or prediction of breast cancer comprising the microarray according to the present invention, and one or more reference profiles according to the present invention. Alternatively, said kit of the invention comprises means for analyzing the methylation status of one or more CpG regions from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, or 13b, and one or more reference profiles according to the present invention.

The present invention further provides tools for refining breast cancer tumour taxonomy, typing and/or classification, based on the identification of specific clusters of CpG regions that are differentially methylated in different breast cancer subtypes.

The invention identifies two major clusters of CpG regions, called cluster I and II herein, that enable distinguishing between ER-positive (cluster II) and ER-negative (cluster I) breast cancers and between ESR1 positive (cluster I) or ESR1 negative (cluster II) breast cancers (Tables 5b and 5c).

In addition, using a classifier comprising the methylation data of 86 CpG regions (Table 2), the invention identifies 6 CpG methylation subclusters, called clusters 1 to 6, that enable the classification of breast cancers into HER2 positive (cluster 2), Basal-like (cluster 3) and Luminal A-type (cluster 6) cancers.

The present invention thus provides for methods of classifying breast cancers or stratifying breast cancer patients into subgroups of specific types of breast cancer, based on their methylation profile, using any one or more of the above indicated clusters. Based on this classification or stratification, the treatment of the cancer can be adapted, or the prognosis can be predicted.

In addition, the present invention has identified 11 immune prognostic markers for HER2 overexpressing and Luminal B tumours, namely: LCK, CD3D, CD6, ICOS, CD3G, SIT1, CCL5, HCLS1, CD79B, UBASH3A, and LAX1. Increased expression, which is coupled to decreased methylation results in better clinical outcome and thus a good prognosis. In total, 13 CpG islands or regions were identified in these genes that are differentially methylated in breast cancer versus healthy breast tissue (cf. SEQ ID Nos 500 to 512, Table 13b).

The present invention further provides tools to trace distinct groups of breast cancers back to specific stem cell/progenitor populations, likely to reflect their cellular origins.

The present invention further provides DNA methylation profiling which can contribute to cancer screening and prognosis, revealing strong survival markers.

The present invention showed that the immune component is important in the prognosis of breast cancer, notably T-cell markers whose expression is associated with a better clinical outcome.

The present invention and its alternative embodiments is further defined by the following description and examples section. The skilled person would be able to design alternative embodiments, building further on the knowledge provided by the present invention.

### DESCRIPTION OF THE DRAWINGS

FIG. 1. High-throughput DNA methylation profiling in human frozen breast tissues. a, Pie chart depicting the number of CpGs differentially methylated between breast tumour and normal samples of the main set, in terms of : (i) CpG location vs CGI (as defined by Bock et al. 2007 PLoS Comput. Biol. 3, 1055-1070) as well as CpG island shores (as defined by Irizarry et al. 2009 Nat. Genet. 41, 178-186); (ii) CpG location vs promoter classes (as defined by Weber et al. 2007 Nat. Genet. 39, 457-466). b, Validation of the bead array method by conventional Bisulphite Genomic Sequencing (BGS). Panel b shows exemplative analysed loci from CDK3, GSTP1, TWIST1 and RIMBP2 in 1 normal (N1) and 3 tumour samples (BCs). Grey arrows indicate the location of the CpG investigated by the bead array, which seems representative of the surrounding CpGs. Data representation was done according to Bock et al., 2005 (Bioinformatics 21, 4067-4068). Black circle, methylated CpG; white circle, unmethylated CpG; no circle, undetermined sequence. Panel c shows a significant positive correlation (Spearman's rho=0.82; p<0.001) between the Infinium Methylation and BGS data for the CDK3 locus.

FIG. 2. DNA methylation profiling identifies two main breast tumour categories with different ER statuses. a, ER status is a main discriminator of the two broad tumour groups. Selected clinical data: oestrogen receptor (ER) and HER2 receptor status determined by IHC, tumour grade, tumour size, nodal status, patient's age, and relapse within 5 years. b, Box plots of ESR1 module scores show that the genes of the ESR1-positive module (left part) showed higher methylation and lower expression in cluster I than in cluster II. The opposite was observed for the ESR1-negative module (right part). The ESR1 module has been previously described Desmedt, C. et al., 2008 (Clin. Cancer Res. 14, 5158-5165) and indicated p-values refer to a Mann-Whitney test. c, Barcode plots of the ESR1 module (provided by GSEA analysis) showing an anti-correlation of DNA methylation and expression data. Upper and lower bars designate the positions of ESR1 module genes in methylation and expression rankings, respectively. Dotted lines depict the zero. d, Association between methylation clusters I and II of the main patient set and the clinical data. ERpositive tumours were predominant in cluster II, whereas cluster I seemed to contain a moderately higher number of HER2-positive tumours. Grade 1 tumours were grouped in cluster II. No significant association with tumour size, nodal status, or age was found.

FIG. 3 Complexity and heterogeneity of breast cancers as revealed by DNA methylation. a, DNA methylation profiling of the main set identifies 6 groups of tumours, termed clusters 1 to 6, displaying differences in terms of “expression subtype composition” and clinical characteristics (see also Table 6). b, Comparison of the methylation group assigned to each tumour of the main set by the unsupervised cluster analysis and the 86 CpG-classifier established by the nearest centroid classification method. c, Correlation plot of main set of tumours with the 6 centroids. Each sample displays the colour of its methylation group assigned by the unsupervised clustering of FIG. 3a. d, Classification of each tumour of the validation set into one of the six methylation groups by means of the 86 CpG-classifier. e, Correlation plot of validation set tumours with the 6 centroids. Each sample was placed in the group with which it presented the highest correlation). Note that the 6 groups obtained for the validation set presented the same “expression subtype composition” and clinical characteristics as the groups obtained for the main set. f, Shows the association between the 6 groups of tumours of the validation set and the clinical data. Clusters 5 and 6 contained exclusively ER-positive tumours, whereas clusters 3 were composed principally of ERnegative tumours. HER2-positive tumours were predominant in clusters 1 and 2. Cluster 6 contained majorly grade 1 tumours. No significant association with tumour size or age was found. g, Characteristics of the 86 CpG-classifier in terms of CpG location vs CGI and vs promoter classes. h, Comparison of gene expression signatures of several normal mammary epithelial subpopulations with gene expression and DNA methylation profiles of our six DNA methylation-based groups of patients in the main set (see section Module/signature scores of additional online Methods). a, b, c, Box plots of mammary stem cell (MaSC), luminal progenitor, and luminal mature signature scores respectively for each of the six methylation breast cancer groups, based on their gene expression profiles. i, Histograms showing the heterogeneity of breast tumours in terms of the number of CpGs differentially methylated compared to normal samples. j, Differential methylation of genes involved in immunity as revealed by GO analysis, with high hypomethylation content in clusters 2 and 3. k, Histologic patterns of breast tumours displaying no lymphocyte infiltration (1) or both stromal and intratumoral infiltration (2). Panel 3 provides a closer look at the intratumoral infiltration presented in panel 2. Black arrows indicate epithelial cells, whereas green and blue arrows indicate stromal and intratumoral lymphocytes, respectively. I, Box plots depicting the higher lymphocyte infiltration in main set tumours belonging to clusters 2 and 3 as compared to tumours belonging to other clusters. m, Box plots illustrating the inverse correlation between LCK and ITGAL methylation and lymphocyte infiltration (Jonckheere-Terpstra test for trends; see also Table 8). n, Methylation status, as assessed by DNA methylation profiling, of immune genes highlighted by GO analysis in breast epithelial cell lines as well as in ex vivo lymphocytes and lymphoid cell lines. o, Association between methylation clusters 1 to 6 of the main patient set and the clinical data. Cluster 6 contained almost exclusively ER-positive tumours, whereas clusters 2 and 3 were composed principally of ER-negative tumours. HER2-positive tumours were predominant in cluster 2 and HER2-negative tumours were predominant in clusters 3 and 6. Cluster 6 contained almost exclusively grade 1 tumours. No significant association with tumour size, nodal status or age was found.

FIG. 4. Epigenetically regulated immune components are good clinical outcome markers for breast cancers. a, Pie chart depicting the high proportion of immune genes, and in particular of genes involved in T cell biology, among all the genes that appeared significant prognostic markers (FDR<0.1) (univariate Cox regression analysis was performed as described in the Methods and Table 10). b, Box plots illustrating the correlation of methylation (in red) and expression (in blue) status of LAX1 and CD3D with stromal lymphocyte infiltration (Jonckheere-Terpstra test for trends; see also Tables 11 and 12). c, Anti-correlation between the methylation and expression status of the 11 prognostic immune markers in breast epithelial cell lines as well as in ex vivo lymphocytes and lymphoid cell lines, as determined by DNA methylation and gene expression profiling. d, High expression of 11 immune genes is associated with a better clinical outcome in breast cancer. Forest plots showing the log 2 hazard ratio (squares) with the 95% confidence interval (bars) of the relapse-free survival analysis. A negative hazard ratio reveals that a high expression level of the indicated variable is associated with a good outcome, and conversely. e, Subtype-specific prognostic value of immune markers for breast cancer. Exemplative Kaplan-Meier curves for different levels of expression of the LAX1 and CD3D genes in each known “expression subtype” (see also Table 15 for the detailed continuous univariate survival analysis for each subtype).

### DETAILED DESCRIPTION OF THE PREFERRED EMBODIMENTS

As used herein, the singular forms “a”, “an”, and “the” include both singular and plural referents unless the context clearly dictates otherwise. By way of example, “an antibody” refers to one or more than one antibody; “an antigen” refers to one or more than one antigen.

The terms “comprising”, “comprises” and “comprised of” as used herein are synonymous with “including”, “includes” or “containing”, “contains”, and are inclusive or open-ended and do not exclude additional, non-recited members, elements or method steps.

The term “and/or” as used in the present specification and in the claims implies that the phrases before and after this term are to be considered either as alternatives or in combination.

As used herein, the term “level” or “expression level” refers to the expression level data that can be used to compare the expression levels of different genes among various samples and/or subjects.

The term “amount” or “concentration” of certain proteins refers respectively to the effective (i.e. total protein amount measured) or relative amount (i.e. total protein amount measured in relation to the sample size used) of the protein in a certain sample.

All documents cited in the present specification are hereby incorporated by reference in their entirety. In particular, the teachings of all documents herein specifically referred to are incorporated herein by reference.

The term “CpG region” or “CpG site” is a region of genome DNA which shows higher frequency of 5′-CG-3′ (CpG) dinucleotides than other regions of genome DNA. Methylation of DNA at CpG dinucleotides, in particularly, the addition of a methyl group to position 5 of the cytosine ring at CpG dinucleotides, is one of the epigenetic modifications in mammalian cells. CpG regions or sites encompass the so called “CpG islands”, which often occur in the promoter regions of genes and play a pivotal role in the control of gene expression. In normal tissues CpG islands are usually unmethylated, but a subset of islands becomes differentially methylated (hyper- or hypomethylated) during the development of a disease.

Detection of methylation state of CpG regions can be done by any known assay currently used in scientific research. Some non-limiting examples are: Methylation-Specific PCR (MSP), which is based on a chemical reaction of sodium bisulfite with DNA, converting unmethylated cytosines of CpG dinucleotides to uracil (UpG), followed by traditional PCR. Methylated cytosines will not be converted by the sodium bisulfite, and specific nucleotide primers designed to overlap with the CpG site of interest will allow determining the methylation status as methylated or unmethylated, based on the amount of PCR product formed. Alternatively, the HELP assay can be used, which is based on the differential ability of restriction enzymes to recognize and cleave methylated and unmethylated CpG DNA sites. Furthermore, ChIP-on-chip assays, based on the ability of commercially prepared antibodies to bind to DNA methylation-associated proteins like MCP2, can be used to determine the methylation status. Also restriction landmark genomic scanning, also based upon differential recognition of methylated and unmethylated CpG sites by restriction enzymes can be used. Methylated DNA immunoprecipitation (MeDIP), analogous to chromatin immunoprecipitation, can be used to isolate methylated DNA fragments for input into DNA detection methods such as DNA microarrays (MeDIP-chip) or DNA sequencing (MeDIP-seq). The unmethylated DNA is not precipitated. Alternatively, molecular break light assay for DNA adenine methyltransferase activity can be used. This is an assay that uses the specificity of the restriction enzyme DpnI for fully methylated (adenine methylation) GATC sites in an oligonucleotide labeled with a fluorophore and quencher. The adenine methyltransferase methylates the oligonucleotide making it a substrate for DpnI. Cutting of the oligonucleotide by DpnI gives rise to a fluorescence increase. Further, methylated-CpG island recovery assay (MIRA) can be used.

These techniques require the presence of methylated cytosine residues within the recognition sequence that affect the cleavage activity of restriction endonucleases (e.g., HpaII, HhaI) (Singer et al. (1979)). Southern blot hybridization and polymerase chain reaction (PCR)-based techniques can be used with along with this approach.

In another embodiment, a bisulfite dependent methylation assay is known as a combined bisulfite-restriction analysis (COBRA assay) whereas PCR products obtained from bisulfite-treated DNA can also be analyzed by using restriction enzymes that recognize sequences containing 5′CG, such as TaqI (5′TCGA) or BstUI (5′CGCG) such that methylated and unmethylated DNA can be distinguished.

In another embodiment, a methylation detection technique is based on the ability of the MBD domain of the MeCP2 protein to selectively bind to methylated DNA sequences. The bacterially expressed and purified His-tagged methyl-CpG-binding domain is immobilized to a solid matrix and used for preparative column chromatography to isolate highly methylated DNA sequences. Restriction endonuclease-digested genomic DNA is loaded onto the affinity column and methylated-CpG island-enriched fractions are eluted by a linear gradient of sodium chloride. PCR or Southern hybridization techniques are used to detect specific sequences in these fractions. In addition, one can make use of MALDI-TOF for DNA methylation analysis. Using a combination of four base specific cleavage reactions, each CpG of a target region can be analyzed individually and is represented by multiple indicative mass signals. Another exemplary method for detecting the methylation status of a gene makes use of a bead chip such as the Infinium® bead chip sold by Illumina Inc. San Diego (US).

In selected embodiments, the methods for determining the methylation state of (one or more) target gene regions may include treating a target nucleic acid molecule with a reagent that modifies nucleotides of the target nucleic acid molecule as a function of the methylation state of the target nucleic acid molecule, amplifying treated target nucleic acid molecule, fragmenting amplified target nucleic acid molecule, and detecting one or more amplified target nucleic acid molecule fragments, and based upon the fragments, such as size and/or number thereof, identifying the methylation state of a target nucleic acid molecule, or a nucleotide locus in the nucleic acid molecule, or identifying the nucleic acid molecule or a nucleotide locus therein as methylated or unmethylated. Fragmentation can be performed, for example, by treating amplified products under base specific cleavage conditions. Detection of the fragments can be effected by measuring or detecting a mass of one or more amplified target nucleic acid molecule fragments, for example, by mass spectrometry such as MALDI-TOF mass spectrometry. Detection also can be affected, for example, by comparing the measured mass of one or more target nucleic acid molecule fragments to the measured mass of one or more reference nucleic acid, such as measured mass for fragments of untreated nucleic acid molecules. In an exemplary method, the reagent modifies unmethylated nucleotides, and following modification, the resulting modified target is specifically amplified. In some embodiments, the methods for determining the methylation state of (one or more) target gene regions may include treating a target nucleic acid molecule with a reagent that modifies a selected nucleotide as a function of the methylation state of the selected nucleotide to produce a different nucleotide. In particular embodiments, the reagent that modifies unmethylated cytosine to produce uracil is bisulfite. In certain embodiments, the methylated or unmethylated nucleic acid base is cytosine. In another embodiment, a non-bisulfite reagent modifies unmethylated cytosine to produce uracil.

As used herein, a “nucleic acid target gene region” is a nucleic acid molecule that is examined using the methods disclosed herein. For the purposes of the application, “nucleic acid target gene region”, “target gene”, “target region”, “region” and “gene” may be used interchangeably. A nucleic acid target gene region includes genomic DNA or a fragment thereof, which may or may not be part of a gene, a segment of mitochondrial DNA of a gene or RNA of a gene and a segment of RNA of a gene. Examples of “targets” as defined herein are listed in Tables 2, 5b, 5c or 13 by means of their gene name or Gene ID number. A nucleic target gene region may be further defined by its chromosome position range as is e.g. done in Tables 2, 5b, 5c or 13 for each target sequence identified herewith. The chromosome position ranges provided herein were gathered from the human reference sequence (genome Build hg18/NCBI36, March 2006), which was produced by the International Human Genome Sequencing Consortium.

As used herein, a “nucleic acid target gene molecule” is a molecule comprising a nucleic acid sequence of the nucleic acid target gene region. The nucleic acid target gene molecule may contain less than 10%, less than 20%, less than 30%, less than 40%, less than 50%, greater than 50%, greater than 60%, greater than 70% greater than 80%, greater than 90% or up to 100% of the sequence of the nucleic acid target gene region. A “target peptide” refers to a peptide encoded by a nucleic acid target gene.

As used herein, the “methylation state” or “methylation status” of a nucleic acid target gene region refers to the presence or absence of one or more methylated nucleotide bases or the ratio of methylated cytosine to unmethylated cytosine for a methylation site in a nucleic acid target gene region as defined herein.

For example, a nucleic acid target gene region containing at least one methylated cytosine can be considered methylated (i.e. the methylation state of the nucleic acid target gene region is methylated). A nucleic acid target gene region that does not contain any methylated nucleotides can be considered unmethylated.

Similarly, the methylation state of a nucleotide locus in a nucleic acid target gene region refers to the presence or absence of a methylated nucleotide at a particular locus in the nucleic acid target gene region.

For example, the methylation state of a cytosine at the 10th nucleotide in a nucleic acid target gene region is methylated when the nucleotide present at the 10th nucleotide in the nucleic acid target gene region is 5-methylcytosine. Similarly, the methylation state of a cytosine at the 10th nucleotide in a nucleic acid target gene region is unmethylated when the nucleotide present at the 10th nucleotide in the nucleic acid target gene region is cytosine (and not 5-methylcytosine).

Correspondingly the ratio of methylated cytosine to unmethylated cytosine for a methylation site(s) or locus can provide a methylation state of a nucleic acid target gene region. In certain embodiments the methylation state or status may be expressed as a percentage of methylateable nucleotides (e.g., cytosine) in a nucleic acid (e.g., amplicon or gene region) that are methylated (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% methylated; greater than 80% methylated, between 20% to 80% methylated, or less than 20% methylated). A nucleic acid may be “hypermethylated,” which refers to the nucleic acid having a greater number of methylateable nucleotides that are methylated relative to a control or reference. A nucleic acid may be “hypomethylated,” which refers to the nucleic acid having a smaller number of methylateable nucleotides that are methylated relative to a control or reference. The methylation status or state is determined in a CpG island or region in certain embodiments. Examples of target CpG islands or regions according to the present invention are listed in Tables 2, 5b, 5c or 13 and in SEQ ID Nos 1-512.

As used herein, a “characteristic methylation state” refers to a unique, or specific data set comprising the methylation state of at least one of the methylation sites of one or more nucleic acid(s), nucleic acid target gene region(s), gene(s) or group of genes of a sample obtained from a subject. It can be the combined data of the methylation state of a panel of multiple target genes according to the present invention in said sample, as compared to a reference sample from e.g. a healthy subject.

As used herein, “methylation ratio” refers to the number of instances in which a molecule or locus is methylated relative to the number of instances the molecule or locus is unmethylated.

Methylation ratio can be used to describe a population of individuals or a sample from a single individual.

For example, a nucleotide locus having a methylation ratio of 50% is methylated in 50% of instances and unmethylated in 50% of instances. Such a ratio can be used, for example, to describe the degree to which a nucleotide locus or nucleic acid region is methylated in a population of individuals. Thus, when methylation in a first population or pool of nucleic acid molecules is different from methylation in a second population or pool of nucleic acid molecules, the methylation ratio of the first population or pool will be different from the methylation ratio of the second population or pool. Such a ratio also can be used, for example, to describe the degree to which a nucleotide locus or nucleic acid region is methylated in a single individual. For example, such a ratio can be used to describe the degree to which a nucleic acid target gene region of a group of cells from a tissue sample are methylated or unmethylated at a nucleotide locus or methylation site.

As used herein, a “methylated nucleotide” or a “methylated nucleotide base” refers to the presence of a methyl moiety on a nucleotide base, where the methyl moiety is not present in a recognized typical nucleotide base. Cytosine does not contain a methyl moiety on its pyrimidine ring, however 5-methylcytosine contains a methyl moiety at position 5 of its pyrimidine ring. In this respect, cytosine is not a methylated nucleotide and 5-methylcytosine is a methylated nucleotide.

As used herein, a “methylation site” is a nucleotide within a nucleic acid, nucleic acid target gene region or gene that is susceptible to methylation either by natural occurring events in vivo or by an event instituted to chemically methylate the nucleotide in vitro.

As used herein, a “methylated nucleic acid molecule” refers to a nucleic acid molecule that contains one or more methylated nucleotides that is/are methylated.

As used herein “CpG island” refers to a G:C-rich region of genomic DNA containing a greater number of CpG dinucleotides relative to total genomic DNA, as defined in the art. It should be noted that differential methylation of the target genes according to the invention is not limited to CpG islands only, but can be in so-called “shores” or can be lying completely outside a CpG island region, called herein more generally a “CpG region” or “CpG site”.

As used herein, a first nucleotide that is “complementary” to a second nucleotide refers to a first nucleotide that base-pairs, under high stringency conditions to a second nucleotide. An example of complementarity is Watson-Crick base pairing in DNA (e.g., A to T and C to G) and RNA (e.g., A to U and C to G). Thus, for example, G base-pairs, under high stringency conditions, with higher affinity to C than G base-pairs to G, A or T, and, therefore, when C is the selected nucleotide, G is a nucleotide complementary to the selected nucleotide.

As used herein, the term “correlates” as between a specific diagnosis or a therapeutic outcome of a sample or of an individual and the changes in methylation state of a nucleic acid target gene region refers to an identifiable connection between a particular diagnosis or therapy of a sample or of an individual and its methylation state.

As used herein, a “subject” includes, but is not limited to, an animal, plant, bacterium, virus, parasite and any other organism or entity that has nucleic acid. Among animal subjects are mammals, including primates, such as humans. As used herein, “subject” may be used interchangeably with “patient” or “individual”.

As used herein, a “methylation” or “methylation state” correlated with a disease, disease outcome or outcome of a treatment regimen refers to a specific methylation state of a nucleic acid target gene region or nucleotide locus that is present or absent more frequently in subjects with a known disease, disease outcome or outcome of a treatment regimen, relative to the methylation state of a nucleic acid target gene region or nucleotide locus than otherwise occur in a larger population of individuals (e.g., a population of all individuals).

As used herein, “sample” refers to a composition containing a material to be detected, and includes e.g. “biological samples”, which refer to any material obtained from a living source, for example an animal such as a human or other mammal that can suffer from breast cancer. The biological sample can be in any form, including a solid material such as a tissue, cells, a cell pellet, a cell extract, a surgical sample, a biopsy or fine needle aspirate, or it can be in the form of a biological fluid such as urine, whole blood, plasma, or serum, or any other fluid sample produced by the subject such as ductal fluids, lymph node fluids, tumour exudates or tumour cavity fluids. In addition, the sample can be solid samples of tissues or organs, such as collected tissues, including breast tissue. Samples can include pathological samples such as a formalin-fixed sample embedded in paraffin. If desired, solid materials can be mixed with a fluid or purified or amplified or otherwise treated. Samples examined using the methods described herein can be treated in one or more purification steps in order to increase the purity of the desired cells or nucleic acid in the sample. Samples also can be examined using the methods described herein without any purification steps to increase the purity of desired cells or nucleic acid. In particular, herein, the samples include a mixture of matrix used for mass spectrometric analyses and a biopolymer, such as a nucleic acid. Preferably, said sample is a breast cancer biopsy, or is whole blood, plasma or serum of a subject. The sample can furthermore be a test cell obtainable from tissues or fluids including detached tumour cells or free nucleic acids that are released from dead tumour cells. Nucleic acids include RNA, genomic DNA, mitochondrial DNA, and possibly protein-associated nucleic acids. Any nucleic acid specimen in purified or non-purified form obtained from such test cell can be utilized in the methods of the present invention.

The term “breast cancer” described in the methods or uses or kits of the invention encompasses in principle all cancers of breast-related tissue, including ducts, glands or lobules and infiltrating lymph and/or blood vessels. Specific examples of breast cancer are for example: Ductal Carcinoma In-Situ (DCIS), a type of early breast cancer confined to the inside of the ductal system. Infiltrating Ductal Carcinoma (IDC) is the most common type of breast cancer representing 78% of all malignancies. These lesions appear as stellate (star like) or well-circumscribed (rounded) areas on mammograms. The stellate lesions generally have a poorer prognosis. Medullary Carcinoma accounts for 15% of all breast cancer types. It most frequently occurs in women in their late 40s and 50s, presenting with cells that resemble the medulla (gray matter) of the brain. Infiltrating Lobular Carcinoma (ILC) is a type of breast cancer that usually appears as a subtle thickening in the upper-outer quadrant of the breast. This breast cancer type represents 5% of all diagnosis. Often positive for estrogen and progesterone receptors, these tumors respond well to hormone therapy. Tubular Carcinoma makes up about 2% of all breast cancer diagnosis, tubular carcinoma cells have a distinctive tubular structure when viewed under a microscope. Typically this type of breast cancer is found in women aged 50 and above. It has an excellent 10-year survival rate of 95%. Mucinous Carcinoma (Colloid) represents approximately 1% to 2% of all breast carcinoma. This type of breast cancer's main differentiating features are mucus production and cells that are poorly defined. It also has a favorable prognosis in most cases. Inflammatory Breast Cancer (IBC) is a rare and very aggressive type of breast cancer that causes the lymph vessels in the skin of the breast to become blocked. This type of breast cancer is called “inflammatory” because the breast often looks swollen and red, or “inflamed”. IBC e.g. accounts for 1% to 5% of all breast cancer cases in the United States. Breast cancer subtypes can furthermore be identified on the basis of gene expression by applying the Subtype Classification Model as described by Desmedt et al., 2008 (Clin. Cancer Res. 14, 5158-5165) and Wirapati et al.,2008 (Breast Cancer Res. 10:R65).

The invention is illustrated by the following non-limiting examples.

### EXAMPLES

Materials and Methods

Breast Tissues Selection Criteria

The main sample set is constituted of 119 archival frozen breast cancer samples from patients diagnosed at the Jules Bordet Institute in Brussels between 1995 and 2003. These samples were selected according to the following criteria:

1/ sufficient presence of invasive cells as defined by pathologist. The current practice of pathologists is to examine by microscopy a representative slide of a given tumour sample and to estimate the proportion of the tumour that contains epithelial cancer cells (measured as <<% area>>). Any sample below an arbitrary threshold of an estimated value of “90%” was rejected. Although this is a current practice of pathologists and has been for many years, it is important to notice that this “area” criterion is not quantitatively accurate;

2/ >2 pg yield of high quality DNA available;

3/ balanced distribution of the four main “breast cancer expression subtypes” determined by IHC; and

4/ balanced distribution of patients with and without relapses within each subtype. Four samples of normal breast tissues with sufficient high-quality DNA were selected as well for this main series.

The validation sample set is constituted of 117 frozen breast cancer samples from patients diagnosed at the Jules Bordet Institute in Brussels between 2004 and 2009. For patient data, see Table 1. The Ethics committee of the Jules Bordet Institute approved the present research project.

TABLE 1
Characteristics of breast tissue samples of the main patient set.

| Characteristic | | Number of patients |
|---|---|---|
| Tumour size | ≦2 cm | 44 |
| | >2 cm | 75 |
| Nodal status | Negative | 64 |
| | Positive | 55 |
| Grade | 1 | 25 |
| | 2 | 9 |
| | 3 | 85 |
| ER | Negative | 54 |
| | Positive | 64 |
| | Unknown | 1 |
| HER2 | Negative | 88 |
| | Positive | 31 |
| Subtype IHC | Basal-like | 31 |
| | HER2+ | 31 |
| | Luminal A | 25 |
| | Luminal B | 32 |
| Subtype GEP | Basal-like | 22 |
| | HER2+ | 21 |
| | Luminal A | 23 |
| | Luminal B | 22 |
| | Unknown | 31 |
| Age | <50 years | 38 |
| | >years | 81 |
| Relapse | No | 68 |
| | Yes | 51 |

DNA Methylation Profiling

Genomic DNA from the clinical frozen samples was extracted from twenty 10-μm sections using the Qiagen-DNeasy Blood &Tissue Kit according to the supplier's instructions (Qiagen, Hilden, Germany). This included a proteinase K digestion at 55° C. overnight. For breast epithelial cell lines and lymphocyte samples, genomic DNA was extracted with the QIAamp DNA Mini Kit (Qiagen, Hilden, Germany) including the recommended proteinase K and RNase A digestions. DNA was quantitated with the NanoDrop® ND-1000 UV-Vis Spectrophotometer (NanoDrop Technologies, Wilmington, Del., USA). Site-specific CpG methylation was analysed using Infinium® HumanMethylation27 beadarray-based technique. This array was developed to assay 27,578 CpG sites selected from more than 14,000 genes. Genomic DNA (1 μg) was treated with sodium bisulphite using the Zymo EZ DNA Methylation Kit™ (Zymo Research, Orange, USA) according to the manufacturer's procedure, with the alternative incubation conditions recommended when using the Illumina Infinium® Methylation Assay. The methylation assay was performed from 4 μL converted gDNA at 50 ng/μL according to the Infinium® Methylation Assay Manual protocol. The quality of bead array data was checked with the GenomeStudio™ Methylation Module software. All samples passed this quality control. Methylation raw data are available online (http://www.ncbi.nlm.nih.gov/geo/query/acc.cgi?token=bvonpyugyawqqto&acc=GSE20713).

Gene Expression Profiling

For tumours of the main set as well as cell lines and ex vivo samples, RNA was isolated by the Trizol method (Invitrogen) or the Tripure method (Roche) according to manufacturers' instructions and purified on RNeasy mini-columns (Qiagen). The quality of the RNA obtained from each tumour sample was assessed on the basis of the RNA profile generated by the Bioanalyzer (Agilent Inc.). Total RNA (100 ng) was first reverse-transcribed into doublestranded cDNA. This cDNA was transcribed in vitro. After purification of the aRNA, 12.5 μg were fragmented and labelled prior to hybridisation to the Affymetrix HG133 Plus 2.0 GeneChip. Among the clinical samples of the main set, thirty initially profiled for DNA methylation were not profiled for gene expression because of low tumour-cell content (<70% tumour cells, n=11), no tumour left at all in the samples (n=4), low-quality RNA (n=13), or low RNA quantity (n=2). In addition, the CD4+ lymphocyte clone R12C9 was not profiled for gene expression because of low RNA quantity. The quality of the microarray data was checked using the ‘yaqcaffy’ package of the R statistical software (http://www.r-project.org/). On the basis of the results, two samples were excluded from further analysis. Gene expression raw data are available online (http://www.ncbi.nlm.nih.gov/geo/query/acc.cgi?token=bvonpyugyawqqto&acc=GSE20713).

Histopathologic Analysis of the Lymphocyte Infiltration

Histopathologic analysis of tumours in order to evaluate both stromal and intratumoral lymphocyte infiltration was performed on hematoxylin and eosin-stained sections, as previously described (Denkert, C. et al., 2010 J. Clin. Oncol. 28, 105-113).

Culture of Breast Epithelial and Lymphoid Cell Lines

MCF10A cells were cultured in DMEM/F12 (1:1) medium (Gibco); MCF-7, SKBR3 and MDA-MB-231 were cultured in DMEM medium (Gibco); T47D, ZR-75-1 and MDA-MB-361 were cultured in RPMI medium (Gibco); and BT20 were cultured in MEM medium (Gibco). For all breast epithelial cell lines, media were supplemented with 10% fetal calf serum (Gibco). The lymphoid clones CD4+ R12C9 and CD8+ WEIS3E5 were maintained in Isocove Dubelcco medium supplemented with 10% human serum HS54, L-Arginine, LAsparagine, L-glutamine, 2-mercaptoethanol and methyltryptophane and 10 ng/mL of IL-7 and 50 U/mL of IL-2.

Isolation of ex vivo Lymphocytes

Blood mononuclear cells from an hemochromatosis patient were isolated with density gradient centrifugation using Lymphoprep (Axis-Shield PoCAS, Oslo, Norway), and extensively washed in cold phosphate-buffered saline containing 2 mM EDTA, to eliminate platelets. CD3+ and CD20+ cells were purified with magnetic microbeads using the CD3 Isolation Kit or CD20 Isolation Kit (Miltenyi Biotec, Bergisch Gladbach, Germany) in an AUTOMACS magnetic sorter (Miltenyi), following the manufacturer's instructions. Cell purities were higher than 99 and 92% for the CD3+ and CD20+ cells, respectively, as determined with standard flow cytometry.

Unsupervised Clustering

In a first step, as a completely unsupervised approach, hierarchical clustering was performed on all 123 breast tissues of the main set (119 IDCs and 4 normal breast tissues) on the basis of the 10% most variant CpGs between all samples. This has been done also for all samples of the validation set. In both cases, the normal samples were in a single cluster, distinguishable from the breast cancer samples. In a second step, hierarchical clustering was performed only on the 119 IDCs of the main set on the basis of a reduced list of CpGs differentially methylated between IDC and normal tissues. Among the 6,309 CpGs identified as being differentially methylated between IDC and normal samples, those showing a 20% methylation difference in at least 30% of the IDCs as compared to the normal breast samples were chosen. This ensured selection of a reasonable number of CpGs (2,985) having potentially informative variance in our dataset and yielded clusters showing good stability. Complete linkage and distance correlations were used for clustering arrays and CpGs. The stability of the clustering was estimated with the ‘pvclust’ R package (Suzuki, R. & Shimodaira, H. 2006 Bioinformatics 22, 1540-1542), available on CRAN (http://cran.r-project.org/web/packages/pvclust/). The uncertainty in hierarchical clustering was measured by bootstrap stability probabilities ranging from 0 to 1, with 0 indicating poor stability and 1 indicating a very high stability. The bootstrap probability value of a cluster is the frequency that it appears in the bootstrap replicates. These stability values quantify how strong a cluster is supported by data. The criteria used to select the 6 methylation clusters defined in the present invention were: (i) a stability probability of minimum 0.75, and (ii) a minimum number of samples of 8.

Module/Signature Scores

The calculation of module/signature scores is described in Desmedt et al., 2008 (Clin. Cancer Res. 14, 5158-5165) and Wirapati et al., 2008 (Breast Cancer Res. 10:R65). Briefly, a signature score, denoted by Rs, was defined as the weighted combination of all the gene expressions in the corresponding signature:

$${R}_{s} = \frac{{\sum }_{i ∈ Q} {w}_{i} {x}_{i}}{{n}_{Q}}$$

where Q is the set of genes in the signature, nQ is the number of genes in Q, xi is the expression of gene i, and wi is either −1 or +1 depending on the sign of the statistic/coefficient published in the original study. For the particular cases of the two divided “ESR1 positive” and “ESR1 negative” modules, wi is always equal to +1. For DNA methylation data, signature scores were calculated in a manner similar to that of gene expression data with an additional mapping procedure: each CpG probe was mapped to the corresponding gene through Entrez Gene ID. Each signature score was scaled so that quantiles 2.5% and 97.5% equaled −1 and +1, respectively. This scaling was robust to outliers and ensured that the signature score lay approximately within the [−1,+1] interval, allowing comparison of datasets based on different microarray technologies and normalizations.

Breast Cancer “Expression Subtype” Determination

Two approaches were used to determine “breast cancer expression subtypes”. First, on the basis of an IHC determination, basal-like tumours were defined as negative for ER and HER2 receptors and as histological grade 3, HER2 tumours as overexpressing the HER2 receptor, and luminal tumours as ER positive and HER2 negative. This last group was divided into luminal A and B tumours corresponding respectively to histological grade 1 and grade 3 tumours. Secondly, the subtypes were identified on the basis of gene expression by applying the Subtype Classification Model as described by Desmedt et al., 2008 (Clin. Cancer Res. 14, 5158-5165) and Wirapati et al.,2008 (Breast Cancer Res. 10:R65). The only difference was in the use of the single probes “205225_at”, “216836_s_at” and “208079_s_at” instead of the full ESR1, ERBB2 and AURKA modules, respectively. This simplified version of the Subtype Classification Model was chosen as this model showed excellent performance when applied to the Affymetrix dataset, while reducing the number of genes in the clustering model (data not shown). The ‘genefu’ R package was used, available on CRAN (http://cran.r-project.org/web/packages/genefu/).

Establishment of the 86 CpG-Classifier

To transfer class discovery results from one data set to another in order to independently confirm the results, the nearest centroid classification method was used (Sørlie, T. et al., 2003 Proc. Natl Acad. Sci. USA 100, 8418-8423; Lusa, L. et al., 2007 J. Natl Cancer Inst. 99, 1715-1723) for assigning new samples of the validation set to one of the 6 clusters. This method is based on the similarity of the DNA methylation profile of a new sample to the DNA methylation profile of the previously identified clusters. A centroid was defined as the vector containing the median methylation values of all the samples assigned to that cluster in the original hierarchical clustering in the main set. For each new sample, a Spearman rank correlation was calculated between its methylation data and the six centroids; the predicted cluster was defined as the category having the highest correlation value. For training the classifier, those patients in the main set not belonging to any of the 6 most robust clusters were excluded. The Kruskal-Wallis non parametric test was used to find the differently methylated CpGs between the six clusters.

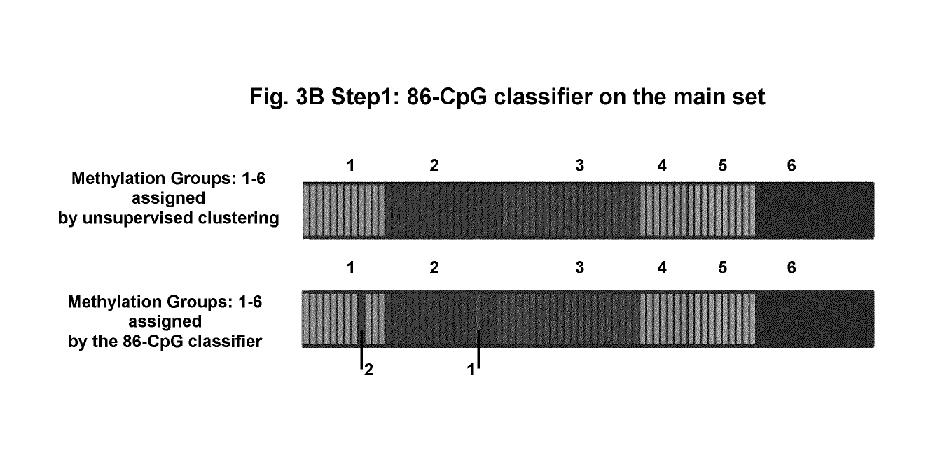

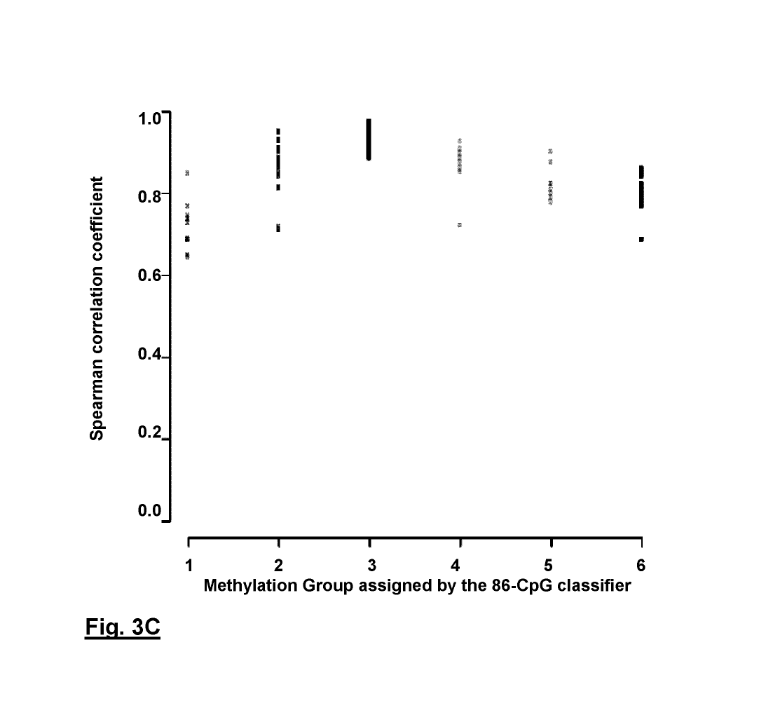

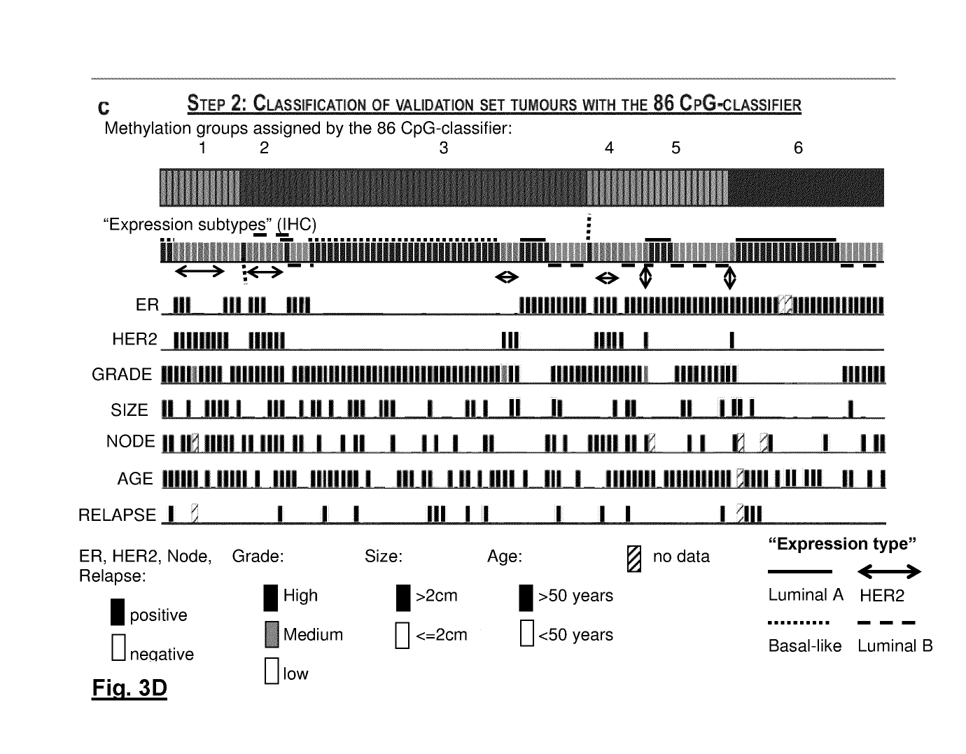

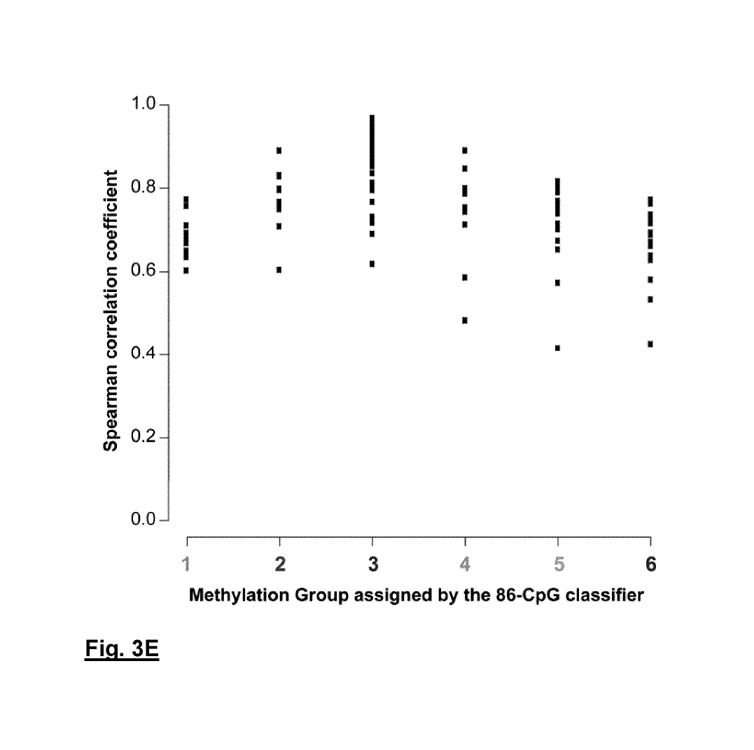

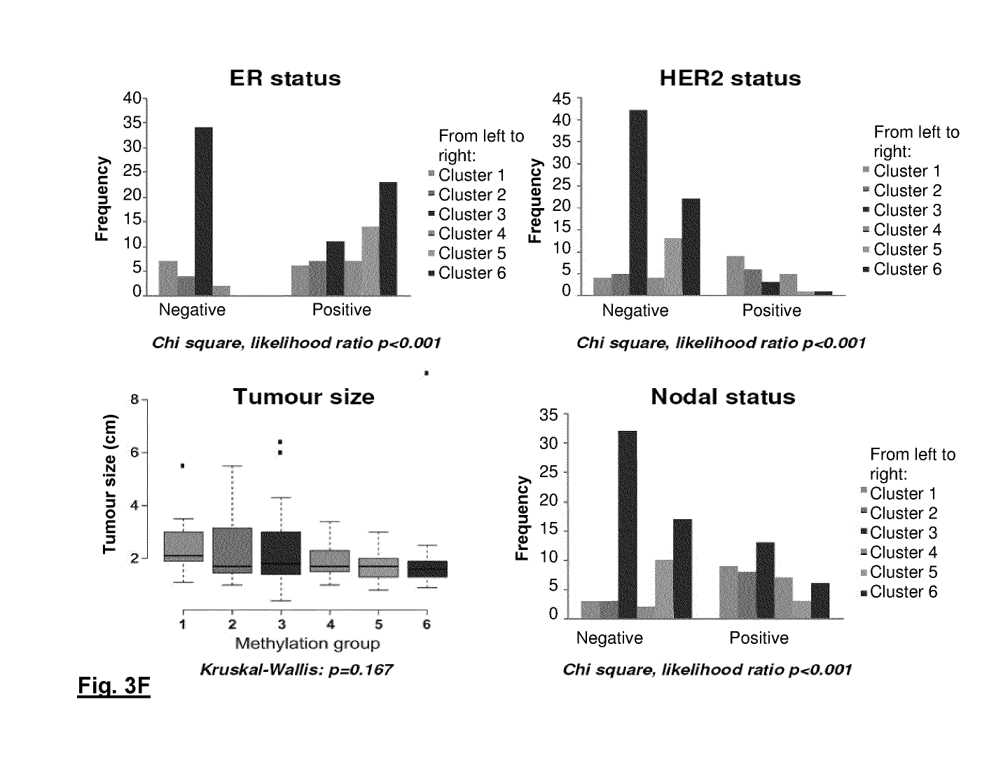

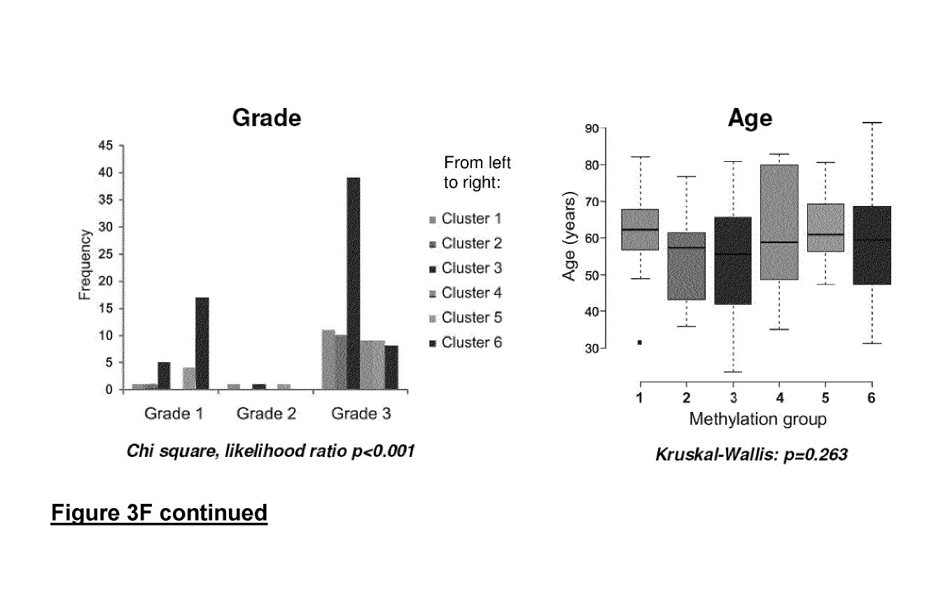

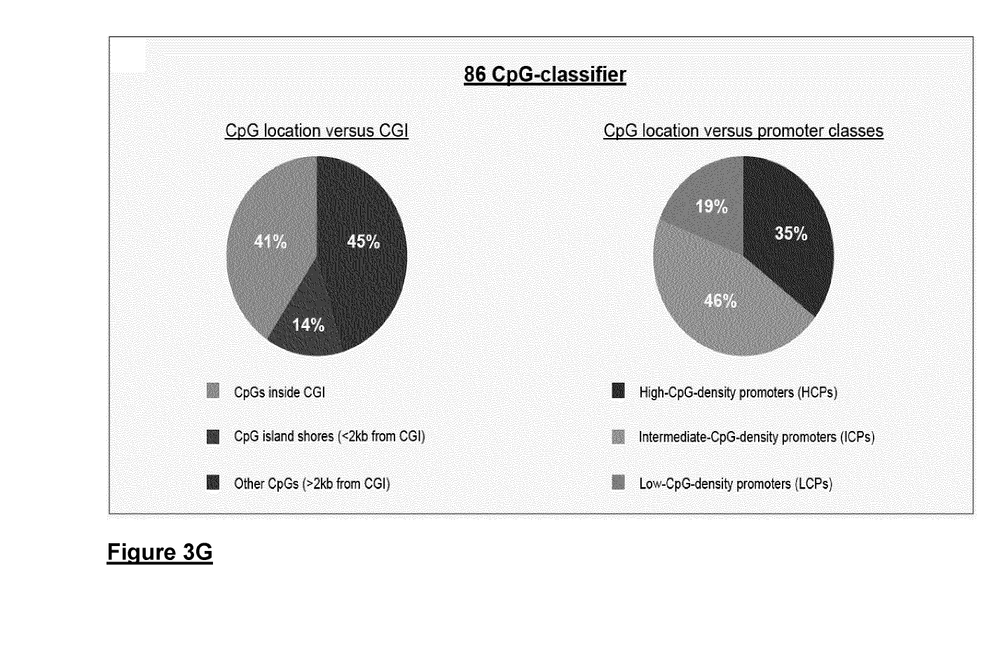

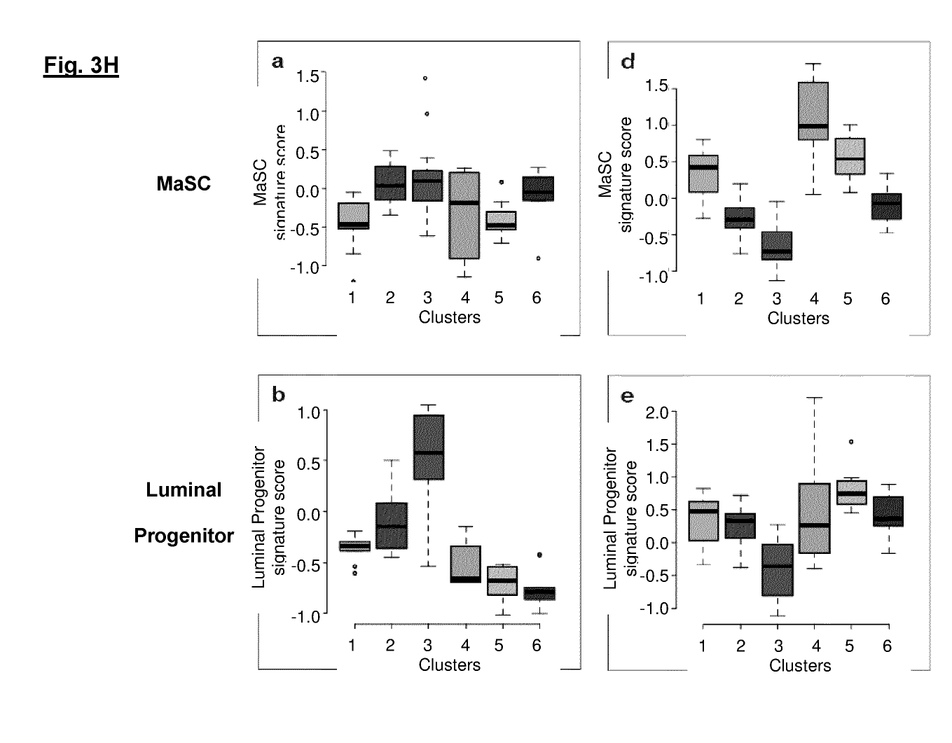

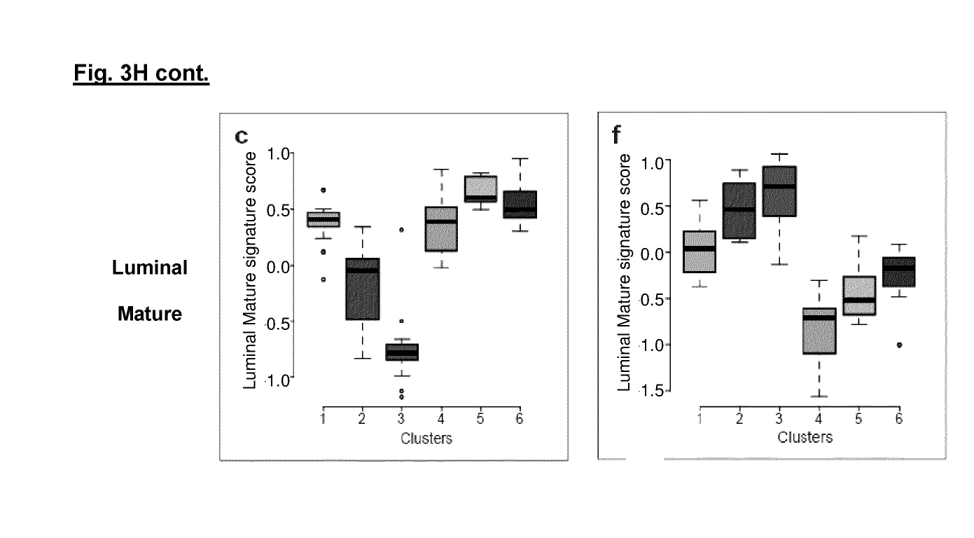

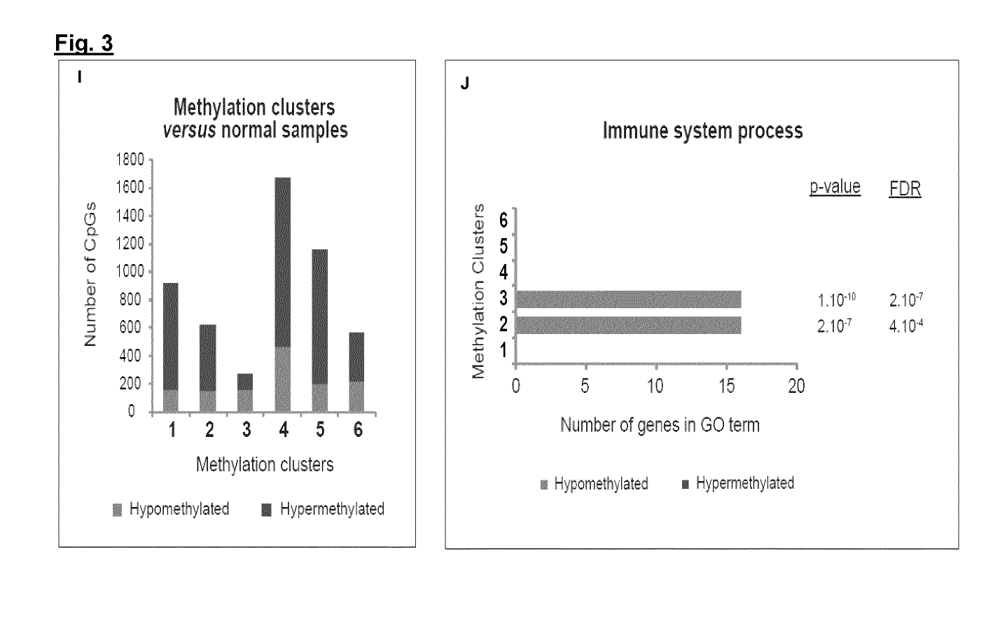

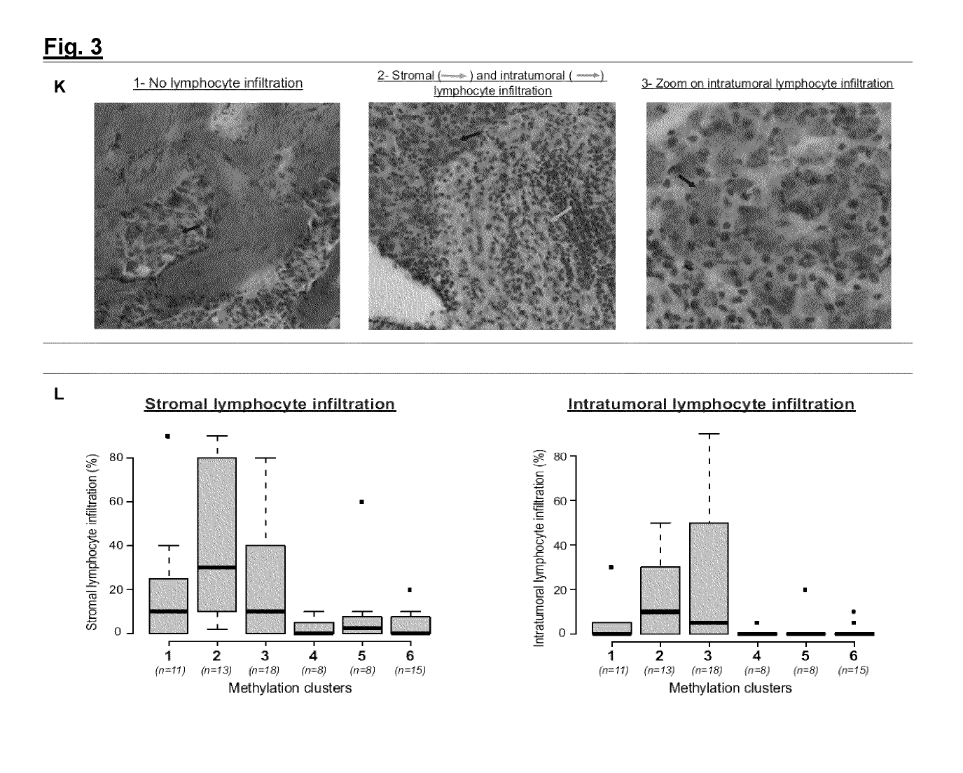

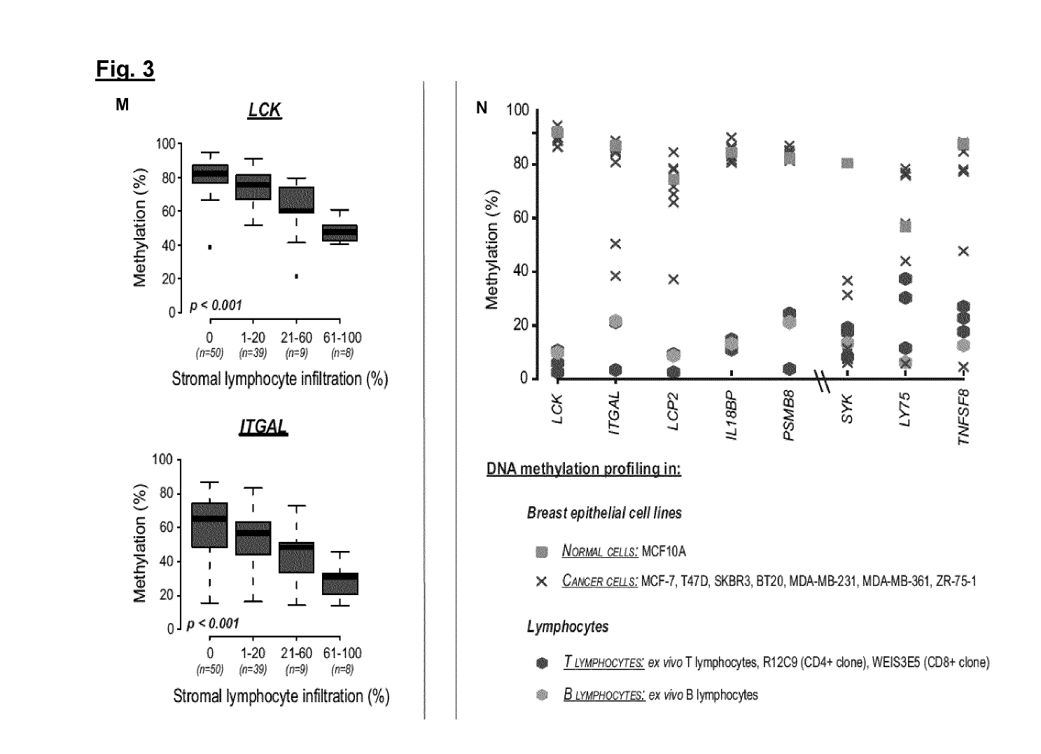

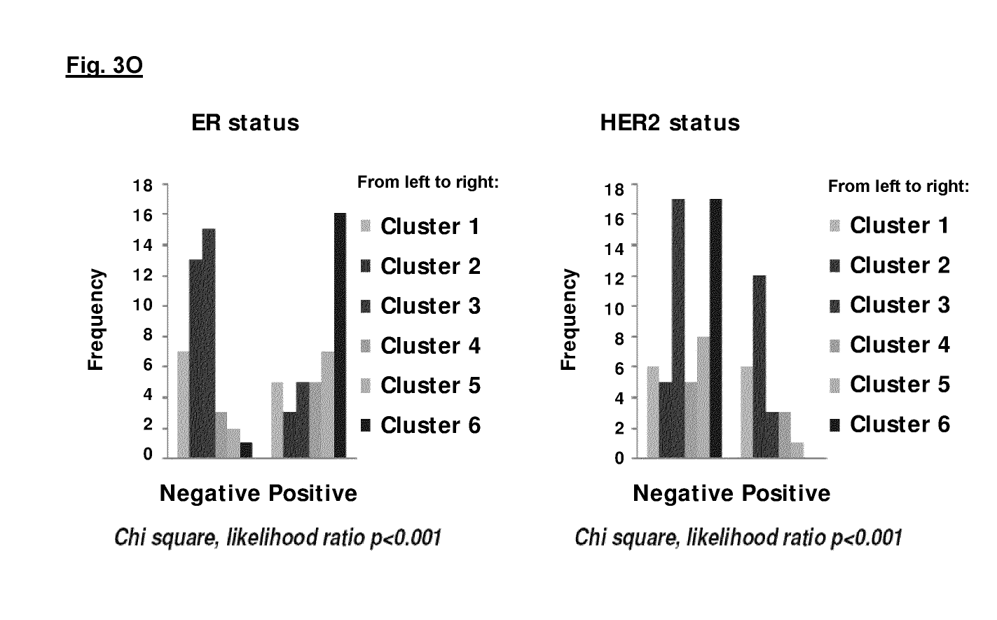

A ranked CpG list was constructed according to the Kruskal-Wallis test statistic values. In order to find the minimal number of CpGs to be used for the nearest centroid classifier, different classifiers were created from this list and the proportion of correctly classified samples from the main set as compared to the original clustering was calculated. We started with a classifier using the top 5 CpGs most differentially methylated CpGs between the 6 clusters from this list and added one by one an additional CpG from this list up to a total of 1519 (the number of CpGs for which the FDR-adjusted pvalue was 0). At the end, the minimal number of CpGs that yielded the maximum percentage of correct classification (96.38%) was given by 86 (see FIG. 3n and Table 2). Finally, the resulting 86-CpG classifier was applied to the validation dataset to classify the new patients into one of the 6 clusters.

TABLE 2

| SEQ | | | | | |
|---|---|---|---|---|---|
| ID | | | | | |
| NO | Name | Symbol | Gene_ID | Synonym | Accession |
| 1 | cg27610561 | SLC2A10 | GeneID: 81031 | GLUT10; | NM_030777.3 |
| | | | | MGC126706; | |
| 2 | cg21570818 | FUT5 | GeneID: 2527 | FUC-TV; | NM_002034.2 |
| 3 | cg08887581 | C1orf64 | GeneID: 149563 | MGC24047; RP11- | NM_178840.2 |
| | | | | 5P18.4; | |
| 4 | cg14023451 | GPLD1 | GeneID: 2822 | GPIPLD; PIGPLD; | NM_001503.2 |
| | | | | GPIPLDM; | |
| | | | | PIGPLD1; | |
| | | | | MGC22590; | |
| 5 | cg05215575 | FLJ25410 | GeneID: 124404 | | NM_144605.1 |
| 6 | cg11037787 | PLA2G2A | GeneID: 5320 | MOM1; PLA2; | NM_000300.2 |
| | | | | PLA2B; PLA2L; | |
| | | | | PLA2S; PLAS1; | |
| | | | | sPLA2; | |
| 7 | cg02671171 | RPH3AL | GeneID: 9501 | NOC2; | NM_006987.2 |
| 8 | cg00294382 | IL23A | GeneID: 51561 | P19; SGRF; IL-23; | NM_016584.2 |
| | | | | IL-23A; IL23P19; | |
| | | | | MGC79388; | |
| 9 | cg02643667 | TFF1 | GeneID: 7031 | pS2; BCEI; HPS2; | NM_003225.2 |
| | | | | HP1.A; pNR-2; | |
| | | | | D21S21; | |
| 10 | cg21137417 | SPP2 | GeneID: 6694 | SPP24; | NM_006944.2 |
| 11 | cg05089968 | MGC35308 | GeneID: 285800 | | NM_175922.3 |
| 12 | cg19456540 | SIX6 | GeneID: 4990 | Six9; OPTX2; | NM_007374.1 |
| 13 | cg14430151 | FLJ35725 | GeneID: 152992 | FLJ12891; | NM_152544.1 |
| 14 | cg04457051 | SCOC | GeneID: 60592 | SCOCO; | NM_032547.1 |
| | | | | HRIHFB2072; | |
| 15 | cg08097882 | POU4F1 | GeneID: 5457 | BRN3A; RDC-1; | NM_006237.2 |
| | | | | FLJ13449; | |
| 16 | cg25942450 | TLX3 | GeneID: 30012 | RNX; HOX11L2; | NM_021025.2 |
| | | | | MGC29804; | |
| 17 | cg08658594 | TAS2R13 | GeneID: 50838 | TRB3; T2R13; | NM_023920.2 |
| 18 | cg02170525 | CD8A | GeneID: 925 | CD8; MAL; p32; | NM_001768.4 |
| | | | | Leu2; | |
| 19 | cg02880679 | MBTD1 | GeneID: 54799 | SA49P01; | NM_017643.1 |
| | | | | FLJ20055; | |
| | | | | MGC126785; | |
| 20 | cg13271951 | FAM57B | GeneID: 83723 | FP1188; | NM_031478.3 |
| | | | | DKFZP434I2117; | |
| 21 | cg08285151 | HDAC9 | GeneID: 9734 | HD7; HDAC; | NM_058176.1 |
| | | | | HDRP; MITR; | |
| | | | | HDAC7; HDAC7B; | |
| | | | | HDAC9B; | |
| | | | | HDAC9FL; | |
| | | | | KIAA0744; | |
| | | | | DKFZp779K1053; | |
| 22 | cg05436658 | PRKCB1 | GeneID: 5579 | PKCB; PRKCB; | NM_002738.5 |
| | | | | PRKCB2; | |
| | | | | MGC41878; PKC- | |
| | | | | beta; | |
| 23 | cg02148642 | RGPD5 | GeneID: 84220 | RGP5; BS-63; | NM_032260.2 |
| | | | | DKFZp686I1842; | |
| 24 | cg26189983 | TNFRSF1B | GeneID: 7133 | p75; TBPII; | NM_001066.2 |
| | | | | TNFBR; TNFR2; | |
| | | | | CD120b; TNFR80; | |
| | | | | TNF-R75; | |
| | | | | p75TNFR; TNF-R- | |
| | | | | II; | |
| 25 | cg10707565 | CUBN | GeneID: 8029 | IFCR; MGA1; | NM_001081.2 |
| | | | | gp280; cubilin; | |
| 26 | cg23801057 | P2RX7 | GeneID: 5027 | P2X7; MGC20089; | NM_002562.4 |
| 27 | cg23092823 | PODN | GeneID: 127435 | PCAN; SLRR5A; | NM_153703.3 |
| | | | | MGC24995; | |
| 28 | cg03503295 | DNAH5 | GeneID: 1767 | HL1; PCD; CILD3; | NM_001369.1 |
| | | | | DNAHC5; | |
| | | | | KIAA1603; | |
| 29 | cg09448880 | PGLYRP3 | GeneID: 114771 | PGRPIA; PGRP- | NM_052891.1 |
| | | | | Ialpha; | |
| 30 | cg22194129 | CLEC4C | GeneID: 170482 | DLEC; HECL; | NM_130441.2 |
| | | | | BDCA2; CD303; | |
| | | | | CLECSF7; | |
| | | | | CLECSF11; | |
| | | | | PRO34150; | |
| | | | | MGC125791; | |
| | | | | MGC125792; | |
| | | | | MGC125793; | |
| 31 | cg17108819 | CD8A | GeneID: 925 | CD8; MAL; p32; | NM_001768.4 |
| | | | | Leu2; | |
| 32 | cg01017147 | DNM3 | GeneID: 26052 | Dyna III; | NM_015569.2 |
| | | | | KIAA0820; | |
| | | | | MGC70433; | |
| 33 | cg18752854 | TNS1 | GeneID: 7145 | TNS; MGC88584; | NM_022648.3 |
| 34 | cg19589427 | TNFSF18 | GeneID: 8995 | TL6; AITRL; | NM_005092.2 |
| | | | | GITRL; hGITRL; | |
| | | | | MGC138237; | |
| 35 | cg21475402 | BCAN | GeneID: 63827 | BEHAB; CSPG7; | NM_198427.1 |
| | | | | MGC13038; | |
| 36 | cg10300684 | FOXG1B | GeneID: 2290 | BF1; QIN; FKH2; | NM_005249.3 |
| | | | | HFK1; FKHL1; | |
| | | | | FKHL4; HBF-1; | |
| 37 | cg17095936 | TBX19 | GeneID: 9095 | TPIT; TBS19; TBS19; | NM_005149.1 |
| | | | | dJ747L4.1; | |
| 38 | cg01335367 | C12orf34 | GeneID: 84915 | FLJ14721; | NM_032829.1 |
| 39 | cg24525573 | C1orf64 | GeneID: 149563 | MGC24047; RP11- | NM_178840.2 |
| | | | | 5P18.4; | |
| 40 | cg15604467 | POU4F1 | GeneID: 5457 | BRN3A; RDC-1; | NM_006237.2 |
| | | | | FLJ13449; | |
| 41 | cg05181279 | RIG | GeneID: 10530 | | XM_932493.1 |
| 42 | cg19018097 | FLJ30934 | GeneID: 254122 | MGC42112; | NM_152760.2 |
| | | | | MGC57276; | |
| 43 | cg06119575 | TAL2 | GeneID: 6887 | | NM_005421.1 |
| 44 | cg14686321 | FLJ31951 | GeneID: 153830 | DKFZp686M11215; | NM_144726.1 |
| 45 | cg10541755 | EIF5A2 | GeneID: 56648 | EIF-5A2; eIF5AII; | NM_020390.5 |
| 46 | cg10334928 | STON2 | GeneID: 85439 | STN2; STNB; | NM_033104.2 |
| | | | | STNB2; | |
| 47 | cg11354906 | SFRP2 | GeneID: 6423 | | NT_016354.18 |
| 48 | cg06436504 | DOC1 | GeneID: 11259 | GIP90; | NM_182909.1 |
| 49 | cg17619823 | ADRB3 | GeneID: 155 | BETA3AR; | NM_000025.1 |
| 50 | cg27196745 | PTPRO | GeneID: 5800 | PTPU2; GLEPP1; | NM_002848.2 |
| | | | | PTP-U2; | |
| 51 | cg02399455 | SRI | GeneID: 6717 | SCN; | NM_198901.1 |
| 52 | cg11802013 | CCND1 | GeneID: 595 | BCL1; PRAD1; | NT_078088.3 |
| | | | | U21B31; | |
| | | | | D11S287E; cyclin | |
| | | | | D1; | |
| 53 | cg02595219 | KCNE3 | GeneID: 10008 | HOKPP; MiRP2; | NM_005472.3 |
| | | | | MGC129924; | |
| | | | | DKFZp781H21101; | |
| 54 | cg00596686 | STS | GeneID: 412 | ES; ASC; ARSC; | NM_000351.3 |
| | | | | SSDD; ARSC1; | |
| 55 | cg27491887 | KCNQ1 | GeneID: 3784 | LQT; RWS; WRS; | NT_009237.17 |
| | | | | LQT1; ATFB1; | |
| | | | | KCNA8; KCNA9; | |
| | | | | Kv1.9; Kv7.1; | |
| | | | | KVLQT1; | |
| 56 | cg05158615 | NPY | GeneID: 4852 | PYY4; | NM_000905.2 |
| 57 | cg20980592 | MEP1A | GeneID: 4224 | PPHA; | NM_005588.1 |
| 58 | cg13696012 | BPIL1 | GeneID: 80341 | RYSR; LPLUNC2; | NM_025227.1 |
| | | | | C20orf184; | |
| | | | | dJ726C3.2; | |
| 59 | cg00953256 | CCND1 | GeneID: 595 | BCL1; PRAD1; | NT_078088.3 |
| | | | | U21B31; | |
| | | | | D11S287E; cyclin | |
| | | | | D1; | |
| 60 | cg07426960 | CCND1 | GeneID: 595 | BCL1; PRAD1; | NT_078088.3 |
| | | | | U21B31; | |
| | | | | D11S287E; cyclin | |
| | | | | D1; | |
| 61 | cg01109219 | RASGRP3 | GeneID: 25780 | GRP3; KIAA0846; | NM_170672.1 |
| 62 | cg10968815 | BPIL1 | GeneID: 80341 | RYSR; LPLUNC2; | NM_025227.1 |
| | | | | C20orf184; | |
| | | | | dJ726C3.2; | |
| 63 | cg15046693 | CEBPG | GeneID: 1054 | GPE1BP; IG/EBP- | NM_001806.2 |
| | | | | 1; | |
| 64 | cg23391785 | DNM3 | GeneID: 26052 | Dyna III; | NM_015569.2 |
| | | | | KIAA0820; | |
| | | | | MGC70433; | |
| 65 | cg00051623 | CASP1 | GeneID: 834 | ICE; P45; IL1BC; | NM_033294.2 |
| 66 | cg13755070 | FLI1 | GeneID: 2313 | EWSR2; SIC-1; | NM_002017.2 |
| 67 | cg02657438 | STON2 | GeneID: 85439 | STN2; STNB; | NM_033104.2 |
| | | | | STNB2; | |
| 68 | cg13144783 | CCR1 | GeneID: 1230 | CD191; CKR-1; | NM_001295.2 |
| | | | | HM145; CMKBR1; | |
| | | | | MIP1aR; SCYAR1; | |
| 69 | cg18129786 | ZNF445 | GeneID: 353274 | ZNF168; | NM_181489.4 |
| | | | | MGC126535; | |
| 70 | cg02723533 | CCND1 | GeneID: 595 | BCL1; PRAD1; | NT_078088.3 |
| | | | | U21B31; | |
| | | | | D11S287E; cyclin | |
| | | | | D1; | |
| 71 | cg10964421 | TNFRSF10D | GeneID: 8793 | DCR2; CD264; | NT_023666.17 |
| | | | | TRUNDD; | |
| | | | | TRAILR4; | |
| 72 | cg24199834 | POU4F2 | GeneID: 5458 | BRN3B; BRN3.2; | NM_004575.1 |
| | | | | Brn-3b; | |
| 73 | cg14003512 | PLGLB2 | GeneID: 5342 | PLGP1; | NM_002665.3 |
| 74 | cg23642747 | INA | GeneID: 9118 | NEF5; NF-66; | NM_032727.2 |
| | | | | TXBP-1; | |
| | | | | MGC12702; | |
| 75 | cg01424107 | CDX2 | GeneID: 1045 | CDX3; CDX-3; | NM_001265.2 |
| 76 | cg02100848 | C3orf32 | GeneID: 51066 | | NM_015931.1 |
| 77 | cg05056120 | EBF | GeneID: 1879 | COE1; EBF1; | NM_024007.2 |
| | | | | OLF1; O/E-1; | |
| 78 | cg00839584 | IL1A | GeneID: 3552 | IL1; IL-1A; IL1F1; | NM_000575.3 |
| | | | | IL1-ALPHA; | |
| 79 | cg02681442 | FOXG1B | GeneID: 2290 | BF1; QIN; FKH2; | NM_005249.3 |
| | | | | HFK1; FKHL1; | |
| | | | | FKHL4; HBF-1; | |
| 80 | cg06653796 | LIME1 | GeneID: 54923 | LIME; LP8067; | NM_017806.1 |
| | | | | FLJ20406; | |
| | | | | dJ583P15.4; RP4- | |
| | | | | 583P15.5; | |
| 81 | cg21296230 | GREM1 | GeneID: 26585 | DRM; PIG2; | NM_013372.5 |
| | | | | DAND2; IHG-2; | |
| | | | | GREMLIN; | |
| | | | | CKTSF1B1; | |
| | | | | MGC126660; | |
| 82 | cg11547724 | HPX | GeneID: 3263 | | NM_000613.1 |
| 83 | cg17240454 | SPDEF | GeneID: 25803 | PDEF; bA375E1.3; | NM_012391.1 |
| | | | | RP11-375E1_A.3; | |
| 84 | cg08047907 | C1orf114 | GeneID: 57821 | FLJ25846; RP1- | NM_021179.1 |
| | | | | 206D15.2; | |
| 85 | cg17667972 | KRT4 | GeneID: 3851 | K4; CK4; CYK4; | NM_002272.1 |
| | | | | FLJ31692; | |
| 86 | cg07935264 | IL1B | GeneID: 3553 | IL-1; IL1F2; IL1- | NM_000576.2 |
| | | | | BETA; | |

Relapse-Free Survival Analysis

For the meta-analysis performed on publicly available gene expression data, only the genes displaying a high anti-correlation between their methylation and expression status (Pearson's coefficient below than −0.7) in our main set of patients were selected. Among the 85 genes meeting this criterion, several were eliminated because they were not represented on the microarray platforms (9) or because information for these genes was available for less than 700 patients (15). Six other genes were excluded from this meta-analysis because they did not display differential methylation between normal breast samples and IDCs in our population. The prognostic value of individual CpGs or genes was estimated by univariate Cox regression. Multivariate Cox regression was used to test the independent prognostic values of CpGs or genes of interest in the presence of traditional clinical variables. Cox models were stratified by datasets to account for the possible heterogeneity in patient selection or other potential confounders, as implemented in the ‘survival’ R package available on CRAN (http://cran.r-project.org/web/packages/survival). The significance of individual hazard ratios was estimated by Wald's test. For univariate analysis, the p-values were corrected for multiple testing by means of the false discovery rate (FDR) and variables with a FDR below than 0.1 were considered prognostic. For multivariate analysis, variables with a p-value below than 0.05 were considered prognostic.

Annotation of Infinium Array in Terms of CpG Location

Additional annotations of the Infinium array were added to the ones provided by Illumina regarding the location of the CpG (i) versus CGI (CpG inside a CGI, CpG island shore, other CpG) and (ii) versus promoter classes (High-, Intermediated or Low-CpG-density promoter).

CpG Location Versus CGI

CpGs were classified according to their position relatively to CpG islands (i.e. CpG inside a CGI, CpG island shore or other CpG). Two classifications were established, and this in function of the CGI definition used: the UCSC definition (CpG_Island_UCSC classification) or the improved and revisited definition of Bock et al., 2007 PLoS Comput. Biol. 3, 1055-1070 (CpG_Island_Revisited classification). A CpG was considered as a CpG island shore if it was located inside a 2 kb region around a CGI (as defined by Irizarry et al., 2009 Nat. Genet. 41, 178-186). A CpG located neither in a CGI nor in a 2 kb region around a CGI was considered as other CpG. The revisited classification by Bock et al. for all analyses.

CpG Location Versus Promoter Classes

Promoters represented on the Infinium array were categorized using their CpG content as defined by Weber et al., 2007 (Nat. Genet. 39, 457-466). First, regions from −700 to +500 bp surrounding the transcription start site (TSS) were extracted using the UCSC genome browser data (Rhead et al., 2010 Nucleic Acids Res. 38, D613-619). Then, using the DNA sequences corresponding to those promoter fragments, the CpG ratio and the GC content were calculated in sliding windows of 500 bp with 5 bp offsets. Finally, according to the definition provided by Weber et al., 2007, the promoters were classified as HCPs (High-CpG-density promoters) if a least one 500 by window contains a CpG ratio >0.75 and a GC content >0.55 was found; as LCPs (Low-CpGdensity promoters) if no 500 bp window has reached a CpG ratio of 0.48; or as ICPs (Intermediate-CpG-density promoters) otherwise.

Methylation Difference Criterion

Several indications led us to choose 20% as the methylation difference criterion. First, it seemed that the Infinium assay gave values ranging from 0 to 0.2 for unmethylated CpGs. Second, a recent study has shown that for more than 90% of the loci, the sensitivity of methylation difference detection is 0.2 (Bibikova, M. et al., 2009 Epigenomics 1, 177-200).

Class Comparison Analyses in the Main Set of Patients

A two-sided Mann-Whitney test (also called Wilcoxon-Mann-Whitney test) was employed to test the null hypothesis (HO) assumption of equality of the methylation values in two defined groups of data. The loss of power induced by multiple tests was corrected by the false discovery rate (FDR) approach (Benjamini, Y. & Hochberg, Y. 1995 J R Stat Soc Series B 57, 289-300). For normal samples we considered the mean of methylation values, because of the small sample size and the low variance. For tumour samples, because of their higher heterogeneity, we considered the median value, less sensitive to extreme values.

Between IDCs and Normal Breast Tissue Samples

A particular CpG was considered hyper- or hypo-methylated in IDCs as compared to normal breast tissue samples according to the following two criteria: 1/ the CpG had to show at least a 20% methylation difference in IDCs as compared to normal breast tissue samples in at least 10% of the IDCs; 2/ to be considered hypermethylated, the CpG had to show at least ten times more hypermethylation events than hypomethylation events in breast cancer. Conversely, to be considered hypomethylated, it had to show at least ten times more hypomethylation events than hypermethylation events in breast cancer.

Between the Two Main Clusters, I and II

CpGs differentially methylated between clusters I and II were determined according to these two criteria: 1/ they had to show a methylation difference of at least 20% between the two groups; 2/ the FDR-corrected Wilcoxon p-value for the concerned CpGs had to be lower than 0.1.

Between Each Methylation Subcluster and Normal Breast Tissue Samples

The criteria for determining that a given methylation subcluster showed differential methylation with respect to normal breast tissue samples were: 1/ The CpGs concerned had to show a difference in methylation of at least 20% between the two groups; 2/ the Wilcoxon p-value for the CpGs concerned had to be lower than 0.01. Here, the FDR criterion as described above was not used, because of the small number of samples composing each group.

Bisulphite Genomic Sequencing

Methylation status of four CpG sites—cg07471052, cg11566244, cg22498251 and cg09847584—located respectively near the transcription start sites of the CDK3, GSTP1, TWIST1 and RIMBP2 genes, was examined by bisulphite genomic sequencing applied to 1 normal (N1) and 3 breast cancer (BC10, BC32 and BC109) samples. Primers were designed manually and sequences are provided in Table 3. The PCR amplified fragments were purified by QIAquick® Gel Extraction kit (Qiagen), cloned into the pCR®II-TOPO® vector (Invitrogen, Carlsbad, Calif., USA), and used to transform competent Escherichia coli TOP10 cells. Clones were selected by blue/white colonie screening and amplified. Plasmids were purified with the Qiagen-MiniPrep kit (Qiagen). The PCR products were sequenced by Genoscreen (Lille, France) and CpG methylation status were analysed with the BiQ Analyzer software as described by Bock et al.,2005 (Bioinformatics 21, 4067-4068).

TABLE 3
Primers used for bisulphite genomic sequencing
(Respective SEQ ID Nos 513-529)

| | | | Annealing |
|---|---|---|---|
| Gene | PCR round | Sequence 5′-3′ | temperature |
| CDK3 | PCR1 | Forward: gtttagaggggttttttgattatttg | 50° C. |
| | | Reverse: aactcctacaactccaaaaaattc | |
| | PCR2 | Forward: gagggaatagttggaatgtattttg | 45° C. |
| | | Reverse: ctaaactactatttcctactaactac | |
| GSTP1 | PCR1 | Forward: ggtttagagtttttagtatggggtt | 50° C. |
| | | Reverse: actctaaccctaatctaccaacaa | |
| | PCR2 | Forward: aggtaggagtatgtgtttggtag | 50° C. |
| | | Reverse: tcaaaaatacaaaaaaaaaacaaaa | |
| TWIST1 | PCR1 | Forward: ggtttggtttttggaattttaaggg | 50° C. |
| | | Reverse: aaaacaacaatatcattaacctaac | |
| | PCR2 | Forward: gtttatttgattattgggtgggttt | 50° C. |
| | | Reverse: ctataacaacaacaataacaacaac | |
| RIMBP2 | PCR1 | Forward: aaatatgggggtattattttatatg | 50° C. |
| | | Reverse: ccttactattaaaaatacaaatacc | |
| | PCR2 | Forward: atgaattgaaggatgttatttaggg | 50° C. |
| | | Reverse: aaacttccaaacaaaaataaccaac | |

Bisulphite Pyrosequencing

750 ng of genomic DNA were bisulphite-converted using the EZ DNA Methylation™ kit (Zymo Research) as for DNA methylation profiling. One third of the converted DNA was used as template for each subsequent PCR. To ensure sufficient amount of PCR product for sequencing nested PCRs were performed. PCR primers for pre-amplification (EF, ER primers) were deduced manually or with the help of “BiSearch Primer Design and Search Tool” (http://bisearch.enzim.hu) and checked for tendency to form oligomers, hairpin loops etc. using the Generunner software (version 3.05, Hastings Software Inc.). Primers for nested amplification and sequencing were deduced manually or using PyroMark® Assay Design 2.0 software (Qiagen). Pre-amplification PCRs were conducted with 3 mM MgCl2, 1 mM of each dNTP, 12% (v/v) DMSO, 500 nM of each primer (EF+ER primers, see Table 4) and optionally 500 mM Betaine in heated-lid thermocyclers under the following conditions: 95° C. 3:00; 25 cycles of [94° C. 0:30; 51° C. 0:40; 72° C. 1:30]; 72° C. 5:00. Nested amplifications (F, RBio primers) were performed with the HotStarTaq PCR kit (Qiagen) using 2% (v/v) of the pre-amplification PCR as template under the following conditions: 95° C. 15:00; 45 cycles of [94° C. 0:30; 55° C. 0:30; 72° C. 0:30]; 72° C. 10:00. Amplification success was assessed with agarose gel electrophoresis and pyrosequencing of the PCR products (S primers) was performed with the Pyromark™ Q24 system (Qiagen).

TABLE 4
Primers used for bisulphite pyrosequencing
(Respective SEQ ID Nos 530 to 575)

| primer name | primer sequence (5′ to 3′) |
|---|---|
| CD3D_EF | TGTGTAAATGTGGTTGTATTGTTAATAGG |
| CD3D_ER | CATCATATTACTCAAACTAATCTCAAACTCC |
| CD3D-F2 | GTGATTTGGTTTTATTTATTGGATGAGT |
| CD3D-R2Bio | [Btn]AATAAACCTCACTCCCATCAAT |
| CD3-S2 | GGTTTTATTTATTGGATGAGTTT |
| CD3D-S2A-cg077 | GGTTTGGTATTGGTTATTTTTT |
| CD3G_EF | GGTATTTGTATTTGTAGTTTTGTTGAGG |
| CD3G_ER | TTCTCCTCCATAAAACACTATTTCTCTC |
| CD3G-F1 | TGATGGGTGGAGTTAGTTTAGT |
| CD3G-R1Bio | [Btn]AAACCCTTCCCCTATTCCATA |
| CD3G-S1 | GGTTGGTTGTTAAGGG |
| CD6_EF2 | GGGGAAGTGTGTTTGTATGGATG |
| CD6_ER | AAACCACATATCTAAAACTATCTCTAACTACTAC |
| CD6-F1 | AGGTAGTTGGGGTTTTTTTTATTAG |
| CD6-R1Bio | [Btn]CTACCCTTTACTATTCTTATTCCTATATC |
| CD6-S1 | ATATTTATAGGTTGGGTTTG |
| CD79B_EF | TAGGTAGGAGAGGAATTGGGGTTATAG |
| CD79B_ER | CATCCACAAAAAACCCCAACTATACTAC |
| CD79B-F1 | AGTTGGAGATGAGAGTAAATTTTATAGG |
| CD79B-R1Bio | [Btn]AATACCTCCCCTAAATCCCAATTTACAT |
| CD79B-S1 | GGTTGGGTATAGGAGATA |
| HCLS1_EF | TTATTGTTAAAATTTTGTAAAAGATTAGGTATAG |
| HCLS1_ER | TTCCTCCTCAACTCTTACTCTATATTTCC |
| HCLS1-F1 | AGGATGGGGTGGTAGGAAAT |
| HCLS1-R1Bio | [Btn]CCTCCACCTATACAAACCTCTATTCTA |
| HCLS1-S1 | GGGTGGTAGGAAATG |
| ICOS_EF | TAAGTAGGTAATTTAAAAATTTAATGGTTTGATG |
| ICOS_ER | CCTCTATCTTCAAAATCATCAATAATCCATAC |
| ICOS-F1 | GAGGTTTGATTTTATGTTTGTTAGAAATAG |
| ICOS-R1Bio | [Btn]TCCCAAAAAACCCACTTCC |
| ICOS-S1 | TTTGTTAGAAATAGTTAATAGTTTT |
| LCK_EF | GGTTTATGGTGGTAGGAAGTTTGG |
| LCK_ER | TTAACACCTAACTATCCATATACCTAATATCC |
| LCK-F1 | GTTAGGTTAGGTTAGGAGGATTAT |
| LCK-R1Bio | [Btn]CCAACCACAAAAAACTACTACATC |
| LCK-S2 | GAGAGTTGGTATTGGGGG |
| SIT1_EF | GTAGTGTGTTTGTGGATTTTTATATTTGTAG |
| SIT1_ER | ATCTAATCAACAACTTATCCTTCCTCCTAC |
| SIT1-F1 | GTGGGTTTTTTTAGGGGTTGTGA |
| SIT1-R1Bio | [Btn]TCTCAATCAACCCATCCCTATTA |
| SIT1-S1 | GTTGTGAAGTTGTTATTTTTTATTT |
| UBASH3A-EF2 | TGGTGGAAATAGTTAGGATTGGTG |
| UBASH3A-ER | CAATATCTTACCCTACAAAATACACTACTTTAAC |
| UBASH3A-F1 | GGTTTAAGGGTAGGAAGAGATGG |
| UBASH3A-R1Bio | [Btn]ACTAACTAAACCCCCAAATCTCTAAACAAT |
| UBASH3A-S1 | GTAGGAAGAGATGGTAG |

Gene Set Enrichment Analysis (GSEA)

GSEA is a powerful analytical method first developed to determine if the members of a given gene set are significantly enriched among the genes most differentially expressed between two sample groups (Mootha, V. K. et al.2003 Nat. Genet. 34, 267-273). Here this method was applied to both the methylation and expression data to assess the possibility that ER biology might be regulated by DNA methylation. For this, it was hypothesized that the ESR1 module genes were more highly methylated in cluster I (“ER-negative tumours”) than in cluster II (“ER-positive tumours”). For this analysis, the ESR1 module described by Desmedt et al., 2008 (Clin. Cancer Res. 14, 5158-5165) had to be divided into two submodules: an ESR1-positive module, containing all ESR1 module genes whose expression correlates positively with ESR1 expression, and an ESR1-negative module containing those whose expression correlates negatively with ESR1 expression. All 14,475 genes represented on the bead array were ranked from the most hypermethylated to the most hypomethylated in cluster I with respect to cluster II. The signal-to-noise ratio (the difference in means of the two classes divided by the sum of the standard deviations of the two classes) was used to perform the ranking. When a gene was represented by several probes on the bead array, the most variant one was selected for this analysis. The 20,606 genes represented on the Affymetrix array were ranked according to the same method. The goal of this GSEA analysis was to determine whether the ESR1 module genes are randomly distributed throughout the ranked lists (suggesting no enrichment of these gene sets in one of the two clusters) or primarily found at the top or bottom (suggesting an enrichment of these gene sets in one of the two clusters). A running sum statistic, corresponding to the enrichment score, was calculated for each gene set on the basis of the ranks of the investigated gene set members, relative to those of the non-members. The significance of such enrichments was estimated by calculating a permutation-based p-value corrected for multiple tests by the false discovery rate (FDR) approach. This analysis was performed with the freely accessible software GSEA-P, provided by the Broad Institute (http://www.broadinstitute.org/gsea/). This GSEA technique has been described in detail by Subramanian et al., 2005 (Proc. Natl Acad. Sci. USA 102, 15545-15550).

Correlation Between Methylation and Expression Data

The correlation between methylation and expression data in the main set of patients was evaluated by Pearson's correlation test between each Infinium methylation probe and the most variant Affymetrix expression probe for the gene concerned. Infinium methylation probes presenting values with a range lower than 20% were excluded from this analysis. The range was calculated by subtracting the smallest methylation value from the greatest one for each probe.

Gene Ontology Analysis

Gene ontology analysis was done with DAVID (http://david.abcc.ncifcrf.gov/), a web-accessible program providing a comprehensive set of functional annotation tools for understanding the biological meaning of large lists of genes (Huang, D. W. et al., 2009 Nat. Protoc. 4, 44-57). Only genes differentially methylated between each subcluster and normal breast samples and displaying an acceptable anti-correlation between their methylation and expression status (Pearson's coefficient below than −0.4) were selected for this analysis. This ensured the selection of genes whose expression is affected by methylation changes, facilitating the biological interpretation of results.

Collection of Publicly Available Gene Expression Datasets

Gene expression datasets were retrieved from public databases or authors' websites. We used normalized data (log2 intensity in single-channel platforms or log 2 ratio in dual-channel platforms). Hybridization probes were mapped to Entrez GeneID as described33 using RefSeq and Entrez database version 2007 Jan. 21. When multiple probes were mapped to the same GeneID, the one with the highest variance in a particular dataset was selected. Ten breast cancer microarray datasets were used. Distant metastasis-free survival (DMFS) was used as survival endpoint. We censored the survival data at 10 years in order to have comparable follow-up across the different studies as described (Desmedt, C. et al., 2008 Clin. Cancer Res. 14, 5158-516517,34; Haibe-Kains, B. et al., 2008 Bioinformatics 24, 2200-2208).

Treatment of Breast Cancer Epithelial Cell Lines with 5-aza-2′-deoxycytidine

Breast cancer epithelial cell lines MCF-7, MDA-MB-231, MDA-MB-361, T47D, SKBR3, BT20 and ZR-75-1 were treated with 1 μM of 5-aza-2′-deoxycytidine (Sigma) during 4 days. Medium containing the drug was refreshed every day.

Additional Statistical Analyses

Spearman's correlation was used to compare Infinium data with bisulphite genomic sequencing or pyrosequencing data. The Mann-Whitney U test and the Kruskal-Wallis test were used to test for differences of a continuous variable between two or multiple subgroups, respectively. Chi-square tests were used to compare discrete variables and the p-values were estimated by the likelihood ratio or Fisher's Exact test (for comparison of binary variables). The Phi coefficient was used to determine the strength of associations between the “known expression subtypes” of breast cancer and our DNA methylation-based clusters. The values range from 0 to 1, and can be interpreted in a similar way to Spearman's rank correlation coefficient. The significance of such associations was computed by means of a chi-square test.

### Example 1

### Infinium Methylation Platform Analysis of DNA Methylation Profiling of Two Independent Sets of Frozen Breast Tissue Samples

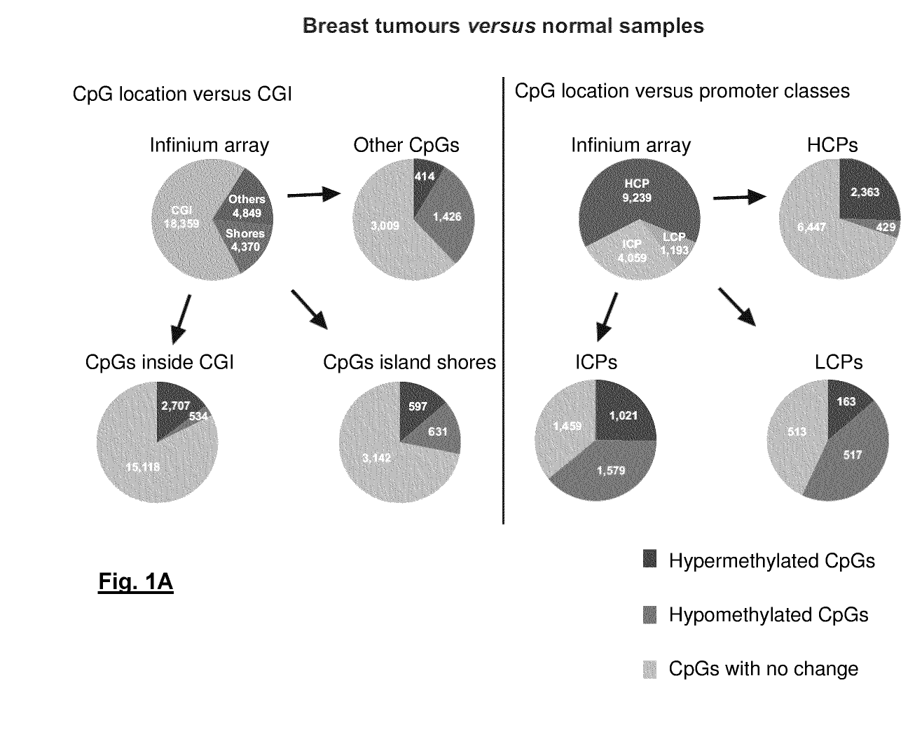

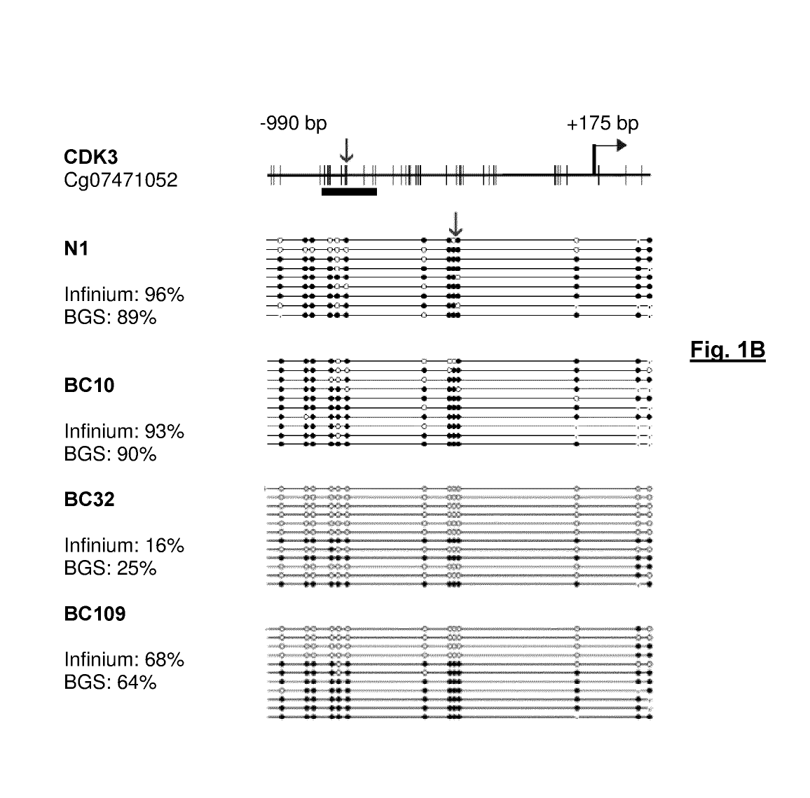

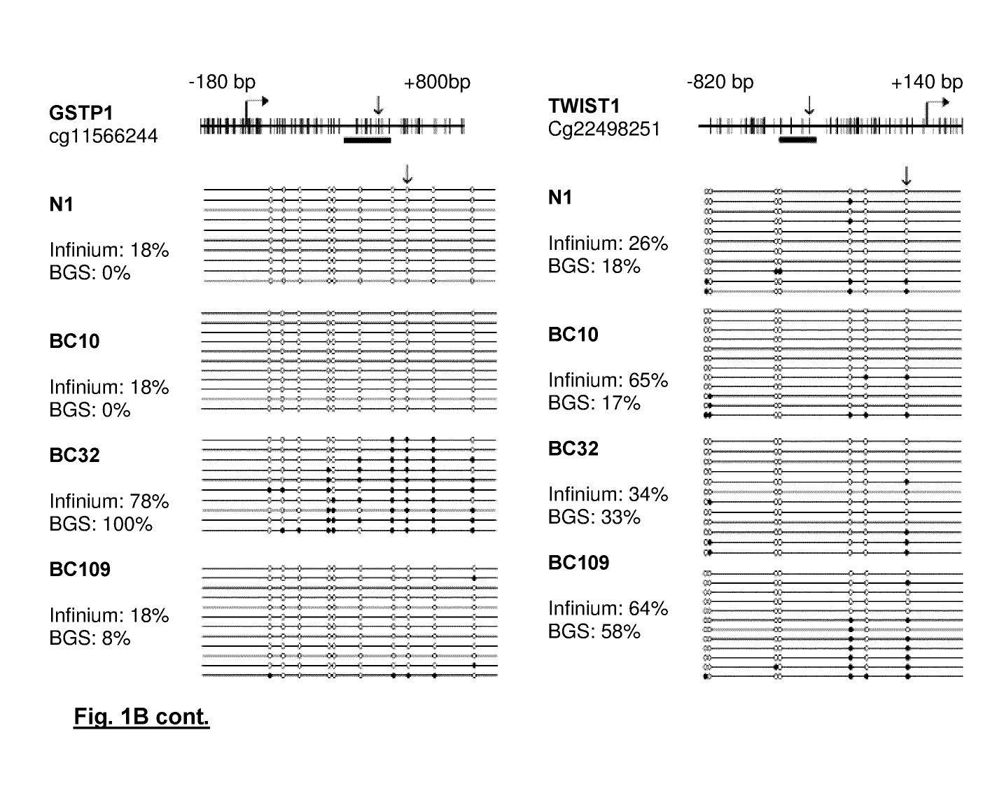

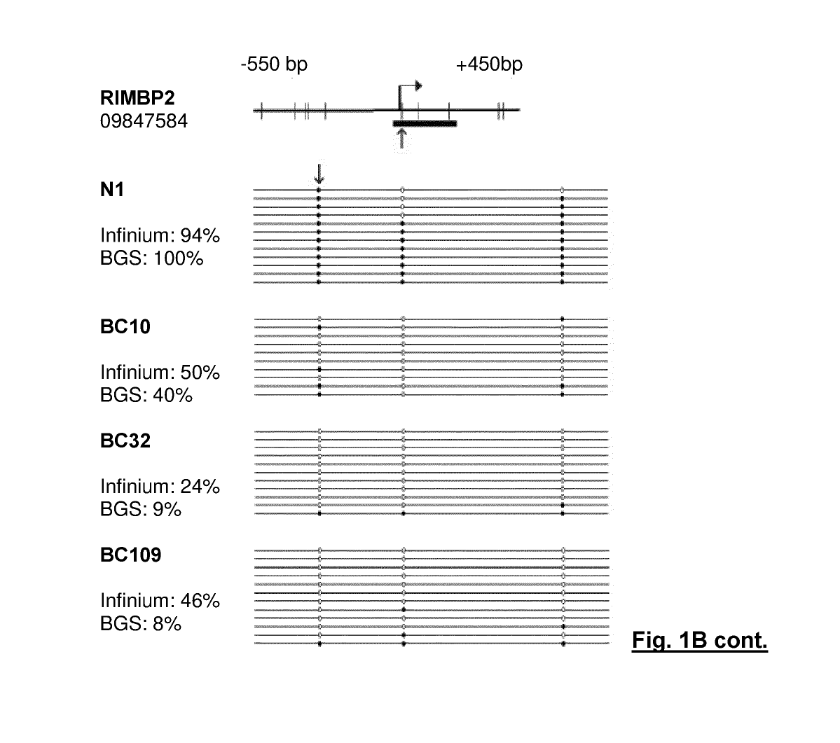

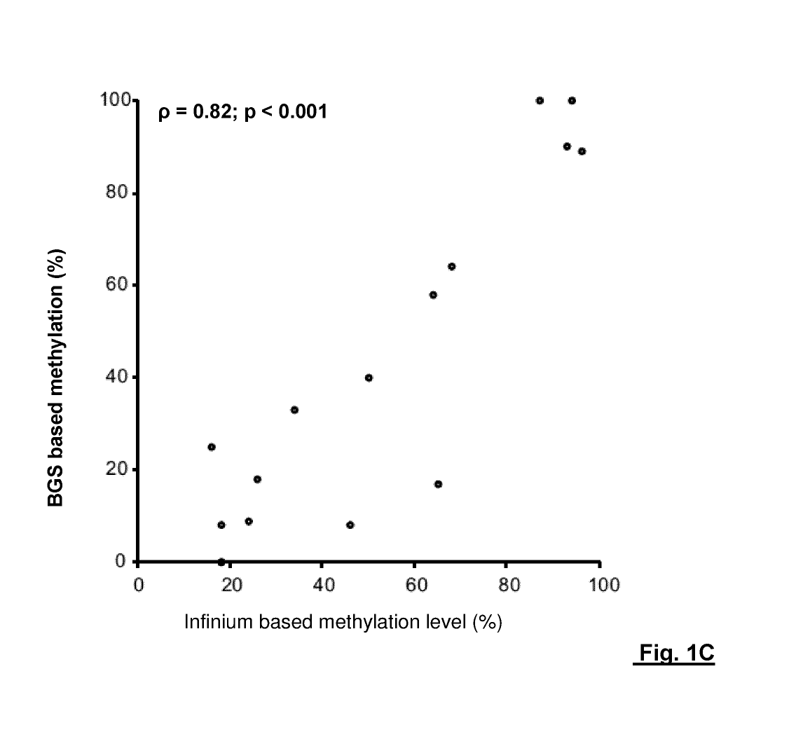

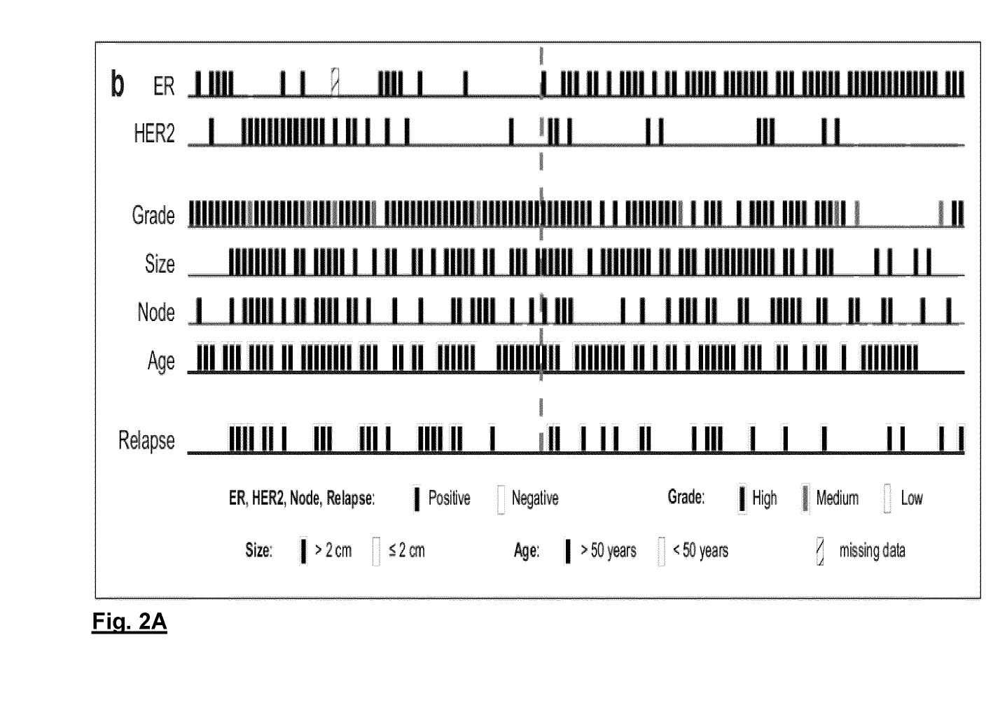

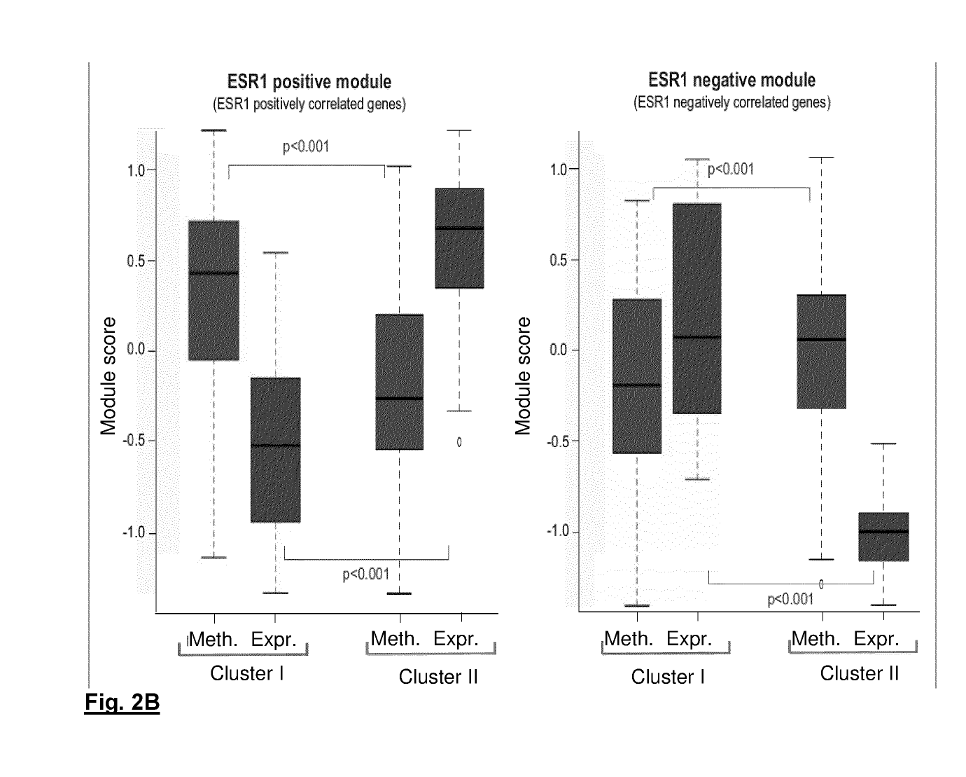

A “main set” of 123 samples (4 normal and 119 infiltrating ductal carcinomas, IDCs), and a “validation set” of 125 samples (8 normal and 117 IDCs) (FIG. 1a; see Supplementary Tables S1, S2 and S15) were analysed using the Infinium® methylation platform. The high-throughput Infinium technique, based on hybridization of bisulphite-converted gDNA on methylation-specific DNA oligomers, allows quantification of methylation levels at 27,578 CpG sites located within the promoter regions (and preferentially within CpG islands) of 14,475 consensus coding sequences and well-known cancer genes (Bibikova, M. et al. 2009 Epigenomics 1, 177-200).

When applied to the main set of breast tissues, this method revealed 6,309 CpGs showing differential methylation between normal samples and IDCs. Validation of these data is depicted in Table 5 and FIG. 1b-c. In terms of CpG location with respect to CpG islands (CGI), we found the hypermethylated CpGs to be mostly located inside CGI, whereas the hypomethylated CpGs were located principally outside of CGI (FIG. 1a, left part). More than a fourth of the CpG island shores presented on the array displayed differential methylation between normal samples and IDCs, suggesting an important role of differential methylation of CpG island shores in cancer, consistently with earlier work Irizarry, (R. A. et al., 2009 Nat. Genet. 41, 178-186). Further, besides the well-described differential methylation of High-CpG-density promoters (HCPs)1, we found even more pronounced methylation changes at Intermediate- and Low- CpG-density promoters (ICPs and LCPs, respectively) (FIG. 1a, right part). Notably, ICPs (also called weak HCPs) seem to be highly susceptible to de novo DNA methylation (FIG. 1a, right part), in agreement with previous studies (Weber, M. et al., 2007 Nat. Genet. 39, 457-466).

TABLE 5
Methylation frequencies of representative CpGs provided by this
Infinium study and their correlation with previously reported data.

| | | | | | Reported | Correlation |
|---|---|---|---|---|---|---|
| | | Strand | | Infinium | methylation | Infinium |
| | | analysed | | methylation | data frequency, | vs. reported |
| | Illumina | by | Coding | frequency, % | % (number); | methylation |
| Gene | ID | Infinium | strand | (number)^{Δ} | technique° | data* |
| RASSF1A | cg00777121 | Top | Bottom | 71 (85/119) | 70 (19/27); MSP⁴² | ++ |
| | | | | | 56 (14/25); MSP⁴³ | ++ |
| | | | | | 58 (52/90); MSP⁸ | ++ |
| | cg08047457 | Top | Bottom | 72 (86/119) | 65 (11/17); MSP⁴⁴ | ++ |
| | cg21554552 | Bottom | Bottom | 70 (83/119) | 65 (11/17); MSP⁴⁴ | ++ |
| CCND2 | cg25425078 | Bottom | Top | 9 (11/119) | 46 (49/106); MSP⁴⁵ | + |
| | | | | | 28 (10/36); MSP⁴⁶ | + |
| | | | | | 55 (71/130); MSP [symbol] | + |
| APC | cg16970232 | Top | Top | 39 (46/119) | 45 (19/42); MSP⁴ [symbol] | ++ |
| | | | | | 28 (15/54); MSP⁴⁸ | ++ |
| | | | | | 39 (51/130); MSP⁷ | ++ |
| | | | | | 49(74/151) MSP⁴⁹ | ++ |
| | cg20311501 | Bottom | Top | 35 (42/119) | 45 (19/42); MSP⁴⁷ | ++ |
| | | | | | 28 (15/54); MSP⁴⁸ | ++ |
| | | | | | 39 (51/130); MSP [symbol] | ++ |
| | | | | | 49 (74/151); MSP⁴⁹ | ++ |
| RARβ2 | cg27486427 | Top | Top | 12 (14/119) | 17 (15/90); BPS⁸ | ++ |
| | | | | | 0 (0/21); BPS⁵⁰ | + |
| | cg26124016 | Bottom | Top | 4 (5/119) | 23 (37/160); MSP⁵¹ | + |
| CDH13 | cg08747377 | Top | Top | 17 (20/119) | 33 (18/55); MSP⁵² | ++ |
| SDHB | cg24305835 | Top | Bottom | 0 (0/119) | 0 (0/72); MS−HRM⁵³ | ++ |
| | cg03861428 | Bottom | Bottom | 0 (0/119) | 0 (0/72); MS−HRM⁵³ | ++ |
| FH | cg06806184 | Top | Bottom | 0 (0/119) | 0 (0/72); MS−HRM⁵³ | ++ |

^{Δ}Each tumour identified as positive shows at least 20% hypermethylation of the indicated CpG site as compared to the mean methylation level of normal samples.
°For MSP data, to avoid any discrepancy due to a different location of PCR primers and of the CpG investigated by the Infinium technique, we selected only CpGs included in the primer sequences used for the MSP analyses.
*Based on the hypothesis that all reference papers check methylation on the coding strand and that methylation is symmetrical between the two strands.
MSP: Methylation-Specific PCR;
BPS: Bisulphite PyrosSequencing;
MS-HRM: Methylation-Sensitive High Resolution Melting
MSP: Methylation-Specific PCR;
BPS: Bisulphite PyroSequencing;
MS-HRM: Methylation-Sensitive High Resolution Melting [symbol]
indicates data missing or illegible when filed

### Example 2

### Establishing DNA Methylation Profiles That Might Have Biological and Clinical Relevance

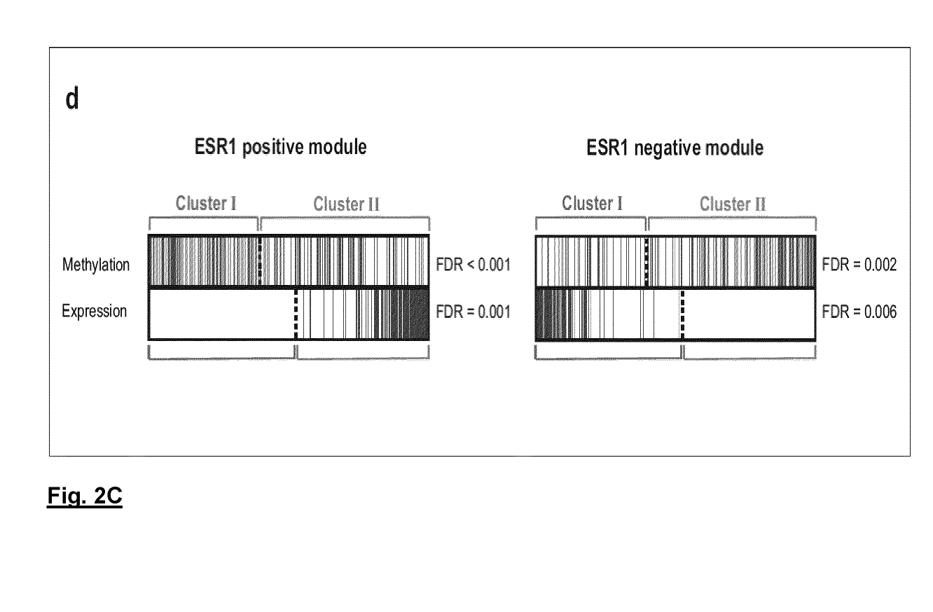

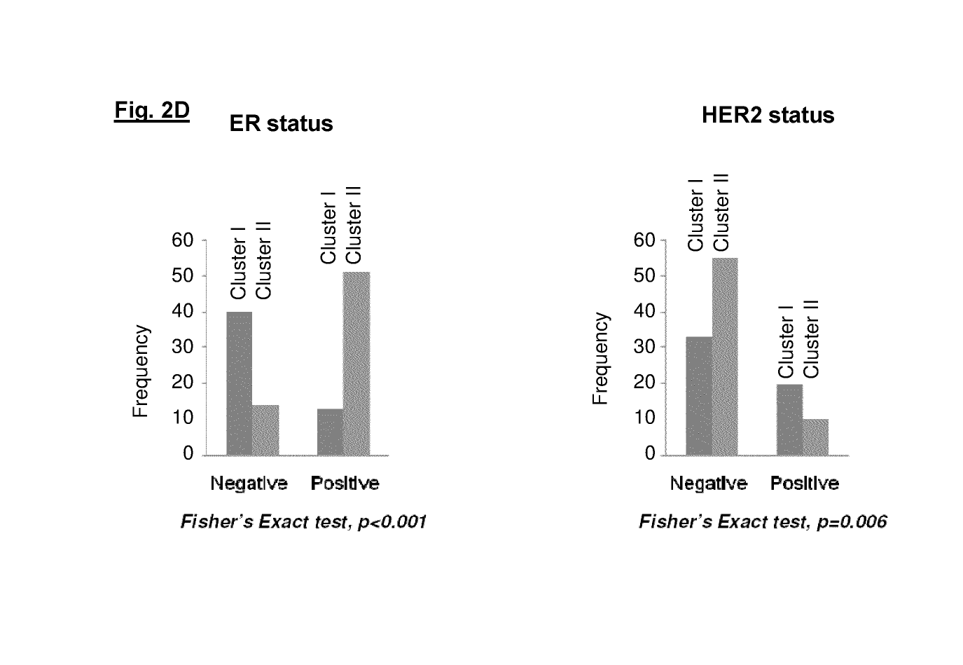

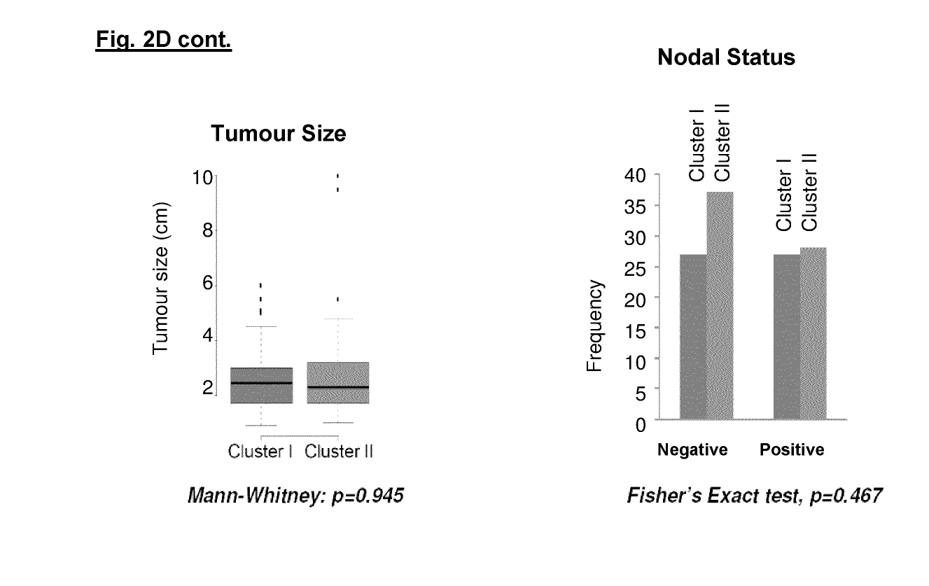

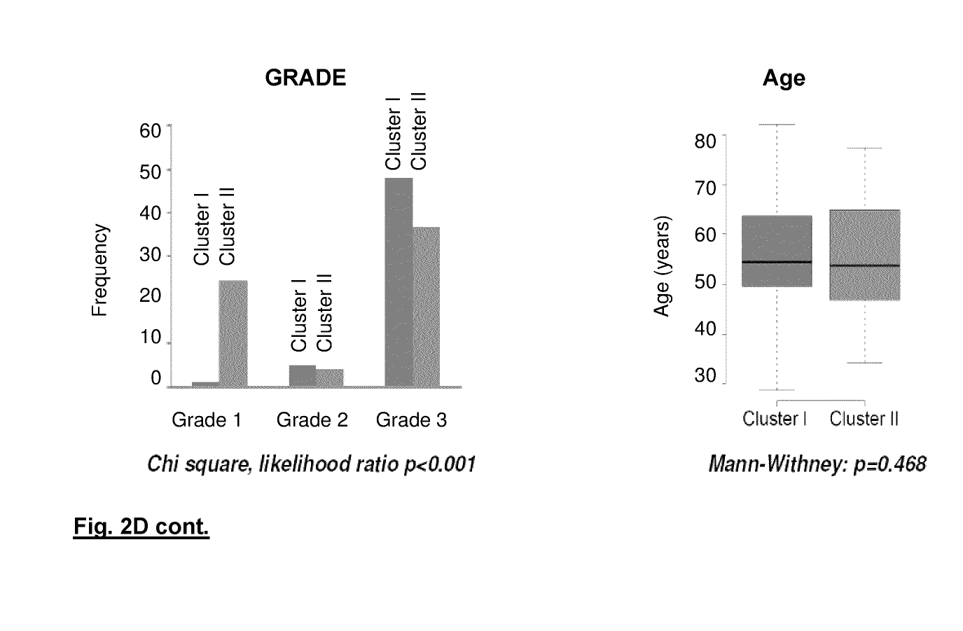

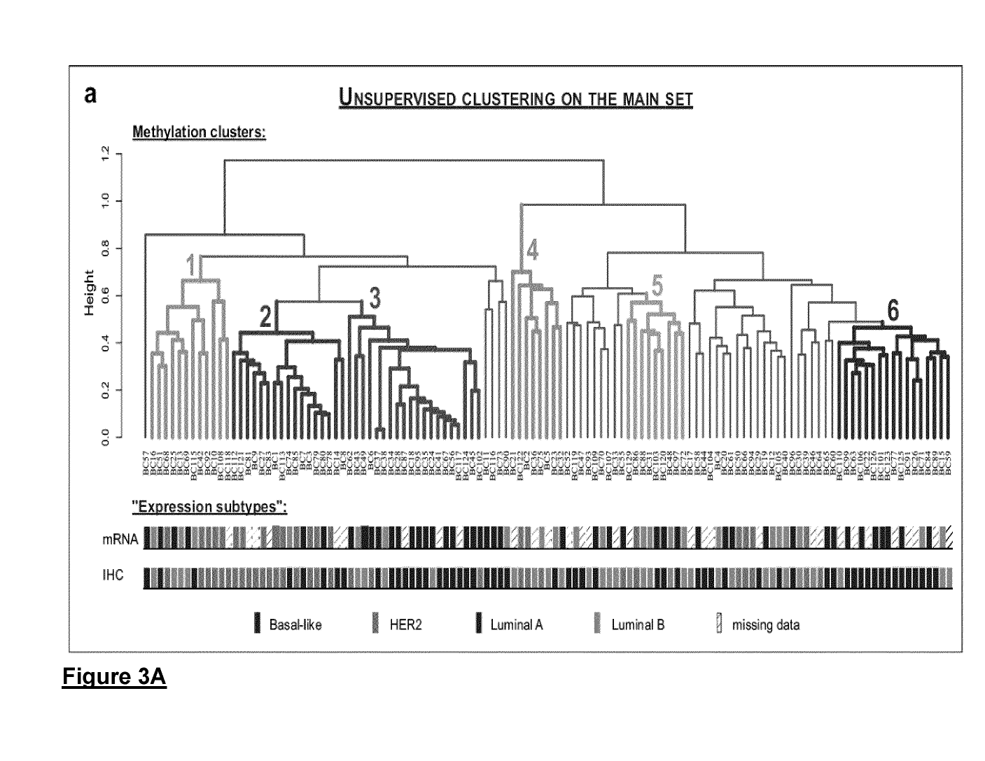

An unsupervised hierarchical cluster analysis was performed of the 119 IDCs of the main set, using a reduced list of CpGs showing differential methylation between normal samples and IDCs (2,985 of them). There emerged two major clusters (I and II), with a significant correlation between cluster membership and both tumour grade and oestrogen receptor (ER) status (FIG. 2). Clusters I and II were enriched in ER-negative and ER-positive tumours, respectively. Importantly, gene expression studies have revealed that clinical biomarkers like ER and HER2 are just the tip of the iceberg, reflecting whole sets of tumour features not obviously related to the marker status. This reality can be captured with gene co-expression modules, i.e. comprehensive lists of genes connected to different biological processes and showing highly correlated expression. One of the most discriminating co-expression modules is the ESR1 module (Desmedt, C. et al., 2008 Clin. Cancer Res. 14, 5158-5165). It comprises ERpathway genes but also genes involved in other biological processes distinguishing ERpositive from ER-negative tumours. We therefore next examined to what extent ESR1 genes might be regulated at the epigenetic level. We divided the previously described ESR1 module in two sub-modules, an “ESR1-positive” and an “ESR1-negative” module comprising, respectively, the genes whose expression correlates positively or negatively with that of ESR1 (cf. Tables 5b and 5c). As shown in box plots and barcode plots derived from Gene Set Enrichment Analysis, ESR1-positive-module genes showed higher methylation levels in cluster I than in cluster II (Mann-Whitney test: p<0.001; see FIG. 2c,d). Conversely, ESR1-negative-module genes showed significantly higher methylation levels in cluster II than in cluster I (Mann-Whitney test: p<0.001; see FIG. 2b,c). Gene expression microarray analysis revealed a significant anti-correlation between the DNA methylation levels of these genes and their corresponding gene expression levels (FIG. 2b,c). Overall, the above results are striking: they suggest, for the first time, that whole sets of genes, involved in processes far beyond ER biology and whose expression status distinguishes ER-positive from ER-negative tumours, are epigenetically regulated. In FIG. 2d, the clinical parameters were linked to the methylation-based clustering identified above, showing that ERpositive tumours were predominant in cluster II, whereas cluster I seemed to contain a moderately higher number of HER2-positive tumours. Grade 1 tumours were grouped in cluster II. No significant association with tumour size, nodal status, or age was found.

TABLE 5B
CpG islands of the ESR1-positive module:

| SEQ | Entrez | | | | | | | |
|---|---|---|---|---|---|---|---|---|
| ID | Gene | | | | | Methylation | Expression | |
| No. | ID | SYMBOL | Affy_ID | coefficient | Illumina_ID | Enrichment | Enrichment | |
| 87 | 60481 | ELOVL5 | 208788_at | 0.58255236 | cg00024396 | | Cluster II | |
| 88 | 55163 | PNPO | 218511_s_at | 0.25550698 | cg00177698 | | Cluster II | |
| 89 | 1389 | CREBL2 | 201990_s_at | 0.46886638 | cg00261552 | | | |
| 90 | 5193 | PEX12 | 205094_at | 0.46553499 | cg00425792 | | Cluster II | |
| 91 | 2013 | EMP2 | 204975_at | 0.42107786 | cg00451635 | Cluster I | Cluster II | |
| 92 | 7764 | ZNF217 | 203739_at | 0.27600069 | cg00476577 | | | |
| 93 | 79921 | TCEAL4 | 202371_at | 0.54197015 | cg00662775 | | Cluster II | |
| 94 | 26504 | CNNM4 | 218900_at | 0.29928358 | cg00711916 | | Cluster II | |
| 95 | 21 | ABCA3 | 204343_at | 0.47676852 | cg00949442 | | Cluster II | |
| 96 | 57758 | SCUBE2 | 219197_s_at | 0.70630729 | cg01081263 | Cluster I | Cluster II | |
| 97 | 6834 | SURF1 | 204295_at | 0.36049855 | cg01309153 | | | |
| 98 | 51181 | DCXR | 217973_at | 0.29980425 | cg01350700 | | Cluster II | |
| 99 | 55224 | ETNK2 | 219268_at | 0.40059475 | cg01566404 | | Cluster II | |
| 100 | 4682 | NUBP1 | 203978_at | 0.24451989 | cg01808090 | | | |
| 101 | 5241 | PGR | 208305_at | 0.5079683 | cg01987509 | | Cluster II | |
| 102 | 4255 | MGMT | 204880_at | 0.30601436 | cg02330106 | | | |
| 103 | 214 | ALCAM | 201951_at | 0.3571957 | cg02582608 | | Cluster II | |
| 104 | 7031 | TFF1 | 205009_at | 0.6449711 | cg02643667 | Cluster I | Cluster II | |
| 105 | 9501 | RPH3AL | 221614_s_at | 0.48934572 | cg02671171 | | Cluster II | |
| 106 | 6019 | RLN2 | 214519_s_at | 0.34013126 | cg02875297 | | Cluster II | |
| 107 | 10307 | APBB3 | 204650_s_at | 0.3461012 | cg02995853 | | Cluster II | |
| 108 | 51368 | TEX264 | 218548_x_at | 0.43540945 | cg03019000 | Cluster I | Cluster II | |
| 109 | 3169 | FOXA1 | 204667_at | 0.74774031 | cg03026462 | Cluster I | Cluster II | |
| 110 | 64080 | RBKS | 57540_at | 0.50109894 | cg03177025 | | Cluster II | |
| 111 | 10267 | RAMP1 | 204916_at | 0.33122019 | cg03270167 | | Cluster II | |
| 112 | 60686 | C14orf93 | 219009_at | 0.24607044 | cg03565081 | | Cluster II | |
| 113 | 5191 | PEX7 | 205420_at | 0.3969911 | cg03807235 | | | |
| 114 | 582 | BBS1 | 218471_s_at | 0.60797534 | cg03851112 | | Cluster II | |
| 115 | 54847 | SIDT1 | 219734_at | 0.45717531 | cg03977782 | | Cluster II | |
| 116 | 126353 | C19orf21 | 212925_at | 0.4486083 | cg04245402 | | Cluster II | |
| 117 | 9633 | MTL5 | 219786_at | 0.56176337 | cg04438497 | | Cluster II | |
| 118 | 11122 | PTPRT | 205948_at | 0.44195895 | cg04541293 | | Cluster II | |
| 119 | 50865 | HEBP1 | 218450_at | 0.44656123 | cg04588079 | Cluster I | Cluster II | |
| 120 | 753 | C18orf1 | 207996_s_at | 0.42386263 | cg04633384 | | Cluster II | |
| 121 | 10614 | HEXIM1 | 202815_s_at | 0.5516074 | cg04700814 | Cluster I | Cluster II | |
| 122 | 7033 | TFF3 | 204623_at | 0.61621987 | cg04806409 | | Cluster II | |
| 123 | 8187 | ZNF239 | 206261_at | 0.27306458 | cg04825431 | | | |
| 124 | 771 | CA12 | 204508_s_at | 0.76966447 | cg04826883 | | Cluster II | |
| 125 | 51207 | DUSP13 | 219963_at | 0.29595767 | cg04834572 | | Cluster II | |
| 126 | 55188 | RIC8B | 219446_at | 0.34248633 | cg04916200 | | Cluster II | |
| 127 | 22885 | ABLIM3 | 205730_s_at | 0.44622382 | cg05026186 | | Cluster II | |
| 128 | 81563 | C1orf21 | 221272_s_at | 0.48956231 | cg05135156 | | Cluster II | |
| 129 | 10265 | IRX5 | 210239_at | 0.44423877 | cg05266781 | Cluster I | Cluster II | |
| 130 | 79603 | LASS4 | 218922_s_at | 0.44467496 | cg05346899 | | Cluster II | |
| 131 | 79885 | HDAC11 | 219847_at | 0.50364052 | cg05446471 | Cluster I | | |
| 132 | 11226 | GALNT6 | 219956_at | 0.3952831 | cg05565537 | | Cluster II | |
| 133 | 79669 | C3orf52 | 219474_at | 0.38844228 | cg05570980 | | Cluster II | |
| 134 | 10519 | CIB1 | 201953_at | 0.31818779 | cg05641961 | | | |
| 135 | 23171 | GPD1L | 212510_at | 0.54491467 | cg05662500 | | Cluster II | |
| 136 | 819 | CAMLG | 203538_at | 0.47069771 | cg05705583 | | Cluster II | |
| 137 | 1632 | DCI | 209759_s_at | 0.5213171 | cg05824432 | | Cluster II | |
| 138 | 10079 | ATP9A | 212062_at | 0.32828286 | cg05851042 | | | |
| 139 | 23107 | MRPS27 | 212145_at | 0.40636664 | cg05903630 | | Cluster II | |
| 140 | 12 | SERPINA3 | 202376_at | 0.43012865 | cg06190732 | | Cluster II | |
| 141 | 2625 | GATA3 | 209602_s_at | 0.80840445 | cg06230736 | | Cluster II | |
| 142 | 8405 | SPOP | 208927_at | 0.27075407 | cg06291334 | | | |
| 143 | 6652 | SORD | 201563_at | 0.3946522 | cg06424894 | | Cluster II | |
| 144 | 55793 | FAM63A | 221856_s_at | 0.58660889 | cg06433658 | Cluster I | | |
| 145 | 9052 | GPRC5A | 203108_at | 0.34643392 | cg06776256 | Cluster I | Cluster II | |
| 146 | 8722 | CTSF | 203657_s_at | 0.43611 | cg06817264 | | Cluster II | |
| 147 | 5269 | SERPINB6 | 211474_s_at | 0.46113414 | cg06945625 | | Cluster II | |
| 148 | 1101 | CHAD | 206869_at | 0.5267707 | cg06958829 | Cluster I | Cluster II | |
| 149 | 2066 | ERBB4 | 214053_at | 0.70552413 | cg07015629 | | Cluster II | |
| 150 | 51306 | C5orf5 | 218518_at | 0.5288126 | cg07048066 | | Cluster II | |
| 151 | 25915 | C3orf60 | 209177_at | 0.27572801 | cg07109801 | | Cluster II | |
| 152 | 7138 | TNNT1 | 213201_s_at | 0.33161148 | cg07189381 | | Cluster II | |
| 153 | 51604 | PIGT | 217770_at | 0.51423124 | cg07294870 | | Cluster II | |
| 154 | 8416 | ANXA9 | 210085_s_at | 0.6000835 | cg07337598 | Cluster I | Cluster II | |
| 155 | 55218 | EXDL2 | 218363_at | 0.40149833 | cg07366967 | | Cluster II | |
| 156 | 22977 | AKR7A3 | 206469_x_at | 0.49969396 | cg07447773 | Cluster I | Cluster II | |
| 157 | 10002 | NR2E3 | 208388_at | 0.40777521 | cg07890954 | | Cluster II | |
| 158 | 89927 | C16orf45 | 212736_at | 0.49149582 | cg07977490 | | Cluster II | |
| 159 | 54820 | NDE1 | 218414_s_at | 0.28208014 | cg08081725 | Cluster I | | |
| 160 | 8310 | ACOX3 | 204242_s_at | 0.2875821 | cg08083689 | | Cluster II | |
| 161 | 6787 | NEK4 | 204634_at | 0.43835459 | cg08090396 | | Cluster II | |
| 162 | 55450 | CAMK2N1 | 218309_at | 0.37066024 | cg08398233 | Cluster I | Cluster II | |
| 163 | 10309 | UNG2 | 210021_s_at | 0.34040691 | cg08514736 | | Cluster II | |
| 164 | 55733 | HHAT | 219687_at | 0.57829406 | cg09276883 | | Cluster II | |
| 165 | 25790 | CCDC19 | 220308_at | 0.2863511 | cg09451092 | Cluster I | | |
| 166 | 3295 | HSD17B4 | 201413_at | 0.49793269 | cg09486093 | | Cluster II | |
| 167 | 5016 | OVGP1 | 205432_at | 0.34020467 | cg09558502 | | | |
| 168 | 1877 | E4F1 | 218524_at | 0.40033795 | cg09615982 | | | |
| 169 | 5816 | PVALB | 205336_at | 0.22735879 | cg09863066 | | Cluster II | |
| 170 | 5825 | ABCD3 | 202850_at | 0.47855837 | cg09869791 | | Cluster II | |
| 171 | 3667 | IRS1 | 204686_at | 0.57148821 | cg10098888 | Cluster I | Cluster II | |
| 172 | 2530 | FUT8 | 203988_s_at | 0.50553001 | cg10225525 | | Cluster II | |
| 173 | 7993 | UBXD6 | 215983_s_at | 0.38287893 | cg10301990 | | Cluster II | |
| 174 | 5174 | PDZK1 | 205380_at | 0.54605106 | cg10321723 | Cluster I | Cluster II | |
| 175 | 1501 | CTNND2 | 209618_at | 0.27327605 | cg10331779 | Cluster I | Cluster II | |
| 176 | 3622 | ING2 | 205981_s_at | 0.29062248 | cg10348863 | | Cluster II | |
| 177 | 6926 | TBX3 | 219682_s_at | 0.4677582 | cg10530281 | | Cluster II | |
| 178 | 54903 | MKS1 | 218630_at | 0.24804067 | cg10728503 | | | |
| 179 | 51004 | COQ6 | 218760_at | 0.40443291 | cg10784821 | | Cluster II | |
| 180 | 79170 | ATAD4 | 219127_at | 0.37327143 | cg10878307 | Cluster I | Cluster II | |
| 181 | 2954 | GSTZ1 | 209531_at | 0.33474043 | cg11193041 | | Cluster II | |
| 182 | 4602 | MYB | 204798_at | 0.72436025 | cg11579069 | | Cluster II | |
| 183 | 23158 | TBC1D9 | 212956_at | 0.81885393 | cg11843691 | | Cluster II | |
| 184 | 9120 | SLC16A6 | 207038_at | 0.54887717 | cg11879514 | | Cluster II | |
| 185 | 9674 | KIAA0040 | 203143_s_at | 0.53208827 | cg11908570 | | Cluster II | |
| 186 | 23245 | ASTN2 | 215407_s_at | 0.43227295 | cg12024292 | | Cluster II | |
| 187 | 5327 | PLAT | 201860_s_at | 0.44627615 | cg12091331 | Cluster I | Cluster II | |
| 188 | 1345 | COX6C | 201754_at | 0.53994131 | cg12125691 | | Cluster II | |
| 189 | 56521 | DNAJC12 | 218976_at | 0.65414762 | cg12315959 | | Cluster II | |
| 190 | 2813 | GP2 | 214324_at | 0.3462389 | cg12554476 | Cluster I | Cluster II | |
| 191 | 5783 | PTPN13 | 204201_s_at | 0.39210976 | cg12647643 | | Cluster II | |
| 192 | 7286 | TUFT1 | 205807_s_at | 0.32428768 | cg12729048 | | Cluster II | |
| 193 | 4485 | MST1 | 205614_x_at | 0.35745042 | cg12788313 | | Cluster II | |
| 194 | 55650 | PIGV | 51146_at | 0.42058252 | cg12806381 | | Cluster II | |
| 195 | 79818 | ZNF552 | 219741_x_at | 0.61082014 | cg12983442 | | Cluster II | |
| 196 | 6833 | ABCC8 | 210246_s_at | 0.43299799 | cg13185308 | | Cluster II | |
| 197 | 4036 | LRP2 | 205710_at | 0.35025477 | cg13436799 | | Cluster II | |
| 198 | 55699 | IARS2 | 217900_at | 0.23087069 | cg13530946 | | | |
| 199 | 54898 | ELOVL2 | 213712_at | 0.52925655 | cg13562911 | | Cluster II | |
| 200 | 427 | ASAH1 | 210980_s_at | 0.47414718 | cg13563405 | | Cluster II | |
| 201 | 347902 | AMIGO2 | 222108_at | 0.36104055 | cg13640200 | | Cluster II | |
| 202 | 23613 | PRKCBP1 | 209049_s_at | 0.29980727 | cg13699808 | | Cluster II | |
| 203 | 8309 | ACOX2 | 205364_at | 0.4083166 | cg13705284 | Cluster I | Cluster II | |
| 204 | 8382 | NMES | 206197_at | 0.55521067 | cg13707560 | Cluster I | Cluster II | |
| 205 | 863 | CBFA2T3 | 208056_s_at | 0.34439279 | cg13745346 | | Cluster II | |
| 206 | 64087 | MCCC2 | 209624_s_at | 0.46285733 | cg13793354 | | Cluster II | |
| 207 | 323 | APBB2 | 213419_at | 0.5072429 | cg13842258 | | Cluster II | |
| 208 | 25823 | TPSG1 | 220339_s_at | 0.37387841 | cg13997068 | | Cluster II | |
| 209 | 56674 | TMEM9B | 218065_s_at | 0.52812741 | cg14205126 | | Cluster II | |
| 210 | 29116 | MYLIP | 220319_s_at | 0.37379359 | cg14298379 | | Cluster II | |
| 211 | 23541 | SEC14L2 | 204541_at | 0.44986387 | cg14452140 | Cluster I | Cluster II | |
| 212 | 10140 | TOB1 | 202704_at | 0.36762247 | cg14494812 | Cluster I | | |
| 213 | 64428 | NARFL | 218742_at | 0.20385725 | cg14711016 | | | |
| 214 | 6720 | SREBF1 | 202308_at | 0.41745005 | cg14808739 | | Cluster II | |
| 215 | 79622 | C16orf33 | 218493_at | 0.31308351 | cg14820573 | | Cluster II | |
| 216 | 6548 | SLC9A1 | 209453_at | 0.26654189 | cg15076659 | | | |
| 217 | 51097 | SCCPDH | 201825_s_at | 0.59486345 | cg15210596 | | Cluster II | |
| 218 | 2099 | ESR1 | 205225_at | 1 | cg15626350 | Cluster I | Cluster II | |
| 219 | 64215 | DNAJC1 | 218409_s_at | 0.30939108 | cg15818800 | | Cluster II | |
| 220 | 4350 | MPG | 203686_at | 0.34167694 | cg16003913 | | Cluster II | |
| 221 | 25980 | C20orf4 | 218089_at | 0.20311663 | cg16016641 | | Cluster II | |
| 222 | 79602 | ADIPOR2 | 201346_at | 0.29463646 | cg16245844 | | Cluster II | |
| 223 | 3306 | HSPA2 | 211538_s_at | 0.3956746 | cg16319578 | | Cluster II | |
| 224 | 23552 | CCRK | 205271_s_at | 0.28188064 | cg16386080 | | | |
| 225 | 55316 | RSAD1 | 218307_at | 0.3299015 | cg16413777 | | | |
| 226 | 5002 | SLC22A18 | 204981_at | 0.498451 | cg16873863 | | Cluster II | |
| 227 | 9518 | GDF15 | 221577_x_at | 0.40270729 | cg16929104 | Cluster I | Cluster II | |
| 228 | 5104 | SERPINA5 | 209443_at | 0.55261579 | cg16937611 | | Cluster II | |
| 229 | 8870 | IER3 | 201631_s_at | 0.29324048 | cg17067528 | | | |
| 230 | 9722 | NOS1AP | 215153_at | 0.22934089 | cg17096191 | | Cluster II | |
| 231 | 83464 | APH1B | 221036_s_at | 0.38272656 | cg17207590 | Cluster I | | |
| 232 | 10273 | STUB1 | 217934_x_at | 0.41337688 | cg17328659 | | | |
| 233 | 58495 | OVOL2 | 211778_s_at | 0.50985425 | cg17404915 | Cluster I | Cluster II | |
| 234 | 4285 | MIPEP | 36830_at | 0.35646337 | cg17436805 | | Cluster II | |
| 235 | 9851 | KIAA0753 | 204711_at | 0.33776741 | cg17452257 | | | |
| 236 | 2737 | GLI3 | 205201_at | 0.52149467 | cg17530977 | | Cluster II | |
| 237 | 81539 | SLC38A1 | 218237_s_at | 0.2417025 | cg17726022 | | | |
| 238 | 629 | CFB | 202357_s_at | 0.32594788 | cg17741572 | Cluster I | Cluster II | |
| 239 | 27239 | GPR162 | 205056_s_at | 0.26732712 | cg17805404 | | | |
| 240 | 2203 | FBP1 | 209696_at | 0.66601785 | cg17814481 | Cluster I | Cluster II | |
| 241 | 23528 | ZNF281 | 218401_s_at | 0.37912728 | cg17918239 | | Cluster II | |
| 242 | 1153 | CIRBP | 200810_s_at | 0.64437699 | cg18194038 | | Cluster II | |
| 243 | 51706 | CYB5R1 | 202263_at | 0.48001447 | cg18275051 | | Cluster II | |
| 244 | 25864 | ABHD14A | 210006_at | 0.4312276 | cg18328933 | Cluster I | Cluster II | |
| 245 | 2743 | GLRB | 205280_at | 0.48052565 | cg18344745 | Cluster I | Cluster II | |
| 246 | 7163 | TPD52 | 201691_s_at | 0.26346165 | cg18459342 | | | |
| 247 | 4435 | CITED1 | 207144_s_at | 0.37530465 | cg18468467 | | Cluster II | |
| 248 | 51466 | EVL | 217838_s_at | 0.65340496 | cg18621299 | | Cluster II | |
| 249 | 51103 | NDUFAF1 | 204125_at | 0.35312245 | cg18705301 | | Cluster II | |
| 250 | 23303 | KIF13B | 202962_at | 0.5418989 | cg18875839 | | Cluster II | |
| 251 | 8537 | BCAS1 | 204378_at | 0.47126093 | cg18917378 | Cluster I | Cluster II | |
| 252 | 7494 | XBP1 | 200670_at | 0.70660634 | cg18940763 | Cluster I | Cluster II | |
| 253 | 11094 | C9orf7 | 219223_at | 0.43895474 | cg19123107 | | Cluster II | |
| 254 | 283232 | TMEM80 | 221951_at | 0.33473355 | cg19515518 | Cluster I | Cluster II | |
| 255 | 1733 | DIO1 | 206457_s_at | 0.27714605 | cg19526600 | | Cluster II | |
| 256 | 10202 | DHRS2 | 214079_at | 0.39469825 | cg19538485 | | Cluster II | |
| 257 | 55663 | ZNF446 | 219900_s_at | 0.50264354 | cg19649173 | | Cluster II | |
| 258 | 123872 | LRRC50 | 222068_s_at | 0.42313282 | cg19706682 | | Cluster II | |
| 259 | 1555 | CYP2B6 | 206754_s_at | 0.63122768 | cg19756068 | | | |
| 260 | 7905 | REEP5 | 208873_s_at | 0.52513099 | cg19863003 | | | |
| 261 | 6697 | SPR | 203458_at | 0.37404256 | cg19889780 | Cluster I | Cluster II | |
| 262 | 10421 | CD2BP2 | 202257_s_at | 0.43847209 | cg19981839 | | | |
| 263 | 185 | AGTR1 | 205357_s_at | 0.44871963 | cg20530314 | Cluster I | Cluster II | |
| 264 | 18 | ABAT | 209459_s_at | 0.68431164 | cg20587543 | Cluster I | Cluster II | |
| 265 | 23635 | SSBP2 | 203787_at | 0.26127225 | cg20757912 | | Cluster II | |
| 266 | 987 | LRBA | 212692_s_at | 0.66720446 | cg20850582 | | Cluster II | |
| 267 | 9185 | REPS2 | 205645_at | 0.44296576 | cg20855303 | | Cluster II | |
| 268 | 27165 | GLS2 | 205531_s_at | 0.25483734 | cg20877313 | Cluster I | Cluster II | |
| 269 | 51364 | ZMYND10 | 205714_s_at | 0.46588534 | cg20881888 | | Cluster II | |
| 270 | 10551 | AGR2 | 209173_at | 0.68249398 | cg21201572 | Cluster I | Cluster II | |
| 271 | 9 | NAT1 | 214440_at | 0.68994857 | cg21363706 | Cluster I | Cluster II | |
| 272 | 7802 | DNALI1 | 205186_at | 0.72206464 | cg21488617 | Cluster I | Cluster II | |
| 273 | 55859 | BEX1 | 218332_at | 0.31558982 | cg21509846 | | Cluster II | |
| 274 | 9368 | SLC9A3R1 | 201349_at | 0.4058525 | cg21922841 | Cluster I | Cluster II | |
| 275 | 3572 | IL6ST | 204863_s_at | 0.56616896 | cg21950518 | | Cluster II | |
| 276 | 10827 | C5orf3 | 218588_s_at | 0.42777389 | cg22230395 | | Cluster II | |
| 277 | 54961 | SSH3 | 219919_s_at | 0.58016018 | cg22285621 | Cluster I | Cluster II | |
| 278 | 1917 | EEF1A2 | 204540_at | 0.430875 | cg22463915 | | Cluster II | |
| 279 | 112398 | EGLN2 | 220956_s_at | 0.39209521 | cg22671726 | | Cluster II | |
| 280 | 11098 | PRSS23 | 202458_at | 0.40863082 | cg23214764 | | Cluster II | |
| 281 | 51161 | C3orf18 | 219114_at | 0.55310088 | cg23320649 | | Cluster II | |
| 282 | 10127 | ZNF263 | 203707_at | 0.45998317 | cg23412875 | | Cluster II | |
| 283 | 10884 | MRPS30 | 218398_at | 0.47959606 | cg23455614 | | Cluster II | |
| 284 | 55614 | C20orf23 | 219570_at | 0.48672644 | cg23455897 | | Cluster II | |
| 285 | 2947 | GSTM3 | 202554_s_at | 0.47749254 | cg23472215 | | Cluster II | |
| 286 | 2232 | FDXR | 207813_s_at | 0.35785196 | cg23727583 | | Cluster II | |
| 287 | 2674 | GFRA1 | 205696_s_at | 0.58482365 | cg23898073 | Cluster I | Cluster II | |
| 288 | 6666 | SOX12 | 204432_at | 0.2889763 | cg23922081 | | Cluster II | |
| 289 | 9091 | PIGQ | 204144_s_at | 0.44802235 | cg24014020 | Cluster I | Cluster II | |
| 290 | 54880 | BCOR | 219433_at | 0.22960544 | cg24183173 | | Cluster II | |
| 291 | 54970 | TTC12 | 219587_at | 0.2915526 | cg24264506 | | Cluster II | |
| 292 | 2155 | F7 | 207300_s_at | 0.29179115 | cg24269657 | Cluster I | Cluster II | |
| 293 | 5357 | PLS1 | 205190_at | 0.24732622 | cg24278076 | | Cluster II | |
| 294 | 27250 | PDCD4 | 212593_s_at | 0.42229844 | cg24371157 | | Cluster II | |
| 295 | 1960 | EGR3 | 206115_at | 0.37300819 | cg24403722 | | Cluster II | |
| 296 | 2800 | GOLGA1 | 203384_s_at | 0.43241773 | cg24412846 | | | |
| 297 | 786 | CACNG1 | 206612_at | 0.32528848 | cg24459563 | | Cluster II | |
| 298 | 3760 | KCNJ3 | 207142_at | 0.28982426 | cg24693368 | Cluster I | Cluster II | |
| 299 | 54894 | RNF43 | 218704_at | 0.28044127 | cg24835159 | Cluster I | Cluster II | |
| 300 | 55245 | C20orf44 | 217935_s_at | 0.29225728 | cg24906992 | | Cluster II | |
| 301 | 2891 | GRIA2 | 205358_at | 0.32540262 | cg25148589 | | Cluster II | |
| 302 | 1047 | CLGN | 205830_at | 0.36939216 | cg25323711 | | Cluster II | |
| 303 | 11001 | SLC27A2 | 205768_s_at | 0.50448727 | cg25417405 | Cluster I | Cluster II | |
| 304 | 56683 | C21orf59 | 218123_at | 0.30298336 | cg25505974 | | Cluster II | |
| 305 | 1847 | DUSP5 | 209457_at | 0.27703245 | cg25524473 | Cluster I | | |
| 306 | 1718 | DHCR24 | 200862_at | 0.38017698 | cg25536676 | Cluster I | | |
| 307 | 5441 | POLR2L | 202586_at | 0.29070545 | cg25748127 | | Cluster II | |
| 308 | 10406 | WFDC2 | 203892_at | 0.31031891 | cg25799986 | Cluster I | Cluster II | |
| 309 | 80347 | COASY | 201913_s_at | 0.44198549 | cg25831111 | | Cluster II | |
| 310 | 26018 | LRIG1 | 211596_s_at | 0.59172338 | cg26131019 | | Cluster II | |
| 311 | 1360 | CPB1 | 205509_at | 0.34649378 | cg26361780 | | Cluster II | |
| 312 | 5860 | QDPR | 209123_at | 0.46688046 | cg26689483 | | Cluster II | |
| 313 | 55333 | SYNJ2BP | 219156_at | 0.35415298 | cg26709859 | | Cluster II | |
| 314 | 27134 | TJP3 | 213412_at | 0.54277553 | cg27022827 | | Cluster II | |
| 315 | 4488 | MSX2 | 205555_s_at | 0.29546364 | cg27096144 | Cluster I | Cluster II | |
| 316 | 25837 | RAB26 | 219562_at | 0.52616496 | cg27176536 | | Cluster II | |
| 317 | 10040 | TOM1L1 | 204485_s_at | 0.38262454 | cg27210390 | Cluster I | Cluster II | |
| 318 | 27124 | PIB5PA | 213651_at | 0.49391158 | cg27324619 | Cluster I | Cluster II | |
| 319 | 6583 | SLC22A4 | 205896_at | 0.32318426 | cg27372468 | | Cluster II | |
| 320 | 3315 | HSPB1 | 201841_s_at | 0.40616865 | cg27376817 | | Cluster II | |
| 321 | 51809 | GALNT7 | 218313_s_at | 0.49150358 | cg27433088 | | Cluster II | |
| | 57496 | MKL2 | 218259_at | 0.64903192 | NA | | Cluster II | |
| | 55638 | NA | 218692_at | 0.62980086 | NA | | Cluster II | |
| | 54463 | NA | 218532_s_at | 0.60166971 | NA | | Cluster II | |
| | 54502 | NA | 218035_s_at | 0.59729022 | NA | | Cluster II | |
| | 57613 | KIAA1467 | 213234_at | 0.59084268 | NA | | Cluster II | |
| | 55686 | MREG | 219648_at | 0.57186844 | NA | | Cluster II | |
| | 23324 | MAN2B2 | 214703_s_at | 0.55505861 | NA | | Cluster II | |
| | 8100 | IFT88 | 204703_at | 0.55028445 | NA | | Cluster II | |
| | 79641 | ROGDI | 218394_at | 0.54629249 | NA | | Cluster II | |
| | 400451 | NA | 51158_at | 0.53742018 | NA | | Cluster II | |
| | 28958 | CCDC56 | 218026_at | 0.52364146 | NA | | Cluster II | |
| | 122616 | C14orf79 | 213512_at | 0.50858013 | NA | | Cluster II | |
| | 23327 | NEDD4L | 212448_at | 0.50237131 | NA | | | |
| | 7568 | ZNF20 | 213916_at | 0.47419152 | NA | | Cluster II | |
| | 54812 | AFTPH | 217939_s_at | 0.45517045 | NA | | Cluster II | |
| | 8399 | PLA2G10 | 207222_at | 0.44184663 | NA | | Cluster II | |
| | 399665 | FAM102A | 212400_at | 0.4260898 | NA | | Cluster II | |
| | 80223 | RAB11FIP1 | 219681_s_at | 0.40904171 | NA | | Cluster II | |
| | 92104 | TTC30A | 213679_at | 0.40345151 | NA | | Cluster II | |
| | 79629 | OCEL1 | 205441_at | 0.40233192 | NA | | Cluster II | |
| | 55184 | C20orf12 | 219951_s_at | 0.39674387 | NA | | Cluster II | |
| | 54458 | PRR13 | 217794_at | 0.39227943 | NA | | | |
| | 11042 | NA | 215043_s_at | 0.38838153 | NA | | Cluster II | |
| | 374 | AREG | 205239_at | 0.37561015 | NA | | | |
| | 79719 | NA | 202851_at | 0.36402063 | NA | | Cluster II | |
| | 55258 | NA | 219044_at | 0.35827387 | NA | | Cluster II | |
| | 55293 | UEVLD | 220775_s_at | 0.34468884 | NA | | Cluster II | |
| | 51735 | RAPGEF6 | 219112_at | 0.32626789 | NA | | | |
| | 22976 | PAXIP1 | 212825_at | 0.3149759 | NA | | | |
| | 23059 | CLUAP1 | 204577_s_at | 0.30808191 | NA | | Cluster II | |
| | 80279 | CDK5RAP3 | 218740_s_at | 0.29508624 | NA | | | |
| | 7769 | ZNF226 | 219603_s_at | 0.29151808 | NA | | Cluster II | |
| | 55101 | NA | 218038_at | 0.26654972 | NA | | Cluster II | |
| | 8987 | NA | 203986_at | 0.24350432 | NA | | Cluster II | |
| | 57586 | SYT13 | 221859_at | 0.23947239 | NA | | Cluster II | |
| | 23366 | NA | 213424_at | 0.23429518 | NA | | Cluster II | |
| | 58513 | EPS15L1 | 221056_x_at | 0.23324627 | NA | | Cluster II | |
| | 29104 | N6AMT1 | 220311_at | 0.22248446 | NA | | Cluster II | |
| | 79446 | WDR25 | 219609_at | 0.2086421 | NA | | Cluster II | |
| SEQ | CpG | | SEQ | CpG | | SEQ | CpG | |
| ID | Island | Promoter | ID | Island | Promoter | ID | Island | Promoter |
| No. | Revisited | Class | No. | Revisited | Class | No. | Revisited | Class |
| 87 | true | HCP | 101 | shore | LCP | 115 | true | HCP |
| 88 | true | HCP | 102 | true | HCP | 116 | true | ICP |
| 89 | true | HCP | 103 | true | HCP | 117 | shore | ICP |
| 90 | true | HCP | 104 | true | ICP | 118 | true | HCP |
| 91 | shore | HCP | 105 | shore | ICP | 119 | true | HCP |
| 92 | shore | LCP | 106 | true | ICP | 120 | true | HCP |
| 93 | true | ICP | 107 | true | HCP | 121 | shore | HCP |
| 94 | true | HCP | 108 | shore | HCP | 122 | shore | ICP |
| 95 | true | HCP | 109 | true | HCP | 123 | false | ICP |
| 96 | true | HCP | 110 | true | HCP | 124 | true | HCP |
| 97 | true | HCP | 111 | true | HCP | 125 | false | ICP |
| 98 | true | HCP | 112 | shore | HCP | 126 | true | HCP |
| 99 | shore | HCP | 113 | true | HCP | 127 | true | HCP |
| 100 | true | HCP | 114 | shore | ICP | 128 | true | HCP |
| 129 | true | HCP | 175 | true | HCP | 221 | true | HCP |
| 130 | true | HCP | 176 | true | HCP | 222 | true | HCP |
| 131 | true | HCP | 177 | true | HCP | 223 | true | |
| 132 | false | ICP | 178 | shore | LCP | 224 | true | HCP |
| 133 | true | HCP | 179 | false | HCP | 225 | true | HCP |
| 134 | true | HCP | 180 | false | ICP | 226 | shore | ICP |
| 135 | true | HCP | 181 | true | HCP | 227 | true | ICP |
| 136 | true | HCP | 182 | true | HCP | 228 | false | ICP |
| 137 | true | HCP | 183 | true | HCP | 229 | true | HCP |
| 138 | shore | HCP | 184 | true | HCP | 230 | true | HCP |
| 139 | true | HCP | 185 | true | HCP | 231 | true | HCP |
| 140 | false | ICP | 186 | true | HCP | 232 | true | HCP |
| 141 | true | HCP | 187 | false | ICP | 233 | true | HCP |
| 142 | true | HCP | 188 | shore | HCP | 234 | true | HCP |
| 143 | shore | HCP | 189 | false | LCP | 235 | shore | HCP |
| 144 | false | ICP | 190 | false | ICP | 236 | true | ICP |
| 145 | true | ICP | 191 | true | HCP | 237 | true | HCP |
| 146 | true | HCP | 192 | true | HCP | 238 | shore | ICP |
| 147 | shore | HCP | 193 | shore | ICP | 239 | shore | ICP |
| 148 | true | HCP | 194 | true | HCP | 240 | shore | HCP |
| 149 | true | HCP | 195 | true | HCP | 241 | shore | HCP |
| 150 | true | HCP | 196 | true | HCP | 242 | true | HCP |
| 151 | true | ICP | 197 | true | HCP | 243 | true | HCP |
| 152 | false | ICP | 198 | shore | HCP | 244 | true | HCP |
| 153 | true | HCP | 199 | true | HCP | 245 | true | HCP |
| 154 | false | ICP | 200 | true | ICP | 246 | true | HCP |
| 155 | false | LCP | 201 | true | HCP | 247 | true | HCP |
| 156 | true | HCP | 202 | shore | ICP | 248 | false | ICP |
| 157 | shore | ICP | 203 | false | ICP | 249 | true | HCP |
| 158 | true | HCP | 204 | true | ICP | 250 | true | HCP |
| 159 | shore | HCP | 205 | true | ICP | 251 | false | ICP |
| 160 | true | HCP | 206 | true | HCP | 252 | true | HCP |
| 161 | true | HCP | 207 | true | HCP | 253 | true | HCP |
| 162 | true | HCP | 208 | false | ICP | 254 | true | HCP |
| 163 | true | HCP | 209 | true | HCP | 255 | true | ICP |
| 164 | true | HCP | 210 | true | HCP | 256 | false | ICP |
| 165 | shore | ICP | 211 | true | ICP | 257 | true | HCP |
| 166 | true | HCP | 212 | false | LCP | 258 | true | HCP |
| 167 | false | ICP | 213 | true | HCP | 259 | false | ICP |
| 168 | true | HCP | 214 | shore | HCP | 260 | true | HCP |
| 169 | shore | ICP | 215 | false | HCP | 261 | true | HCP |
| 170 | true | HCP | 216 | true | HCP | 262 | true | HCP |
| 171 | true | HCP | 217 | true | HCP | 263 | true | HCP |
| 172 | false | LCP | 218 | true | | 264 | true | HCP |
| 173 | true | HCP | 219 | true | HCP | 265 | true | HCP |
| 174 | false | ICP | 220 | shore | ICP | 266 | true | HCP |
| 267 | true | HCP | 287 | true | HCP | 307 | true | HCP |
| 268 | true | HCP | 288 | true | HCP | 308 | true | ICP |
| 269 | true | HCP | 289 | shore | HCP | 309 | shore | ICP |
| 270 | false | ICP | 290 | true | HCP | 310 | true | HCP |
| 271 | false | ICP | 291 | true | ICP | 311 | false | LCP |
| 272 | true | ICP | 292 | shore | ICP | 312 | shore | HCP |
| 273 | true | ICP | 293 | false | LCP | 314 | true | ICP |
| 274 | true | HCP | 294 | true | HCP | 315 | true | HCP |
| 275 | true | HCP | 295 | true | HCP | 316 | true | HCP |
| 276 | true | HCP | 296 | shore | HCP | 317 | true | HCP |
| 277 | true | ICP | 297 | true | ICP | 318 | false | ICP |
| 278 | true | HCP | 298 | true | HCP | 319 | true | HCP |
| 279 | true | HCP | 299 | false | ICP | 320 | true | HCP |
| 280 | true | HCP | 300 | true | HCP | 321 | shore | HCP |
| 281 | shore | ICP | 301 | shore | HCP | | | |
| 282 | true | HCP | 302 | true | HCP | | | |
| 283 | true | HCP | 303 | shore | HCP | | | |
| 284 | true | HCP | 304 | true | HCP | | | |
| 285 | true | HCP | 305 | true | HCP | | | |
| 286 | true | HCP | 306 | true | HCP | | | |

TABLE 5C
CpG islands of the ESR1-negative module:

| SEQ | Entrez | | | | | |
|---|---|---|---|---|---|---|
| ID | Gene | | | | | Methylation |
| NO. | ID | SYMBOL | Affy_ID | coefficient | Illumina_ID | Enrichment |
| 322 | 51442 | VGLL1 | 215729_s_at | −0.66129561 | cg21462299 | |
| 323 | 26227 | PHGDH | 201397_at | −0.64928809 | cg07090813 | Cluster II |
| 324 | 6648 | SOD2 | 215223_s_at | −0.62622708 | cg14515483 | |
| 325 | 221061 | C10orf38 | 212771_at | −0.61911622 | cg04451988 | |
| 326 | 53335 | BCL11A | 219497_s_at | −0.61751635 | cg22166290 | Cluster II |
| 327 | 4478 | MSN | 200600_at | −0.59183487 | cg09778422 | Cluster II |
| 328 | 6664 | SOX11 | 204914_s_at | −0.57838974 | cg20008332 | Cluster II |
| 329 | 10950 | BTG3 | 205548_s_at | −0.57803585 | cg14380517 | Cluster II |
| 330 | 83439 | TCF7L1 | 221016_s_at | −0.57685166 | cg02508567 | Cluster II |
| 331 | 8543 | LMO4 | 209204_at | −0.56711672 | cg10912077 | Cluster II |
| 332 | 2617 | GARS | 208693_s_at | −0.56419322 | cg15693363 | |
| 333 | 2296 | FOXC1 | 213260_at | −0.56246613 | cg04504095 | |
| 334 | 2568 | GABRP | 205044_at | −0.55883521 | cg21652012 | Cluster II |
| 335 | 3945 | LDHB | 201030_x_at | −0.55557485 | cg06437004 | Cluster II |
| 336 | 5613 | PRKX | 204061_at | −0.55539077 | cg09094355 | Cluster II |
| 337 | 1054 | CEBPG | 204203_at | −0.55314581 | cg15046693 | Cluster II |
| 338 | 4783 | NFIL3 | 203574_at | −0.55143972 | cg15919045 | |
| 339 | 3868 | KRT16 | 209800_at | −0.54949798 | cg27478659 | Cluster II |
| 340 | 55765 | C1orf106 | 219010_at | −0.54180004 | cg15250507 | |
| 341 | 5937 | RBMS1 | 207266_x_at | −0.53974436 | cg14325649 | |
| 342 | 3898 | LAD1 | 203287_at | −0.53550815 | cg25947945 | |
| 343 | 2173 | FABP7 | 205029_s_at | −0.52941225 | cg05798712 | |
| 344 | 9435 | CHST2 | 203921_at | −0.5239671 | cg00995327 | Cluster II |
| 345 | 6663 | SOX10 | 209842_at | −0.52250076 | cg06614002 | Cluster II |
| 346 | 1476 | CSTB | 201201_at | −0.52228528 | cg14095850 | |
| 347 | 10982 | MAPRE2 | 202501_at | −0.5193823 | cg07020962 | |
| 348 | 8685 | MARCO | 205819_at | −0.51838499 | cg02431964 | |
| 349 | 7371 | UCK2 | 209825_s_at | −0.51709149 | cg03036064 | |
| | 85377 | MICALL1 | 221779_at | −0.51653462 | NA | |
| 350 | 79650 | C16orf57 | 218060_s_at | −0.51270039 | cg07398350 | |
| 351 | 1116 | CHI3L1 | 209395_at | −0.5075254 | cg07423149 | Cluster II |
| 352 | 8645 | KCNK5 | 219615_s_at | −0.50676541 | cg02128567 | Cluster II |
| 353 | 23321 | TRIM2 | 202341_s_at | −0.50510712 | cg12793610 | Cluster II |
| 354 | 25841 | ABTB2 | 213497_at | −0.50152319 | cg01888411 | Cluster II |
| 355 | 5806 | PTX3 | 206157_at | −0.50095406 | cg15565872 | Cluster II |
| 356 | 4953 | ODC1 | 200790_at | −0.50017862 | cg05741384 | Cluster II |
| 357 | 8842 | PROM1 | 204304_s_at | −0.49873779 | cg20576510 | |
| 358 | 6715 | SRD5A1 | 211056_s_at | −0.49787464 | cg16935609 | Cluster II |
| 359 | 8581 | LY6D | 206276_at | −0.49652701 | cg07572435 | Cluster II |
| 360 | 3613 | IMPA2 | 203126_at | −0.49271114 | cg00008713 | Cluster II |
| 361 | 3383 | ICAM1 | 202638_s_at | −0.4921546 | cg22874046 | |
| 362 | 1410 | CRYAB | 209283_at | −0.49071498 | cg15227610 | Cluster II |
| 363 | 22929 | SEPHS1 | 208941_s_at | −0.49031224 | cg17854497 | |
| 364 | 7851 | MALL | 209373_at | −0.48905517 | cg09113530 | Cluster II |
| 365 | 375035 | SFT2D2 | 214838_at | −0.48888168 | cg12739647 | |
| 366 | 1824 | DSC2 | 204750_s_at | −0.48878224 | cg00566759 | |
| 367 | 6280 | S100A9 | 203535_at | −0.48574767 | cg16139316 | Cluster II |
| | 55544 | RBM38 | 212430_at | −0.48523095 | NA | |
| 368 | 8531 | CSDA | 201161_s_at | −0.48379436 | cg03876622 | |
| | 11013 | TMSL8 | 205347_s_at | −0.48243815 | NA | |
| 369 | 7545 | ZIC1 | 206373_at | −0.47973354 | cg05073035 | Cluster II |
| 370 | 5317 | PKP1 | 221854_at | −0.47574048 | cg09009380 | Cluster II |
| 371 | 7368 | UGT8 | 208358_s_at | −0.47320635 | cg25892041 | |
| 372 | 11254 | SLC6A14 | 219795_at | −0.46793656 | cg00894577 | |
| 373 | 8326 | FZD9 | 207639_at | −0.46571299 | cg20692569 | Cluster II |
| 374 | 59342 | SCPEP1 | 218217_at | −0.46539062 | cg07833382 | |
| 375 | 7388 | UQCRH | 202233_s_at | −0.46334012 | cg21576698 | |
| 376 | 10479 | SLC9A6 | 203909_at | −0.46218527 | cg06657741 | |
| 377 | 6769 | STAC | 205743_at | −0.46154415 | cg19055231 | Cluster II |
| 378 | 23 | ABCF1 | 200045_at | −0.45941767 | cg18015044 | Cluster II |
| 379 | 9929 | JOSD1 | 201751_at | −0.45878624 | cg26380756 | Cluster II |
| 380 | 54149 | C21orf91 | 220941_s_at | −0.45741133 | cg01284306 | |
| 381 | 1827 | DSCR1 | 208370_s_at | −0.45318343 | cg20206574 | |
| 382 | 57348 | TTYH1 | 219415_at | −0.45165274 | cg10187559 | |
| | 64764 | CREB3L2 | 212345_s_at | −0.44888154 | NA | |
| 383 | 55975 | KLHL7 | 220238_s_at | −0.44715312 | cg09234859 | Cluster II |
| 384 | 6376 | CX3CL1 | 203687_at | −0.44647627 | cg20427865 | Cluster II |
| 385 | 4851 | NOTCH1 | 218902_at | −0.44628024 | cg20042228 | Cluster II |
| 386 | 4321 | MMP12 | 204580_at | −0.44026565 | cg03179866 | |
| 387 | 8884 | SLC5A6 | 204087_s_at | −0.43982908 | cg01620785 | |
| 388 | 51806 | CALML5 | 220414_at | −0.43692661 | cg24392574 | |
| 389 | 1299 | COL9A3 | 204724_s_at | −0.43453156 | cg06497752 | |
| 390 | 419 | ART3 | 210147_at | −0.43304415 | cg22252999 | Cluster II |
| 391 | 2919 | CXCL1 | 204470_at | −0.43103914 | cg02029926 | |
| 392 | 57110 | HRASLS | 219984_s_at | −0.43040468 | cg17878972 | Cluster II |
| 393 | 25825 | BACE2 | 217867_x_at | −0.42961248 | cg16334795 | Cluster II |
| 394 | 8190 | MIA | 206560_s_at | −0.42956164 | cg25152942 | Cluster II |
| 395 | 2824 | GPM6B | 209170_s_at | −0.42759793 | cg21229055 | Cluster II |
| 396 | 4828 | NMB | 205204_at | −0.42674501 | cg19517291 | |
| 397 | 3066 | HDAC2 | 201833_at | −0.42527142 | cg18387216 | |
| | 5321 | PLA2G4A | 210145_at | −0.42416523 | NA | |
| 398 | 10477 | UBE2E3 | 210024_s_at | −0.42413489 | cg00949554 | |
| 399 | 136 | ADORA2B | 205891_at | −0.42306361 | cg03729431 | Cluster II |
| 400 | 3576 | IL8 | 202859_x_at | −0.422638 | cg18302652 | |
| 401 | 5971 | RELB | 205205_at | −0.42058475 | cg02727285 | Cluster II |
| 402 | 55240 | STEAP3 | 218424_s_at | −0.41466295 | cg04749104 | |
| 403 | 25818 | KLK5 | 222242_s_at | −0.41340419 | cg04349727 | |
| | 2171 | FABP5 | 202345_s_at | −0.41219044 | NA | |
| 404 | 23650 | TRIM29 | 211002_s_at | −0.41153904 | cg13625403 | |
| | 79627 | OGFRL1 | 219582_at | −0.41147589 | NA | |
| 405 | 7436 | VLDLR | 209822_s_at | −0.4101615 | cg05523047 | |
| | 3892 | KRT86 | 215189_at | −0.40898783 | NA | |
| 406 | 10874 | NMU | 206023_at | −0.40879552 | cg01943185 | Cluster II |
| | 79605 | PGBD5 | 219225_at | −0.40705584 | NA | |
| 407 | 8985 | PLOD3 | 202185_at | −0.40629339 | cg25527547 | |
| | 60487 | TRMT11 | 218877_s_at | −0.40566142 | NA | |
| 408 | 1381 | CRABP1 | 205350_at | −0.40429027 | cg19777470 | Cluster II |
| 409 | 1356 | CP | 204846_at | −0.40404337 | cg17439694 | Cluster II |
| 410 | 3097 | HIVEP2 | 212641_at | −0.40364447 | cg22858308 | Cluster II |
| 411 | 10656 | KHDRBS3 | 209781_s_at | −0.40340408 | cg25945374 | |
| 412 | 10575 | CCT4 | 200877_at | −0.40322219 | cg19716462 | Cluster II |
| 413 | 4071 | TM4SF1 | 215034_s_at | −0.4024996 | cg08124030 | |
| 414 | 6948 | TCN2 | 204043_at | −0.40164819 | cg04081402 | |
| 415 | 10644 | IGF2BP2 | 218847_at | −0.40137448 | cg18234011 | |
| 416 | 3418 | IDH2 | 210046_s_at | −0.40013914 | cg17925542 | Cluster II |
| 417 | 9200 | PTPLA | 219654_at | −0.39972249 | cg23868119 | |
| 418 | 3872 | KRT17 | 205157_s_at | −0.39795768 | cg27236973 | Cluster II |
| 419 | 7159 | TP53BP2 | 203120_at | −0.3957261 | cg16028934 | |
| 420 | 10200 | MPHOSPH6 | 203740_at | −0.39554753 | cg16119274 | Cluster II |
| | 706 | TSPO | 202096_s_at | −0.39169845 | NA | |
| 421 | 688 | KLF5 | 209211_at | −0.39113342 | cg12848131 | |
| 422 | 1672 | DEFB1 | 210397_at | −0.39076646 | cg19033555 | |
| 423 | 23336 | DMN | 212730_at | −0.39034362 | cg13191049 | Cluster II |
| 424 | 57180 | ACTR3B | 218868_at | −0.38659759 | cg10896886 | |
| 425 | 3294 | HSD17B2 | 204818_at | −0.38270805 | cg20373326 | |
| 426 | 28960 | DCPS | 218774_at | −0.38267717 | cg03830408 | |
| 427 | 2982 | GUCY1A3 | 221942_s_at | −0.38254572 | cg02210887 | |
| 428 | 54619 | CCNJ | 219470_x_at | −0.3811175 | cg04590978 | Cluster II |
| 429 | 57211 | GPR126 | 213094_at | −0.37693751 | cg11176095 | Cluster II |
| 430 | 1117 | CHI3L2 | 213060_s_at | −0.37689236 | cg10045881 | Cluster II |
| 431 | 7345 | UCHL1 | 201387_s_at | −0.37679195 | cg24715245 | Cluster II |
| 432 | 54913 | RPP25 | 219143_s_at | −0.37237191 | cg09619786 | |
| 433 | 2627 | GATA6 | 210002_at | −0.37081347 | cg19496782 | |
| 434 | 875 | CBS | 212816_s_at | −0.36357167 | cg22633722 | Cluster II |
| 435 | 6364 | CCL20 | 205476_at | −0.36319472 | cg09425228 | |
| | 934 | CD24 | 209772_s_at | −0.36282951 | NA | |
| 436 | 274 | BIN1 | 210202_s_at | −0.36200933 | cg25228746 | |
| 437 | 11202 | KLK8 | 206125_s_at | −0.35998705 | cg19149785 | |
| 438 | 11170 | FAM107A | 209074_s_at | −0.35901803 | cg06638451 | Cluster II |
| 439 | 5271 | SERPINB8 | 206034_at | −0.35808395 | cg27100123 | |
| 440 | 5268 | SERPINB5 | 204855_at | −0.35802733 | cg20837735 | |
| | 8563 | THOC5 | 209418_s_at | −0.35724536 | NA | |
| 441 | 5100 | PCDH8 | 206935_at | −0.35519567 | cg20366906 | Cluster II |
| 442 | 56938 | ARNTL2 | 220658_s_at | −0.35442683 | cg01986577 | Cluster II |
| 443 | 10525 | HYOU1 | 200825_s_at | −0.35389917 | cg07330718 | |
| 444 | 23532 | PRAME | 204086_at | −0.35189188 | cg05208878 | Cluster II |
| 445 | 6261 | RYR1 | 205485_at | −0.35082856 | cg15517609 | |
| 446 | 6723 | SRM | 201516_at | −0.3457862 | cg21379816 | Cluster II |
| 447 | 3595 | IL12RB2 | 206999_at | −0.34467894 | cg01356829 | Cluster II |
| 448 | 3574 | IL7 | 206693_at | −0.34389077 | cg23538854 | |
| 449 | 6564 | SLC15A1 | 207254_at | −0.34318347 | cg10694152 | Cluster II |
| 450 | 2591 | GALNT3 | 203397_s_at | −0.34242172 | cg15739581 | |
| 451 | 2770 | GNAI1 | 209576_at | −0.34021112 | cg05806233 | Cluster II |
| 452 | 8986 | RPS6KA4 | 204632_at | −0.33810477 | cg24970539 | |
| 453 | 54438 | GFOD1 | 219821_s_at | −0.3377583 | cg00194146 | |
| 454 | 25984 | KRT23 | 218963_s_at | −0.33772871 | cg06378617 | |
| 455 | 51302 | CYP39A1 | 220432_s_at | −0.33695618 | cg19557537 | Cluster II |
| 456 | 7037 | TFRC | 207332_s_at | −0.33653368 | cg22956956 | |
| 457 | 390 | RND3 | 212724_at | −0.33533047 | cg11626656 | |
| 458 | 8324 | FZD7 | 203706_s_at | −0.33206439 | cg12618251 | Cluster II |
| 459 | 9982 | FGFBP1 | 205014_at | −0.33016268 | cg13929970 | Cluster II |
| 460 | 827 | CAPN6 | 202965_s_at | −0.32896134 | cg19688503 | Cluster II |
| 461 | 2348 | FOLR1 | 204437_s_at | −0.32727835 | cg03699566 | |
| 462 | 6271 | S100A1 | 205334_at | −0.32519543 | cg14467840 | |
| 463 | 9258 | MFHAS1 | 213457_at | −0.3244714 | cg15819853 | Cluster II |
| 464 | 9510 | ADAMTS1 | 222162_s_at | −0.31714081 | cg00472814 | Cluster II |
| 465 | 22943 | DKK1 | 204602_at | −0.31707767 | cg07684796 | Cluster II |
| 466 | 2861 | GPR37 | 209631_s_at | −0.31562942 | cg23428445 | |
| 467 | 55506 | H2AFY2 | 218445_at | −0.31488076 | cg17163751 | |
| 468 | 6277 | S100A6 | 217728_at | −0.31127446 | cg09413557 | |
| 469 | 65983 | GRAMD3 | 218706_s_at | −0.31070593 | cg08704509 | |
| 470 | 3096 | HIVEP1 | 204512_at | −0.30420168 | cg07782113 | |
| 471 | 8792 | TNFRSF11A | 207037_at | −0.30152349 | cg01765461 | |
| 472 | 3400 | ID4 | 209291_at | −0.29901729 | cg17252960 | Cluster II |
| 473 | 1475 | CSTA | 204971_at | −0.29629654 | cg26928972 | Cluster II |
| 474 | 26278 | SACS | 213262_at | −0.29589301 | cg25206802 | |
| 475 | 4188 | MDFI | 205375_at | −0.29462263 | cg05345286 | |
| 476 | 1525 | CXADR | 203917_at | −0.29399348 | cg00744433 | Cluster II |
| 477 | 9022 | CLIC3 | 219529_at | −0.29342331 | cg15387123 | |
| 478 | 9508 | ADAMTS3 | 214913_at | −0.29195187 | cg13643796 | |
| 479 | 23318 | ZCCHC11 | 212704_at | −0.2874469 | cg07347137 | Cluster II |
| 480 | 202 | AIM1 | 212543_at | −0.28250629 | cg24194539 | |
| 481 | 83988 | NCALD | 211685_s_at | −0.27863454 | cg01484156 | |
| | 79745 | CLIP4 | 219944_at | −0.27836222 | NA | |
| 482 | 64849 | SLC13A3 | 205243_at | −0.27379455 | cg18468842 | |
| 483 | 5562 | PRKAA1 | 209799_at | −0.27248266 | cg10786880 | Cluster II |
| 484 | 79852 | ABHD9 | 220013_at | −0.27078394 | cg05488632 | Cluster II |
| 485 | 6496 | SIX3 | 206634_at | −0.2645826 | cg13163729 | Cluster II |
| 486 | 5803 | PTPRZ1 | 204469_at | −0.26445918 | cg25167643 | |
| 487 | 4691 | NCL | 200610_s_at | −0.25948109 | cg26862286 | |
| 488 | 1644 | DDC | 205311_at | −0.25539982 | cg04144768 | |
| 489 | 23266 | LPHN2 | 206953_s_at | −0.25295037 | cg08235271 | |
| | 55790 | NA | 219049_at | −0.25042614 | NA | |
| 490 | 1783 | DYNC1LI2 | 203590_at | −0.24622451 | cg21610192 | |
| | 4139 | MARK1 | 221047_s_at | −0.24475937 | NA | |
| | 926 | CD8B | 215332_s_at | −0.24348476 | NA | |
| 491 | 10331 | B3GNT3 | 204856_at | −0.24063883 | cg03316864 | |
| 492 | 6304 | SATB1 | 203408_s_at | −0.23571514 | cg00674922 | |
| 493 | 2920 | CXCL2 | 209774_x_at | −0.23251798 | cg16890267 | Cluster II |
| 494 | 2588 | GALNS | 206335_at | −0.23243233 | cg08781448 | |
| 495 | 50805 | IRX4 | 220225_at | −0.23224835 | cg03963198 | |
| 496 | 5737 | PTGFR | 207177_at | −0.2231448 | cg03495868 | Cluster II |
| 497 | 3779 | KCNMB1 | 209948_at | −0.21564509 | cg22646937 | |
| 498 | 8785 | MATN4 | 207123_s_at | −0.20822884 | cg14448104 | |
| 499 | 10810 | WASF3 | 204042_at | −0.18215567 | cg07744166 | Cluster II |

| SEQ | CpG_ | | | SEQ | CpG_ | | |
|---|---|---|---|---|---|---|---|
| ID | Island_ | Promoter_ | Expression | ID | Island_ | Promoter_ | Expression |
| No. | Revisited | Class | Enrichment | No. | Revisited | Clas | Enrichment |
| 322 | false | ICP | Cluster I | 331 | true | HCP | Cluster I |
| 323 | true | ICP | Cluster I | 332 | true | HCP | Cluster I |
| 324 | true | HCP | Cluster I | 333 | true | HCP | Cluster I |
| 325 | true | HCP | Cluster I | 334 | false | ICP | Cluster I |
| 326 | shore | HCP | Cluster I | 335 | true | ICP | Cluster I |
| 327 | shore | HCP | Cluster I | 336 | shore | HCP | Cluster I |
| 328 | true | HCP | Cluster I | 337 | shore | HCP | |
| 329 | true | HCP | Cluster I | 338 | true | HCP | Cluster I |
| 330 | true | HCP | Cluster I | 339 | true | ICP | Cluster I |
| 340 | true | HCP | Cluster I | 388 | true | HCP | Cluster I |
| 341 | shore | HCP | | 389 | true | HCP | Cluster I |
| 342 | true | HCP | Cluster I | 390 | false | LCP | Cluster I |
| 343 | false | ICP | Cluster I | 391 | true | ICP | Cluster I |
| 344 | true | HCP | Cluster I | 392 | true | HCP | Cluster I |
| 345 | true | ICP | Cluster I | 393 | false | HCP | Cluster I |
| 346 | true | HCP | Cluster I | 394 | false | ICP | Cluster I |
| 347 | true | HCP | Cluster I | 395 | shore | HCP | Cluster I |
| 348 | false | ICP | Cluster I | 396 | shore | HCP | Cluster I |
| 349 | true | HCP | Cluster I | 397 | true | HCP | Cluster I |
| | | | Cluster I | | | | Cluster I |
| 350 | true | ICP | Cluster I | 398 | true | HCP | Cluster I |
| 351 | false | ICP | Cluster I | 399 | true | HCP | |
| 352 | true | HCP | Cluster I | 400 | false | LCP | Cluster I |
| 353 | false | ICP | Cluster I | 401 | shore | HCP | Cluster I |
| 354 | true | HCP | Cluster I | 402 | true | HCP | Cluster I |
| 355 | true | ICP | Cluster I | 403 | shore | ICP | Cluster I |
| 356 | true | HCP | Cluster I | | | | Cluster I |
| 357 | false | ICP | Cluster I | 404 | true | ICP | Cluster I |
| 358 | false | HCP | Cluster I | | | | Cluster I |
| 359 | shore | ICP | Cluster I | 405 | true | HCP | Cluster I |
| 360 | true | HCP | Cluster I | | | | Cluster I |
| 361 | true | HCP | Cluster I | 406 | true | HCP | Cluster I |
| 362 | shore | ICP | Cluster I | | | | Cluster I |
| 363 | true | HCP | Cluster I | 407 | shore | HCP | |
| 364 | shore | HCP | Cluster I | | | | Cluster I |
| 365 | true | HCP | Cluster I | 408 | true | | Cluster I |
| 366 | true | HCP | Cluster I | 409 | false | LCP | Cluster I |
| 367 | false | ICP | Cluster I | 410 | shore | ICP | Cluster I |
| | | | Cluster I | 411 | true | HCP | Cluster I |
| 368 | true | HCP | Cluster I | 412 | true | HCP | |
| | | | Cluster I | 413 | true | ICP | Cluster I |
| 396 | true | HCP | Cluster I | 414 | false | ICP | Cluster I |
| 370 | true | HCP | Cluster I | 415 | true | HCP | Cluster I |
| 371 | false | LCP | Cluster I | 416 | true | HCP | Cluster I |
| 372 | false | ICP | Cluster I | 417 | true | HCP | Cluster I |
| 373 | true | HCP | Cluster I | 418 | true | ICP | Cluster I |
| 374 | true | HCP | Cluster I | 419 | true | HCP | Cluster I |
| 375 | true | HCP | Cluster I | 420 | true | HCP | Cluster I |
| 376 | true | HCP | Cluster I | | | | Cluster I |
| 377 | true | HCP | Cluster I | 421 | true | HCP | Cluster I |
| 378 | true | HCP | | 422 | false | ICP | Cluster I |
| 379 | true | HCP | | 423 | true | HCP | Cluster I |
| 380 | shore | HCP | Cluster I | 424 | true | HCP | Cluster I |
| 381 | true | HCP | Cluster I | 435 | false | ICP | Cluster I |
| 382 | shore | HCP | Cluster I | 426 | true | HCP | Cluster I |
| | | | Cluster I | 427 | false | ICP | Cluster I |
| 383 | true | HCP | | 428 | true | HCP | Cluster I |
| 384 | false | ICP | Cluster I | 429 | true | HCP | Cluster I |
| 385 | true | HCP | Cluster I | 430 | false | ICP | Cluster I |
| 386 | false | LCP | Cluster I | 431 | true | HCP | Cluster I |
| 387 | shore | HCP | Cluster I | 432 | true | HCP | Cluster I |
| 433 | true | HCP | Cluster I | 482 | false | ICP | |
| 434 | true | HCP | Cluster I | 483 | true | HCP | |
| 435 | false | LCP | Cluster I | 484 | true | HCP | Cluster I |
| | | | | 485 | true | ICP | Cluster I |
| 436 | shore | HCP | Cluster I | 486 | true | HCP | Cluster I |
| 437 | true | ICP | Cluster I | 487 | shore | HCP | |
| 438 | false | ICP | Cluster I | 488 | false | ICP | |
| 439 | shore | ICP | Cluster I | 489 | true | HCP | Cluster I |
| 440 | shore | ICP | Cluster I | | | | Cluster I |
| | | | Cluster I | 490 | true | HCP | |
| 441 | true | HCP | Cluster I | | | | |
| 442 | true | HCP | Cluster I | | | | Cluster I |
| 443 | true | HCP | Cluster I | 491 | true | ICP | Cluster I |
| 444 | true | ICP | Cluster I | 492 | true | ICP | |
| 445 | shore | ICP | Cluster I | 493 | true | HCP | Cluster I |
| 446 | true | HCP | Cluster I | 494 | true | HCP | |
| 447 | shore | HCP | Cluster I | 495 | true | HCP | |
| 448 | true | ICP | Cluster I | 496 | true | HCP | Cluster I |
| 449 | true | HCP | Cluster I | 497 | false | ICP | Cluster I |
| 450 | false | LCP | Cluster I | 498 | true | ICP | Cluster I |
| 451 | true | HCP | Cluster I | 499 | true | HCP | |
| 452 | true | HCP | | | | | |
| 453 | shore | HCP | Cluster I | | | | |
| 454 | false | ICP | Cluster I | | | | |
| 455 | true | HCP | Cluster I | | | | |
| 456 | true | HCP | Cluster I | | | | |
| 457 | true | ICP | Cluster I | | | | |
| 458 | true | HCP | Cluster I | | | | |
| 459 | false | ICP | Cluster I | | | | |
| 460 | false | ICP | Cluster I | | | | |
| 461 | false | ICP | | | | | |
| 462 | false | ICP | Cluster I | | | | |
| 463 | true | HCP | Cluster I | | | | |
| 464 | true | HCP | Cluster I | | | | |
| 465 | true | HCP | Cluster I | | | | |
| 466 | true | HCP | Cluster I | | | | |
| 467 | true | HCP | | | | | |
| 468 | true | HCP | | | | | |
| 469 | shore | HCP | | | | | |
| 470 | true | HCP | | | | | |
| 471 | true | HCP | Cluster I | | | | |
| 472 | true | HCP | Cluster I | | | | |
| 473 | false | LCP | Cluster I | | | | |
| 474 | false | LCP | Cluster I | | | | |
| 475 | true | HCP | Cluster I | | | | |
| 476 | false | HCP | Cluster I | | | | |
| 477 | true | ICP | Cluster I | | | | |
| 478 | true | HCP | Cluster I | | | | |
| 479 | true | HCP | Cluster I | | | | |
| 480 | true | ICP | Cluster I | | | | |
| 481 | false | LCP | Cluster I | | | | |
| | | | Cluster I | | | | |

### Example 3

### Refining the Methylation-Based Taxonomy of the Tumour Set

As shown in FIG. 3a, the unsupervised analysis of recurrent methylation patterns yielded 6 distinct entities (clusters 1 to 6). These methylation clusters were next compared to known breast cancer “expression subtypes”. Currently, on the basis of gene expression profiles, four subtypes are distinguished: basal-like breast cancers (corresponding mostly to ER-negative and HER2-negative), HER2-positive cancers characterized by increased expression of several genes of the HER2 amplicon, and two luminal-like subtypes, low-grade luminal A and high-grade luminal B, which are predominantly ER-positive (Sotiriou, C. & Piccart, M. J. 2007 Nat. Rev. Cancer 7, 545-553). IHC and gene expression profiling (FIG. 3a and Table 6) revealed a significant preponderance of HER2-overexpressing tumours in cluster 2, basal-like tumours in cluster 3, and luminal A tumours in cluster 6. Interestingly, no single “expression subtype” appeared to dominate in methylation clusters 1, 4, and 5: cluster 1 contained HER2, basal-like as well as luminal B tumours; cluster 4 appeared to be a mix of HER2 and luminal B tumours; and cluster 5 contained both luminal A and B tumours (FIG. 3a). In FIG. 3f, the correlation with clinical parameters was made. Clusters 5 and 6 contained exclusively ER-positive tumours, whereas clusters 3 were composed principally of ERnegative tumours. HER2-positive tumours were predominant in clusters 1 and 2. Cluster 6 contained majorly grade 1 tumours. No significant association with tumour size or age was found.

TABLE 6
Association between the 6 methylation clusters identified in the main set of patients and the
“known expression subtypes”. Upper table indicates the p-values provided by Fisher's Exact test to
evaluate the association between each methylation group and each “known expression subtype”
determined by immunochemistry (IHC) as well as the Phi value in brackets. Lower table indicates
the likelihood ratio pvalues provided by Chi square test to evaluate the association between each
methylation group and each “known expression subtype” determined by gene expression (GE) as
well as the Phi value in brackets.

| | | HER2 | | Basal-like | | Luminal A | | Luminal B | |
|---|---|---|---|---|---|---|---|---|---|
| | | “Known expression subtypes” (IHC) | | | | | | | |
| Methylation | Cluster 1 | 0.17 | (Phi = 0.178) | 0.502 | (Phi = −0.092) | 0.111 | (Phi = −0.201) | 0.471 | (Phi = 0.089) |
| groups | Cluster 2 | <0.001 | (Phi = 0.448) | 1 | (Phi = −0.034) | 0.172 | (Phi = −0.172) | 0.009 | (Phi = −0.286) |
| | Cluster 3 | 0.103 | (Phi = −0.186) | <0.001 | (Phi = 0.491) | 0.009 | (Phi = −0.275) | 0.769 | (Phi = −0.054) |
| | Cluster 4 | 0.692 | (Phi = 0.053) | 0.675 | (Phi = −0.104) | 0.344 | (Phi = −0.160) | 0.091 | (Phi = 0.198) |
| | Cluster 5 | 0.266 | (Phi = −0.144) | 0.433 | (Phi = −0.122) | 1 | (Phi = 0.026) | 0.033 | (Phi = 0.257) |
| | Cluster 6 | 0.002 | (Phi = −0.333) | 0.033 | (Phi = −0.237) | <0.001 | (Phi = 0.736) | 0.751 | (Phi = −0.077) |
| | | “Known expression subtypes” (GE) | | | | | | | |
| Methylation | Cluster 1 | 0.1 | (Phi = 0.238) | 0.059 | (Phi = 0.250) | 0.266 | (Phi = 0.163) | 0.253 | (Phi = 0.168) |
| groups | Cluster 2 | <0.001 | (Phi = 0.445) | 0.499 | (Phi = 0.123) | 0.038 | (Phi = 0.219) | 0.327 | (Phi = 0.149) |
| | Cluster 3 | 0.001 | (Phi = 0.366) | <0.001 | (Phi = 0.735) | 0.004 | (Phi = 0.315) | 0.189 | (Phi = 0.196) |
| | Cluster 4 | 0.592 | (Phi = 0.113) | 0.119 | (Phi = 0.177) | 0.723 | (Phi = 0.092) | 0.477 | (Phi = 0.134) |
| | Cluster 5 | 0.297 | (Phi = 0.165) | 0.027 | (Phi = 0.256) | 0.273 | (Phi = 0.185) | 0.098 | (Phi = 0.261) |
| | Cluster 6 | 0.004 | (Phi = 0.318) | 0.003 | (Phi = 0.323) | <0.001 | (Phi = 0.503) | 0.087 | (Phi = 0.254) |

To validate these six methylation clusters, the Infinium methylation assay was applied to an independent validation set of 117 breast tumours and the efficient nearest centroid classification method (Sørlie, T. et al., 2003 Proc. Natl Acad. Sci. USA 100, 8418-8423; Lusa, L. et al., 2007 J. Natl Cancer Inst. 99, 1715-1723) was used to assign, on the basis of DNA methylation profile similarities, each new sample to one of the 6 clusters. Focusing first on the main set, an 86 CpG-classifier was established that consists of a list of 86 key CpGs, this being the minimum number of CpGs required to retrieve the 6 unsupervised-analysis-based clusters (FIGS. 3b and 3c, Table 2). From this list of 86 CpGs, we calculated 6 centroids (i.e. profiles consisting of the median methylation value for each of the 86 CpGs) for each of the 6 methylation groups. Then, by computing the Spearman correlation of each tumour of the 6 validation set with each calculated centroid, each new sample was classified into one of the 6 methylation clusters (Supplementary FIG. 3c). Remarkably essentially all tumours of the validation set showed a strong correlation with one of the 6 methylation groups (FIG. 3d and FIG. 3e). Furthermore, IHC performed on the independent validation set showed a very similar “expression subtype composition” for each of the 6 groups as in the case of the main set (FIG. 3d, FIG. 3f and Table 7). It is noteworthy that the 86 CpG-classifier contained CpGs related to genes well-known to be implicated in breast cancer, such as: the oestrogen-inducible gene (TFF1), cyclin D1 (CCND1), secreted frizzled-related protein 2 (SFRP2), caspase 1 (CASP1), POU class 4 homeobox 1 (POU4F1) and interleukin 1, alpha and beta (IL1A and IL1B) (see Table 2 for the full list). Note also that this classifier contained majorly CpGs located in ICPs as well as LCPs (FIG. 3g). Taken together, these results reveal the existence of breast cancer groups that go beyond the currently known “expression subtypes” and suggest that methylation profiling may provide a basis for improving tumour taxonomy. Further, these observations suggest that methylation patterns distinguished here reflect the cell type of origin of the studied tumours (see FIG. 3h). Cluster 3 displayed the highest luminal progenitor signature score (p=0.001 versus clusters 2 and 4; p<0.001 versus other clusters; b), whereas the luminal mature signature score was higher for clusters 1, 4, 5, and 6 (p<0.001 for each of these clusters versus clusters 2 and 3, except for cluster 4 versus cluster 2 where p=0.019; c). Cluster 2 was not associated with any of the 3 signatures. d, e, f, Box plots of MaSC, luminal progenitor, and luminal mature signature scores, respectively, for each of the six methylation breast cancer groups, based on their DNA methylation profiles. A strong anti-correlation was observed between gene expression and DNA methylation data for the luminal progenitor and mature signatures (compare e with b and f with c, respectively) (respective Pearson's coefficients: −0.59, p=1.10-9 and −0.70, p=6.10-14). It was weaker for the MaSC signature (compare d with a; Pearson's coefficient: −0.47, p=4.10-6).

TABLE 7
Association between the 6 methylation groups obtained for the validation set of tumours
and the “known expression subtypes”. The table indicates the p-values provided by Fisher's Exact
test to evaluate the association between each methylation group of the validation set and each
“known expression subtype” determined by immunochemistry (IHC) as well as the Phi value in
brackets.

| | | “Known expression subtypes” (IHC) | | | | | | | |
|---|---|---|---|---|---|---|---|---|---|
| | | HER2 | | Basal-like | | Luminal A | | Luminal B | |
| Methylation | Cluster 1 | <0.001 | (Phi = 0.413) | 0.339 | (Phi = −0.112) | 0.037 | (Phi = −0.194) | 0.511 | (Phi = −0.083) |
| groups | Cluster 2 | 0.012 | (Phi = 0.261) | 0.170 | (Phi = −0.147) | 0.453 | (Phi = −0.107) | 1 | (Phi = 0.012) |
| | Cluster 3 | 0.002 | (Phi = −284) | <0.001 | (Phi = 0.673) | 0.023 | (Phi = −0.225) | 0.017 | (Phi = −0.223) |
| | Cluster 4 | 0.021 | (Phi = 0.241) | 0.276 | (Phi = −0.119) | 0.115 | (Phi = −0.158) | 0.692 | (Phi = −0.051) |
| | Cluster 5 | 0.296 | (Phi = −0.128) | 0.01 | (Phi = −0.241) | 0.735 | (Phi = 0.048) | 0.001 | (Phi = 0.326) |
| | Cluster 6 | 0.014 | (Phi = −0.221) | <0.001 | (Phi = −0.341) | <0.001 | (Phi = 0.556) | 0.798 | (Phi = 0.028) |

### Example 4

### Probing the Biological Significance of the Six Methyaltion Clusters

For this, the number of differentially methylated targets (as compared to normal samples) was quantified characterizing each of the above clusters in the main set. The number of targets was found to vary greatly between clusters, being lowest for cluster 3 (276 CpGs) and highest for cluster 4 (1,378 CpGs; FIG. 3i). Next, a gene ontology (GO) analysis was performed focusing on the genes in each cluster showing both differential methylation (as compared to normal samples) and a significant anti-correlation between methylation and expression. This revealed differential methylation of several genes involved in immunity, with different clusters showing distinct “epigenetic immune profiles” (FIG. 3j). In particular, tumours of clusters 2 (HER2-enriched) and 3 (basallike-enriched) showed hypomethylation of several immune genes (FIG. 3j). Because in this study whole tumour tissues were considered, the samples were constituted principally of epithelial cells, but also of cells from the surrounding stroma, including immune cells. Hence, the observed hypomethylation of immune genes in clusters 2 and 3 could indicate an infiltration of these tumours by immune cells, such as lymphocytes. This hypothesis proved correct. As shown in FIG. 3k, histologic analysis was performed, as previously described (Denkert, C. et al., 2010 J. Clin. Oncol. 28, 105-113), to determine stromal and intratumoral lymphocyte infiltration. Remarkably, the tumours of clusters 2 and 3 were much more infiltrated by lymphocytes than those of the other clusters (FIG. 3l). Furthermore, the methylation status of most of the immune genes highlighted by the GO analysis correlated inversely with the level of lymphocyte infiltration (FIG. 3m and Table 8).

TABLE 8
Spearman correlation between methylation status of immune genes
described in FIG. 3 and the stromal and intratumoral lymphocyte
infiltration.

| | | intratumoral | | stromal | |
|---|---|---|---|---|---|
| | | lymphocyte | | lymphocyte | |
| | | infiltration | | infiltration | |
| Gene_Name | Illumina_ID | rho | p-value | rho | p-value |
| AIM2 | cg10636246 | −0.378 | <0.001 | −0.309 | 0.001 |
| PSMB8 | cg16890093 | −0.447 | <0.001 | −0.457 | <0.001 |
| TNFSF8 | cg27631256 | −0.451 | <0.001 | −0.436 | <0.001 |
| LCP2 | cg17127769 | −0.288 | 0.003 | −0.237 | 0.014 |
| ITGAL | cg14176836 | −0.484 | <0.001 | −0.452 | <0.001 |
| HCLS1 | cg00141162 | −0.508 | <0.001 | −0.534 | <0.001 |
| CD6 | cg09902130 | −0.586 | <0.001 | −0.635 | <0.001 |
| CD79B | cg07973967 | −0.461 | <0.001 | −0.468 | <0.001 |
| LCK | cg17078393 | −0.554 | <0.001 | −0.584 | <0.001 |
| EBI2 | cg09626634 | −0.243 | 0.012 | −0.377 | <0.001 |
| GBP4 | cg27285720 | −0.379 | <0.001 | −0.343 | <0.001 |
| CST7 | cg11804789 | −0.436 | <0.001 | −0.412 | <0.001 |
| BST2 | cg16363586 | −0.163 | 0.095 | −0.144 | 0.141 |
| IL2RA | cg11733245 | −0.324 | 0.001 | −0.287 | 0.003 |
| PTPN22 | cg00916635 | −0.391 | <0.001 | −0.365 | <0.001 |
| IL18BP | cg16749930 | −0.61 | <0.001 | −0.626 | <0.001 |
| ADA | cg20622019 | −0.408 | <0.001 | −0.33 | 0.001 |
| IL21R | cg19423311 | −0.377 | <0.001 | −0.173 | 0.076 |
| LY75 | cg10107725 | −0.37 | <0.001 | −0.28 | 0.004 |
| HLA-DOB | cg04576021 | −0.399 | <0.001 | −0.305 | 0.001 |
| LAIR1 | cg06238491 | −0.455 | <0.001 | −0.317 | 0.001 |
| SYK | cg23447996 | −0.264 | 0.006 | −0.238 | 0.014 |
| CEBPG | cg15046693 | −0.406 | <0.001 | −0.366 | <0.001 |
| GAL | cg04464446 | −0.283 | 0.003 | −0.265 | 0.006 |
| GBP4 | cg21365602 | −0.503 | <0.001 | −0.426 | <0.001 |
| CCL5 | cg10315334 | −0.572 | <0.001 | −0.559 | <0.001 |
| TLR9 | cg21578541 | −0.412 | <0.001 | −0.395 | <0.001 |
| TLR1 | cg03430998 | −0.567 | <0.001 | −0.526 | <0.001 |

In addition, DNA methylation profiling of normal and breast cancer epithelial cell lines as well as ex vivo T and B lymphocytes and lymphoid cell lines revealed that a high number of the studied immune genes were highly methylated in breast cancer and normal epithelial cell lines but barely methylated in lymphocytes (FIG. 3n). These data strongly suggest that hypomethylation of immune genes detected in cluster-2 and -3 tumours reflect the celltype composition of the tumour microenvironment, and in particular a lymphocyte infiltration of these tumours. A closer look at these genes revealed, in cluster 2, hypomethylation of genes involved in T cell biology, e.g. genes encoding T cell markers, like the CD6 antigen, and T cell activation markers, like the LCK tyrosine kinase or the PTPN22 tyrosine phosphatase involved in T cell receptor signalling. These data might indicate that cluster-2 tumours, more readily than those of the other clusters, induce an antitumour T-cell response, with mobilization of T lymphocytes in the neoplastic environment.

Next, the clinical relevance of the above-mentioned epigenetic changes in breast carcinogenesis was analysed. To this end, a univariate survival analysis was performed of all 6,309 CpGs identified in the present invention (i.e. as being differentially methylated between normal breast samples and tumours). As suspected, the main set appeared too small to allow interpretable results. Therefore the more abundant gene expression data publicly available was used and only untreated patients were selected in order to evaluate the true prognostic value of biomarkers (between 730 and 952 samples, depending on the gene considered; Table 9).

TABLE 9
Publicly available gene expression data sets used for the
meta-analysis.

| Reference | Dataset | Technology | Survival | Patients | Probes |
|---|---|---|---|---|---|
| 54 | VDX | Affymetrix | RFS, DMFS | 344 | 22,283 |
| 55 | NKI | Agilent | RFS, DMFS, OS | 345 | 24,481 |
| 56 | MSK | Affymetrix | DMFS | 99 | 22,283 |
| 57 | UNT | Affymetrix | RFS, DMFS | 137 | 22,283 |
| 58 | CAL | Affymetrix | RFS, DMFS, OS | 118 | 22,283 |
| 59 | TBG | Affymetrix | RFS, DMFS, OS | 198 | 22,283 |
| 60 | NCH | Agilent | RFS, DMFS, OS | 135 | 17,086 |
| 61 | MAINZ | Affymetrix | DMFS | 200 | 22,283 |
| 62 | EMC2 | Affymetrix | DMFS | 204 | 54,675 |
| 63 | DFHCC | Affymetrix | DMFS | 115 | 54,675 |

The column “Survival” indicates the type of survival data available for each dataset.
RFS: Relapse-Free Survival,
DMFS: Distant Metastasis-Free Survival,
OS: Overall Survival.

Next, 55 genes were selected showing a strong anti-correlation between their methylation and expression status, and subjected to a univariate Cox regression analysis. Strikingly, no less than 32 of these genes (58%) emerged as significant prognostic markers (Table 10).

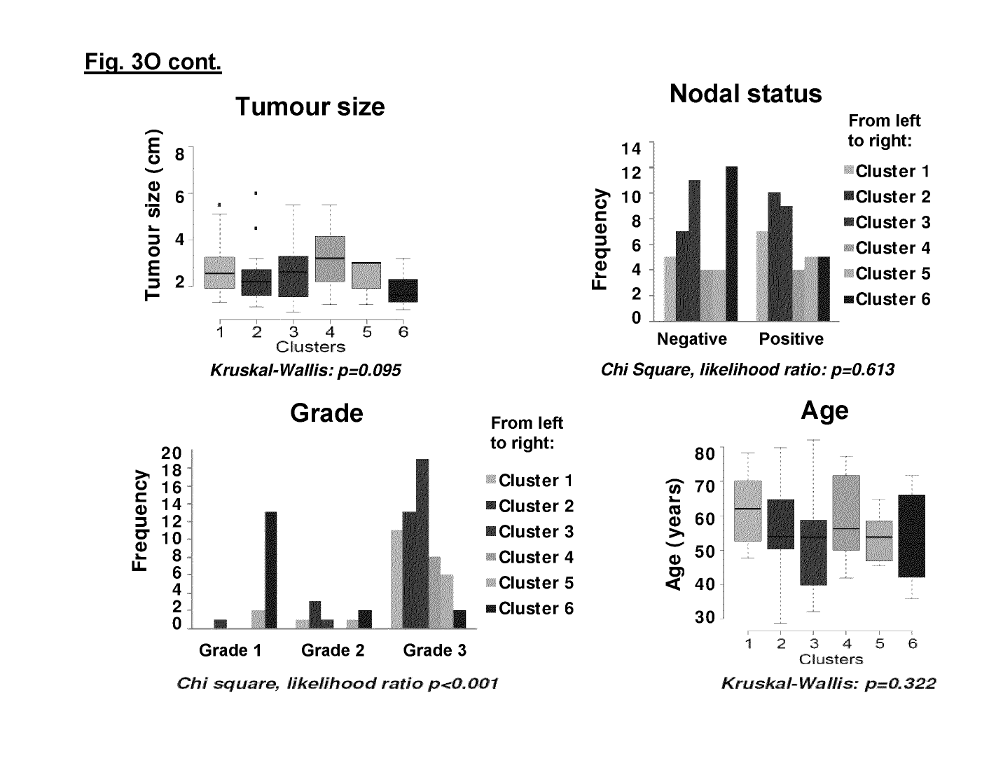

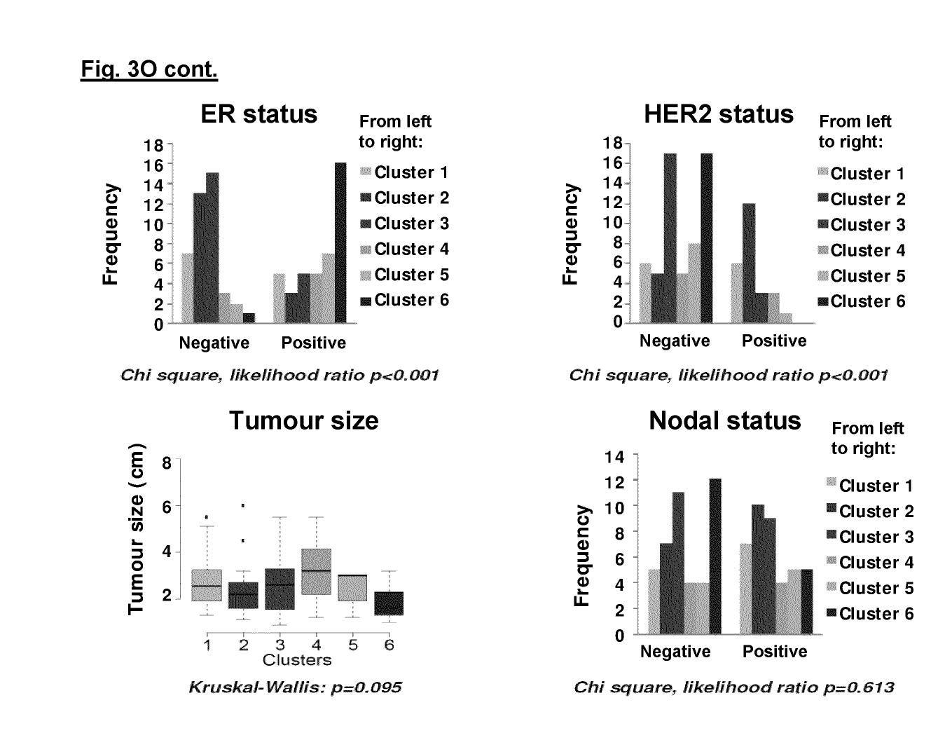

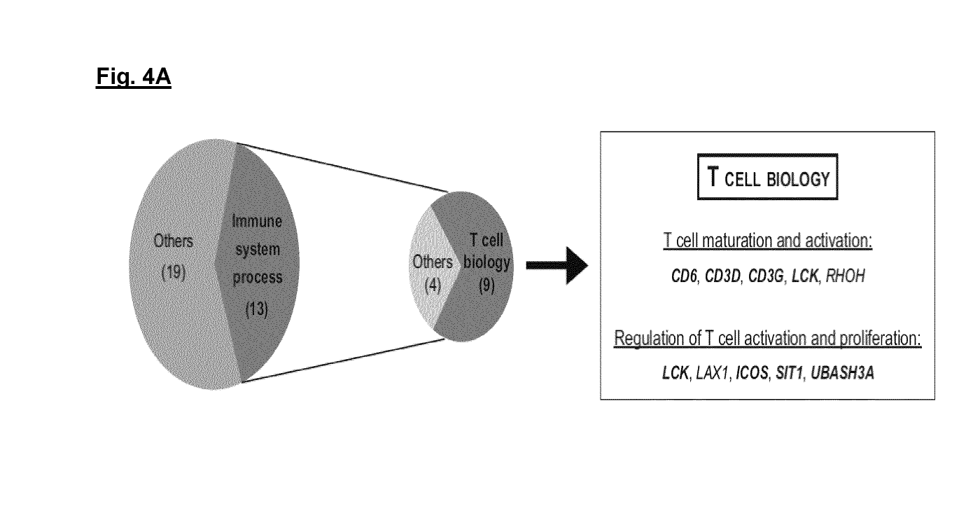

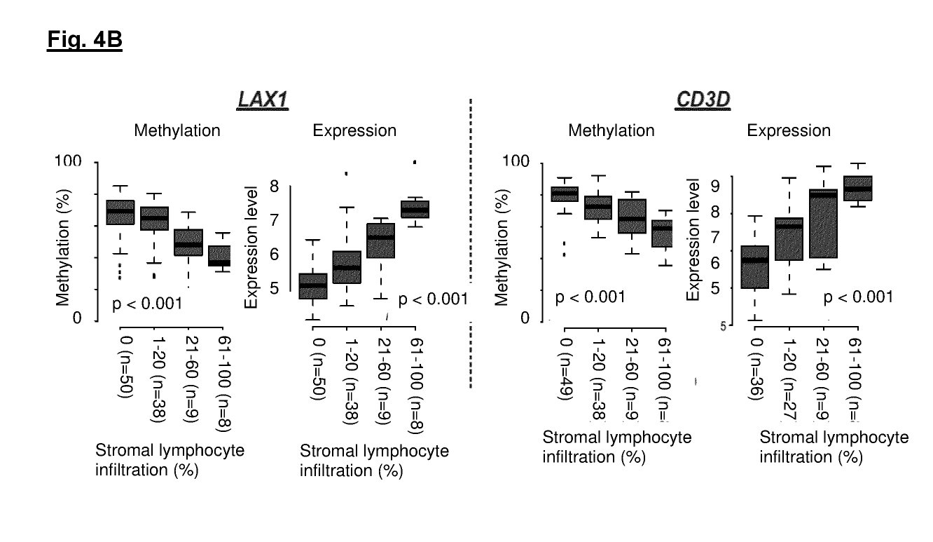

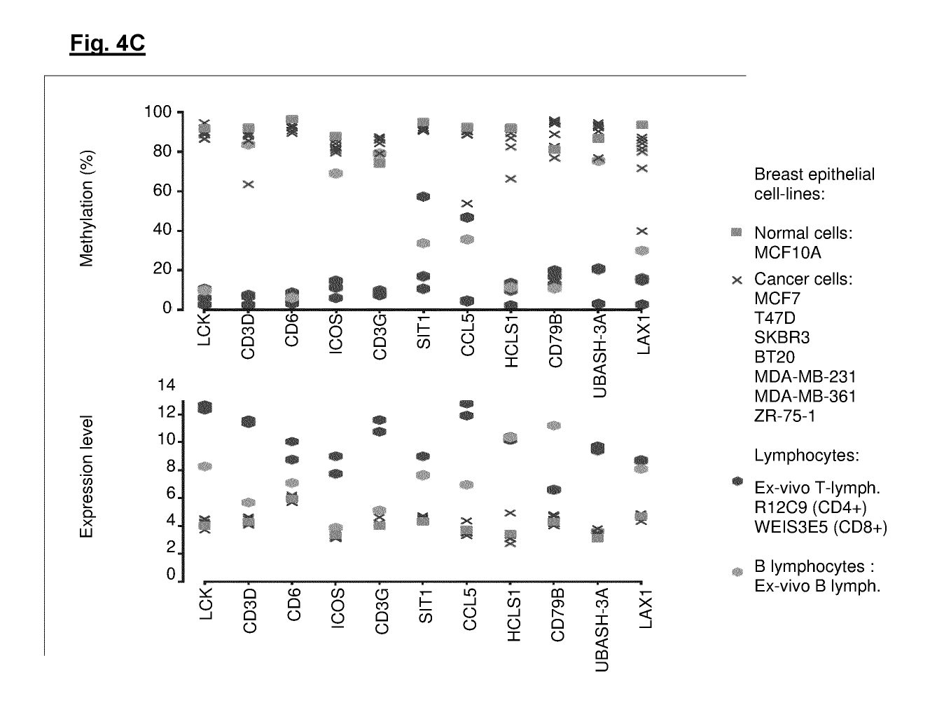

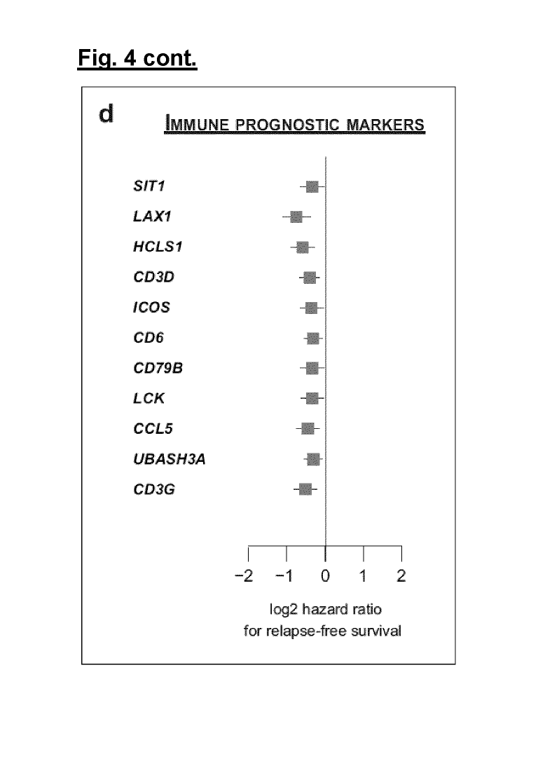

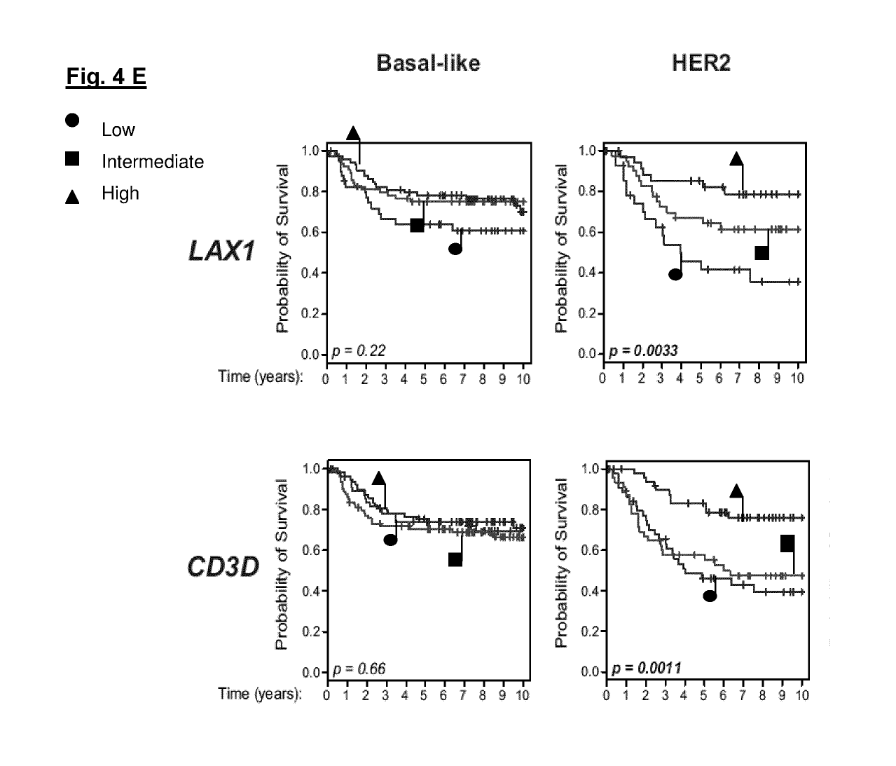

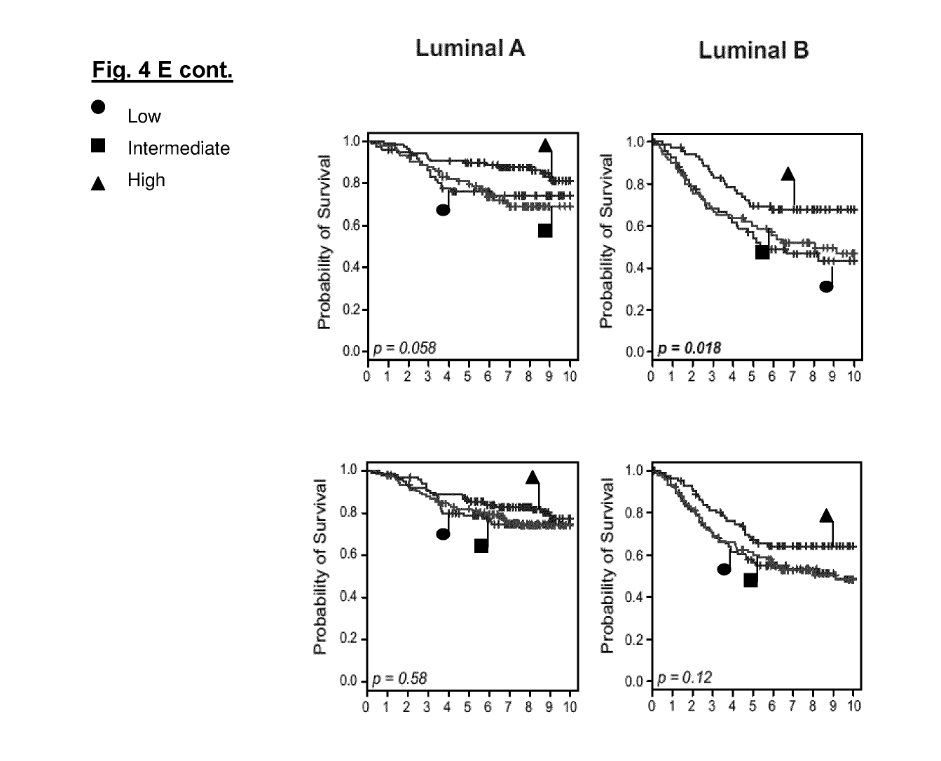

Furthermore, 13 of the 32 genes are involved in immunity and 9, particularly, in T lymphocyte biology (CD3D, CD3G, CD6, LCK, LAX1, SIT1, RHOH, UBASH3A and ICOS; FIG. 4a). Several of them, like for example LAX1, SIT1, or UBASH3A, have never been highlighted before as survival markers in breast cancer.

Consistently with the data presented in FIG. 3k-n, low methylation of the above genes correlated with high lymphocyte infiltration (except for RHOH and BST2, so these were not subsequently considered) (FIG. 4b and Table 11). When looking at the expression levels of these genes, the opposite was found, that is, high gene expression correlated with high lymphocyte infiltration (FIG. 4b and Table12). This anti-correlation between the methylation and expression status of the immune genes was also found in breast epithelial cell lines as well as in ex vivo lymphocytes and T lymphoid cell lines, as determined by DNA methylation and gene expression profiling (FIG. 4c). This is in keeping with the strong anti-correlation observed between methylation and expression status of these genes in the whole tumour samples. Furthermore, some of these genes (CD3D, CD3G, ICOS and UBASH3A) appeared highly methylated in ex vivo B lymphocytes and not in T lymphocytes samples (FIG. 4c), again indicating that the observed lymphocyte infiltration (FIG. 4b) mostly involves T lymphocytes, as suggested in FIG. 4a.

TABLE 10
Univariate Cox regression meta-analysis on
publicly available gene expression data sets.

| Variable | Hazard.Ratio | lower.95 | upper.95 | P.value | fdr | n |
|---|---|---|---|---|---|---|
| grade | 4.319051475 | 2.70533636 | 6.895336906 | 8.81E−10 | 0 | 730 |
| CD37 | 0.637528005 | 0.508909569 | 0.798652612 | 9.02E−05 | 0.003 | 951 |
| LAX1 | 0.607735237 | 0.469490691 | 0.786686777 | 0.000155589 | 0.003 | 755 |
| HCLS1 | 0.66628668 | 0.534778159 | 0.830134762 | 0.000295162 | 0.004 | 951 |
| size | 1.775376859 | 1.283496655 | 2.455762528 | 0.00052471 | 0.005 | 832 |
| RHOH | 0.670647193 | 0.535050445 | 0.840607948 | 0.000527206 | 0.005 | 952 |
| CD3G | 0.704601714 | 0.56878791 | 0.87284481 | 0.001351572 | 0.012 | 952 |
| PTPRCAP | 0.693100838 | 0.549253821 | 0.874620717 | 0.002010176 | 0.015 | 952 |
| CCR7 | 0.717640112 | 0.578403622 | 0.890394373 | 0.002571111 | 0.017 | 887 |
| ARHGAP25 | 0.79414017 | 0.679183693 | 0.928553814 | 0.003863567 | 0.02 | 950 |
| CCL5 | 0.733823788 | 0.594450738 | 0.905873806 | 0.003978873 | 0.02 | 952 |
| BST2 | 0.747004293 | 0.61181789 | 0.912061288 | 0.004187743 | 0.02 | 945 |
| PSCDBP | 0.738332573 | 0.599602639 | 0.909160421 | 0.004279438 | 0.02 | 890 |
| CD3D | 0.769590125 | 0.639626249 | 0.925960999 | 0.005519609 | 0.022 | 952 |
| NME5 | 0.7465137 | 0.607158777 | 0.91785333 | 0.005553296 | 0.022 | 951 |
| HEM1 | 0.745091977 | 0.603876135 | 0.919331005 | 0.006061245 | 0.022 | 951 |
| CENTB1 | 0.753031335 | 0.61460319 | 0.922637891 | 0.00620265 | 0.022 | 952 |
| SLC44A4 | 0.716555934 | 0.562123142 | 0.91341624 | 0.00711915 | 0.024 | 755 |
| ICOS | 0.776943611 | 0.644775259 | 0.936204307 | 0.007980999 | 0.024 | 950 |
| PPP1R16B | 0.757698984 | 0.616947476 | 0.930561794 | 0.008136743 | 0.024 | 887 |
| CIDEB | 0.765412525 | 0.618428587 | 0.947330614 | 0.01399867 | 0.04 | 952 |
| UBASH3A | 0.816472324 | 0.693874277 | 0.960731761 | 0.014584306 | 0.04 | 952 |
| CD6 | 0.791045558 | 0.653436134 | 0.957634637 | 0.016220318 | 0.042 | 944 |
| TRAF3IP3 | 0.79027337 | 0.648137351 | 0.963579706 | 0.019981307 | 0.05 | 881 |
| DNALI1 | 0.803318339 | 0.666106667 | 0.968794318 | 0.021922321 | 0.053 | 952 |
| PADI3 | 1.282586832 | 1.027770903 | 1.600579446 | 0.027639763 | 0.064 | 950 |
| SIT1 | 0.786510638 | 0.632504795 | 0.978014693 | 0.030779914 | 0.064 | 950 |
| CD52 | 0.798287393 | 0.65008143 | 0.980281442 | 0.031552946 | 0.064 | 949 |
| node | 1.854933997 | 1.051885878 | 3.271058394 | 0.032782279 | 0.064 | 273 |
| GPR171 | 0.797959507 | 0.64844202 | 0.981952673 | 0.033006747 | 0.064 | 950 |
| MAGEA10 | 1.251763319 | 1.018281633 | 1.538779996 | 0.033009551 | 0.064 | 951 |
| LCK | 0.80314799 | 0.652889033 | 0.987988251 | 0.038050335 | 0.071 | 951 |
| SP140 | 0.801792991 | 0.648901416 | 0.990708273 | 0.040712689 | 0.074 | 886 |
| CD79B | 0.796167392 | 0.638244197 | 0.993166126 | 0.043305166 | 0.076 | 951 |
| BIN2 | 0.814941986 | 0.664344694 | 0.999677496 | 0.049639411 | 0.085 | 946 |
| PTPN7 | 0.792341795 | 0.626269948 | 1.002451932 | 0.05243348 | 0.087 | 951 |
| PDZK1 | 0.813311899 | 0.654827403 | 1.010153578 | 0.061677068 | 0.1 | 952 |
| HMGCS2 | 0.823324053 | 0.6700983 | 1.011586651 | 0.064267705 | 0.101 | 946 |
| TRAF1 | 0.860049164 | 0.714185188 | 1.035704152 | 0.111836932 | 0.172 | 952 |
| PIK3CG | 0.852864273 | 0.693732209 | 1.048498915 | 0.130918607 | 0.196 | 952 |
| CCBP2 | 0.851353503 | 0.684907289 | 1.058249487 | 0.147091806 | 0.215 | 952 |
| CALML5 | 1.152320561 | 0.948006825 | 1.400667843 | 0.154512732 | 0.221 | 946 |
| SCRG1 | 1.186854771 | 0.928265972 | 1.517479138 | 0.171850684 | 0.24 | 952 |
| age | 0.843892288 | 0.634787305 | 1.121878442 | 0.242671976 | 0.331 | 832 |
| er | 0.879914817 | 0.674422359 | 1.148019599 | 0.34581516 | 0.461 | 885 |
| S100A1 | 1.100038426 | 0.877702372 | 1.378695761 | 0.407879927 | 0.532 | 887 |
| ACTG2 | 1.102117932 | 0.858132785 | 1.415473174 | 0.446300424 | 0.561 | 952 |
| SCNN1A | 0.919786588 | 0.740823935 | 1.141981688 | 0.448825642 | 0.561 | 946 |
| CRYAB | 1.09273719 | 0.860375019 | 1.3878536 | 0.467187455 | 0.572 | 952 |
| LDHC | 1.076690314 | 0.874736682 | 1.325269714 | 0.485677672 | 0.583 | 950 |
| MIA | 0.935507087 | 0.744206524 | 1.175982045 | 0.56789208 | 0.668 | 952 |
| SYCP2 | 1.050297885 | 0.852423577 | 1.294105041 | 0.644966227 | 0.744 | 945 |
| KRT20 | 1.031559368 | 0.878831436 | 1.210829161 | 0.703897252 | 0.797 | 951 |
| TNS4 | 1.030114858 | 0.842888781 | 1.258928396 | 0.771886907 | 0.852 | 952 |
| SOX10 | 0.969305349 | 0.777727696 | 1.208074322 | 0.781407858 | 0.852 | 952 |
| CHRNA9 | 0.973691818 | 0.790085795 | 1.199965577 | 0.802531225 | 0.855 | 948 |
| TDRD1 | 1.033987152 | 0.784876022 | 1.362163451 | 0.812158367 | 0.855 | 690 |
| RBP1 | 0.980931649 | 0.789362527 | 1.218992372 | 0.862125942 | 0.892 | 952 |
| TFF1 | 0.988606991 | 0.822817223 | 1.187801805 | 0.902625469 | 0.918 | 942 |
| TFF3 | 1.010010328 | 0.830061805 | 1.228969766 | 0.92074585 | 0.921 | 952 |

The meta-analysis in table 10 above was performed on the genes displaying high anti-correlation between their methylation and expression status (Pearson's coefficient below than −0.7), as described in the Supplementary Methods. The prognostic value of the classical markers (grade, tumour size, nodal status, age of the patient at diagnosis, ER status) was also evaluated. Lower.95 and Upper.95 indicate the 95% confidence interval of the hazard ratio, and n, the number of patients.

TABLE 11
Spearman correlation between methylation
status of immune genes described in Figure 4
and the stromal and intratumoral lymphocyte infiltration.

| | | intratumoral | | stromal | |
|---|---|---|---|---|---|
| | | lymphocyte | | lymphocyte | |
| | | infiltration | | infiltration | |
| Gene_Name | Illumina_ID | rho | p-value | rho | p-value |
| LCK | cg17078393 | −0.554 | <0.001 | −0.584 | <0.001 |
| CD3D | cg24841244 | −0.480 | <0.001 | −0.563 | <0.001 |
| CD3D | cg07728874 | −0.548 | <0.001 | −0.622 | <0.001 |
| CD6 | cg07380416 | −0.589 | <0.001 | −0.649 | <0.001 |
| CO6 | cg09902130 | −0.586 | <0.001 | −0.635 | <0.001 |
| ICOS | cg15344028 | −0.583 | <0.001 | −0.579 | <0.001 |
| CD3G | cg15880738 | −0.480 | <0.001 | −0.514 | <0.001 |
| SIT1 | cg15518883 | −0.536 | <0.001 | −0.598 | <0.001 |
| BST2 | cg16363586 | −0.163 | 0.095 | −0.144 | 0.141 |
| CCL5 | cg10315334 | −0.572 | <0.001 | −0.559 | <0.001 |
| HCLS1 | cg00141162 | −0.508 | <0.001 | −0.534 | <0.001 |
| RHOH | cg00804392 | −0.123 | 0.212 | −0.262 | 0.007 |
| RHOH | cg11903057 | −0.068 | 0.489 | −0.198 | 0.041 |
| CD79B | cg07973967 | −0.461 | <0.001 | −0.468 | <0.001 |
| UBASH3A | cg00134539 | −0.360 | <0.001 | −0.310 | 0.001 |
| LAX1 | cg10117369 | −0.404 | <0.001 | −0.434 | <0.001 |

TABLE 12
Spearman correlation between expression status of
immune genes described in Figure 4 and
the stromal and intratumoral lymphocyte infiltration.

| | | intratumoral | | stromal | |
|---|---|---|---|---|---|
| | | lymphocyte | | lymphocyte | |
| | | infiltration | | infiltration | |
| Gene_Name | Affy_ID | rho | p-value | rho | p-value |
| LCK | 204891_s_at | 0.508 | <0.001 | 0.624 | <0.001 |
| CD3D | 213539_at | 0.472 | <0.001 | 0.606 | <0.001 |
| CD6 | 213958_at | 0.451 | <0.001 | 0.582 | <0.001 |
| ICOS | 210439_at | 0.571 | <0.001 | 0.63 | <0.001 |
| CD3G | 206804_at | 0.423 | <0.001 | 0.54 | <0.001 |
| SIT1 | 205484_at | 0.545 | <0.001 | 0.642 | <0.001 |
| BST2 | 201641_at | 0.033 | 0.77 | 0.118 | 0.297 |
| CCL5 | 1405_i_at | 0.545 | <0.001 | 0.634 | <0.001 |
| HCLS1 | 202957_at | 0.471 | <0.001 | 0.542 | <0.001 |
| RHOH | 204951_at | −0.013 | 0.907 | 0.173 | 0.124 |
| CD79B | 205297_s_at | 0.563 | <0.001 | 0.613 | <0.001 |
| UBASH3A | 220418_at | 0.434 | <0.001 | 0.551 | <0.001 |
| LAX1 | 207734_at | 0.526 | <0.001 | 0.646 | <0.001 |

Next, the association between the above 11 immune genes and clinical outcome was analysed. High expression of all of them was associated with a better outcome (FIG. 4d), and interestingly, a multivariate analysis revealed that all of them, except CD6, seem to have an independent prognostic value to currently used clinical indicators (Tables 13 and 14). A detailed survival analysis of the 11 immune genes revealed a subtype-specific prognostic value of these genes.

TABLE 13
Multivariate Cox regression meta-analysis on publicly available gene
expression data sets.
This analysis was performed on the 11 immune genes appearing as good
prognostic markers in the univariate Cox regression provided in Supplementary
Table S25 and displaying a good correlation with stromal and intratumoral
infiltration (Supplementary Tables S26 and S27). Lower.95 and Upper.95
indicate the 95% confidence interval of the hazard ratio, and n, the number
of patients.

| Variable | Hazard.Ratio | Lower.95 | Upper.95 | P. value | n |
|---|---|---|---|---|---|
| age | 0.782098169 | 0.57957839 | 1.055383632 | 0.107962559 | 741 |
| size | 1.340020576 | 0.961479484 | 1.867595902 | 0.083981212 | 741 |
| grade | 4.398033207 | 2.686723253 | 7.199363041 | 3.85E−09 | 741 |
| er | 0.925961144 | 0.676930243 | 1.266606197 | 0.63032068 | 741 |
| node | 1.993075765 | 1.136034208 | 3.496682561 | 0.016187435 | 741 |
| SIT1 | 0.6599917 | 0.502365102 | 0.867076638 | 0.002842138 | 741 |
| age | 0.947747159 | 0.666485182 | 1.347703897 | 0.765118789 | 546 |
| size | 1.296223628 | 0.813921483 | 2.064321596 | 0.274489122 | 546 |
| grade | 4.923533758 | 2.464824018 | 9.834854125 | 6.32E−06 | 546 |
| er | 0.824491233 | 0.558241611 | 1.217726842 | 0.33207764 | 546 |
| node | 5.23442121 | 1.237767511 | 22.13595458 | 0.024455015 | 546 |
| LAX1 | 0.446127817 | 0.310119717 | 0.641784505 | 1.36E−05 | 546 |
| age | 0.815730376 | 0.605709362 | 1.098573158 | 0.179926027 | 742 |
| size | 1.350261099 | 0.968961036 | 1.881608204 | 0.076108607 | 742 |
| grade | 4.270712254 | 2.62015025 | 6.961044754 | 5.74E−09 | 742 |
| er | 0.898932232 | 0.655768704 | 1.232262462 | 0.507900025 | 742 |
| node | 1.985456613 | 1.130239988 | 3.487788438 | 0.017039196 | 742 |
| HCLS1 | 0.602372212 | 0.460056401 | 0.788712603 | 0.000227835 | 742 |
| age | 0.791016381 | 0.586069628 | 1.067632386 | 0.125464002 | 743 |
| size | 1.336212924 | 0.957464668 | 1.864784192 | 0.088312944 | 743 |
| grade | 4.447305084 | 2.707212296 | 7.305863133 | 3.81E−09 | 743 |
| er | 0.883656243 | 0.644025948 | 1.212448594 | 0.44346137 | 743 |
| node | 2.028490613 | 1.15797223 | 3.553430785 | 0.013408473 | 743 |
| CD3D | 0.667293158 | 0.543518382 | 0.819255013 | 0.000111334 | 743 |
| age | 0.814972815 | 0.603243078 | 1.101016677 | 0.182534825 | 741 |
| size | 1.455661468 | 1.04379377 | 2.030046903 | 0.026929076 | 741 |
| grade | 4.396887623 | 2.686037542 | 7.197449948 | 3.87E−09 | 741 |
| er | 0.869706949 | 0.63578294 | 1.189698764 | 0.382491166 | 741 |
| node | 1.855844417 | 1.061416677 | 3.244869404 | 0.030079032 | 741 |
| ICOS | 0.640822787 | 0.520023632 | 0.789683042 | 2.97E−05 | 741 |
| age | 0.843106773 | 0.623527268 | 1.140012743 | 0.267567194 | 735 |
| size | 1.400276591 | 1.000264809 | 1.960255439 | 0.049819954 | 735 |
| grade | 4.103756115 | 2.4933814 | 6.754207057 | 2.79E−08 | 735 |
| er | 0.98494381 | 0.718402528 | 1.350377081 | 0.924928239 | 735 |
| node | 1.96365591 | 1.107469501 | 3.481761375 | 0.020927592 | 735 |
| CD6 | 0.875910603 | 0.739643346 | 1.037282885 | 0.124615675 | 735 |
| age | 0.810235146 | 0.599268909 | 1.0954698 | 0.171489956 | 742 |
| size | 1.350831988 | 0.967991343 | 1.885086135 | 0.076955251 | 742 |
| grade | 4.097163474 | 2.511916282 | 6.682845544 | 1.61E−08 | 742 |
| er | 0.909139677 | 0.664161613 | 1.244478657 | 0.552087671 | 742 |
| node | 2.037337019 | 1.162122985 | 3.571689214 | 0.012972722 | 742 |
| CD79B | 0.664381808 | 0.502243714 | 0.878862541 | 0.004175719 | 742 |
| age | 0.781222718 | 0.577860841 | 1.05615209 | 0.108527271 | 742 |
| size | 1.355296369 | 0.971945329 | 1.889847293 | 0.073098388 | 742 |
| grade | 4.268909828 | 2.609544229 | 6.983438303 | 7.49E−09 | 742 |
| er | 0.874992826 | 0.63607609 | 1.20364915 | 0.411792841 | 742 |
| node | 1.986145103 | 1.13538492 | 3.474392075 | 0.016173634 | 742 |
| LCK | 0.673584038 | 0.518662828 | 0.874779203 | 0.003044328 | 742 |
| age | 0.793768255 | 0.587825226 | 1.071862885 | 0.131780585 | 743 |
| size | 1.361230624 | 0.980008306 | 1.89074807 | 0.065840561 | 743 |
| grade | 4.645701264 | 2.839822777 | 7.599960255 | 9.58E−10 | 743 |
| er | 0.777853284 | 0.561584487 | 1.077408201 | 0.130686899 | 743 |
| node | 1.944247797 | 1.112078104 | 3.399131305 | 0.019665701 | 743 |
| CCL5 | 0.551404359 | 0.428004708 | 0.710381828 | 4.11E−06 | 743 |
| age | 0.81183076 | 0.601704913 | 1.095336216 | 0.172537127 | 743 |
| size | 1.353550939 | 0.969870861 | 1.889014526 | 0.07506301 | 743 |
| grade | 4.307262419 | 2.625996736 | 7.064940063 | 7.30E−09 | 743 |
| er | 0.926305947 | 0.678170929 | 1.265230741 | 0.630383585 | 743 |
| node | 1.944462487 | 1.1116814 | 3.401095279 | 0.019747903 | 743 |
| UBASH3A | 0.741503992 | 0.62442346 | 0.880537337 | 0.000647399 | 743 |
| age | 0.792286599 | 0.587059106 | 1.069258699 | 0.127966947 | 743 |
| size | 1.305194443 | 0.936821995 | 1.818416458 | 0.115431743 | 743 |
| grade | 4.52739965 | 2.77339849 | 7.390696887 | 1.55E−09 | 743 |
| er | 0.833481525 | 0.606620946 | 1.145182104 | 0.261157201 | 743 |
| node | 1.863800138 | 1.06402145 | 3.264737712 | 0.029485291 | 743 |
| CD3G | 0.552580273 | 0.423133705 | 0.721627594 | 1.33E−05 | 743 |

TABLE 13b
Further info on the Immune genes and the Illumina ID's found
to be correlating to Breast cancer as described above:

| Seq id | | | | |
|---|---|---|---|---|
| no | Gene_Name | Affy_ID | Illumina ID | GeneID |
| 500 | LCK | 204891_s_at | cg17078393 | 3932 |
| 501 | CD3D | 213539_at | cg24841244 | 915 |
| 502 | CD3D | 213539_at | cg07728874 | 915 |
| 503 | CD6 | 213958_at | cg07380416 | 923 |
| 504 | CD6 | 213958_at | cg09902130 | 923 |
| 505 | ICOS | 210439_at | cg15344028 | 29851 |
| 506 | CD3G | 206804_at | cg15880738 | 917 |
| 507 | SIT1 | 205484_at | cg15518883 | 27240 |
| 508 | CCL5 | 1405_i_at | cg10315334 | 6352 |
| 509 | HCLS1 | 202957_at | cg00141162 | 3059 |
| 510 | CD79B | 205297_s_at | cg07973967 | 974 |
| 511 | UBASH3A | 220418_at | cg00134539 | 52247 |
| 512 | LAX1 | 207734_at | cg10117369 | 54900 |

TABLE 14
Immune markers appear significant in a multivariate analysis with
all the classical markers used clinically, as shown for the LAX1 and
CD3D genes used as examples (see also Table 15 for
the complete analysis).

| | | Lower | | | |
|---|---|---|---|---|---|
| Variable | Hazard ratio | 95% CI | Upper 95% CI | P-value | n |
| Age | 0.948 | 0.666 | 1.348 | 0.765 | 546 |
| Size | 1.296 | 0.814 | 2.064 | 0.274 | 546 |
| Grade | 4.923 | 2.465 | 9.835 | 6 · 10⁻⁶ | 546 |
| ER | 0.824 | 0.558 | 1.218 | 0.332 | 546 |
| Node | 5.234 | 1.238 | 22.136 | 0.024 | 546 |
| LAX1 | 0.446 | 0.31 | 0.642 | 1 · 10⁻⁵ | 546 |
| Age | 0.791 | 0.586 | 1.068 | 0.125 | 743 |
| Size | 1.336 | 0.957 | 1.865 | 0.088 | 743 |
| Grade | 4.447 | 2.707 | 7.306 | 4 · 10⁻⁹ | 743 |
| ER | 0.884 | 0.644 | 1.212 | 0.443 | 743 |
| Node | 2.028 | 1.158 | 3.553 | 0.013 | 743 |
| CD3D | 0.667 | 0.543 | 0.819 | 1 · 10⁻⁴ | 743 |

n, Number of patients;
CI, Confidence interval.

Most of these markers showed high prognostic value in HER2-overexpressing and luminal B tumours, but none of them had an impact in luminal A tumours; only a few seemed to have prognostic value in basal-like tumours (FIG. 4e and Table 15). Overall, these results show that the presence of these markers, associated with a better prognosis, reflects an antitumour T-cell response, specific for certain tumour categories. In addition, these data highlight the importance of DNA methylation analyses in revealing components of breast cancers, like the immune component described here, that were not that apparent on the basis of classical gene expression analyses (the latter having revealed principally the cell proliferation component as the major prognostic marker for breast cancer).

TABLE 15
Univariate Cox regression meta-analysis on publicly
available gene expression data sets specific for each “known
expression subtype”. Lower.95/upper.95, 95% confidence
interval of the hazard ratio; n, number of patients.

| | | | | | | |
|---|---|---|---|---|---|---|
| Variable | Hazard.Ratio | Lower.95 | Upper.95 | P.value | fdr | n |

BASAL-LIKE

| | | | | | | |
|---|---|---|---|---|---|---|
| CD6 | 0.571415127 | 0.35980797 | 0.907470858 | 0.017721616 | 0.032784991 | 213 |
| CCL5 | 0.601220984 | 0.379386705 | 0.952765786 | 0.030315366 | 0.053412788 | 213 |
| CD3G | 0.614974481 | 0.393006583 | 0.962308592 | 0.033325393 | 0.056047253 | 213 |
| LAX1 | 0.552834594 | 0.319001003 | 0.958072497 | 0.03463195 | 0.055712264 | 178 |
| CD3D | 0.599642986 | 0.363138343 | 0.99017831 | 0.045658689 | 0.070390478 | 213 |
| age | 0.557241661 | 0.295973189 | 1.049143235 | 0.070085346 | 0.103726313 | 172 |
| LCK | 0.632048217 | 0.376236164 | 1.061793059 | 0.083020423 | 0.113768734 | 213 |
| HCLS1 | 0.694316555 | 0.449956311 | 1.071382857 | 0.099266112 | 0.131173074 | 213 |
| grade | 2.333835064 | 0.60915775 | 8.941503419 | 0.216206627 | 0.266654849 | 155 |
| ICOS | 0.765441762 | 0.47602165 | 1.230828665 | 0.270037378 | 0.322302669 | 213 |
| er | 1.325149161 | 0.603157506 | 2.911379334 | 0.483286797 | 0.55880034 | 208 |
| UBASH3A | 0.84970099 | 0.528860792 | 1.365183019 | 0.500797496 | 0.561500251 | 213 |
| SIT1 | 0.851938648 | 0.532926849 | 1.361911981 | 0.5031992 | 0.547599137 | 213 |
| CD79B | 0.864632082 | 0.524298487 | 1.425883645 | 0.568758172 | 0.601258636 | 213 |
| node | 0.631158808 | 0.081569127 | 4.883728148 | 0.659341077 | 0.677656114 | 211 |
| size | 0.93955348 | 0.449321006 | 1.964654956 | 0.86842147 | 0.868421495 | 172 |

HER2

| | | | | | | |
|---|---|---|---|---|---|---|
| ICOS | 0.665653573 | 0.520062316 | 0.85200305 | 0.001230088 | 0.002167298 | 142 |
| node | 4.604533941 | 1.787955465 | 11.85808776 | 0.001556726 | 0.00261813 | 142 |
| LAX1 | 0.379778681 | 0.20236605 | 0.712727492 | 0.002575214 | 0.004142736 | 105 |
| CD3D | 0.517574299 | 0.306380997 | 0.87434651 | 0.013820016 | 0.020453623 | 142 |
| LCK | 0.533630219 | 0.318779166 | 0.893286769 | 0.01688217 | 0.024024626 | 142 |
| CD3G | 0.574943427 | 0.345611487 | 0.956449529 | 0.033053232 | 0.045295168 | 142 |
| size | 1.904053799 | 1.009143609 | 3.592571797 | 0.046804702 | 0.061849073 | 126 |
| UBASH3A | 0.639066456 | 0.399576092 | 1.022098029 | 0.061659162 | 0.078668587 | 142 |
| HCLS1 | 0.651479447 | 0.405250274 | 1.047316924 | 0.076877637 | 0.094815753 | 142 |
| CCL5 | 0.637778183 | 0.387309781 | 1.050221372 | 0.077159864 | 0.092094034 | 142 |
| SIT1 | 0.656499672 | 0.410184716 | 1.050726179 | 0.079472098 | 0.091889612 | 141 |
| CD79B | 0.720339802 | 0.411022928 | 1.262434273 | 0.251839036 | 0.282364994 | 142 |
| CD6 | 0.875933541 | 0.692310708 | 1.108258994 | 0.269768688 | 0.2935718 | 138 |
| age | 1.410285548 | 0.750438055 | 2.650325787 | 0.285499481 | 0.301813751 | 126 |
| er | 1.106033277 | 0.63703866 | 1.920306706 | 0.720323254 | 0.740332246 | 136 |
| grade | 1.137095166 | 0.400598853 | 3.22763135 | 0.809271597 | 0.809271574 | 106 |

Luminal A

| | | | | | | |
|---|---|---|---|---|---|---|
| grade | 5.162337792 | 2.065135769 | 12.90459053 | 0.000445859 | 0.000824839 | 275 |
| size | 1.850306583 | 0.961583288 | 3.560413844 | 0.065378974 | 0.115191519 | 318 |
| CD3D | 0.697135966 | 0.472866537 | 1.027771088 | 0.068507829 | 0.115217708 | 345 |
| UBASH3A | 0.768113097 | 0.566321462 | 1.041807117 | 0.089776717 | 0.14442341 | 345 |
| SIT1 | 0.663341846 | 0.408478686 | 1.077222434 | 0.09706223 | 0.14963761 | 345 |
| CCL5 | 0.672449535 | 0.410573335 | 1.101358365 | 0.114925908 | 0.170090348 | 345 |
| CD79B | 0.741453969 | 0.470759597 | 1.167801977 | 0.196817333 | 0.280086219 | 344 |
| HCLS1 | 0.74338516 | 0.437839466 | 1.262155511 | 0.272229064 | 0.373054653 | 345 |
| CD3G | 0.792669997 | 0.498933534 | 1.259337528 | 0.325256661 | 0.429803461 | 345 |
| LAX1 | 0.753425631 | 0.414668811 | 1.368924226 | 0.352748307 | 0.450058192 | 270 |
| CD6 | 0.871687669 | 0.520960507 | 1.458535496 | 0.601065641 | 0.741314292 | 344 |
| LCK | 1.080613746 | 0.681066064 | 1.714556239 | 0.742025194 | 0.857966661 | 344 |
| er | 1.123321638 | 0.342705919 | 3.682024241 | 0.847750681 | 0.950508296 | 319 |
| age | 0.968467546 | 0.541901248 | 1.730812379 | 0.913873178 | 0.994509041 | 318 |
| node | 1.046039154 | 0.288465738 | 3.793164203 | 0.945400879 | 0.999423802 | 344 |
| ICOS | 0.993065905 | 0.572015048 | 1.724045364 | 0.98027602 | 1.007505894 | 344 |

Luminal B

| | | | | | | |
|---|---|---|---|---|---|---|
| LAX1 | 0.44407418 | 0.283660793 | 0.695203153 | 0.000385645 | 0.000713443 | 209 |
| CD3G | 0.529767867 | 0.354645182 | 0.791365587 | 0.001917346 | 0.003378181 | 255 |
| HCLS1 | 0.565073005 | 0.387754045 | 0.823479484 | 0.002970425 | 0.004995715 | 254 |
| CD3D | 0.609672758 | 0.432610365 | 0.85920473 | 0.00470061 | 0.007561851 | 255 |
| LCK | 0.603241335 | 0.420086816 | 0.866249772 | 0.006187718 | 0.009539398 | 255 |
| UBASH3A | 0.553322892 | 0.350383338 | 0.873803601 | 0.011128892 | 0.01647076 | 255 |
| CCL5 | 0.626047812 | 0.430208929 | 0.911036093 | 0.014415646 | 0.020514574 | 255 |
| grade | 2.774788889 | 1.191228926 | 6.463454012 | 0.018002961 | 0.024670724 | 210 |
| SIT1 | 0.617616772 | 0.411098071 | 0.927881943 | 0.020320012 | 0.025925532 | 254 |
| ICOS | 0.666539915 | 0.46455092 | 0.956354706 | 0.027648847 | 0.034100246 | 255 |
| CD6 | 0.757102121 | 0.544668538 | 1.052389814 | 0.097710234 | 0.116621897 | 255 |
| CD79B | 0.764181861 | 0.529362845 | 1.10316378 | 0.151056463 | 0.174659044 | 255 |
| size | 1.475566638 | 0.834659682 | 2.608604382 | 0.180809598 | 0.196763396 | 233 |
| age | 0.777738033 | 0.503583487 | 1.201144327 | 0.257001758 | 0.271687567 | 233 |
| er | 1.524385366 | 0.6055743 | 3.837267771 | 0.370748167 | 0.381046712 | 239 |
| node | 1.321194737 | 0.438253574 | 3.982980711 | 0.620797266 | 0.620797276 | 255 |

1. A method for the stratification and prognosis of breast cancer comprising the steps of:
a) analyzing the methylation status of one or more of the genes selected from the group consisting of: LCK, CD3D, CD6, ICOS, CD3G, SIT1, CCL5, HCLS1, CD79B, UBASH3A, and LAX1, in a sample of the subject, and
b) comparing the methylation status of said one or more genes obtained from step a) with the methylation status of a control sample,
wherein a difference in methylation status as detected in step b) indicates the subject has a good or a bad clinical outcome.

2. The method according to claim 1, wherein the methylation status of one or more CpG regions of said immune genes as defined by SEQ ID Nos 500-512 is analysed.

3. The method according to claim 1, wherein a decreased methylation of said immune genes indicates a better clinical outcome and thus a good prognosis.

4. A method for the classification, stratification, diagnosis, prognosis or prediction of breast cancer comprising the steps of:
a) analyzing the methylation status of all 86 CpG regions defined in Table 2 (SEQ ID Nos 1 to 86) in a sample of the subject, and
b) comparing the methylation status of said one or more regions obtained from step a) with the methylation status of a control sample,
wherein a difference in methylation status as detected in step b) indicates the subject has or is at risk of developing breast cancer.

5. The method according to claim 4, wherein a classifier comprising the methylation profile of the 86 CpG islands identified in Table 2 is used.

6. The method according to claim 5, wherein said breast cancers are classified into one of the six methylation subtypes according to said 86 CpG island classifier.

7. A method for the stratification, prognosis or prediction of breast cancer, or for providing an indication for susceptibility to hormonotherapy comprising the steps of:
a) analyzing the methylation status of one or more of the CpG regions defined in Table 5b (SEQ ID Nos 87 to 321) and 5c (SEQ ID Nos 322 to 499), in a sample of the subject, and
b) comparing the methylation status of said one or more regions obtained from step a) with the methylation status of a control sample,
wherein a difference in methylation status as detected in step b) indicates the susceptibility of the subject to respond to homotherapy.

8. The method according to claim 7, wherein all CpG regions defined in Table 5b (SEQ ID Nos 87 to 321) and/or all CpG regions defined in Table 5c (SEQ ID Nos 322 to 499) are analysed.

9. The method according to claim 7, used to establish whether or not said tumor belongs to the ER-positive or ER-negative subtype.

10. The method according to claim 1, wherein the difference in methylation status is due to hypermethylation or hypomethylation.

11. The method according to claim 1, wherein the sample of the subject is selected from the group comprising: a tissue, cells, a cell pellet, a cell extract, a surgical sample, a biopsy or fine needle aspirate, or is a biological fluid such as: urine, whole blood, plasma, serum, ductal fluid, lymph node fluid, tumour exudate or tumour cavity fluid.

12. The method according to claim 1, wherein the methylation status is analysed by one or more techniques selected from the group consisting of nucleic acid amplification, polymerase chain reaction (PCR), methylation specific PCR (MCP), methylated-CpG island recovery assay (MIRA), combined bisulfite-restriction analysis (COBRA), bisulfite pyrosequenceing, single-strand conformation polymorphism (SSCP) analysis, restriction analysis, microarray analysis, or bead-chip technology.

13. A method of treating breast cancer by targeting one or more genes having aberrant methylation in breast cancer, defined by one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c.

14. The method according to claim 13, wherein said targeting implies changing the methylation status by using demethylating or methylating agents, by changing the expression level, or by changing the protein activity of the protein encoded by said one or more genes.

15. The method according to claim 14, wherein said methylating agents are methyl donors such as folic acid, methionine, choline or any other chemicals capable of elevating DNA methylation.

16. A method for identifying an agent that modulates the methylation status of one or more of the genes or gene products having aberrant methylation in breast cancer, defined by one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, comprising the steps of:
a) contacting the candidate agent with said one or more genes, and
b) analysing the modulation of said one or more gene by the candidate agent.

17. The method according to claim 16, wherein said agent modulates the methylation status, the expression level or the activity of said one or more gene.

18. A method for establishing a reference methylation status profile comprising the steps of: measuring the methylation status of one or more genes having aberrant methylation in breast cancer, defined by one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c in a sample of subject.

19. The method according to claim 18, wherein said subject is healthy, thereby producing a reference profile of a healthy subject, or wherein said subject is suffering from breast cancer, or Basal-like, Luminal A, luminal B, HER2-plus or HER2-minus breast cancer, thereby producing a specific breast cancer type reference profile.

20. A methylation status reference profile for the stratification, prognosis, diagnosis or prediction of breast cancer comprising the methylation status of one or more CpG regions from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, obtainable according to claim 17.

21. A microarray or chip comprising one or more breast cancer specific CpG regions from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c.

22. A method of treating breast cancer comprising determining the methylation status of one or more of the CpG islands from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c in a patient sample, stratifying, prognosticating, diagnosing or predicting clinical outcome for breast cancer based upon the methylation status, selecting patients having a poor clinical outcome, and treating the patients having a poor clinical outcome.

23. A method of stratifying breast cancer patients comprising the steps of:
a) analyzing the methylation status of one or more of the CpG islands from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, in a sample of the subject, and
b) comparing the methylation status of said one or more genes obtained from step a) with the methylation status of a control sample selected from the group of healthy, or Basal-like, Luminal A, luminal B, HER2-plus or HER2-minus breast cancer,
wherein a corresponding methylation status in steps a) and b) results in the identification of the type of breast cancer.

24. A method of selecting a breast cancer therapy comprising the steps of
a) analyzing the methylation status of one or more of the CpG islands from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, in a sample of the subject, and
b) comparing the methylation status of said one or more genes obtained from step a) with the methylation status of a control sample selected from the group of healthy, or Basal-like, Luminal A, luminal B, HER2-plus or HER2-minus breast cancer,
wherein a corresponding methylation status in steps a and b results in the identification of the type of breast cancer, and
c) identifying the appropriate treatment of the breast cancer in view of the type of cancer identified.

25. A kit for the stratification, prognosis, diagnosis or prediction of breast cancer comprising the microarray according to claim 21, and one or more reference profiles comprising the methylation status of one or more CpG regions from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c.

26. A kit for the stratification, prognosis, diagnosis or prediction of breast cancer comprising means for analyzing the methylation status of one or more CpG regions from one or more of the genes selected from the group comprising: LCK, CD3D, CD6, ICOS, CD3G, SIT1, CCL5, HCLS1, CD79B, UBASH3A, and LAX1, or CpG regions defined in Tables 2, 5b or 5c, and one or more reference profiles according to claim 20.


SEQUENCE LISTING (574 sequences)

SEQ ID NO: 1 - DNA, 122 bp, Homo Sapiens
tggattcatccctcattcactccacgcttgggtttggacaccaccacataccaggcactacgctaggcttgggatgggccgaagaacaaaacagaacaggtcttttgtttaccttctaggag

SEQ ID NO: 2 - DNA, 122 bp, Homo Sapiens
atcacatcaccagcaatctccctcctaggtagaagccccaaagaattaacaacaaaaggacgcaaacagatacttgtacacccatggtcacagcagcctgactcacaacagaacaaacagcc

SEQ ID NO: 3 - DNA, 122 bp, Homo Sapiens
tagctctggcaggagaggagctgtcctaggccactcttccaggatgtggctcccccactgcgggaccagaccttttatactgaccggatccggattctctgagtgcattccaccaggagcca

SEQ ID NO: 4 - DNA, 122 bp, Homo Sapiens
accgcttctctctaagcaggtcactgaccgaggaaaccaagcctcatttcaaaataatatcgttttccaatgaagtcaactgtccaactactgtttagcttcaccagaacagcaaacaaact

SEQ ID NO: 5 - DNA, 122 bp, Homo Sapiens
acagcacttacttcacagggcttgattattaaacaagttaacatctttgggctcctagaacgatgccatgtgcagagtcaggactgggtaaatatttgctaaagcaatctcttcatgttttt

SEQ ID NO: 6 - DNA, 122 bp, Homo Sapiens
ttcataaagtaaatatctttttcattgctctccagcacatgctaagaatttagcttccaccgccttaagtaaacactctgcgataccagtcctaaaaaagtcactgtggcaggggctgtggt

SEQ ID NO: 7 - DNA, 122 bp, Homo Sapiens
gagagatccttgtcatcttccttctccctgcaggagggtgtcagggtgtaagtgctccctcgctgtgcaggggttcatttcattcatttcattacccttgccctcctcgaggtacctccggg

SEQ ID NO: 8 - DNA, 122 bp, Homo Sapiens
gaaaaacaacaggaagcagcttacaaactcggtgaacaactgagggaaccaaaccagagacgcgctgaacagagagaatcaggctcaaagcaagtggaagtgggcagagattccaccaggac

SEQ ID NO: 9 - DNA, 122 bp, Homo Sapiens
aatcagtggagattattgtctcagaggatccccgggcctccttaggcaaatgttatctaacgctctttaagcaaacagagcctgccctataaaatccggggctcgggcggcctctcatccct

SEQ ID NO: 10 - DNA, 122 bp, Homo Sapiens
acctgagcaagaccagtagttcattccaagagcaaacataatcaatatcttcatcatcatcgtcatcttctccattctggaaatcatgattgtaatcgagagacagagagtgtctctctatg

SEQ ID NO: 11 - DNA, 122 bp, Homo Sapiens
tccacaaagtactttccatcagatacacttttctgatggaaaccaggtgtgtgatggttacggccccaggttagctccagagcacattcaactgtgggtaaacacaaatgtgccctgtgcca

SEQ ID NO: 12 - DNA, 122 bp, Homo Sapiens
tggttttccaggatatgatagagctcgcggtagttgccaccgtgaaaggccacgatggctcgtgcgcgtagcaccgactcattcttgttgagggcctcgcaggccgcaggggccacgggcag

SEQ ID NO: 13 - DNA, 122 bp, Homo Sapiens
ctggcagctcaggacaagtagaccagccagcaggacacagatcttcctacccggctgattcggagacgagaccaagctgttgctgaggacgacagccctgctctggtcactccggaggctga

SEQ ID NO: 14 - DNA, 122 bp, Homo Sapiens
ctagtgtaaattgtgcttagaccaataacacatttaaaattatagagcttttcatcactacggagatgctcttgcctatgataggaacgatggctacatctgaagctgagcaaatttatctt

SEQ ID NO: 15 - DNA, 122 bp, Homo Sapiens
gccagccgccgcgaccgccgcggctgcagcctccgaagggaggccgggtgagccggcgtacgcactttcccgcggactttcggagtgtttgtggatatacatgccaagccgccacgatgatg

SEQ ID NO: 16 - DNA, 122 bp, Homo Sapiens
acggcgcggccatggagcgcccggcgcctctcgtcgcactgaaactttgccaagagtgcgcgggcccgacaggctctgtgtcatctttcttgaaccgaacctggagtttccgctgctaattc

SEQ ID NO: 17 - DNA, 122 bp, Homo Sapiens
tgtccagtggagttcttcttccttctcctttttctgctccttctttcattgttggctcaacgtcaaagcagaaatctctaaagtttgctgatcgatcttcacataactgttctggtgatatc

SEQ ID NO: 18 - DNA, 122 bp, Homo Sapiens
cttttcagccctcagtgcccattttgccaataaaaagtcccaaggtgacagtacaagagacgcctttagtgaaggcaaaggaagggacactcccctcctttgctgcctactctcgccctcac

SEQ ID NO: 19 - DNA, 122 bp, Homo Sapiens
ctcaaatgatccacccacctcagcctcccaaagtgctgggaatacaggtgtgagccacagcgcccagccagaagatttttaatagaagtaaatagaaagtgaagcaataagtgagtaaataa

SEQ ID NO: 20 - DNA, 122 bp, Homo Sapiens
gtggtgttccccggactcttcctcctctccaagaacacgctccagcggctgccccagctacgctgggaggaggccgacgcagtcattgtctcagccaggtaaaccactttctttcccctcag

SEQ ID NO: 21 - DNA, 122 bp, Homo Sapiens
gtattggttctaaaataatactggaagcttttgctcataatgtggtttctgcctcacccacgcacagcaatcctggtgttctatttttgttgcctgtttctcaggctacactgcagttggct

SEQ ID NO: 22 - DNA, 122 bp, Homo Sapiens
ggctgcggggccgccgccgagcgagggcgaggagagcaccgtgcgcttcgcccgcaaaggcgccctccggcagaagaacgtgcatgaggtcaagaaccacaaattcaccgcccgcttcttca

SEQ ID NO: 23 - DNA, 122 bp, Homo Sapiens
atttgtttgctttaatttttcagactgactccctagaggtcgcttcttgtttgcatagttcgtcagtcaaacatcagttaaagccaatatggtttccaccatctgctgctggatctatgtgc

SEQ ID NO: 24 - DNA, 122 bp, Homo Sapiens
acacctggtcctgctcttcagacacaggaccctggtatttctaagaaatcggaaaaccgtcgcccagaggaaaaggaagtcttttgaagagggtgcccggcgtgggcagtggggcagcgccc

SEQ ID NO: 25 - DNA, 122 bp, Homo Sapiens
ttacttactgttggagattgatgcttcttttttgtctctgcagctcaagttctccagcttcgccatttacttcagcaaatattaataaggtaagcaaactccaaagaaaaggtaaagacatg

SEQ ID NO: 26 - DNA, 122 bp, Homo Sapiens
tccaacaaagttaggtttgttgacctattcagtgagggacaccacccaccagaagagctacgcacatcaccaaacaaaggaaaacagcaatttattataggattccgaggaaagtggagttg

SEQ ID NO: 27 - DNA, 122 bp, Homo Sapiens
cccccaccccttcctccctccagctgtcccggctgggaagcagccgcgtagacagatagacggactccagaccgccagctgagacctttagctcaactagtggttggcactaagctggggcg

SEQ ID NO: 28 - DNA, 122 bp, Homo Sapiens
ctacaatgtttaggattgggaggagacagctctggaagcatagcgtcactcgagttttaacggtaaggtgctgttgtctgtacctttgcatgtgtgttttggattatcatctgtcacacctg

SEQ ID NO: 29 - DNA, 122 bp, Homo Sapiens
ctgggaaacttaggacaagtgactcaaactctctgaggcccagtgttctgatgataagtgcgggtctcataggtcattgtgagactacttagtgaatatccatggagtcctcagcaaaaacc

SEQ ID NO: 30 - DNA, 122 bp, Homo Sapiens
tcctgcctcaggccgtgagtacatgcagctgaactgctgtcagtcttatctgtccagtgtcgggtgttgtgtgtccagccacccataaccctgggttgggactccctccctcaccaaccggg

SEQ ID NO: 31 - DNA, 122 bp, Homo Sapiens
accggctcccgcgcgcctcccctcgcgcccgagcttcgagccaagcagcgtcctggggagcgcgtcatggccttaccagtgaccgccttgctcctgccgctggccttgctgctccgtgagtt

SEQ ID NO: 32 - DNA, 122 bp, Homo Sapiens
tgacatccatctaaggccctgagtggtctgaccttacttaattcttcatctgtaatgactcgtcctccccagccccgcataagcacaggtagagtctgtagggtttaccgccccgatacacc

SEQ ID NO: 33 - DNA, 122 bp, Homo Sapiens
gtgatgatgataataataataatatagcaactctcagcttttctacagcactctgctttccgctgtttccaactcattttgtgactggaggccaggaatctcacttctgaacctgctttttt

SEQ ID NO: 34 - DNA, 122 bp, Homo Sapiens
cagctaacatttagcattttctgataccttttatctcaaaacctttagataaacttcaaacgccatgtcatgacaatattagaattgtatttacattatgtatacacatctcttccttccaa

SEQ ID NO: 35 - DNA, 122 bp, Homo Sapiens
gcagccccgccgcccgccgtggggtcggggacagggagaagggagtgcctgcctggtctgcgccccccgcctgtcagcccttgcctcgaggctctggggcacccaactcgtcgactcctgac

SEQ ID NO: 36 - DNA, 122 bp, Homo Sapiens
ttttttttttttttctaattcctgaggggtggttgctgcttttgctacatgacttgccagcgcccgagcctgcggtccaactgcgctgctgccggagcgctcagtgccgccgctgccgcccg

SEQ ID NO: 37 - DNA, 122 bp, Homo Sapiens
cttggcaggagagatgagaccccttgaatattgaagaggctgccgctcttcactcagaggcggcgggatctgattaaacatgcttacaggaacgactgtctccaatggctttcctccagcca

SEQ ID NO: 38 - DNA, 122 bp, Homo Sapiens
cccagaggcttggaaatccaggagccacaggaagcagtgagctttctgcggagccggatccggtggagccaattcaccagcaggtcccggcagcaactcttctacaacagattcctgcccgt

SEQ ID NO: 39 - DNA, 122 bp, Homo Sapiens
gggagccacatcctggaagagtggcctaggacagctcctctcctgccagagctaggcaggcgccgaagtagccgcatggccccgtcagaagaccccagggactggagagccaacctcaaagg

SEQ ID NO: 40 - DNA, 122 bp, Homo Sapiens
ctcgtccggccgcgaccagcgcgtgggagccgctcttatagtgaccaccagcaaggacagcgcaggtgatgcacctgtagctacccgggcatgcgcactgcccgcctcacctttcccaccgt

SEQ ID NO: 41 - DNA, 122 bp, Homo Sapiens
attatttacctgacagggcaaaagagattttgcagatgcaattaaggttaaggaccttgacgtgggaagattgtgattatttacctgacagggcaaaagagattttgcagatgcaattaagg

SEQ ID NO: 42 - DNA, 122 bp, Homo Sapiens
gtgtcccttaaaagctggggcctgggacaggaacgacagacaatgcagccaatggcgtcacgcgcggtgccccgctacccaatcgaaaggcgtggctgagggaaacgcggtgggaaccgccc

SEQ ID NO: 43 - DNA, 122 bp, Homo Sapiens
ccaagctgaggaagctcatccccactcaccctccagacaaaaagctgagcaaaaatgaaacgcttcgcctggcaatgaggtatatcaacttcttggtcaaggtcttgggggagcaaagcctg

SEQ ID NO: 44 - DNA, 122 bp, Homo Sapiens
tctctctgttccttaagttgaaagatggcctttgtattcagagtagactccctgtctttgcggtttgggagatgatgagaaaccacagaattgctagtagtttatgtggagatcaggtacat

SEQ ID NO: 45 - DNA, 122 bp, Homo Sapiens
tcgccggcttctgcggtggggcgatgggggccgcaacctgctgtttccagcacggaggagcgcccagggcgggtgtccccaccctcagcgagctcctctgcgacttctcatcccacctcccc

SEQ ID NO: 46 - DNA, 122 bp, Homo Sapiens
aaaaattaaaaaggataaccatcacattacactgaatccacataggtctagggcttttagcgctgcccacaagaaggcttggggtggtttccaacttgccttacttcactctgttcctgtca

SEQ ID NO: 47 - DNA, 122 bp, Homo Sapiens
aatccgaggagggctggtcactactttctgggtctggttttgcgttgagaatgcccctcacgcgcttgctggaagggaattctggctgcgccccctcccctagatgccgccgctcgcccgcc

SEQ ID NO: 48 - DNA, 122 bp, Homo Sapiens
ggcatgaccccttctttgacacccataacccaaggcactgtaaacattcgctattattaacgctgtgttggactcaaacttgaatgcctgcctgttccatttagtgtcacaggagacacgag

SEQ ID NO: 49 - DNA, 122 bp, Homo Sapiens
agcgctgcgcccggacagctgtggtcctggtgtgggtcgtgtcggccgcggtgtcgtttgcgcccatcatgagccagtggtggcgcgtaggggccgacgccgaggcgcagcgctgccactcc

SEQ ID NO: 50 - DNA, 122 bp, Homo Sapiens
gctgctgtcattttatgggtcggcagccagagtgagagtgtccctgctgccagaggactacggcgggctgggcgcggggtccccgcctctcgctcaccacacagaccccgcgcctcctctgg

SEQ ID NO: 51 - DNA, 122 bp, Homo Sapiens
gcttggttattgcccatccccattcgcatctcagtccctttatccagccaatcttgatgtcgcaaagtttacacagaaccagagataaatagagaagccagacttgtggagagaggagggga

SEQ ID NO: 52 - DNA, 122 bp, Homo Sapiens
ggcggctgggtctgtgcatttctggttgcaccgcggcgcttcccagcaccaacatgtaaccggcatgtttccagcagaagacaaaaagacaaacatgaaagtctagaaataaaactggtaaa

SEQ ID NO: 53 - DNA, 122 bp, Homo Sapiens
cctccgcccagctccttggcctcccccggctgtgggctccgacccttccgtcccctgcagcgcctgcttcctggaccagagaccgaaagcctctcgctccgctgggcctcagcaagcccagc

SEQ ID NO: 54 - DNA, 122 bp, Homo Sapiens
tggcccatgtcactggcctgttcctgctgtaagtttcataacactgctgctagcataaaccgcttgggtatcagattgtgtctgcatttatctttgacacaaatcataccgaaggggagaag

SEQ ID NO: 55 - DNA, 122 bp, Homo Sapiens
tcctccctgggaggccttgaggaaatctccctgcccagcagatttccgacgtggtgttcacgtggtgttcacggtgccggccggaccccagccgagaccagattcctcccgaaatggctttt

SEQ ID NO: 56 - DNA, 122 bp, Homo Sapiens
aaggaaagcagggatcgggcactgcccgagggcagatacttgggctttggtgttgtccagcgcgctcggagtgcgctgcctcgctcacgcggtcccaggccccgcttcttcaggcagtgcct

SEQ ID NO: 57 - DNA, 122 bp, Homo Sapiens
cttagtgtttgtcctcgaaagagtcaatgatgttatctttgacaacctttattggcactacggtacatatggaagggtcaaagtttctaatacgaccgtcagggtaaagtctaaacggtatt

SEQ ID NO: 58 - DNA, 122 bp, Homo Sapiens
tggttgcaagtgttttccctgcaaacatggcctttgccaaagtgtctcatcccacatgtgcgctggggctgccacgtagccagtagccaggcccagctcccaaccactacaagcccctgggg

SEQ ID NO: 59 - DNA, 122 bp, Homo Sapiens
gcgggcccacgtggttggggccctgccctggcagggtcatcctgtgctcggaggccatctcgggcacaggcccaccccgccccacccctccagaacacggctcacgcttacctcaaccatcc

SEQ ID NO: 60 - DNA, 122 bp, Homo Sapiens
tggtaaaaccccagcgtggtgcctgcctctttgcttcctgggctggccgtgagccagggacgcgtgtcctggtgccctagaaccagggcagggtggcaggcttggcggatgtgggaggccgc

SEQ ID NO: 61 - DNA, 122 bp, Homo Sapiens
ctgtctgtcaacagtgtttgttttagccacccagtaagtgtttgttttagctgctctttacgtgagtaacactttgagaagaagcacaaggaatttctccataggaggggctacttaaagag

SEQ ID NO: 62 - DNA, 122 bp, Homo Sapiens
ccatagctgcctagccccaggggcttgtagtggttgggagctgggcctggctactggctacgtggcagccccagcgcacatgtgggatgagacactttggcaaaggccatgtttgcagggaa

SEQ ID NO: 63 - DNA, 122 bp, Homo Sapiens
cgggttctcctggcgagttttaagtcacactggatcgctgcttattttcagagatcacctcgcacccatgcacctggaaggcacccaaagagaatcattttaagaaattcacttcatctcac

SEQ ID NO: 64 - DNA, 122 bp, Homo Sapiens
tgcccgtccacgttccaacggcccgcagcggggtcgccccactgccgcatccttactccgcgccctgcgcgcttacctgcccacgaagttctcgagcaccgagctcttgccggcgctctggc

SEQ ID NO: 65 - DNA, 122 bp, Homo Sapiens
gtttatcttttgttagaatcctcaagcagtgcatctagtcattcatttagcaaacatatacggacgacattgctctaggtgccagaggttcagtcaaaacatatcatggtcctttccccttt

SEQ ID NO: 66 - DNA, 122 bp, Homo Sapiens
tcttatctcccaggagcaagtatcctgtgtgcgcagcgcatgaatgtgtctgggcatctccgcgtatatttatatagtgtgtgatgcgaaaagcaggaccagcaggggaggaagagggggtg

SEQ ID NO: 67 - DNA, 122 bp, Homo Sapiens
cctttttaattttttgtttgtttgtttgttaaacagatttctgcctggcagattaaccaacggcaagagaggctgatctgtctacattctgagtactgcccttggcaagacacctagaacag

SEQ ID NO: 68 - DNA, 122 bp, Homo Sapiens
gtcaggatcttgaaacttgaattcataaaaacccagaaagccccagaaacaaagacttcacggacaaagtcccttggaaccagagtaagtgtcacttgtcttttctgtcttatctgttactg

SEQ ID NO: 69 - DNA, 122 bp, Homo Sapiens
gagtaaagcaaagcctcagggcacagatgggcatcatcaatgcccatctgaaagatcaatcgtcaacatgttgcagcctgccactaggaagcacaaaccagaggactaccaggacagcagct

SEQ ID NO: 70 - DNA, 122 bp, Homo Sapiens
ccaccagcctttggcctctcgatacacacaacatccaggacttgtgcccttgccccatcacgacagacaaagcgtccctcaaggcccccgcgtggttcagacagacgccgcagccaggatgg

SEQ ID NO: 71 - DNA, 122 bp, Homo Sapiens
tcaggggaagagtggggtgcccgggctgggcaggagcgaccgggccgcgagggagcagagcgcgcgctccccctggctgtcccgtgcggcgaggaccgcactgagctgccccagaggaagtt

SEQ ID NO: 72 - DNA, 122 bp, Homo Sapiens
cagctagcggcaagcggagtcaggcatccgttcagactgacagcagaggcggcgaaggagcgcgtagccgagatcaggcgtacagagtccggaggcggcggcgggtgagctcaacttcgcac

SEQ ID NO: 73 - DNA, 122 bp, Homo Sapiens
gagtaaacgcatccacagaccagatagtcaaattaagttaatgattctctcagacacccacgccactggctcagcagggttcaaacatgaccttgaataaagggtccttacaaaatgattga

SEQ ID NO: 74 - DNA, 122 bp, Homo Sapiens
gagtcgcccctttccgcagcgcttctccccaagaatcgactgcttcggaagtgactcgagcggaggggtgcctaaacgactgaggtttcccatccggaaaccacacagctctgtcgcatccc

SEQ ID NO: 75 - DNA, 122 bp, Homo Sapiens
agttctgcggcgccaggttgaggccgccagagtggcgcacggagctagggtacatgctcacgtccttgtccaggaggtagctcacgtacatggtggcgagggtccgggagcagacctcacca

SEQ ID NO: 76 - DNA, 122 bp, Homo Sapiens
ccgaggagggctctgtgtgcttggatcaaggactcaacagggaaggtgggagagttctgtcgcactttggacactgtgtgtcatcctgatcatgtgattgtttgattttccttctggggatg

SEQ ID NO: 77 - DNA, 122 bp, Homo Sapiens
cttcctcaggaatttgagctggggatctgcatcctggccattgcagtcctttagcatcctcgccgcgccctgagcgcgctggaggctcgcaggctgcgccctcccagggctgatgccgcgtc

SEQ ID NO: 78 - DNA, 122 bp, Homo Sapiens
caaacgccaatgaaatgactccctctctggctggcagcttaagcctgagtcagtcttcttcgccttttgtaattgtcttaagtaggcgtggctacgtggctacaagtgcgtcgtcaaaacgg

SEQ ID NO: 79 - DNA, 122 bp, Homo Sapiens
aaaatttttttacagccctagtgtgcgcctgtagctcggaaaattaattgtggctatagccgcctcgatcgctgtctccccagcctcgccgcggccgctccgggacgcgcccgcccgccgcc

SEQ ID NO: 80 - DNA, 122 bp, Homo Sapiens
gtgcgcctctagcccgcactgttctgacagcctaactcacaacccctccacacacgaagacgtgagcagagctggcacagaacccccaaacccacagggccaggtccgcctgcctcaccaag

SEQ ID NO: 81 - DNA, 122 bp, Homo Sapiens
ggtgcgttgttcgcgggggtgaattgtgaagaaccatcgcggggtccttcctgctgaggccgcggacaccgtgacctcgctgctctgggtctgcagggaaacgtaggaaaaaaagttgtcag

SEQ ID NO: 82 - DNA, 122 bp, Homo Sapiens
ggcagctgccccccaacccccttcagcaattttcaagcagtaactgggctacactgctgacgtccagctgtgactctcacactggcaatgggtccaggcatccataagtcagggaagtgagt

SEQ ID NO: 83 - DNA, 122 bp, Homo Sapiens
gtctccccctccttgctgggggatccatgccctgacagctaggggactcattcattcactcgctcattcactcgttcattggttgctgcatagccactcagctcatattacgtgcaaggcgc

SEQ ID NO: 84 - DNA, 122 bp, Homo Sapiens
aagctgaggtttctaaggggacgactaaggaatccacgcatcactaacgttatagagactcgcgaagtgccgctgtgttggttgtgaaatttccctcttgtagctgtctctcgtcagattta

SEQ ID NO: 85 - DNA, 122 bp, Homo Sapiens
acaccccatcacccatcctctcaccccacagaatgtggagttagacagcctgagtttgagcgcacagcctgccacctcctagctctgcaaaccggagcaagtcaccctctgaggcccagttg

SEQ ID NO: 86 - DNA, 122 bp, Homo Sapiens
ccagagcagcctgttgtgccttgtgcctcgaagaggtttggtatctgccagtttctccctcgctgtttttatggctttcaaaagcagaagtaggaggctgagaaatttctctgttgaatacc

SEQ ID NO: 87 - DNA, 122 bp, Homo Sapiens
cggttccacgtgtctctgggaaatttttttcccctttcttcctaataagcctgcgtggaacgcagagcctgcgcaatagccccggtgaagttgtcgatgccctgaactctggtttttttact

SEQ ID NO: 88 - DNA, 122 bp, Homo Sapiens
gccagtcgcccaaccaagctaacaggtcctaagtcctcggagccaatgagtttccaggatcggaggagggagacagacacggagaggtcattggttggagcttttcggtaacgaggccaatt

SEQ ID NO: 89 - DNA, 122 bp, Homo Sapiens
gtcacggggacgagggaccagttcaggaatggcagagcggaccctggaggcggccccaaacgcctcccttcgccgccgccgcctcctcctccccctccatgacagtctctgggtgtttacag

SEQ ID NO: 90 - DNA, 122 bp, Homo Sapiens
tcgtgccactgtgagcccgggacacaagggctgactccagctgttggctttgagtatccccgcccgcgcccgtccttgctcgctgaccccaggcgaggcgcaaacaacccacgacgccacgt

SEQ ID NO: 91 - DNA, 122 bp, Homo Sapiens
tataggtcggaaggtggtaatacgtccacaatgtttggctcatggtacctgctccatagacgtcagccattgttatttttatcatctcacctggagtcctggccttggtgcgagatgcccag

SEQ ID NO: 92 - DNA, 122 bp, Homo Sapiens
ttttggttcactggatattcgaaacctcaatccactgttcaaatcccagcttgctttcaacgactttctgccttcaacaagcactgggtggggcttcctagtccaaacaacaaatagggaaa

SEQ ID NO: 93 - DNA, 122 bp, Homo Sapiens
ggaagcggctggctaggaggcggggcgtggggcggtggaactcctgggctgcggcattcacgtgatctgcacgggcgcagatgtaggcaccggtccgagtgcctgccctctgtccccgcggc

SEQ ID NO: 94 - DNA, 122 bp, Homo Sapiens
tgccagcctaagccctcaggtttcctttttctttttctacatcaggaaaagcacttttctcgccctgcctgcagagcgggccacccctagctgcaggtgagggacgggagaggagcctccca

SEQ ID NO: 95 - DNA, 122 bp, Homo Sapiens
gacgggagtggggaggtgggtcggggtgtccctgggccggaccgggtcgtgggactgggtcgccccaggcaggagagcctgggaggggcgggcccccttggcgtggcagggagaggtggagt

SEQ ID NO: 96 - DNA, 122 bp, Homo Sapiens
cggcaagttctgctgatggcttcggggtgggctccagagacttttctgtcagcggaacagcgcctgttccgatctgggaattaccctgaagcagcaacaagcctaggttttcagcagagaac

SEQ ID NO: 97 - DNA, 122 bp, Homo Sapiens
gatggcgatgcactgatgggggtccactgggacttacatttacacagagctcgctggtaccgccggccctgggtgtgcctcagcacgtgttctgggcacttgttgatgtgccgcagggtgag

SEQ ID NO: 98 - DNA, 122 bp, Homo Sapiens
accacatcgtctttttgcaataatcttggcagggaaaaatatgctagccagggtccactacgtcgatttcaagcacggaagatgggtcacaccaggcacttcaaaagacccgtccctgaggc

SEQ ID NO: 99 - DNA, 122 bp, Homo Sapiens
tcaccatgccttgctttggcccttagctgcgccaggcatttggctgtcctccccaggacgcgcgagggtctgtccacttcacaccccgaatgccaagcaaagcactgggctcgggtacaccg

SEQ ID NO: 100 - DNA, 122 bp, Homo Sapiens
gttcctgttgaggccttgcccttttctcacccgtgtccggagtggcccccgctccagaagcgcacagccgctggttggggcatccctgacatgaagcccctctgcccgcctgggcgctgtcg

SEQ ID NO: 101 - DNA, 122 bp, Homo Sapiens
ttttgtaacatgagtatgactctaaaatctcaatacccactagcagttattccacatttccgcctaaatctcccagcagccactaatatgctttttgtacctgtgatttggctattttggat

SEQ ID NO: 102 - DNA, 122 bp, Homo Sapiens
ggagagcggctgagtcaggctctggcagtgtctaggccatcggtgactgcagcccctggacggcatcgcccaccacaggccctggaggctgcccccacggccccctgacagggtctctgctg

SEQ ID NO: 103 - DNA, 122 bp, Homo Sapiens
tcctcctccctcccggtcaacaccagcccacgggcccttcggctgctcctccggggacaacgaccctctgtttccaggagacacttgagtaattcggaaaggtgtaagcggttcttttcgct

SEQ ID NO: 104 - DNA, 122 bp, Homo Sapiens; identical to SEQ ID NO: 9

SEQ ID NO: 105 - DNA, 122 bp, Homo Sapiens; identical to SEQ ID NO: 7

SEQ ID NO: 106 - DNA, 122 bp, Homo Sapiens
ggacaggcacacaggcccaggtgtgtaggccacagcagctgcagtcctgaaaggctgcaacgtcccgacctccaggagagaccaggcccaggatgcctcgcctgttttttttccacctgcta

SEQ ID NO: 107 - DNA, 122 bp, Homo Sapiens
catcgcgccccggtcgtgagtgcgctcacacgcagcctgagactcgacgggagggggtcacgtggaagtatctgagagaggcgtacttggccactaggaaagcacctccccctttccaaaaa

SEQ ID NO: 108 - DNA, 122 bp, Homo Sapiens
cctcaaatttctgaggacctacaaactacaaagcactcttctaggggcccaaagataaaacgctcccctcgtgaaaatcccggacttttggtgggaggtggggaaggtgacaactatgctca

SEQ ID NO: 109 - DNA, 122 bp, Homo Sapiens
cgcgggaagtgagcgggctgcctctgcgaggcaagtgcagttgagctgatgtggatcttacgtcgcttgagtgcccacctcctcgtcctctccccatttgtccgccgcacaaagacgctcgc

SEQ ID NO: 110 - DNA, 122 bp, Homo Sapiens
gcaggtctttggggagcgctttgagtaatattagagctttcactaaaccctggcctattacgtcccctcccctgagatttacctttatatactcaagttctttcatgccaggaaggtaggct

SEQ ID NO: 111 - DNA, 122 bp, Homo Sapiens
tatggaaattagtcaacactgacaatctgtcgcctgcagcagttgcttgtttgcaaatcacgtcttttgcagaaatgaggccagcccttttcgggacacttccatcgctttttttctttttt

SEQ ID NO: 112 - DNA, 122 bp, Homo Sapiens
atttttgtggttgagggactttaaaaatttagccgcttgggaagtacagaattttttaagcgggctgaagggataatgtaaatttctgccacaggcaacatcgaaggaagcagagagatttg

SEQ ID NO: 113 - DNA, 122 bp, Homo Sapiens
cagttgtgtgcaagtgcatatacgtgtagcctagtagcccgtgtccttgttccttgagaccgcgctgcctccgccacgtgtcacacgcccaggcacacgtccccgtagggagagccggggga

SEQ ID NO: 114 - DNA, 122 bp, Homo Sapiens
aagctggtacaggtcactctgaacataaggtacagaacgtctgggtacaagtccaggctccgccactggctcaccgagcgacctccagacacacatatgcacatcttaggcctcggtttccc

SEQ ID NO: 115 - DNA, 122 bp, Homo Sapiens
aacccagccggccagggctggcagcgtgctgacgaagggccgatcaaatactgatgctgccgcctggggtctccactggaggtcacgggtggagagtagggcggactggagaagtggagatg

SEQ ID NO: 116 - DNA, 122 bp, Homo Sapiens
ccctggatcccaagtgccgccaggcaagcagaccccgcccacagacacatgacgctcccacgccaaatgtggatccaaccgccccacacaggatccccgcccggcccggcccggggcgcccc

SEQ ID NO: 117 - DNA, 122 bp, Homo Sapiens
tggccagaaaaatataactgaagcccaaggtcagaagccaagctaggcagtcccacttagcgctgcccgagttgatggggacctcagtttccccacctgagcctggatcggctgaacctggg

SEQ ID NO: 118 - DNA, 122 bp, Homo Sapiens
ggcaggcggctgctggccagggtctcgggcacaccctgagatgagaatttcatctaagagcgcgcgcgcgcacacacacacaacccactcacactcttctgggcgcatcgctgaccgcgcca

SEQ ID NO: 119 - DNA, 122 bp, Homo Sapiens
ggcagccctgcgggcctaccttgtcccctttgcttaggacctgccaaggccacgtctctacgcttccgaacagcgagttcttgatcatgcccaacatgttgtagccgagacgctcccgacgc

SEQ ID NO: 120 - DNA, 122 bp, Homo Sapiens
gctgggtgcttgtcgcttcctgtttattccaaggtgaggggcagagagaaattcaacacacgcctgcttcctccggggctcggagacccagcctaagtgcaacagcagcagccccagacaat

SEQ ID NO: 121 - DNA, 122 bp, Homo Sapiens
ctggaaaataagtgacttgtccaaggtcacatgacaaccatggggcagttctggctagagcgctggtctttgtgaccccccccgtgccatttttctttctgtcacaaacacaaaacccattt

SEQ ID NO: 122 - DNA, 122 bp, Homo Sapiens
ccaggaccagccccagcatgcagagcgctctggcagccatgaccaccgtgggctccgggacgcagctcaggactcgcttcatggtccaggaggcctcatttatgcaccgttgtttgcacagc

SEQ ID NO: 123 - DNA, 122 bp, Homo Sapiens
cttaatctccacagtgccaatgatctcagccacatttcatcaaactgtaataaccataatcgggactagtccactacctggacccctctactcttaaaatatgaatctctctaatatcctac

SEQ ID NO: 124 - DNA, 122 bp, Homo Sapiens
accgttcactggggccgggctggaaggctgttcctttaagatcaccagcaggagcacggccgccgcgtgcaggctgcgccggggcatcttcgcgggctcctgcggggcgggcgcgggctgtg

SEQ ID NO: 125 - DNA, 122 bp, Homo Sapiens
gcgaccaagaactgcctctccacgtccccacccccatgccccttggccagctaccactcacgcatctcctatgaaaaggttgggccaaacttcgtccacacggctgcaagaagacttccctg

SEQ ID NO: 126 - DNA, 122 bp, Homo Sapiens
tgaggggggtggggaaggatgcgatgtgcaagcacggagggcggcccgggggtgcgataccgcacgcccggggccaggactctttaccttgtcgctgtaatccctcaggacccgctcgatag

SEQ ID NO: 127 - DNA, 122 bp, Homo Sapiens
acggcccctcctaatgatgtttcttttcctatcattgtcgagcccttagaaccccaggatcgagggagggaaaacaagcgctcaagcactccctcgttggtcccaggacacaaggcctcgtc

SEQ ID NO: 128 - DNA, 122 bp, Homo Sapiens
ggtccacaaagaactggacggatgcagtggtggatggcgatctctctggaccactccggacgcgcccagcagacgcagcctacgcctttaggtcctaggaggctgagaagggtggggagagg

SEQ ID NO: 129 - DNA, 122 bp, Homo Sapiens
cggctctcttttgcgagcaactcttgagtccaaggcctgggcgaagctggcggagacaatcgcccaagtttgaaggggccgcgaagcttggcgggggaggtcggtgttggcgcctaggcccg

SEQ ID NO: 130 - DNA, 122 bp, Homo Sapiens
tgggttcaaaggggagagataaggcgcagactagaaacgctaggctggacccgggccaggcgcgccgcggggcagagatgggcggagcgggaacgggggcgggggcgagaatgggggggcgg

SEQ ID NO: 131 - DNA, 122 bp, Homo Sapiens
ctcgggagggagctgctttctgccgaggctgccccagctgtgcgtctgtctttttccacacggtaggctacacacaacccagctgtaccagcatgtgccagagacacgctggccaatcgtgt

SEQ ID NO: 132 - DNA, 122 bp, Homo Sapiens
cagcagagaggaggccacagagaagccgtggctgaagtccctggtgagccggaaggatcacgtcctggacctcatgctggaggccatgaacaaccttagagattcaatgcccaagctccaaa

SEQ ID NO: 133 - DNA, 122 bp, Homo Sapiens
ctccagagccgaaggctgtttttctgatttgttcagggcgctgcctgctatgcctttccccgcgcctgcagatgcagaccatctccgtttaggggctgcctgggtgccgggctgtggtaagg

SEQ ID NO: 134 - DNA, 122 bp, Homo Sapiens
cggggtcggaggcagggttcaaggcagcacttacaggaggatctcctgcttcgtcaggaacgtcaagtcctggaaggcaaagccagagcggggattagcggaccgctgggaggccgcggccg

SEQ ID NO: 135 - DNA, 122 bp, Homo Sapiens
cagcctccaccgggaaccaggggcgcgagtcgctgctcagtggcctgggcctgcatgtggcgcggcgggagctgctgcctagggccgcgcagcgacggggggtgaggactacatgtcccgac

SEQ ID NO: 136 - DNA, 122 bp, Homo Sapiens
ttcctcatccttaacttctctacggcaaagtatacggctgatgtgttcctaggctttcctcgcgttttgtgcgtttttattcaaattagaatttcccattgaaacctttcgtgcacattcta

SEQ ID NO: 137 - DNA, 122 bp, Homo Sapiens
gggtcaggacccgcgggggtccgcgctcgcggggtgctgtcggagaaccacgtctgtcgacgctcggtttgtgtttagaacccaagtttcgaaacagaaccttgccgctctacctgggttcg

SEQ ID NO: 138 - DNA, 122 bp, Homo Sapiens
ccactgcactccagcctgggcgacagagggagattctgtttcaaaaagaaaaaaaagaaacggcgtccaacatgatcagtcattagatgtaactatgtaaaagtcagctttagattagttta

SEQ ID NO: 139 - DNA, 122 bp, Homo Sapiens
tctctcccatccctcccctccttccctaggctaaagccgcggatacctccgagagggcagcggcagctgacagagccggagcctcccaccccaggatacaccaaaatctgcagcgcctgcag

SEQ ID NO: 140 - DNA, 122 bp, Homo Sapiens
tcctgcagagaactcctccccctgcttgggaaatgccaggacaaccaagtgttctgttctcggactggaaatgaccagatgataatttctggtaatacgggcttgttagacctactcaaata

SEQ ID NO: 141 - DNA, 122 bp, Homo Sapiens
tttttacagggaatgcattctttctgaaagtatcaagacggcgccaggcagctcagtgttcgcagacagctgtggcgcgacgcaacttaaggaggttctagtgtcatccgcgccggggggga

SEQ ID NO: 142 - DNA, 122 bp, Homo Sapiens
cccgctgcgaggtgcgaggaccaaatagaggcgtaacaagccaagggtcggagtgggcaacggaacagttaaaggcatttcacaacagcgatttaggtctgcaagcagtttatggagtacca

SEQ ID NO: 143 - DNA, 122 bp, Homo Sapiens
gacctagacctactgtatcagaatctctgtgctggtcctgagaatgtgcattctcaacagcgcctcacattccccaccccaattcttctgcagctaacgtttgagagcgctggtctggtgcc

SEQ ID NO: 144 - DNA, 122 bp, Homo Sapiens
aggtgctccaggtcgctcagagagtcttcaggattcctgagtcgccgagtctatggcatgcgctccgggccctgactacaagctggttttcaaaggaagtttgtggtttttcttttcttctc

SEQ ID NO: 145 - DNA, 122 bp, Homo Sapiens
agtggagggaggggaaaccaggcaccaaagcaaattccttcatttcataatttcatctggcgcccccgttcaatcagcctgcattgtcgaggctttaggagaggcagttaggaaaggagaga

SEQ ID NO: 146 - DNA, 122 bp, Homo Sapiens
cccttgacggcgcccaaggccccgcccgtgcggcgctgcccattccccatcaccagagatcggttcgtccagcgggcaggccccatcccaatgttaggtcaaagctcctatcccttggaccc

SEQ ID NO: 147 - DNA, 122 bp, Homo Sapiens
gacctccagtgatccgcctgcctcggcctcccagtgttgggattactggcgtgagccccgcgcccgcccctggctggcttctttgccgcaagctgtttcatcagcaaagtctttgtgacctg

SEQ ID NO: 148 - DNA, 122 bp, Homo Sapiens
gtcgcagatgacgtgctgcaggtcgctgtggcagtggcagttctgggggcaggcggccagcgccggcagcagaccagccaggaggccgaggctgagcaagagcattgggcggaccatggctg

SEQ ID NO: 149 - DNA, 122 bp, Homo Sapiens
gatctgagacttccaaaaaatgaagccggcgacaggactttgggtctgggtgagccttctcgtggcggcggggaccgtccagcccagcgattctcagtcaggtgggttccttctggcactcc

SEQ ID NO: 150 - DNA, 122 bp, Homo Sapiens
gtgtcctctctgtttcccaacctccccataccgaggaccccacaacccgccccgggatggcgcctggctgctcgcaccctaattcaggaccccaattggtgggagaggaaccgcggtcctca

SEQ ID NO: 151 - DNA, 122 bp, Homo Sapiens
tgagggttcccaaccctgctaagcagttgtctccaggtcatgcaccttcagaatggaaagcgctttggaatgacacgatcactcccgttgagtgggcacccgagaagccatcgggaatgtcg

SEQ ID NO: 152 - DNA, 122 bp, Homo Sapiens
cagcaaggctcagcctcaagattcacagcatctcagacacagcctaggtaaggggctctgcggagccctgtggtcttcaaaaaccatgtccttgggggagggggcaggaggcaggtggggaa

SEQ ID NO: 153 - DNA, 122 bp, Homo Sapiens
taagcatttcctggtgggtggaatacgccatgaactaggtgcggcctcaagctggttactcgccctgcctactccctctcgtgtcctcccagtacccccttagacccaccagctctgaggga

SEQ ID NO: 154 - DNA, 122 bp, Homo Sapiens
gcagctggacttgaggctgtctctgcacagagcagcaggtgaacagccctgtggagcccccgccccgccatgcagcaactgctccagcctcacagctggctccctcaggtctaaacacagcc

SEQ ID NO: 155 - DNA, 122 bp, Homo Sapiens
tggcttgcttcattgatttacattaacctgcctggcgctgtggacacttcagtttgcatccgccttctatgcatcttgtatatctctctaagttctacctttttcttttgcttttctgatcc

SEQ ID NO: 156 - DNA, 122 bp, Homo Sapiens
ctggccccggggcagcacccaaagttttgtcccccccaccgcccccacccctgttcacctcgcagccttgggaacaccaggctccttaggcacgtctagctccggacaacttttctctcgtc

SEQ ID NO: 157 - DNA, 122 bp, Homo Sapiens
cgaagcccctcacagcacaggctctggcttgaggagatttctcccggggcagccgctggacgcagtttatgatcctcttaatctttactgtctccacaggggatcaactggccacacctgca

SEQ ID NO: 158 - DNA, 122 bp, Homo Sapiens
tctctctctctccccaaagtgcatgcaaacacatatggaactgacataggcatccactgtcgcacttcaaattaaacagacgcacacgacagatattctcacagaaaagcagtcagccaagc

SEQ ID NO: 159 - DNA, 122 bp, Homo Sapiens
tactcctttacaccaaaagtagaatcttcagcatcattatcttctccagaagcaggagaacgtagtgcaccagaatttggtcataaacttggctttgagtcctagtcctgccccttactaac

SEQ ID NO: 160 - DNA, 122 bp, Homo Sapiens
ctgaggacaatgcgttccacacccgctggctggctgatgtttgattctgtaactataccacgcccagaattctctcaaaagggaataaaacacaggtcaaattcctcacccacacactccac

SEQ ID NO: 161 - DNA, 122 bp, Homo Sapiens
tcagggaaagcttcctctttttgtgcacttgtttgtcctaaggccgttacaagtattgttcgttcattctgcaaatatttgttgaccgcctatgtgccaggccaacaaagtccctgccctcc

SEQ ID NO: 162 - DNA, 122 bp, Homo Sapiens
gttcgcggcccccttgtctgacaccccctttttcccgcgccgcggcctgaacaagggttgcggaggtctcccacccgctggagcccgttcagacctgacggaatcccttcttgcagaattgg

SEQ ID NO: 163 - DNA, 122 bp, Homo Sapiens
ttcgcactttcgagtgcgcgtttgctgcccgccgcagcccgggcgttgaaggtagtaaagcggccacccggccgcatcatggtgaccccctgtcccaccagcccctcgagccccgccgcccg

SEQ ID NO: 164 - DNA, 122 bp, Homo Sapiens
gcaccgaactacctcctttcgcctacaaaacgtaggtggggaccactggtgttggaatgacggcccacctcgagtttcaggtgacttccactctgcaattaacttgcaggcagccccagacc

SEQ ID NO: 165 - DNA, 122 bp, Homo Sapiens
cgggcacattagatgacttacccacagtcaccttgctagttagtggcaaaacaggtaccacgacccattatctggcctcctcaccagtgctttttttttttttttttttttttgagacggag

SEQ ID NO: 166 - DNA, 122 bp, Homo Sapiens
atgctgtttctctaggtcagcaactaaacccagaaaacgtttattgagtgaatgatgaaacgacaggtgaatagatgaacgcaaggtgtcgagttaactattcttctacacaagtcctagca

SEQ ID NO: 167 - DNA, 122 bp, Homo Sapiens
cacacacacacaccccagtgccaggatcagtaatttatgagagtcacagtgaccccagaacgacctggcctccctctccaacaaagcaacagcaccaggttggcactgaataccaggaagag

SEQ ID NO: 168 - DNA, 122 bp, Homo Sapiens
ggggcggggcggcagaaggacctatgttcgaggctgcactcggatttgagagccagagaacgcgaggggcgagcggactggcggctgctaggcagccgcgccccagcgcttccgtacctaag

SEQ ID NO: 169 - DNA, 122 bp, Homo Sapiens
gaatggtctaaggacacatggtaaagcaatagcagagcagggtacacccctggactcactcgtgtggagttcagtgactctctgggtgatcctccctggtgcctgggacccagctctatcag

SEQ ID NO: 170 - DNA, 122 bp, Homo Sapiens
cctggcgaatccaatccccatcctcaatcacacatccacgtccccaaaacaaatccttcacggtctgaagatgagatggcaaaaccagaacacacccagaaagttgacacagaagggggtga

SEQ ID NO: 171 - DNA, 122 bp, Homo Sapiens
gtgcctgggagcctgaggccgaggagggtcccggcgtagtaaaggggcggcaaccccaaacgggccaggcatcaaaatgagacagcattaaaccgagtgacatctctgtgggtccttagtgc

SEQ ID NO: 172 - DNA, 122 bp, Homo Sapiens
gtgatgtttagaaccttttgggggattccttctctctcagaatttaacctggcaagagaacgactgagttctaggaattttcttgtctggagagagtaaaataaatgtattttttaaaagct

SEQ ID NO: 173 - DNA, 122 bp, Homo Sapiens
aggacgtgcgtgttagggggcaggtatttggagccaggccccactttcgagttgtctttccgcaggaaccagagactgcggacggggccaggaaccaggaaccgttagggagataaggaagg

SEQ ID NO: 174 - DNA, 122 bp, Homo Sapiens
ggaaaaggaaaagaaagaaggtagttcaggccccggtggccagcaccctcaaggaggacacgagcagttcttacctggagatggaagaggagctgctctgttcgttcactcaggaattctgt

SEQ ID NO: 175 - DNA, 122 bp, Homo Sapiens
ccctcgcaccctggtcctttgttgcaggctgacatctgccccaccagagtcaactgtcagcgccttgcacgtgaattagggaagcggagtaacctggggttccatcccttcccgggtccctt

SEQ ID NO: 176 - DNA, 122 bp, Homo Sapiens
cgggtttcgtggcccttcccctacgctggaatcttctgtgtcatttagggggaggggatgcgggacggcatctgtttgacaacgcattggtcccacagacagcctttaaaaacaactgcttt

SEQ ID NO: 177 - DNA, 122 bp, Homo Sapiens
agaaggtcgggctgcgctgtgggctgcggggagccggaggcctcttgattggctctttgacgctttcggaccaattgtgttgctgcataggcgtggttttcacaggtcgagtgctgggggat

SEQ ID NO: 178 - DNA, 122 bp, Homo Sapiens
gaagtggtgctggtttggctcaagcatttgattcactctgttgtctttggaggtggacaccgcccagaggaagacgaagaggaggagattgtgattgggtggcaggagaagctctttagcca

SEQ ID NO: 179 - DNA, 122 bp, Homo Sapiens
gtggttttagtatatacacagagttgtgcaactaccatcacaattttagaacaccttcatcgcccttaaaagaaatgccacacgctttagtggtcactccctatttcccctaaccctccaca

SEQ ID NO: 180 - DNA, 122 bp, Homo Sapiens
ctcacagaacccagctggtatggaggggcctctcctgggacacccatgtgcatgctatagcgctggccaccaaagacagggggtggcaggctgtcccatgaggcctttcatggccccaaagc

SEQ ID NO: 181 - DNA, 122 bp, Homo Sapiens
accttgtgatccgcccgcctcggcctcccaaagtgctgggattacaggcgtgagccaccgcgcccggcccggggactcttaaaatacaaacattactggaaggtcacctcctgctagggcca

SEQ ID NO: 182 - DNA, 122 bp, Homo Sapiens
ctcttccattcatcagtctctggagcggcttctgctgccgaggcaggggctccaggtcctcggagggtctgacagcccctcagatgatcccggaacgaattcccacctgctcccgggagcct

SEQ ID NO: 183 - DNA, 122 bp, Homo Sapiens
gctgtggtgtgaattagttggtctaagcttttgggccgcttccaacttctctattgctcacgccctacaggtcaaggaaagaagtgatgtaatgcacttagctaggccactttaattacaat

SEQ ID NO: 184 - DNA, 122 bp, Homo Sapiens
gaggttattcccaaaaatattcgctctgacagttaggaatgcccagggccaccgggatgacgcgctcgcgccggtgtgggtagctgagagttgaatgtccgtgaccctggtacagttgggtc

SEQ ID NO: 185 - DNA, 122 bp, Homo Sapiens
ccccgcgcgtacctgccgctgccgccgagggcagcgccctctgtgttccctgccttcctacgggccgacgcctggcatagttgaggtttcgctccaactctgtcctgcccctgatgtcacag

SEQ ID NO: 186 - DNA, 122 bp, Homo Sapiens
ggggccagaacagcccggcttcagtcagttgtggggtcggcgttagacggtggagttcgacgcccctctgtccattagcaattagtgctagcacaaggcctggatggaggggagaggaaggt

SEQ ID NO: 187 - DNA, 122 bp, Homo Sapiens
agcacagcccatcctggcttcgggccaggctgattattcacagccgtgatgtcattgaatcgcaggaatttggaagtgggatgggaccttaagaatcatcttcctttagagtcctagtgtcc

SEQ ID NO: 188 - DNA, 122 bp, Homo Sapiens
cgcaggtgacagccgaagttcatttcgtggtctgcttctttcctgtgtgaccttgcaaaacgggcttgacctccttatttatcgacctctttatcttcttagtaaattgaagatattagtcg

SEQ ID NO: 189 - DNA, 122 bp, Homo Sapiens
attacaggcatgatccacagcgcccagccaataacacagtcttgatttttatggagcttacgctctgatggaggaagacagctgaaaaacatgcaaaaaaaaaaaaaaaaaaaaaaaacacg

SEQ ID NO: 190 - DNA, 122 bp, Homo Sapiens
aattgcaataaaaagacggcccacagcaggctgcattcccatggctggccagaggaggaacgctttgtgttctcatcggaggtaagtcctgctttaagccccaggaggcttcctcctaatcc

SEQ ID NO: 191 - DNA, 122 bp, Homo Sapiens
tttcgcgttcttcggactagccagaggctcaggttggtgaccgagcggcagagttcctagcgcctgcagtgtggtgaactccaacttttaggccaagttgaaaatgcagccgacgaccccca

SEQ ID NO: 192 - DNA, 122 bp, Homo Sapiens
gctcagggacccagtcgcttgagctcattctcttatgacccaatgtggctgcccaaaagacgcctgagacccgcggcccaagcacgggctcgccggcgccgagtcccaggcaggagccgcag

SEQ ID NO: 193 - DNA, 122 bp, Homo Sapiens
tggcccctcatggctcctgtcaccaggtctcaggtcagggtccagcaggccctgagctgacgtgtggagccagagccacccaatcccgtagggacaggtttcacaacttcccggatggggct

SEQ ID NO: 194 - DNA, 122 bp, Homo Sapiens
tgcgggtcagcctccgactacaagagcctcggcccacaccgggaactcagtgaacgtcagcgggagctggggcaagagcactgactctgagacagcagcctccgctcactatttgaaggcgg

SEQ ID NO: 195 - DNA, 122 bp, Homo Sapiens
agagtcacgtctgtgcaaagagaagaacgactctcgacctcctggatccagtcaccaccacggtcccaacacagcgctccaaggactccctaacgccaatggaaatggtcgctactaaaggg

SEQ ID NO: 196 - DNA, 122 bp, Homo Sapiens
accaccaccacaaggtaacgttctgccccacttttctcatccatgttgggtggagacttgcgccatccctcggctctctacatccatccgcaccacgcccaggtgaactcacactcgggacc

SEQ ID NO: 197 - DNA, 122 bp, Homo Sapiens
tccttccccgggaggtgggcgcgcgtagcacaccgcaccggcagcgcctctgctagcgaacgctcctttaggtctgcacctccgccagctcctagtggccaaaagcctgcccccacgcccga

SEQ ID NO: 198 - DNA, 122 bp, Homo Sapiens
gaaaagcagacgccatttggcttaatggatttggctatcatctctcctgatttcctggaacgcagaccttacaagtaaacagcctagtgcaggagctcagtccaaatcaatgacttcccata

SEQ ID NO: 199 - DNA, 122 bp, Homo Sapiens
gtggcgagagcaggttcgggggtccggagggtcggagggtgggctgcgcgcgggaatggccgccgcgaaggaggggcggcttcctcgggcatcccgggcgcccggccccgtgcatgcaacag

SEQ ID NO: 200 - DNA, 122 bp, Homo Sapiens
tgccgagatttcggcagcgtgctgagcttcatcaaagcattaaaacaatgctgcttgagacgtcagaggctcatccaggaagtgcaacccagagggcaggatttcctgctggactttgaaat

SEQ ID NO: 201 - DNA, 122 bp, Homo Sapiens
aggaatccccggacctcttagagctttgctttggggcaagggccaaggaggctttgcctgcgccaagtgcagttgactgtctgggtttacaaaataaacccagaagcagcctgacgtctccg

SEQ ID NO: 202 - DNA, 122 bp, Homo Sapiens
ttaacataagtgctctgggggtgagtgtggcacggccagccacagagaaatcacactgctcgcccacaattcggatgtctcagagggagggggaaaaaagatggttaaaaagtcgagcttac

SEQ ID NO: 203 - DNA, 122 bp, Homo Sapiens
cctggtcttccctgatctttcactgtgtgcccttcaggcactcaagctgttaatatcacacgagcttaataggctcccctgagatggtcctcatgtgaaaggattgctctgtaaggtgtacg

SEQ ID NO: 204 - DNA, 122 bp, Homo Sapiens
tacagggtctcaataataggattcctactatgtccaaatcgagccccggcttttcgtatacgatgtcaccaaggcaggaaggactatgggctcacataaccagaggccaaggggcgacccga

SEQ ID NO: 205 - DNA, 122 bp, Homo Sapiens
gaggtttcccggcctcagacaggccagaggtgtggagcccgcagacccagggccggctcacgggcatctgtgctattttgatttgatttgatttgagttttaggtcacgtttgcacgcagtt

SEQ ID NO: 206 - DNA, 122 bp, Homo Sapiens
tccctgcttccagacttattctcctgccttaggtacagtcactatcgtgatattatctatcgctgtgtcaacacaacaccaatccaagcccaaccgcagctctctcactgccgcaccgcttc

SEQ ID NO: 207 - DNA, 122 bp, Homo Sapiens
cgctgcagatgccgcggcagctgctgcaggtgtggctgcggctgagcgggcagcagtggccgcggagccccggggccgggagcagaggagaagccgccgggggaacccgcagccgctcgccg

SEQ ID NO: 208 - DNA, 122 bp, Homo Sapiens
tgacaaccactctttgagaagtccctgtccccacgtccggcagcaggtcctgtgctcgtgcggcagatcagatgggcaccacttcacggcccagcctgggttggcccttcctaaccgtcctg

SEQ ID NO: 209 - DNA, 122 bp, Homo Sapiens
accggccttgcggaaactgggtctgccaacctgctccttggcccattcctgacagccatacgcctggcctcacagctccggcagacccccgccgccaccagccccgcccctcccgactgccc

SEQ ID NO: 210 - DNA, 122 bp, Homo Sapiens
taaacacctccaagctgccactgttcttcaaatatcttggatcactctgagcacgcatttcggttgtgaaagcaaaaaggaaaaggagcaacccaaagcctaaagaaaactgttctcgtccg

SEQ ID NO: 211 - DNA, 122 bp, Homo Sapiens
gcccctgccccagggcccggctgccagtcccgaggatgaatgttgcctttgtgccctcagcggcctccggggactcacaagcaaccacttgtccccaggtggcaccgccacggctgctcaga

SEQ ID NO: 212 - DNA, 122 bp, Homo Sapiens
gatcgacccatttgaggtttcttaccaaattggtgaaaagggaccagtgaaggtgctttacgtggatgataataatgaaaatggatgtgagttggataaggagatcaaaaacagctttaacc

SEQ ID NO: 213 - DNA, 122 bp, Homo Sapiens
ccgccctccctggcctcagtttccttgttggtaagaagcgggcctcagtgtctgaggccccgcgcccgggtcgatagagtccctgacggcccagaccgggctcccgggattcggatctcatt

SEQ ID NO: 214 - DNA, 122 bp, Homo Sapiens
cagcttgtgatgatccagggcagcctggctctgatctaaagcacagctacctcttccttgcggcccctatcctggctgctcctgggaataagtgccaaatctggggtcagacagccctgggg

SEQ ID NO: 215 - DNA, 122 bp, Homo Sapiens
gagccaccacgcctggccagaatacctttactttttagcagttagaatatgttggtgctacgtcagtcttaatgtttgcatgctactccagtttgtactgtgttgtgggagtttggagtttg

SEQ ID NO: 216 - DNA, 122 bp, Homo Sapiens
gaatatacagagaacctcactaacgcgagtaaaatgccgactacttacaaaacgatttcacgtccacatctgcagtctcataagggctttcgaataatcctgtgaagaatgtattattagtt

SEQ ID NO: 217 - DNA, 122 bp, Homo Sapiens
ggcagcgcatcttctagccctgctggaagctggaggcggcagccggttgtccttgttgcacgtgggggtggagcaaagagtgcgagaaaatctgcggatggcctcggggatggcccaccccg

SEQ ID NO: 218 - DNA, 122 bp, Homo Sapiens
tgggctggcaggagattatttttaagcacttgtgcttccatatggcttgttttaaactcgcgggacacctacaaatttccggctgtcagaagtcacatgccagcaatccgctccagcgcgga

SEQ ID NO: 219 - DNA, 122 bp, Homo Sapiens
cgccgggcgctgcctctacagctgtgtgtaggcctgggggcgagggtcttcggaacgtagcgctggctgcggccccgcccgcctacccacccgcccgtccggcagccggctcccgccgcctc

SEQ ID NO: 220 - DNA, 122 bp, Homo Sapiens
aaggagcacctgaaaaggccttgaagagcaactgggcacctggccgcgtcaggtgggagacgctgggcaggacagacgcacaggccatgtgtctggagccctcagatgagtgtgacacctgg

SEQ ID NO: 221 - DNA, 122 bp, Homo Sapiens
gctttagtgaccatgactgcttgagcccaaagtaagatggctggtggctgtgtttctggacggggatataaaatgcataggttaatgaaagttgttcacaattcagaagctaaaacgagaaa

SEQ ID NO: 222 - DNA, 122 bp, Homo Sapiens
gcttggggtcggggggaggtgagagctgaggggcgaatgtccctggcgcgggcgggagagcgcgctctcggcccggcccgcaacttgattcctaactctgacctgccgcccggcgcgccttg

SEQ ID NO: 223 - DNA, 122 bp, Homo Sapiens
gggagctgagttgctggtagtgcccgtggtgcttggttcgaggtggccgttagttgactccgcggagttcatctccctggttttcccgtcctaacgtcgctcgcctttcagtcaggatgtct

SEQ ID NO: 224 - DNA, 122 bp, Homo Sapiens
tagcctgggacccagccgctggggtaggggaccaagccagctgtgcggagggaagcgcaacggcccaaagtaggtttagaatatgagtaggttttaaaaactgtactggatgtaaaaaggga

SEQ ID NO: 225 - DNA, 122 bp, Homo Sapiens
gttggcttgcctctctcctgccagaatgcaacctggctttatgcacctagatgtccccagcgcccagttctgagccaggcacatcaaatgtcaaggaattgactgaacaaactaagagctcc

SEQ ID NO: 226 - DNA, 122 bp, Homo Sapiens
tccacatgaacccttactcaatggggcaggtgtgagggactgtccccaacaggttccagacgcctcctgggttccctggggagggcttagaggatgaagcggcaggaggagatgggatgagt

SEQ ID NO: 227 - DNA, 122 bp, Homo Sapiens
gaggagggcgggactgagcaggcggagacggacaaagtccggggactataaaggccggtccggcagcatctggtcagtcccagctcagagccgcaacctgcacagccatgcccgggcaagaa

SEQ ID NO: 228 - DNA, 122 bp, Homo Sapiens
tcataggtaagatggaaataattacaccctctggatggtgtgactgaagattaaatacagcgggtgctctcactcagcacatctggccatgtctgcagacacatttggttgccacaactgga

SEQ ID NO: 229 - DNA, 122 bp, Homo Sapiens
gggtggagacttgacatgcacaatcctaggcgcccaactgcacgttgtgagtgtgtgagtcgtgagtgggggtgggtgagatcccgggctgcaggcacatgtcgaggcatgtggcacctgga

SEQ ID NO: 230 - DNA, 122 bp, Homo Sapiens
ctagctcagcctagtttcctttttttaacggtcaaattctgagccggagctactgatggccgcggcggcatggccttgaagtttttgtttttgtttttggagaggggggaggggccgtgttt

SEQ ID NO: 231 - DNA, 122 bp, Homo Sapiens
agcacctgactgtgcgtctccttctgggagcttggtaaataccggactctggtgtcccatcgcagtcatcacctgcctcttacttgcacataccacatcccccctcctctgatgagtctctc

SEQ ID NO: 232 - DNA, 122 bp, Homo Sapiens
ccgccccgccgatgccacgccccaatgaagacccgcgctacggaggccctgccctcggggcgggccgcccttcacagtcaagctgccaggccagcagccgctccagctccctgtcacatgct

SEQ ID NO: 233 - DNA, 122 bp, Homo Sapiens
ggggaaaaagtttcataaggtggaatagaaaaggcaccaggaaacttgggactgagcatgcgccctgcaacataacggtgtcaacaagctcgctacctgtccgaccggttccggcggccggg

SEQ ID NO: 234 - DNA, 122 bp, Homo Sapiens
gtgctagaatgctgtgcgtcggaaggctgggcggcttgggagccagagcagcagctctgccgccccgccgggcgggccggggaagcctcgaagccgggatccgggcccgaagggtcagcacc

SEQ ID NO: 235 - DNA, 122 bp, Homo Sapiens
catagtcctacatttaattttttagtaatcactgttcttgcaaacacttaaaaaaccttgcggactatggcttaatctactaattaccctgtgcaaagtttcaaagtgtgactaagatataa

SEQ ID NO: 236 - DNA, 122 bp, Homo Sapiens
ccgcccgttttcccgctgctactcaagaaagtaactgtcccctgctgctctgtgtaaacgcggcaccttaaatgattattgcatggaccacggttactttgtgtgtctgtgcgcgctgcccc

SEQ ID NO: 237 - DNA, 122 bp, Homo Sapiens
aatccgccaggccacgccaagctccctgcccaacccttactgacgggggccacattttcccggcctccgcagccagaccttgacacaaaggacatcaaactgccgagggtaaaaaccccgga

SEQ ID NO: 238 - DNA, 122 bp, Homo Sapiens
ctgacagtcttttggtcttgagtcttcagggtgctccaggaccccgtagatctgcaggtacgtgtctgcacagggtacgggtagaagccagaaggacacacgtactccagtgcctggccctc

SEQ ID NO: 239 - DNA, 122 bp, Homo Sapiens
ctagctcctcagtccccaacaacaccagctgcctcttggagcttgtgttcgtgttgaaaacgggaaggggtgggggcaggacatgaaccatttcaccaatgaagccggttcccagccatcct

SEQ ID NO: 240 - DNA, 122 bp, Homo Sapiens
agccacacggggcaggagctgcagagctaaagccaggccatgtggactggctgcgggtttcgctgcctagctggtggcattgagcaagttacttaacctttctgaacttccgttttatgatt

SEQ ID NO: 241 - DNA, 122 bp, Homo Sapiens
gcaacggtaccactaattgcggtttcaaagcaggctcctcccataaatgcaaaggatatgcgcccattattagtgcttccaaattcaaagggataacataagcacctgttttaataaatgtt

SEQ ID NO: 242 - DNA, 122 bp, Homo Sapiens
caagttggcacagctcccagtgccagacttccaagtctagttctcagagccactgggtggcggccattcccagccagtgaatgctttgtctgtgtcagcactgaccgcccccttaaatagcc

SEQ ID NO: 243 - DNA, 122 bp, Homo Sapiens
cacgctaaattgggggattggaaatctcttgagtctgcgggtgctgagcggagagtgtaacgatgaaccagagacaggctcaaagtagagcagaagggcctgaagttaatcattgtgcagtc

SEQ ID NO: 244 - DNA, 122 bp, Homo Sapiens
ccagtagagcgggtcagatgttgccaacctctgcagagtagcaataagcagtaaacgccacgctctgcacagcctcccagtgctgggcctggtcgccacgcggagccttgggctgggacagg

SEQ ID NO: 245 - DNA, 122 bp, Homo Sapiens
accccaagcaatcccagctccttgaaaggttttttctttttgtaccgacactatcattcccgcccttcgtccccaactcctttagtcccctaaggagtgaggggggtacacatttctgctcg

SEQ ID NO: 246 - DNA, 122 bp, Homo Sapiens
atctccaaggcctcggaggaaacttttgattttataaataacttgatggtggaaggcaaacgccatcccggttcccaagcaggctccacgcagcctccatggccgagtccatctcctcgccg

SEQ ID NO: 247 - DNA, 122 bp, Homo Sapiens
ccaccaactcgcccacgagccaggacatgtgctaataatgccctaagccggttataaagacgtggaaattgaggggagaaaaaaaaagggaaaaaaagggtctgtccttcctgggattccta

SEQ ID NO: 248 - DNA, 122 bp, Homo Sapiens
gcacttagaggcttgaggttgccagctgtggggggttgggcctgtgcagtgcctctcagccgcccacgctggtggggctctgcagtgttagcttcttgtttctctattttctacctaggttt

SEQ ID NO: 249 - DNA, 122 bp, Homo Sapiens
gaccaccacgcacctggacagcagcccctcctaccagcccttcccgagtctcgccccacacggtcgccagttgctcagggcatctgtttgtaccttgtgcacacctctcttaaagcactttt

SEQ ID NO: 250 - DNA, 122 bp, Homo Sapiens
tttgctacaacttacctacagcaatgacggcttttgtaaattacacaccattcttgctctcgcagccagtattgagaagtaagccttcaagagctgatacctcttctgaaaaacaattgaat

SEQ ID NO: 251 - DNA, 122 bp, Homo Sapiens
ggcactgggcagcacgcactggagacccaggaccctgtgcaggagcagctccgggtgacacgaggggactgaagatactcccacaggggctcagcaggtaactgctttcagatcctttaggc

SEQ ID NO: 252 - DNA, 122 bp, Homo Sapiens
ggccattgtggcgtgagggtggccaggtgtttgtgaaagaggggcccccagagagcagcacgggagcgatttatacacacgaatattaacatggagaagtccgagaaacccccgtaaatatg

SEQ ID NO: 253 - DNA, 122 bp, Homo Sapiens
tttttcttataagctgtccagacctggcttgaaaacccatcccatggcaaggcagggattcgctggccgcggttggctctatcttgatctgagcaagccgctggacgtccctagttatcttc

SEQ ID NO: 254 - DNA, 122 bp, Homo Sapiens
cgctgccgcagccttcgcaggcaagtgcgcccgccggcctctcgcctcgtccggttcccgcgcgcctaccccgcgcgcctgccccgcacaactgtcccaggtgaccagcccgccacacctgt

SEQ ID NO: 255 - DNA, 122 bp, Homo Sapiens
accaccgggcagtttggggccagaccccctagctcagtcgtgtcctctagtcgctgccaacggaccttcaagacgaaccagaaatactgggtgctgaaaaaggttggtatccagttgtcgtg

SEQ ID NO: 256 - DNA, 122 bp, Homo Sapiens
tgtacggaattgatgggttcttggcctcactgacttcaagaatgaagttgcagacccttgcggtgagtattacggttcttaaagatggtgtgtccagagttgttcattcctctcggtgcatt

SEQ ID NO: 257 - DNA, 122 bp, Homo Sapiens
cgttgcgcggagtcgagggatggccacagcatcccggccctgaaggtctgcagtgaaagacgcccacctggacgggcctgagagaccaggcctgacacccggcctatccgagggatgctcga

SEQ ID NO: 258 - DNA, 122 bp, Homo Sapiens
ataacaataataataatggtagcaagcaacgctctgcagtaggggcttctctcgccatttcgtactgaggaggaaacatacttaagaggttacaaaacttgcaccaaacagataaccctcgg

SEQ ID NO: 259 - DNA, 122 bp, Homo Sapiens
tagggtggcgctgaaccaggagtagcaagagtcctgtgaggagtgcaaggaagaggaggacgctgagttccatggtcctggtctgactgccctgcaccctgctgcagcctccaactgggcct

SEQ ID NO: 260 - DNA, 122 bp, Homo Sapiens
ctgccctccagccccgcgaccctcagcctgggcagcccccgcggggtcctccgatgcccacgctttccgggaggccagcctgatccctgaatatgctgcttgtcccgtctgtctccgactcc

SEQ ID NO: 261 - DNA, 122 bp, Homo Sapiens
acttcaattcctaaacaggcataagcttcattcagggtgttcactggccacgaccagggacgcccagcacccagtgcagggcctgccctggaagaggtcagtagtgcaaagttagggctgga

SEQ ID NO: 262 - DNA, 122 bp, Homo Sapiens
gggcggccctttaagcagatgcgctccagtttttctccagagcctctactgcttgaatgacggactcgaagctacttccggccatttctcacgcccagtcagagtccaggaattcttaggtt

SEQ ID NO: 263 - DNA, 122 bp, Homo Sapiens
ctcacgacccctcgctaggcggggttcgggaccaggtgaacgctgatctgatagttgacacgggacgactgtggcatcatccttgctgccgtcaatatcccgagagggaggaggttgggccg

SEQ ID NO: 264 - DNA, 122 bp, Homo Sapiens
attccgactctggagctccagagaccggatccgagacgcgggtggaggtggagttacactcgcctgcagccctgagggtgtaaatggcccattaagatccaggttaaatcaacctatggagc

SEQ ID NO: 265 - DNA, 122 bp, Homo Sapiens
ggcggagagaacagccagttgtttaaaatccttctagagcagtttctaattccccccttgcgcccgggttcggaggggggagggggaaggagagcgggtggagggaggagagcggagaggga

SEQ ID NO: 266 - DNA, 122 bp, Homo Sapiens
tcaggtgcctgagagaggtgcttcactcctcccactgggccgagcatttagaataatcaccgcccccttcccccgccttttcctgccctggatctccgccgccacctcggtctcgctgctcc

SEQ ID NO: 267 - DNA, 122 bp, Homo Sapiens
cagcactgggcgaggagctgagctgcgaaggaggctggtgtcccccggggacaagggcgtcgccgccatccccggctgcagagtcctctctctcagaactggccgaggcctccgggtccctg

SEQ ID NO: 268 - DNA, 122 bp, Homo Sapiens
ccctccttggcgggggcgtccggcaccacctcagtgaggccgcggcgcagggcagagagacgccacacagccaccagccgcagcaccaggatcagtaaggctcctgggacagggcaggggac

SEQ ID NO: 269 - DNA, 122 bp, Homo Sapiens
cggggtccggcggatcctgggcagcagccggggtggggatgctgtcacattcggggacgacggaccccgacggtgccaaagtctgggacaggacagttgcgggacggttggggagcgtcagt

SEQ ID NO: 270 - DNA, 122 bp, Homo Sapiens
tcaggagccttacctggatttcctcacccacctgccttgtgtgagtcggcggctaggatgcggtccaagcttctgagtgtgccagcacagctgagtctctatttatgcaccagggcatacct

SEQ ID NO: 271 - DNA, 122 bp, Homo Sapiens
ttgcattcaggtagattatttggaagatgatttaaggacgtaccagtgcaggagttgtcgcgggacagtgagaccagggcagtttgacaatcaataaagggtgcatcattggcaagctacct

SEQ ID NO: 272 - DNA, 122 bp, Homo Sapiens
ttccatggtgacggcaaacaaggcccacactggacagggcagctgctgggttgctactctcgcctccgccatgattccgcccgcagactctttgctcaagtacgacaccccagtgctggtga

SEQ ID NO: 273 - DNA, 122 bp, Homo Sapiens
tctccaatctgagggcccacaagtggcatcatcgctttgcggttggagtttacccatcttcggttatttgggagggtctgtacccttcctttcccccaccccaaacccccttcctctggcct

SEQ ID NO: 274 - DNA, 122 bp, Homo Sapiens
gcaacagccgctccctctcaactggagctgcacccaggctttggctaaaggctgttaaaacgttggccaggtgcggtggctcacgtctgtaatcccagggcggatcacctgaggtcaggagt

SEQ ID NO: 275 - DNA, 122 bp, Homo Sapiens
gtcggcctggcaggcgcggcccccggttcagctgcgccggggcggcccagcgcgactccgcgggccttttggctgctcgccccggctccggaacactgtcagatccttctccgcagaggtag

SEQ ID NO: 276 - DNA, 122 bp, Homo Sapiens
tgcgctctcacttcttcccacattcaagaatggggtcccaaacgagagattcgacgtaaacgtccgctcccaactaagttctcaggcgctgacaacgacaagaattcctcacacaaacgtag

SEQ ID NO: 277 - DNA, 122 bp, Homo Sapiens
gagggggccccggggctcgggctggagggcgctgggggcctcgagggtctctggtactggcgccggggcacaatccagagccctgcgcgccgcccgggctgcggaggccccgatctgagcgc

SEQ ID NO: 278 - DNA, 122 bp, Homo Sapiens
gggaaacagcgccagccaagggcgagtcagatccagacagacccaggcccccccgcccagcgccccaccccgctgcacctcagcgccttgtagctgatgctcatatggggtcaggatgccaa

SEQ ID NO: 279 - DNA, 122 bp, Homo Sapiens
gggggcggggagcgcgcgccgggcggaggccgagtggagggggaggggagggggtccgagcggcccttaggccctgcccctgtcgcctactgcgctcagaaggggcctcagggcctgccccg

SEQ ID NO: 280 - DNA, 122 bp, Homo Sapiens
ctttgaacaccgcgcatccccgggtctggcgcgcggcctcctgagcgagactacggccttcgctgctgggagcacgctgccagctcgcaaaaagaccaggtcccctacaaaaggcgacccct

SEQ ID NO: 281 - DNA, 122 bp, Homo Sapiens
ctttgggaggcgatgttaacagagccccagggcacgagttaagggtttggcccaaggtcacgcccgggatcagttgcaaagccaagaaaagaacccgggacttcctatgtgcccagatgtgc

SEQ ID NO: 282 - DNA, 122 bp, Homo Sapiens
ccaggacttccccgaggcaggaaccgagcgccacgcttctcaccctatatagaggacccacgcaagactcagacctctgctcccacgccggaaatctcagcgcgcaagttaggcgtccacaa

SEQ ID NO: 283 - DNA, 122 bp, Homo Sapiens
tggcagcatctcctcccagcgtcaggtctaagcacacaagttcttccttccatctacaaacgcaggaaaccacgactggagaagttatatgattggctaaaccatgaatgacgcaacataac

SEQ ID NO: 284 - DNA, 122 bp, Homo Sapiens
cgatggcatcggtcaaggtggccgtgagggtccggcccatgaatcgcaggtgagtggggccgcccgcgccccgccccctcgtcgcgcccccacccagaggaagcctgtttccagctccccca

SEQ ID NO: 285 - DNA, 122 bp, Homo Sapiens
tgggatattcgtggggtgagtgccgtctcaacggtagagccgctcggtcaaagagactgacgcggagagggcgggtctctgggtccgcgatctccagcaggagcagctctacgcgggagcct

SEQ ID NO: 286 - DNA, 122 bp, Homo Sapiens
cctgcggagcagtagctaggaacagatccacttgcaggttgctgttcccagccatggcttcgcgctgctggcgctggtggggctggtcggcgtggcctcggacccggctgcctcccgccggg

SEQ ID NO: 287 - DNA, 122 bp, Homo Sapiens
gcggcgggaacaggagcaggccgagggtcctctggccagaagaaatctggcctcggaacacgccattctccgcgccgcttccaataaccactaacatccctaacgagcatccgagccgaggg

SEQ ID NO: 288 - DNA, 122 bp, Homo Sapiens
cgctcgggcccaacggacggcggggggagaaggtcagcgcggtctcaggcgggctcggcgcgcccccgccgcctccggcaagttgcgtcctggctcacaacacacattgttttgcgggggcc

SEQ ID NO: 289 - DNA, 122 bp, Homo Sapiens
ttacaggcgtgagccaccgcgccaggttggtgtttatttttcaaacaaggcgctgggaaacgcaaattctccagagctcttgtggagcggtggcagggaggggcggctcagccagagatggc

SEQ ID NO: 290 - DNA, 122 bp, Homo Sapiens
tggcgcgggatggttgcctcgagaaggtcgcagccaggagcaaagctttggggctcacaacggactgggcattccaaacggtgtaatcttcggcacatttcacccgctcccattccaccttc

SEQ ID NO: 291 - DNA, 122 bp, Homo Sapiens
cccgcgcggagcctgccccagacgcgcgctgcccgccgggcgcaaagaggccaggccgcacgcactgttgcccagcaaccgctcccaggcgtccgcaagtcccgggccctccatgcaggtcc

SEQ ID NO: 292 - DNA, 122 bp, Homo Sapiens
tctggaggctctcttcaaatatttacatccacacccaagatacagtcttgagatttgactcgcatgattgctatgggacaagttttcatctgcagtttaaatctgtttcccaacttacatta

SEQ ID NO: 293 - DNA, 122 bp, Homo Sapiens
actggcagaagaaataccaggagacaaattctgcacgaatgtaaggaaatattctgtccacgtggtccagagacaacccggactgcaacagtgaggttctacacctggaacactggacctgt

SEQ ID NO: 294 - DNA, 122 bp, Homo Sapiens
gaaatggaggcagggaccatcaggaagctcaaaacctttgcttcagccgagtttgcagaacgccctgtgaggagaatgggtgagctgggtcgaggaagcttcatcctcgtccccatccccca

SEQ ID NO: 295 - DNA, 122 bp, Homo Sapiens
gagagggccccagggggtaatcctcttgggtgcctcagctggtgcggtatgaggctgggtcgtggggggggtggggcgtgtgcggggggtggggtgggggctgggctggggggggatctcgg

SEQ ID NO: 296 - DNA, 122 bp, Homo Sapiens
tatgaaattgttttttgcctaagagcttcacttacaagcgcatagaacagaattgatcaccgcagcgggtgcctaaattcacttctccctgggagccctgagcaaaacaatttgaatgaggt

SEQ ID NO: 297 - DNA, 122 bp, Homo Sapiens
tggccactaagtttagtgtgcctggctgagccagagtgggctggccttggggccgagggacgcgggacgtgctgagcagctgcccagacgcgtcaggggcctgagggaggctgctattaatg

SEQ ID NO: 298 - DNA, 122 bp, Homo Sapiens
cccaaactgactgcttaaaattaatgagttgacaagcgagtcccaacccacctgccaactcgctggctgcctaagaagctgcctttacctgcctggaccagaccctcccccctccccgccgc

SEQ ID NO: 299 - DNA, 122 bp, Homo Sapiens
accttcttactctgcaaccaaaccaagtgccccatactacaggtaggtgccgagaaattccgcagcctgaaaaaataatccatgcaggtcaaaggcagaaatttactccgggatgagagatg

SEQ ID NO: 300 - DNA, 122 bp, Homo Sapiens
gggacccagagagctaccgtagaaaattactccttcactcacaaggactcgcaccagcaacgccatgttcctcaataaccatttccgggtgaagagtgagtttccggtgtccccttttctac

SEQ ID NO: 301 - DNA, 122 bp, Homo Sapiens
gggtgagtgtgtgtgagtgcatgggagggtgctgaatattccgagacactgggaccacagcggcagctccgctgaaaactgcattcagccagtcctccggacttctggagcggggacagggc

SEQ ID NO: 302 - DNA, 122 bp, Homo Sapiens
ctacctcctgttgcgcaatgattgggtcctgaattttatgtaaatcatatttatgaacaacgtccagtaataagtgcagcaagattagggctctaggttttctccagagttctagatgttaa

SEQ ID NO: 303 - DNA, 122 bp, Homo Sapiens
cggtaataacagtgtcatctccataagccattctattagtggtaacaaataatagccccacgcctgcaatatctcctttaatattcccaacgtccgctaagcgcgtctcatcaccatttaaa

SEQ ID NO: 304 - DNA, 122 bp, Homo Sapiens
ccggctgcgtggcccgcgggtcctgccggccgagggtcgccggatcgccagcagctgcgacgcactaacagccgctcacagtccggaatcccacgctccctagcccgcggtgagcgggggaa

SEQ ID NO: 305 - DNA, 122 bp, Homo Sapiens
ggaacgcgggtggggaagagggggagctctgagtgctgacatttgcagtgccctcccaagcgcacaacaagccacagggtgcagactcctgcaggtggtgactctcccggtcccagggtgta

SEQ ID NO: 306 - DNA, 122 bp, Homo Sapiens
gctcaaatgaattcgtggaaagtgccccctaaaaatgaaaagatgcctctggagaccttgcgcacaccgagtggcacgacctgcagaggttaccgccaggtttccatcctccgcgcccggga

SEQ ID NO: 307 - DNA, 122 bp, Homo Sapiens
ccggggctctcggcgctgagttcgggggtcccattctggacgcggcccagcccctaggggcgccgccctggcctcagctgcgcaattacagcccccgccggcagctccccgccccgggctcc

SEQ ID NO: 308 - DNA, 122 bp, Homo Sapiens
accggggccaagagtgatacctgatcctgggggattgtgaaatgacctcatgtggcagcccgcccgcggtgcccgcaaagccctccaccctccccttccccgctcggctccacccctacccc

SEQ ID NO: 309 - DNA, 122 bp, Homo Sapiens
agcctcctacagcagccaggtacacacaaggcctcagtttttccatctgtgaaatgggtacgtggacagacctgtccagtagcctcccatgtgagaatcctcggactgagagggtactagga

SEQ ID NO: 310 - DNA, 122 bp, Homo Sapiens
gcgccgctcgccttgccttctccttctctggctgcttttgcttcggctggagccggtgaccgccgcggccggcccgcgggcgccctgcgcggccgcctgcacttgcgctggggactcgctgg

SEQ ID NO: 311 - DNA, 122 bp, Homo Sapiens
ccaactcgcggattatgttaatgtgattttcatcttcaacgttaacacggaacaccttctcgctgaataatgagataatagaagcacagagaattggaaatgagtaatgtgtccaaatttgg

SEQ ID NO: 312 - DNA, 122 bp, Homo Sapiens
agagtgtagaaggaaggctctagaatgagacagcccagtcccggttactagtcatgagaccgcgtattgattacctaacgccgtaggacctcggtttccccatttgtacaacggagattctg

SEQ ID NO: 313 - DNA, 122 bp, Homo Sapiens
tcacctgtgctgcactccagctgacccaagtaggaagccagacgagctgtaaaacatgaacggaagagtggattatttggtcactgaggaagagatcaatcttaccagagggccctcaggta

SEQ ID NO: 314 - DNA, 122 bp, Homo Sapiens
tcgcaaagtgctgggatgacgggtgtgagtcactgtgcctggcctggtttctcctctccacgtgccctcccccgccagaacccagctgcctggatactcactgtagcctgccctccgccggc

SEQ ID NO: 315 - DNA, 122 bp, Homo Sapiens
tgggcggcttcttgtcggacatgagcgcctccacgctgaagggcaggctggagaccttgacgcggcgctcctccgcggccccctcggcgcccccaggccccgggcctggtccggccaccact

SEQ ID NO: 316 - DNA, 122 bp, Homo Sapiens
tccagtttgggggtcaccgagctggggtggtgtgcagccagcagaggggattcaggatcccggggtgactcctggcccaggcctgtcaccccacagctggctcctggccccaaggcagccac

SEQ ID NO: 317 - DNA, 122 bp, Homo Sapiens
gccctggagaatgtgcctttaggaataacttcccctaccgagaagtttctggtgtctcctcgtggaggcccaaacactccatgcctgactcccgcggttcacctgtttggttccactcccca

SEQ ID NO: 318 - DNA, 122 bp, Homo Sapiens
atggctgttccagcctcagcagccagtttgaaggattcccagggggtagccaccttgcttcggcctcttccaggctgggaagtgggagcctgtgcctttaaattctcagcaggttgaaatgg

SEQ ID NO: 319 - DNA, 122 bp, Homo Sapiens
tgatgacagcgttctcaggacagtgtcttgtagctggggcgctccccaaggatgttagaacgttcccgggggacaggcaggctgttagaaattggggcgcgaagccggggaccgttcctggg

SEQ ID NO: 320 - DNA, 122 bp, Homo Sapiens
gggtatttttagcaggcggtgtttgaggtctctattaatggcaatgacccgtttgagggccgcccctccccatgcactcctcccccagcgttcaggggccgtgggcggggcgaagagggttc

SEQ ID NO: 321 - DNA, 122 bp, Homo Sapiens
actccaccctcattgcgtccctcagatctgatatttgccacttgatttctgcttttcaaacgccttttctgcctgtgttacctaaattacacagtaaaggatattggcaagtgtttagccaa

SEQ ID NO: 322 - DNA, 122 bp, Homo Sapiens
gagagggaaaaactagagataaaccaaactgactctcagttcctgagactgatgtgacatcggcttgatctaaaccccaaagctgatgttttatttgttgttgactctgtgtatggttttgg

SEQ ID NO: 323 - DNA, 122 bp, Homo Sapiens
ttctccatctcagccaaagctcttacctacacctggtgagcatataaaaagcacaatttacgctgactagctgatgcatatctgaaatgaacggagaaatacacctcatcctgagagaaaaa

SEQ ID NO: 324 - DNA, 122 bp, Homo Sapiens
ggttttggggtatctgggctccaggcagaagcacagcctccccgacctgccctacgactacggcgccctggaacctcacatcaacgcgcagatcatgcagctgcaccacagcaagcaccacg

SEQ ID NO: 325 - DNA, 122 bp, Homo Sapiens
acccccgggcgcatccagacgagcccccgccgcagcggcgacagcagctggagctgcccgcgcccccgcccgacccagcccccgatccccggcccccgaagcctccgcccgggtcaccccgc

SEQ ID NO: 326 - DNA, 122 bp, Homo Sapiens
ttttggatgtcaaaaggcactgatgaagatattttctctggagtctccttctttctaacccggctctcccgatgtgaaccgagccgtcgtccgcccgccgccgccgccgccgccgccgccgc

SEQ ID NO: 327 - DNA, 122 bp, Homo Sapiens
gcctcagggagcagaactgagatccatggatggaagccatagagaaaaggagttcagacacggcaagaactttctaagagccagagctgtctagagaaggaaaagataggtagtgagctccc

SEQ ID NO: 328 - DNA, 122 bp, Homo Sapiens
cgctggaaaatgctgaaggacagcgagaagatcccgttcatccgggaggcggagcggctgcggctcaagcacatggccgactaccccgactacaagtaccggccccggaaaaagcccaaaat

SEQ ID NO: 329 - DNA, 122 bp, Homo Sapiens
agggcccaactaggagtgccaacaaccgcctaaaatacagcagggcggctgcacggttaacgtgcgtgtaattgactacagcaagaaggatgaggatctgtccataaatgccaagttcccct

SEQ ID NO: 330 - DNA, 122 bp, Homo Sapiens
cagcgggaatcgagagtggggggctgtcccagcgggcacataccttcggcgaaatagtcccgcggcttctggaaagtgtcccggacgggctgcgggcgcctctccgcctgcggaggagacac

SEQ ID NO: 331 - DNA, 122 bp, Homo Sapiens
ccccgagtctcgtttcagtcatccctttgtccttccccggattggcaggttttattattccgcctgaacaatccggccgcccagtggctgagggtcgctgacgtcggaggcagagccgggga

SEQ ID NO: 332 - DNA, 122 bp, Homo Sapiens
gtgtcactcgtataaaaacctatgctttgaaggttctcgtgtgtctcggcctgcaggtctcgctcagagctgtgtccctgaacatccaccctgctggggtggcttgacgcacttctgtgcaa

SEQ ID NO: 333 - DNA, 122 bp, Homo Sapiens
ccgagcgagggtggggggcggcgggcggcgcggggcggcggcgagcgggggccatgcaggcgcgctactccgtgtccagccccaactccctgggagtggtgccctacctcggcggcgagcag

SEQ ID NO: 334 - DNA, 122 bp, Homo Sapiens
ctgggaccgagggagggggcaggcctggactctgctgaacacctaatgagcatcagggagcgccactgcaggacgagcagcagagccaagattagagcccctgctgtcctcactacagccac

SEQ ID NO: 335 - DNA, 122 bp, Homo Sapiens
tttggggcgcagggggggccgtgccctggcggaggagcagaaggcagagggtagcagctgcggctcagcggagagacttgttgcatttgcagctaaaaccaggccctacttgtccttggtgg

SEQ ID NO: 336 - DNA, 122 bp, Homo Sapiens
cttccccgccccttccaagtagaaatctagaatcaccagagtcctacagcaaaaccaggacgctgaactcatcccgaagacccggcagcccctcttggggatccgccctcattccaaattct

SEQ ID NO: 337 - DNA, 122 bp, Homo Sapiens; identical to SEQ ID NO: 63

SEQ ID NO: 338 - DNA, 122 bp, Homo Sapiens
aaatgtcccggcgcctctcccgagagccgcgacggcccggagccgggagggaaagctccacgcacaaacaccgccccctgcacaattagggcgaagatgacggctgcaaagttgttgcgtaa

SEQ ID NO: 339 - DNA, 122 bp, Homo Sapiens
gagggccccgccttccccagctgcatataaaggtctctggggttggaggcagccacagcacgctctcagccttcctgagcacctttccttctttcagccaactgctcactcgctcacctccc

SEQ ID NO: 340 - DNA, 122 bp, Homo Sapiens
gtttccgcagcccaggcgggacgaagcttctctcttggtcccagtgccgcctttcattcgcgcgctctggcctggctggggccagttttcattcctggagacccctaagtggaagaatgaaa

SEQ ID NO: 341 - DNA, 122 bp, Homo Sapiens
gcggtgggaagtgaacgattaatcagatttcctcttttcccttatttcccctcccccttacgaagataaaaactcgggaaagcaaaagaggtgcccgccctgcagttcttaagctgggtccc

SEQ ID NO: 342 - DNA, 122 bp, Homo Sapiens
ttccaggtagcacagaaagcctccaccatccttgccctttcccctctttagagacaggagcggggatcttctggtctaattgccactgcagctcagaagaccctttacacctcagcaagcag

SEQ ID NO: 343 - DNA, 122 bp, Homo Sapiens
agtgctgcacacctgggtttccttgcctagagctgtgtgttcggggtcctttggtccagtcggaggctgcggagcggcgggggttgcctgcgctgtccgcccgggcatcctcccggtgatgg

SEQ ID NO: 344 - DNA, 122 bp, Homo Sapiens
ccttaggaagtggaaaaccgtgtcccaaggtgtgcggtccagctcggggctgggaggtgacgctggtgggctgggagggaggggcggggggccctgagcctcaggccacggcctcgttagga

SEQ ID NO: 345 - DNA, 122 bp, Homo Sapiens
cggggccgggacccctgacccctgacccctgcagcgctgcgccccgccctccctcgtgcgcggcccggaccccgccaccctgcaggattgcgcctactccgactgccccttccctatcgtcc

SEQ ID NO: 346 - DNA, 122 bp, Homo Sapiens
ctcctgcggccccgccctctggcagctgacgtcagagcgccggcagcagcaccctggtcacgtggccagcctgttgccatggcaacccgctaactcatcacctcgcctcaattcgcacaggg

SEQ ID NO: 347 - DNA, 122 bp, Homo Sapiens
tgcaaaatgcagcaataagccaaacctagactttctattgacttgtctattttcgtatctcgctagcagaaagactacatttctggtcatttccattattggggaatcgtactttaccattg

SEQ ID NO: 348 - DNA, 122 bp, Homo Sapiens
tgacgcctataaggaagggctcgccgccgttgggctgctggtggttctgcagggtctgctcgctgtccccggccatggttcgcgccgcccctcctcccgctgcctgtgcgaacggacgcacg

SEQ ID NO: 349 - DNA, 122 bp, Homo Sapiens
ggtccgcatcccgtcctcggactcatcctcggagccgctgctgctgtagcccaccaggggcgccgcgctcatggggcctcatccaagaccaccagagcaggtccaccagcaacctcaaccgg

SEQ ID NO: 350 - DNA, 122 bp, Homo Sapiens
tgcatcttcagcattcactccaggcccctggccaggtggaaactgggcacaatttagttacgtaagccacagtgccctaaacaaggctgttgttttcttcccaggctcagcattgccctgct

SEQ ID NO: 351 - DNA, 122 bp, Homo Sapiens
taccagtccgcccgccctccagcctctgaaaacagctgtttgaatttggagctccgcatgcgcagtgcccggtactcaccccccgcaagcaccgctccccggacagagttgcttggccaagt

SEQ ID NO: 352 - DNA, 122 bp, Homo Sapiens
agggccaccctttaactcggcagcttgatgactataatgggcccagttgtctgcgggctgcggggagctaagtccccagattggaggaggctggctctggtcttcgatgcacaggagtggcc

SEQ ID NO: 353 - DNA, 122 bp, Homo Sapiens
ggcccgcgcgcttaccattggcgttcttgccgcagatgagatgctcatagggctgcaagacgccccagagctcggcgtcgttgttgatgaccatctccagggcctcagcagacacctccccg

SEQ ID NO: 354 - DNA, 122 bp, Homo Sapiens
ctgagacatttacatgctgtttttcggaggtgtccttaaagggaaggggaatgccaggaacgggctgcaacttggcaccaccgagggaggtcagcttttacaaagcacatccaaggcaggcc

SEQ ID NO: 355 - DNA, 122 bp, Homo Sapiens
gaactggtccgcgcacgcgcacgacgccgcaggccccggccccggcccgcggcagctgaacggcagagcctgtagctgcacagctgtgcttccacctggcgttcagtacctcgtgcccgaga

SEQ ID NO: 356 - DNA, 122 bp, Homo Sapiens
gtgcgtttggagataaatcctatctttccctgccatcagcacttaccttctgggaaatcacgcggctgtaccacatagagaaagatatgcactagttcaaagagaatgccaatgggtccagc

SEQ ID NO: 357 - DNA, 122 bp, Homo Sapiens
tacaatgtattaacttagttttttagatcagtttgaaacttccttatttttcaatagccacgcaaaagagattctccattgcttaaagaacaaatattttaaggaattggaggacctggagg

SEQ ID NO: 358 - DNA, 122 bp, Homo Sapiens
agtgcgggcccctgcattgccaaggccttataggcacgggctgggcgggggtgggcagtccgccagccagcggcattctgcagggctctgtgcaagcgtcagcccaggacgggcacctgccc

SEQ ID NO: 359 - DNA, 122 bp, Homo Sapiens
gcgcgcttgacccagaacagtacggagttctgcacgagccgggggtggggcctgtctcagcgcgcggcggtggggcggggcttggacacgggcccggctcaacttgagggaggcggggctcg

SEQ ID NO: 360 - DNA, 122 bp, Homo Sapiens
ggttgcaactctgagtagcagaggagctcagcgtcgactggggcgcgtgatcctttatagcgctagccacctgggggccaaggggcggtgctgctttcccggaaacctcgcgccttcccctc

SEQ ID NO: 361 - DNA, 122 bp, Homo Sapiens
ttccctgcagattgtcatgaaccaaaccagaaaggggtatcctgtagagaagttaggggacggaggataaacttgacagtgcatgaaaaaatgtaataaatgggatacagaggactatcaaa

SEQ ID NO: 362 - DNA, 122 bp, Homo Sapiens
tgtccttcctattgtttagctcggctccggagttcacgggagcctcgtgaaatgtggagacgtcctgcaaagtttcttgaacacttcatgtgcgtggtaggaagtcgaggaaaaaggtttac

SEQ ID NO: 363 - DNA, 122 bp, Homo Sapiens
aggccgtggtgggcacccgggctgtctgcagatagcttggctcattgttggtcctcagtacgcagccctcgtagccaagcagcttgggcctacactctgggcccaggggagtggctgtcgct

SEQ ID NO: 364 - DNA, 122 bp, Homo Sapiens
tctcctcctcttcttctgcagggctccgcgaagaggtctggcactacacggggcagtggccgcggcgtccttttctgccccgcgccccttcagcccatcctccctgacaagggggcggcgca

SEQ ID NO: 365 - DNA, 122 bp, Homo Sapiens
actcaggagtgagtctttgtgcagtcagggcttctgctgggcagggccggctgttaatctcgcctggcggagcggacaccggggcgtggtgggggagatgggccttataggtggcgtccgct

SEQ ID NO: 366 - DNA, 122 bp, Homo Sapiens
gtggaaggtgttgatgatggtctctatgttgcgttccagctgcgacattttgcaagtcatcgtcttgcactctgtctgtgtaatggaaaaccaagagttgggacacttagaagaaaatgctg

SEQ ID NO: 367 - DNA, 122 bp, Homo Sapiens
aagcgcagagtagtttgggtgccctattttacctctccttgtcagctgcgggtgtcaagacgggttgagggaggagagccaatgcaccccgctccaacaaagggtcagcagcgcgggctcct

SEQ ID NO: 368 - DNA, 122 bp, Homo Sapiens
ttgtcttttgcggtttatcttcctggggagaaactttcacctcctcagccgggcggtgagcgcgagactgatagcagcaatcattcctgcagataaatgaattgaaaggacgacaccgtccc

SEQ ID NO: 369 - DNA, 122 bp, Homo Sapiens
tacctcgattggagtggctcagggtggacgactgggaagacttggacttctgccgcttgacggtcatcatcacctgctcctgcacgcgctgcctgccagacgtgcctgttttcatcttttgg

SEQ ID NO: 370 - DNA, 122 bp, Homo Sapiens
ctgccaaaatcatcatcgtgccgccaattatgtttgaaagccatatgtacattttcaagacgctagcctcagccttgcacgagagaggccaccatacagtgttcctcctctctgaaggcaga

SEQ ID NO: 371 - DNA, 122 bp, Homo Sapiens
ttgtatttcaaacctaatttgtctgctttcatttgtgtctcacactgctgtcacatttaacgtttttaccaaaatttgaataatcttagctgatggagtagtaaacatatctacctacagaa

SEQ ID NO: 372 - DNA, 122 bp, Homo Sapiens
cctcgccctgcgacgtgtggcccagcaggttgggcatgcgggtcaggttgtagccgatgccgcggcacatggggatctccaccgcctggcacggcgcagccccgcgcccgcgctccgggtcg

SEQ ID NO: 373 - DNA, 122 bp, Homo Sapiens
gctgtttgccaacgaccagctggcttaatctgacttcgcagatgtagcagtgttggaaaacgtcggttcttttgctgagggaaggaagagatgggaggggtagaaagggcggtggttattcc

SEQ ID NO: 374 - DNA, 122 bp, Homo Sapiens
cagcccggcagtccgggatccccgggccgtcgccccgcttggggcctccttggcccttcccgcctgtccgtcattcgagcctccctcgcttgtttaagccgctccgggcccccctccactcg

SEQ ID NO: 375 - DNA, 122 bp, Homo Sapiens
gccgatgtttctttagccaaaggcagtgggctagtttagacggcctttcattcccggcagcgcctgtgggtgcgtgtgacctgggacagaggggcaaaggaacctggcagagcgagcttggg

SEQ ID NO: 376 - DNA, 122 bp, Homo Sapiens
gtgtgaatgtgtgggcgggaaggctgggtccacagggacgtggtccagcatcgtgagcagcgggtcctaccggacgttcaagtcctacagccctgggctaactagcaagtctcagaccccca

SEQ ID NO: 377 - DNA, 122 bp, Homo Sapiens
tttgtttttttaaaaagcatattctagtttctatctgtaactcgtttctagttctgccaccgcgatgccgaaggcgcccaagcagcagctgccggagcccgagtggatcggggacggagaga

SEQ ID NO: 378 - DNA, 122 bp, Homo Sapiens
cggctggaccccaaccctgccggccgccgccgaggtgcgcagcccgcagccccacacccacggcctttgcaacaccccaaccgttgaactgcgcccctacacgcccggcgtctgcggctccc

SEQ ID NO: 379 - DNA, 122 bp, Homo Sapiens
aggcaggttcatgaaattgtgtaactttccttttctgtttcttaataggggcactatgaacgaagaggagcagtttgtaaacattgatttgaatgatgacaacatttgcagtgtttgtaaac

SEQ ID NO: 380 - DNA, 122 bp, Homo Sapiens
gagccacagggagactatgtctcgcttaaattcccaaaagtgggcccctgtgcttcaaaacgtccccgcatgggaaccacaaaaacgttgcctccccagttatcaccccaagggcccaagag

SEQ ID NO: 381 - DNA, 122 bp, Homo Sapiens
tgggagcactttcgtacacagaggacttcttatccatggaattggccgttctttgtaaaacgctctgtacttactagcaacctttacacagacatgaagtttctccataaggataaagggat

SEQ ID NO: 382 - DNA, 122 bp, Homo Sapiens
tctgacttgggagctacatcctcggcagacgagaagcacttttgctcgagaattgttttacgcgcccctctaccaactgcggaatcgctcgcctgcgaaaacggaaggaggggcgaggacag

SEQ ID NO: 383 - DNA, 122 bp, Homo Sapiens
gtgctgtccccttgccgccgccagagatctgtggctttttataatggggttggctgcctccgcctccagcccccgcaggcaggagggtgggaggggagaggaagggagccagtgtgcagccg

SEQ ID NO: 384 - DNA, 122 bp, Homo Sapiens
tgaatagagtcggttttggtttcctgttgcttctccgggccatttatcttctttcttcttcgcctctggcccacgccggggcggatgttggggcgcggagtgtgggctctgcggcgccgcgt

SEQ ID NO: 385 - DNA, 122 bp, Homo Sapiens
gtggtgctgcggtgtgaggatagattctactcaacaccccttcaaatcacaccataagttcgctgcctcctgattgtatggggacggtctggatggtgcggtagggtgcacgtaagagtgtg

SEQ ID NO: 386 - DNA, 122 bp, Homo Sapiens
acccaatgtccagggagcgtgcaaaatttgtcaaaagtggccttttattgtaaaacgacacgagagctaatgctgcatgcccgttgctgcctgaatcagaagggcaccatcttggggtgagg

SEQ ID NO: 387 - DNA, 122 bp, Homo Sapiens
ctggaccacgagctctgagagcagcaggttgagggccggtgggcagcagctcggaggctccgcgaggtgcaggagacgcaggcatggccggtgagctgactcctgaggaggaggcccagtac

SEQ ID NO: 388 - DNA, 122 bp, Homo Sapiens
ctggcggccgccggggcgcaggtgagcgcgagctccgggctctgaggctggacgtggagccgcgaccgccccagccccgaacccgccactccggggtgcccggcgcagcctcgacgccccca

SEQ ID NO: 389 - DNA, 122 bp, Homo Sapiens
ggccaatagttgtataatcttagaattgctgcctacccagttccttcttattcttgttcccgccttcagaaagttatgtaagacacttattttaccctcgggcttctcttgattgtatttgg

SEQ ID NO: 390 - DNA, 122 bp, Homo Sapiens
atgcagcccgcgcctcgcccttcagagtaactcctgtggactctgagactctgggatattcgccttctgccccagatccctggaggggggctgagtggcagcctgcgctgaagataccacct

SEQ ID NO: 391 - DNA, 122 bp, Homo Sapiens
agaagaagaccccggcttgagagtgaggtgtgctgggcggagtgggggaggacctcgaggcgccccggcaaccaaccgtgttctaaccaactggcaagtgggaaaagttgacatgactagag

SEQ ID NO: 392 - DNA, 122 bp, Homo Sapiens
tccccagccagctctctgcggactgtctaattaatactcgtttagccatagacgccaagacgcagaaatgcgttgagtaatgcaagcaacgagggttattttattttagatactaaaggcag

SEQ ID NO: 393 - DNA, 122 bp, Homo Sapiens
tcttctcttgagttggcttaggtacctgggctcctccgctgtcccctctggccacagaagcggaggagcctgtaaagaaaaccaccttggaatttcctgtgctgtggggcaggagaacaagg

SEQ ID NO: 394 - DNA, 122 bp, Homo Sapiens
aagggcacttagtacttagcgttgcctcaatccacagtctcagttccttttgttcagatacgagtcgttacttaattcagtggtctcaaccctggctgcagaggagaaatcagctggggagc

SEQ ID NO: 395 - DNA, 122 bp, Homo Sapiens
aaggggcaccggctggcagtgggatttaggatctagcccagccctgtcttaaactggctgcgcgaccttagccaagtcactgacctgtccgggtctcaagtacatatctattaaattagatg

SEQ ID NO: 396 - DNA, 122 bp, Homo Sapiens
ggaagagggtctcgttctaactgtgccgggccgggaacgggttaagatgcggccaaatgtcggtccctcctccttcccacccctcagccccggcgcccactcgcgacggcagccgcggaacc

SEQ ID NO: 397 - DNA, 122 bp, Homo Sapiens
gggagctcgcgccctcgcccagccgagctcccacccccgcttttttccgaaggcgctgggcggcgccaccctccggccggagcccggcactgcacaaccccctccgactttcaatgttccac

SEQ ID NO: 398 - DNA, 122 bp, Homo Sapiens
tccgagctcgagcgtgacgggaagttggggcaatttgttagttatccgccgccaccaagacgcggcacggcgcctggaccggaggggccccgcgcgggcgcgaactttgggctcgggcgagt

SEQ ID NO: 399 - DNA, 122 bp, Homo Sapiens
acaaataggaagtgtgatgactcaggtttgccctgaggggatgggccatcagttgcaaatcgtggaatttcctctgacataatgaaaagatgagggtgcataagttctctagtagggtgatg

SEQ ID NO: 400 - DNA, 122 bp, Homo Sapiens
gcgttggggttacaacaacgcacaaaacataaattctgaagctgacacgctagctttaaacgttccttaaatatcttcatcctcaatttacggttgaggaaactgaggtgtagcgagacctt

SEQ ID NO: 401 - DNA, 122 bp, Homo Sapiens
ggtcccggagttctgctcacttcagccgtgtgccgggcactgcaaatcaggaagtgttggcgccggctggcgacctcccgcctggggccaggggaggagggtggttggacgctgccaccgct

SEQ ID NO: 402 - DNA, 122 bp, Homo Sapiens
ggcctcaagtgattcacccacctctgcctcccaaagtgctggaattacaggcatgagccacggtgcctggcccaaactgcatatttaaagggtagagtctgatggctttaggtacttgtcac

SEQ ID NO: 403 - DNA, 122 bp, Homo Sapiens; identical to SEQ ID NO: 402

SEQ ID NO: 404 - DNA, 122 bp, Homo Sapiens
tccatgctgaagtagttggagttcttgtcgtccccggactcgacaaactggatgatgggtcgccgccactcattgcccgcgaacagggcgctcctaccttcccctggcttcagggcgctgcc

SEQ ID NO: 405 - DNA, 122 bp, Homo Sapiens
cagcggctgtgcgctccctacagtcattactgtaccctcccggcagcctccggcagaacacgcagcagcagcagcagttagcgctgatgcaactatcgttgctgttgccaccgcctccccac

SEQ ID NO: 406 - DNA, 122 bp, Homo Sapiens
tccaccctctttctctcacctttacttggcttctgggacttcacttggcttctctggtgccgcggggtttcccgcctcccaggcacaggtcgcctccccagggcaactagcgacgtaggagc

SEQ ID NO: 407 - DNA, 122 bp, Homo Sapiens
agcaagtcgcttaggaagccaagttcacagtttggatcttacagagtaataagaaaggatcgaaggctggggaaaggttacattttgaaaggcattatacacactgcccaggagtttgaact

SEQ ID NO: 408 - DNA, 122 bp, Homo Sapiens
catgctgaggaaagtggccgtagcggctgcgtccaagccgcacgtggagatccgccaggacggggatcagttctacatcaagacatccaccacggtgcgcaccactgagatcaacttcaagg

SEQ ID NO: 409 - DNA, 122 bp, Homo Sapiens
atggtcagaggcataatcccaagtcgtttcaataattccaatgtaataatgcttttctttcgcccaggctggggtactacataaaaacagaaaaataccaagtatcaaaatcttcatttttt

SEQ ID NO: 410 - DNA, 122 bp, Homo Sapiens
aaactggcctcccctagtgaagtggtgcagcaagtcgcagagaagcaatatccaccgcatcgtccgagtccttactcatgccaacactcactctctttccctcagcactcattgccacaggg

SEQ ID NO: 411 - DNA, 122 bp, Homo Sapiens
tcctgggttgtcattctcttaggtcataaaccggctgtgcctggacacagctgagtgagccgcgtctgagctcatccgctcctggggagaggccctgggacgccccgcctgtcggtggcaat

SEQ ID NO: 412 - DNA, 122 bp, Homo Sapiens
ctttggcggcggaaatgttgctgaagcggatctgggctggcttgtcgcggtcctgataggcgcctttcccgcggccgccggcagccccggcagtcgccccgctccggggtgccacattctcg

SEQ ID NO: 413 - DNA, 122 bp, Homo Sapiens
gcagaccaccatgtgctatgggaagtgtgcacgatgcatcggacattctctggtggggctcgccctcctgtgcatcgcggctaatattttgctttactttcccaatggggaaacaaagtatg

SEQ ID NO: 414 - DNA, 122 bp, Homo Sapiens
attgtgattgattatatgtttgactcctcaccagacaagatctccgttaattcagtcattcgttcacacattcattcagcgcatactgagccttttctgtgtcaggcccagtgttagccttt

SEQ ID NO: 415 - DNA, 122 bp, Homo Sapiens
ggtgagaggcgagggcaggctgggagacaacaactttcgcagcgacttggccggggctggcgccctgcgcgttcccgggccgtgcgctcgctctgagacccgctccccgccacgctctctga

SEQ ID NO: 416 - DNA, 122 bp, Homo Sapiens
gcgcccggcccaaagtgagatttcaactctctctacaccagctggacctgacagcggactcgcggaggcgcagacgccgccggcggtcttagctcaagaatgaagcagccgcactgggctaa

SEQ ID NO: 417 - DNA, 122 bp, Homo Sapiens
cgcacctgaacttggcgtggctctgccctgcctggccttttgtaaggcagtttctcaatacgcaggcagggagcgggatcttcccgccactcattcttttctttttagagatgcggtctcag

SEQ ID NO: 418 - DNA, 122 bp, Homo Sapiens
gggtggaatgtggtcatgtttcagactgccgatggcttccacttcccagacaggcccagacggccccgccagcagccgagagacattcctcaatagcccagtggctgccaagccaccaaagc

SEQ ID NO: 419 - DNA, 122 bp, Homo Sapiens
acggcaaatgagatgaaaggcgaacaatccgaaagccggatcactgaggagctctgggaacgcgccgcgagctgaagccacacggggaactccattttgtcgggacagggtcggtcctggcc

SEQ ID NO: 420 - DNA, 122 bp, Homo Sapiens
cctccatcccttgccatttcttgagattcttctgtttagcagagatcactcggccagacacgcatcggcagcagcctgggaatgaagccagcctgcctggggcggcccccgccgcgctcccc

SEQ ID NO: 421 - DNA, 122 bp, Homo Sapiens
cggcggaggaggggaggtttcgggtggactcctcagacagcggcaggcagttttcgaaggcgcgcacgaaaagcaaaactcttccgctcttccacacgtaaacgccaacgtcctccgggcgg

SEQ ID NO: 422 - DNA, 122 bp, Homo Sapiens
ttgctggagaacccagcttgaggctcagtttccaggaatgacatccaccctacactgtcacggtggtccaatgagaaccccatactgcagccacatggcatgagacacccagagtggagtga

SEQ ID NO: 423 - DNA, 122 bp, Homo Sapiens
cttgcggcagcagctggacgagctgagctgggccactgcgctggcggagggcgagcgggacgctctgcggcgcgagctgcgggagctgcagcgcctggatgcggaggagcgcgccgcccgcg

SEQ ID NO: 424 - DNA, 122 bp, Homo Sapiens
ccccacccccactccggcgcgccatctttagtgctggcagcgaggcttcttggccccatacgtccaaccttgctgctggagagattggggtttatttttaactgacttacatgaacgtgacc

SEQ ID NO: 425 - DNA, 122 bp, Homo Sapiens
agtgggtaatttaaaccagtgtgcacttcaacataaataccaaggcacttcctatttgaccgctctcgcccctccccaccccctgcatacacaacacatgcacatactcacccacaaacaca

SEQ ID NO: 426 - DNA, 122 bp, Homo Sapiens
gaggtggctgcgcagtgaagagggacccccaggcccacgccccggggtgggagccgaagacgcccttgggccacggtagtgttggagcccttgtttgaatactgacattttaatcatagtta

SEQ ID NO: 427 - DNA, 122 bp, Homo Sapiens
taatggtatcaacagacgaggacaacagcaacaacaaagtctttatacaagcctgcaacacgtgacaactgcggatcaggcatctgaacccggcctatctttccaccgtatcttcctacact

SEQ ID NO: 428 - DNA, 122 bp, Homo Sapiens
ggccactcacctgctgagaaccttcgatggctcctcgatgcccgcggagcaaagttccgacgcctcagtttggcgtttaaaagtcttcacggcatggattcggtttaccttacttcacgttg

SEQ ID NO: 429 - DNA, 122 bp, Homo Sapiens
gctttgcttcacttttctgccactttctttacttaacttttctgtcttctctgactagttcgcttgctcgaaaaaatgaagggattttaacggggggctgggaagatggtcacataacattt

SEQ ID NO: 430 - DNA, 122 bp, Homo Sapiens
tatccttggccagcttcttctgggatacacattctctaggtcttttatccactgaggtttcgacagcctgtcctacacgaaagaagcaagaagtccagggacttcccctggcagacttgtga

SEQ ID NO: 431 - DNA, 122 bp, Homo Sapiens
gggaaacggacggtcgcggccagatgggtacgaaccccccgtgcgcagcgcggagtggtacggccgggcccccaaaccttgcagtctcactcgccggtgagataatctggtggttgtggaga

SEQ ID NO: 432 - DNA, 122 bp, Homo Sapiens
gcctcctttcgaaaaggaccccactggatctcaaggggccaatgttgcctgagcgaattccgcctgggaggaagggggcggccacgttggcaaggcatcgtgcctggctcaccctaaagcgc

SEQ ID NO: 433 - DNA, 122 bp, Homo Sapiens
cccctccccaccctcttttctctcctcccctcgatccctcctcctcctcttcacctccagcgcccagctgctcgctgagcgcagttccgacccacagcctggcacccttcggcgagcgctgt

SEQ ID NO: 434 - DNA, 122 bp, Homo Sapiens
gggcggggggatcaggttcagcctctgaaagtccagagcacgatgtttgggaaacggccccgcccgcggcctccgggacctggcgaaggagccccgcggggcgcccctgctaccaccctgcg

SEQ ID NO: 435 - DNA, 122 bp, Homo Sapiens
aatcaaggtgaagctgaggtttgagccttacttaaaggctgatattttccactctaactgcggacagtactgtagcactgttatagtacctgctctgaatgttagtctagcaactcagggtc

SEQ ID NO: 436 - DNA, 122 bp, Homo Sapiens
ctcgatcccggcgcgttggttgtaaagtaggaacagtaatagcacctacctctagggccacggagaccgaatgaaataatgcctgtcaagtcacttagtacacagtaagcactcagtaatgc

SEQ ID NO: 437 - DNA, 122 bp, Homo Sapiens
aagagaggccgaagggcacggggtagggggttctcgtagggtcccagcctcaatggttcccgccctggacctccagctgccctgactcccctctggacactaagactccgcccctgaggctc

SEQ ID NO: 438 - DNA, 122 bp, Homo Sapiens
ttattctggtatcaataaaaaggaactgttactatagtaacagatattccacttggtgcacggccacttccacgatgcggaacatcatgtccaagccacacgcttgagaggcacaaataaat

SEQ ID NO: 439 - DNA, 122 bp, Homo Sapiens
actgcggagggacacacccaggtttctgagagatgtccagtagtacacaggtaacatctacggctcgagacaggttagagattttgaagaaataagttaaaagttgaaacatgaacctatgg

SEQ ID NO: 440 - DNA, 122 bp, Homo Sapiens
caggcaagcgaggagcacaaagacctggatgtggaggcgaccgtgtctgggcagaagcagcggtggctcacctgggcagcaccgccacgcccactgccagcccagctccacgccccgccccg

SEQ ID NO: 441 - DNA, 122 bp, Homo Sapiens
acggtcatcgggaccctggccgaggacctgcatatgaaagtatcgggtgacacaagcttccgcctgatgaagcaattcaacagctctctgctccgggtgcgcgaaggcgacgggcagctgac

SEQ ID NO: 442 - DNA, 122 bp, Homo Sapiens
agagacgagagtgtagatgacagagagagggtcccgaggaagccgttttgcctcctggaacggttgggtaaatccagaaacaagatcttttgggcatcacttgacagccctacatcctatgg

SEQ ID NO: 443 - DNA, 122 bp, Homo Sapiens
aaaccgagaggagttgtgaagggcgcgggtggggggcgctgccggcctcgtgggtacgttcgtgccgcgtctgtcccagagctggggccgcaggagcggaggcaagaggtagcgggggtgga

SEQ ID NO: 444 - DNA, 122 bp, Homo Sapiens
tgctttccctacattcagggctgctccttttgtcgccaatacaaacctgttgacaggtcacgcctgggaagcgggtggggtgtcccggagcggtgctgaggcgctgcaggcccggcttctgg

SEQ ID NO: 445 - DNA, 122 bp, Homo Sapiens
tctcccatttcacagagggcagactgaggctctgagtggggaagttcttgcaagatatcacgcagcagagcagggatgtgcaccagatctcctgactttaaaaagcaccaaacatagtgatt

SEQ ID NO: 446 - DNA, 122 bp, Homo Sapiens
ccggggtgggacccggggtctcctcgggctctgacgtgtcctgagggctgacacgtggagcggggcgcagactccgactcccccacccaagaggccagcccgcagtcccagccccagcccag

SEQ ID NO: 447 - DNA, 122 bp, Homo Sapiens
gttttccctcttgcctgagaacaccacttataacacgggacctatacgggagtggtgacacgccatctgttcaagtttaacttttttctcattttttagacagcaaatacaaatggaagttc

SEQ ID NO: 448 - DNA, 122 bp, Homo Sapiens
aaaccagctggcctgaatcaaagcaattctgggtaaaagacttcccagatgatctctcaacgccttggcaacgcttggcgactgggagctagaaccatgagttgttaatgtgtgcacggaag

SEQ ID NO: 449 - DNA, 122 bp, Homo Sapiens
agaggcaagacaagtgcaaaggcagccacaaggcttccgccagttagacggtgtttctgccgcccctgccgtcacccgctgggtcccgtcccgctccaagcgcactcccggcccgcagggtg

SEQ ID NO: 450 - DNA, 122 bp, Homo Sapiens
gcttctggtaaagcattcaagacaaccaatttaagtgttgaagagcaaaaggaaaaggaacgtggggaagctaaacactgctttaatgctttcgcaagtgacaggatttctttgcaccgaga

SEQ ID NO: 451 - DNA, 122 bp, Homo Sapiens
ccactgtgcccatctctggtttcaccaggctccattccttccccagttccctgtttctcacggccctgaaaccagccgacagggatttgcattttgaaaaaccttgcgtggctgccggcgct

SEQ ID NO: 452 - DNA, 122 bp, Homo Sapiens
ggcattcccgggcagattcctggcttggggactaatgccccagaggctccaggttacccacgggctcagggatggctcccacggggtgtttccagttcccgggagagtcgagtaaggtcccg

SEQ ID NO: 453 - DNA, 122 bp, Homo Sapiens
ttattgtaaacccattttaccagtgatgtgaatgagccgcaatgaaggctaagggacttgcgcaaggtgacatatataagcaacaggcctgcgattggaatccaggccccagagtctgggca

SEQ ID NO: 454 - DNA, 122 bp, Homo Sapiens
tgagggaactagagagacttgtttggcgagtggtggcaaccagtgttctgtggatgtggacggttcctggtcatcaaagttatggtcagctctggttcctgttcgcctggacccagacttac

SEQ ID NO: 455 - DNA, 122 bp, Homo Sapiens
aggccgggggcggagccgcagagggacccgcggccggccaatcagagacgtagcggagcccggcggactacattgcccagtaacctcctgggctccgctgtgtttttctattctggggtgta

SEQ ID NO: 456 - DNA, 122 bp, Homo Sapiens
atctgttggcgttgacgcttaaatgttggatattgcactgcggcagtgccttgaaatgtacgtgcaggatggaatgttaggtaactattaaagccactcttgtgaagatgtattttgcagcc

SEQ ID NO: 457 - DNA, 122 bp, Homo Sapiens
gaaggagagaagagccagccagaaattatccagcaaatctatcatggatcctaatcagaacgtgaaatgcaagatagttgtggtgggagacagtcagtgtggaaaaactgcgctgctccatg

SEQ ID NO: 458 - DNA, 122 bp, Homo Sapiens
cgccgggggtccagagtcttgggcaaactttgaagggtgccggagccgcttgttgctcctcgcggcggagggagccgcaccgttacgtccccgcggggagagcgaggcggccctcctcggcg

SEQ ID NO: 459 - DNA, 122 bp, Homo Sapiens
attcactcgcactgcctgggattgcactggatccgtgtgctcagaacaaggtaagagcctcgctcctttaacagctttattacctgctgccacttctcactccaccaagggcctggagttcc

SEQ ID NO: 460 - DNA, 122 bp, Homo Sapiens
aatctatcagaatcctcgctgaaagtcttatttctccttgaaaccgaaagagctttcaaacgtggcttcctgagtttttcagtttgctggtgggtgggtctggagtgggcatcccgtctgca

SEQ ID NO: 461 - DNA, 122 bp, Homo Sapiens
cactaccccatggaggcctggctggtgctcacatacaataattaactgctgagtggccttcgcccaatcccaggctccactcctgggctccattcccactccctgcctgtctcctaggccac

SEQ ID NO: 462 - DNA, 122 bp, Homo Sapiens
gaagacccctctccctgaagctggcaggccagggactagcccagtggggcagggtactcacggttggcctgggcctgaggtgggagcaggttctggacagatggccaaggttgcaaatgtgg

SEQ ID NO: 463 - DNA, 122 bp, Homo Sapiens
gggccccgccatggctgggatggacagtggcaacctgaagaccgcgaggctgtggcgggacgccgccctgcgtgccaggaagctgcggagcaacctgcgccagctcacgcttaccgccgccg

SEQ ID NO: 464 - DNA, 122 bp, Homo Sapiens
ctccgccttggctgcgatgttgctcactctgctcagggctctcccctctccgtccggtagcgcaccctggctttgcaatagcccctggctcggagccgctttccagcgagtgcaagaaccgg

SEQ ID NO: 465 - DNA, 122 bp, Homo Sapiens
gagggtggccgccgagagccgccgctaccatcgcgacaaagacccgggtagctcccgctgcgcccagagccatcatctcagaaggactcaagagggagaaagaaagagaaaatgaccgtcac

SEQ ID NO: 466 - DNA, 122 bp, Homo Sapiens
tgggggagggggacttgtttttcttttcctctagagacctcggcttgcaactggatcaaacgctgtcgaaaggatgtaaataggcagagcaactgttaccaagaaggccaccacccccaccc

SEQ ID NO: 467 - DNA, 122 bp, Homo Sapiens
tggttatgtggagaaggagcaatcccttctccctcctcctcctccccccgctactatccgcggcccagagaactgccgcttgccgccattgacacgcacagatagaacccaaagaaaggcaa

SEQ ID NO: 468 - DNA, 122 bp, Homo Sapiens
cgggatgggggagcccagcagtgcccactgcacgcctggtgacgagtctcccctcatctgcgcagctcagtttgctcagtttgctcttcgtgacacgtgactcggcaaggggagcaggagga

SEQ ID NO: 469 - DNA, 122 bp, Homo Sapiens
agggggatttttagagggcagccggctctagataccggaaatggagacgtctgagtgggacgccctgtcacacaaatgcgctgtgtgggctggcctatgccccatcctaaattattcaaaca

SEQ ID NO: 470 - DNA, 122 bp, Homo Sapiens
ggccgccgctccccggtcccccgctccgtccgccaccctgccgccgcccagcagccgccacgctgaaggccggcgactggcgctcacaccacccagcactgaaatattaaacaggggagcgg

SEQ ID NO: 471 - DNA, 122 bp, Homo Sapiens
tcttagccgcaggctttgatgctgtcattttctccaaatgcttcttgagcacctactaagcgcttgcgccgggcggtgccgcgggagacagcgccgtgggcacctcacttgcggcagataac

SEQ ID NO: 472 - DNA, 122 bp, Homo Sapiens
gtgggcggggggtctggagtggccagccaatcaggcgggcagtgccacctcggggaatgacgctcgggccaatgggaagggcactccattccgtcaacgctgtgggtcggggttctgagaaa

SEQ ID NO: 473 - DNA, 122 bp, Homo Sapiens
tgctttaaaaaaaataaaattttgtgttagaagttttaaaacatttggaaattctagttgcggcttcagatttcataattcagatgatgcaacaggatggaaccattgtcaaagagaatgca

SEQ ID NO: 474 - DNA, 122 bp, Homo Sapiens
tcccaaaatagcaaacctctaaaatcaggagtccaatgggcatgcattcaagcctctaatcgtagaatgtatttggctggccggttggctggctggctggttggttggttggttggttggtt

SEQ ID NO: 475 - DNA, 122 bp, Homo Sapiens
ccagacttttcgtccctacccccaccaagctccctggaacggtctccccgcgtctggggacggagaggagaactaataactggccccttcagccgcccacccgcccctgtccagcttcgctc

SEQ ID NO: 476 - DNA, 122 bp, Homo Sapiens
atgcattgaaccagaaaagagaaggggcaaagaaatcaggtatcaagaagaccagaattgcgcaactgcagtttagcctggtgagttgcaattctggctgcacatgaaaattacttggagaa

SEQ ID NO: 477 - DNA, 122 bp, Homo Sapiens
tgggagctgccgcggtcaggcgcgggtggggacctggggagctctttaagtctccctgtgcgccccgccctccctggccgaggggcccgccccgtggagccacctaggcgggcagatgaagc

SEQ ID NO: 478 - DNA, 122 bp, Homo Sapiens
gagggctgggcgcagcgtgcgggcttttgggggtagagggttgagttgcacgggaggttacgactcccactccgggagcccagtcttgtcacacctagggcttcaccagtctgcgacattcg

SEQ ID NO: 479 - DNA, 122 bp, Homo Sapiens
tcggcttccagctcgggggcctcggcagagactaggaaccagactggggcagcactggggcgcccgccacccccacccactccggcaagcgggcgaatcagcgcggctaacccgcggcgccc

SEQ ID NO: 480 - DNA, 122 bp, Homo Sapiens
caaagttagcaccttagcagggtgggcgtgacccttggggcgggctgtgcagtctgcctgcgcggagctggccgtgtgcgctgcgccctcggccggaggctcacctggcaaatcagacgcgc

SEQ ID NO: 481 - DNA, 122 bp, Homo Sapiens
agtttggaatttggtttctatcttgtttgcagcattcatatttagcaacaataggaaccacgggaaataacattataactgaaacaaaagttgggattttgcttgtaagtaaaacctttggt

SEQ ID NO: 482 - DNA, 122 bp, Homo Sapiens
ggggatgcctcagaaaacttataatcatggcagatggggaaacaaacacacccttctccacgtggcagcaggaagtgccgaacaagagggaaaagccccttataaaaccatcagatctcgtg

SEQ ID NO: 483 - DNA, 122 bp, Homo Sapiens
cagccgagaagcagaaacacgacgggcgggtgaagatcggccactacattctgggtgacacgctgggggtcggcaccttcggcaaagtgaagggtgagaaccccgcgggactaggcttggcc

SEQ ID NO: 484 - DNA, 122 bp, Homo Sapiens
gcccaggtgtttacctggcactcaggtgagtggtgcgctctggctgttttctgtcggagccgcccgcctcttccttcagcgcgtcccacaaatcccgacggcacggaggggccccaggccaa

SEQ ID NO: 485 - DNA, 122 bp, Homo Sapiens
gcgagaagttgagggtgggcagctggaacatggacaactcttccgggggggccctggagccgccgccgccgccgccgcctgctccgccagcaccgccgcctcccgcaccgttcccgccgccg

SEQ ID NO: 486 - DNA, 122 bp, Homo Sapiens
cacagaggagctgaatgcaagcgaggaaacgctttaggattcgcatttccagacggtctgcggcccctcgccgtgcggctcctctgcttctcagagtggagagggagggggagcgaaggaaa

SEQ ID NO: 487 - DNA, 122 bp, Homo Sapiens
cctccccggttcatgcgcctcggcctcccaaaatgctgggattgcggatctgagccaccgcgcccggcctgaggtgatttttaacaagtacagccgtagctgcagatatcctggttctgtag

SEQ ID NO: 488 - DNA, 122 bp, Homo Sapiens
tcacactttcctcccttattcacaatagagctctcatttattgagcacctattgtgtgcccgcccctttataaaatcaaacctggtttttactatgatctgggggaggatgctgttaattag

SEQ ID NO: 489 - DNA, 122 bp, Homo Sapiens
gcaatttaaaaaaatgtaactttcattaacgagcgcgccggaaatctcaggtatcaaagacgcattaaccctggagcgtcccccgcgcgccgcagccggacggaggcagccgcggcgaacag

SEQ ID NO: 490 - DNA, 122 bp, Homo Sapiens
atgaaggcatagagggccaagtgactgaaacaatatgccgacaaccccaaagcaccactacgctctccaaagagggcagcatcccaccctcaggcgctggaggcttgaccactctccagctg

SEQ ID NO: 491 - DNA, 122 bp, Homo Sapiens
ggggccaataatctcaacaacacaggtaacagtaacagccaagtctgactaagcggcatccgcctcgggggtctgatcccgcccaatctgggaggggcgccggggtcccgggtcgccagctg

SEQ ID NO: 492 - DNA, 122 bp, Homo Sapiens
ggacagaaagtctacttctggctgtcacttgcaatattattcttcaataaccccgaataacgcgcattgggaaggccacctcgcctcccttccaatcacccccacccccctcgccaacaatc

SEQ ID NO: 493 - DNA, 122 bp, Homo Sapiens
acagccccgctcaatcagcgagtctcttcttccctaggagcgcccctggccactgaactgcgctgccagtgcttgcagaccctgcagggaattcacctcaagaacatccaaagtgtgaaggt

SEQ ID NO: 494 - DNA, 122 bp, Homo Sapiens
ggttgccatggcggcggttgtcgcggcgacgaggtggtggcagctgttgctggtgctcagcgccgcggggatgggggcctcgggcgccccgcagccccccaacatcctgctcctgctcatgg

SEQ ID NO: 495 - DNA, 122 bp, Homo Sapiens
ttgttacccggttagaaaagtttgcggagcgcggggatggactaaccggctctcctgcttcgccctcccagcgcctagaagcctgcagctccggagcagtggccgcgccacgccggccccag

SEQ ID NO: 496 - DNA, 122 bp, Homo Sapiens
cgcggggcgccatggcacaccgagcggctccgtcttctgctcctcagagagcccggctggcggcctgggatgacaaggtaccatccctccagaggctgatcccaatgcatcggcttcgctta

SEQ ID NO: 497 - DNA, 122 bp, Homo Sapiens
gtcactagtccctggcatatccacgctggagcagctacagctccctgcctttggcacttacgtgctgtgtgaccctgagcaagttatggcacctctctgggcctcagtttccttttttgttt

SEQ ID NO: 498 - DNA, 122 bp, Homo Sapiens
cccctaggaaacccaggggaaatgccgctatgccgacataacagcgcccagcacagcaaacgcagattagacagatgcactcgcccggcatgtgcctagcacctgggctaggagatttcaca

SEQ ID NO: 499 - DNA, 122 bp, Homo Sapiens
ttacaggtagaggacctctgtgcaggagccctcaacacccagggaggattaggatataagcgcaaactcaaaggtactgtaatgggcacctagcaatcagaggtgatacaagggtctcttgg

SEQ ID NO: 500 - DNA, 122 bp, Homo Sapiens
aatggggccagagggctcccgggctgggcaggtaaggagcgctggtattgggggcgcaggcgccggggtgagaggcctgatagcagacggctgcagctgtgcgggcccaggctccctaggga

SEQ ID NO: 501 - DNA, 122 bp, Homo Sapiens
agggcagctctcacccaggctgatagttcggtgacctggctttatctactggatgagttccgctgggagatggaacatagcacgtttctctctggcctggtactggctacccttctctcgca

SEQ ID NO: 502 - DNA, 122 bp, Homo Sapiens
tccgctgggagatggaacatagcacgtttctctctggcctggtactggctacccttctctcgcaaggtaaggctactccaggtgggtgggggaagggacctgagagggacattactgatggg

SEQ ID NO: 503 - DNA, 122 bp, Homo Sapiens
tgcagaccaaaaccacaagcagaacaagcaggcgtgagacactcacaggttgggtttgatcgcatgcgtgtcggagaggagagagcagagagagacacaggaacaagaacagcaaagggtag

SEQ ID NO: 504 - DNA, 122 bp, Homo Sapiens
tctgctctaccctttgctgttcttgttcctgtgtctctctctgctctctcctctccgacacgcatgcgatcaaacccaacctgtgagtgtctcacgcctgcttgttctgcttgtggttttgg

SEQ ID NO: 505 - DNA, 122 bp, Homo Sapiens
agaagagaaagaaataccagaggcctgacttcatgtttgccagaaacagttaacagtcctcgcgttcagtgttcaaagctgacagtgaattcaggctctcggttgttttctatttgctggaa

SEQ ID NO: 506 - DNA, 122 bp, Homo Sapiens
tggagccagtctagctgctgcacaggctggctggctggctggctgctaagggctgctccacgcttttgccggaggacagagactgacatggaacaggggaagggcctggctgtcctcatcct

SEQ ID NO: 507 - DNA, 122 bp, Homo Sapiens
taacccaggcctggggactgtgggtcctcttaggggctgtgacgctgctatttctcatctcgctggctgcacacttgtcccagtggaccaggggccggagcaggagccatccggggcaggga

SEQ ID NO: 508 - DNA, 122 bp, Homo Sapiens
taaagggccagcctgagctgcagaggattcctgcagaggatcaagacagcacgtggacctcgcacagcctctcccacaggtaccatgaaggtctccgcggcagccctcgctgtcatcctcat

SEQ ID NO: 509 - DNA, 122 bp, Homo Sapiens
ctcaaatttcaggaagtagctgtgaggatggggtggcaggaaatgacgaaaacctgaatacggaactgaagcagcagctcagtttctcactccgaagtggcagcagccagagagggagtcgg

SEQ ID NO: 510 - DNA, 122 bp, Homo Sapiens
ttctgtacctgagagcagcagcagcaacgccaccatccagtggctgggcacaggagacaacgccagcctggccatggtcaccgctctgtccccgaccccaaacccgtgacaacgtccgaggc

SEQ ID NO: 511 - DNA, 122 bp, Homo Sapiens
atttctgtgtgcaggcgagcttcttggcctaagggcaggaagagatggcagcgggggagacgcagctctacgccaaggtctccaacaagctcaagagccgcagcagcccctcgctcctggag

SEQ ID NO: 512 - DNA, 122 bp, Homo Sapiens
gcagagtgaaaagagcaaaccactttgccagaatggaaacagagagtcagtcaccggaaacgacaccaccaatgtgctaatgaagcacagggcacaggaaactctgctcagctcttaatctg

SEQ ID NO: 513 - DNA, 26 bp, Artificial
  Primer
gtttagaggggttttttgattatttg

SEQ ID NO: 514 - DNA, 24 bp, Artificial
  Primer
aactcctacaactccaaaaaattc

SEQ ID NO: 515 - DNA, 25 bp, Artificial
  Primer
gagggaatagttggaatgtattttg

SEQ ID NO: 516 - DNA, 26 bp, Artificial
  Primer
ctaaactactatttcctactaactac

SEQ ID NO: 517 - DNA, 25 bp, Artificial
  Primer
ggtttagagtttttagtatggggtt

SEQ ID NO: 518 - DNA, 24 bp, Artificial
  Primer
actctaaccctaatctaccaacaa

SEQ ID NO: 519 - DNA, 23 bp, Artificial
  Primer
aggtaggagtatgtgtttggtag

SEQ ID NO: 520 - DNA, 25 bp, Artificial
  Primer
tcaaaaatacaaaaaaaaaacaaaa

SEQ ID NO: 521 - DNA, 25 bp, Artificial
  Primer
ggtttggtttttggaattttaaggg

SEQ ID NO: 522 - DNA, 28 bp, Artificial
  Primer
aaaacaacaacaatatcattaacctaac

SEQ ID NO: 523 - DNA, 25 bp, Artificial
  Primer
gtttatttgattattgggtgggttt

SEQ ID NO: 524 - DNA, 25 bp, Artificial
  Primer
ctataacaacaacaataacaacaac

SEQ ID NO: 525 - DNA, 25 bp, Artificial
  Primer
aaatatgggggtattattttatatg

SEQ ID NO: 526 - DNA, 25 bp, Artificial
  Primer
ccttactattaaaaatacaaatacc

SEQ ID NO: 527 - DNA, 25 bp, Artificial
  Primer
atgaattgaaggatgttatttaggg

SEQ ID NO: 528 - DNA, 25 bp, Artificial
  Primer
aaacttccaaacaaaaataaccaac

SEQ ID NO: 529 - DNA, 29 bp, Artificial
  Primer
tgtgtaaatgtggttgtattgttaatagg

SEQ ID NO: 530 - DNA, 31 bp, Artificial
  Primer
catcatattactcaaactaatctcaaactcc

SEQ ID NO: 531 - DNA, 28 bp, Artificial
  Primer
gtgatttggttttatttattggatgagt

SEQ ID NO: 532 - DNA, 22 bp, Artificial
  Primer
aataaacctcactcccatcaat

SEQ ID NO: 533 - DNA, 23 bp, Artificial
  Primer
ggttttatttattggatgagttt

SEQ ID NO: 534 - DNA, 22 bp, Artificial
  Primer
ggtttggtattggttatttttt

SEQ ID NO: 535 - DNA, 28 bp, Artificial
  Primer
ggtatttgtatttgtagttttgttgagg

SEQ ID NO: 536 - DNA, 28 bp, Artificial
  Primer
ttctcctccataaaacactatttctctc

SEQ ID NO: 537 - DNA, 22 bp, Artificial
  Primer
tgatgggtggagttagtttagt

SEQ ID NO: 538 - DNA, 21 bp, Artificial
  Primer
aaacccttcccctattccata

SEQ ID NO: 539 - DNA, 16 bp, Artificial
  Primer
ggttggttgttaaggg

SEQ ID NO: 540 - DNA, 23 bp, Artificial
  Primer
ggggaagtgtgtttgtatggatg

SEQ ID NO: 541 - DNA, 34 bp, Artificial
  Primer
aaaccacatatctaaaactatctctaactactac

SEQ ID NO: 542 - DNA, 25 bp, Artificial
  Primer
aggtagttggggttttttttattag

SEQ ID NO: 543 - DNA, 29 bp, Artificial
  Primer
ctaccctttactattcttattcctatatc

SEQ ID NO: 544 - DNA, 20 bp, Artificial
  Primer
atatttataggttgggtttg

SEQ ID NO: 545 - DNA, 27 bp, Artificial
  Primer
taggtaggagaggaattggggttatag

SEQ ID NO: 546 - DNA, 28 bp, Artificial
  Primer
catccacaaaaaaccccaactatactac

SEQ ID NO: 547 - DNA, 28 bp, Artificial
  Primer
agttggagatgagagtaaattttatagg

SEQ ID NO: 548 - DNA, 28 bp, Artificial
  Primer
aatacctcccctaaatcccaatttacat

SEQ ID NO: 549 - DNA, 18 bp, Artificial
  Primer
ggttgggtataggagata

SEQ ID NO: 550 - DNA, 34 bp, Artificial
  Primer
ttattgttaaaattttgtaaaagattaggtatag

SEQ ID NO: 551 - DNA, 29 bp, Artificial
  Primer
ttcctcctcaactcttactctatatttcc

SEQ ID NO: 552 - DNA, 20 bp, Artificial
  Primer
aggatggggtggtaggaaat

SEQ ID NO: 553 - DNA, 27 bp, Artificial
  Primer
cctccacctatacaaacctctattcta

SEQ ID NO: 554 - DNA, 15 bp, Artificial
  Primer
gggtggtaggaaatg

SEQ ID NO: 555 - DNA, 34 bp, Artificial
  Primer
taagtaggtaatttaaaaatttaatggtttgatg

SEQ ID NO: 556 - DNA, 32 bp, Artificial
  Primer
cctctatcttcaaaatcatcaataatccatac

SEQ ID NO: 557 - DNA, 30 bp, Artificial
  Primer
gaggtttgattttatgtttgttagaaatag

SEQ ID NO: 558 - DNA, 19 bp, Artificial
  Primer
tcccaaaaaacccacttcc

SEQ ID NO: 559 - DNA, 25 bp, Artificial
  Primer
tttgttagaaatagttaatagtttt

SEQ ID NO: 560 - DNA, 24 bp, Artificial
  Primer
ggtttatggtggtaggaagtttgg

SEQ ID NO: 561 - DNA, 32 bp, Artificial
  Primer
ttaacacctaactatccatatacctaatatcc

SEQ ID NO: 562 - DNA, 24 bp, Artificial
  Primer
gttaggttaggttaggaggattat

SEQ ID NO: 563 - DNA, 24 bp, Artificial
  Primer
ccaaccacaaaaaactactacatc

SEQ ID NO: 564 - DNA, 18 bp, Artificial
  Primer
gagagttggtattggggg

SEQ ID NO: 565 - DNA, 31 bp, Artificial
  Primer
gtagtgtgtttgtggatttttatatttgtag

SEQ ID NO: 566 - DNA, 30 bp, Artificial
  Primer
atctaatcaacaacttatccttcctcctac

SEQ ID NO: 567 - DNA, 23 bp, Artificial
  Primer
gtgggtttttttaggggttgtga

SEQ ID NO: 568 - DNA, 23 bp, Artificial
  Primer
tctcaatcaacccatccctatta

SEQ ID NO: 569 - DNA, 25 bp, Artificial
  Primer
gttgtgaagttgttattttttattt

SEQ ID NO: 570 - DNA, 24 bp, Artificial
  Primer
tggtggaaatagttaggattggtg

SEQ ID NO: 571 - DNA, 34 bp, Artificial
  Primer
caatatcttaccctacaaaatacactactttaac

SEQ ID NO: 572 - DNA, 23 bp, Artificial
  Primer
ggtttaagggtaggaagagatgg

SEQ ID NO: 573 - DNA, 30 bp, Artificial
  Primer
actaactaaacccccaaatctctaaacaat

SEQ ID NO: 574 - DNA, 17 bp, Artificial
  Primer
gtaggaagagatggtag